SEQUENCE LISTING (550 sequences)

SEQ ID NO: 1 - DNA, 669 bp, Arabidopsis thaliana
  misc_feature 1..669: note = AtBBX28 (G1481), AT4G27310.1, paralog of AtBBX29 (G900)
  source 1..669: mol_type = unassigned DNA
atggggaagaagtgtgatttatgtaacggtgttgcaagaatgtattgcgagtcagatcaagctagtttatgttgggattgcgacggtaaagttcacggcgctaatttcttggtagctaaacacacgcgttgtcttctctgtagcgcttgtcagtctcttacgccgtggaaagctactgggcttcgtcttggcccaactttctccgtctgcgagtcatgcgtcgctcttaaaaacgccggcggtggccgtggaaacagagttttatcggagaatcgtggtcaggaggaggttaatagtttcgagtccgaagaagatcggattagagaagatcacggtgacggtgacgacgcggagtcttacgatgatgatgaggaagaagatgaggatgaagagtacagcgacgatgaggatgaggatgatgatgaggatggtgatgatgaggaagcggagaatcaagttgtgccgtggtctgcggcggcgcaagttcctccggtgatgagttcttcatcttctgacggaggaagcggaggttcagtgacgaagaggacgagggctagagagaattcagatcttctctgctccgatgatgagatcggaagctcttcagctcaagggtcaaactattctcggccgttgaagcgatcggcgtttaaatcaacggttgttgtt

SEQ ID NO: 2 - protein, 223 aa, Arabidopsis thaliana
  REGION 1..223: note = AtBBX28 (G1481), AT4G27310.1, domain coordinates 5-41, paralog of AtBBX29 (G900)
MGKKCDLCNGVARMYCESDQASLCWDCDGKVHGANFLVAKHTRCLLCSACQSLTPWKATGLRLGPTFSVCESCVALKNAGGGRGNRVLSENRGQEEVNSFESEEDRIREDHGDGDDAESYDDDEEEDEDEEYSDDEDEDDDEDGDDEEAENQVVPWSAAAQVPPVMSSSSSDGGSGGSVTKRTRARENSDLLCSDDEIGSSSAQGSNYSRPLKRSAFKSTVVV

SEQ ID NO: 3 - DNA, 645 bp, Arabidopsis thaliana
  misc_feature 1..645: note = AtBBX29 (G900), AT5G54470.1 paralog of AtBBX28 (G1481)
  source 1..645: mol_type = unassigned DNA
atggggaagaagaagtgcgagttatgttgtggtgtagcgagaatgtattgtgagtcagatcaagcgagtttatgttgggattgtgacggtaaagttcacggagctaattttctggtggcgaaacacatgcgttgtcttctatgtagcgcgtgtcagtcacacacgccttggaaagcttctgggctgaatcttggcccaactgtttctatctgtgagtcttgtttagctcgtaagaagaataacaacagctccctcgccgggagggatcagaatcttaaccaagaagaagagatcattggttgtaacgacggagctgagtcttatgatgaggaaagcgatgaggatgaagaagaagaagaagtggagaatcaggttgttccggctgcggtggagcaagaacttccggtggtgagttcgtcgtcttcggttagtagtggtgaaggagatcaggtggtgaaaaggacgagacttgatttggatcttaacctctccgatgaggagaaccaatctagaccattgaaaagattatcgagagacgaaggtttgtcaagatcaactgttgtgatgaatagctcaatcgtgaaattacacggagggaggagaaaagcagagggatgtgatacatcatcgtcgtcttcgttttat

SEQ ID NO: 4 - protein, 215 aa, Arabidopsis thaliana
  REGION 1..215: note = AtBBX29 (G900), AT5G54470.1, domain coordinates 11-42, paralog of AtBBX28 (G1481)
MGKKKCELCCGVARMYCESDQASLCWDCDGKVHGANFLVAKHMRCLLCSACQSHTPWKASGLNLGPTVSICESCLARKKNNNSSLAGRDQNLNQEEEIIGCNDGAESYDEESDEDEEEEEVENQVVPAAVEQELPVVSSSSSVSSGEGDQVVKRTRLDLDLNLSDEENQSRPLKRLSRDEGLSRSTVVMNSSIVKLHGGRRKAEGCDTSSSSSFY

SEQ ID NO: 5 - DNA, 789 bp, Zea mays
  misc_feature 1..789: note = ACN33981.1, ortholog of AtBBX28 (G1481), AtBBX29 (G900)
  source 1..789: mol_type = unassigned DNA
atgtcggcggctgcggcagcggaagtggggaaggagaagggcgccgccacccccggccccggccccggccccggtggcgcgtgcgagctgtgcggcgccgcggccagggtgtactgcggcgccgacgaggccacgctctgctggggctgcgacgcgcaggtgcacggcgccaacttcctggtggcgcgccacgcgcgctcgctgctgtgccggggctgcgcgcgccccacgccgtggcgcgccgcgggcccgcgcctcggccccaccgtctccctctgcgtccgctgcgtccgccgcggcccggcgggggccgtgggcgtcggcggcgatgaggagatggggggagcgggcgacggagacgccgaggatgacgacgacgaggacgacgactccgaggaggaggaggaggaggaggaggaggaaggggagggggagaaccaggtcgtgccgtggacggaggacgccgaggccacgccgccgcccgtcgggagctccacgtccagcagcagccgggaggcccccgccaacggcgcagagcgcgcagcgaaggaaaacgccccgtgctccacctcgcagccaggcctgtgccaccacgcgtcgtccgcgcgccacggcggccggagcgacgaggccacctcgtcccggagcggcggccgggtcctcgcctcccgccacaggaagagatcgccctcggatttcttcagctctgatgggtcgccgcagcccgggaatggcacaccggcggcgaggaattgctccgacgccggcattgccaggaatgactttacc

SEQ ID NO: 6 - protein, 263 aa, Zea mays
  REGION 1..263: note = ACN33981.1, domain coordinates 32-64, ortholog of AtBBX28 (G1481), AtBBX29 (G900)
MSAAAAAEVGKEKGAATPGPGPGPGGACELCGAAARVYCGADEATLCWGCDAQVHGANFLVARHARSLLCRGCARPTPWRAAGPRLGPTVSLCVRCVRRGPAGAVGVGGDEEMGGAGDGDAEDDDDEDDDSEEEEEEEEEEGEGENQVVPWTEDAEATPPPVGSSTSSSSREAPANGAERAAKENAPCSTSQPGLCHHASSARHGGRSDEATSSRSGGRVLASRHRKRSPSDFFSSDGSPQPGNGTPAARNCSDAGIARNDFT

SEQ ID NO: 7 - DNA, 735 bp, Populus trichocarpa
  misc_feature 1..735: note = Pt_568988, ortholog of AtBBX28 (G1481), AtBBX29 (G900)
  source 1..735: mol_type = unassigned DNA
atgaaagggtgtgagctttgtgggagttcagcaagaatgttctgtgaatcagacgaagctagtttatgttgggattgtgatgagaaagtacactctgccaactttcttgtagctaaacactgtagaacccttctttgtcaagtctgtcaatctccaactccttggaaagcctcggtgtctaaatttgctcctactgtttccatatgtgaatcttgttttactatccctaacaaaacaaaagaaacagaagagagaatgaaagggtgcgagctttgtgggagttcagcaaggatgtattgtgaatcagaccaagctagtttatgttgggattgtgatgagaaagtgcacactgccaactttcttgttgctaaacattgtagaacccttctttgtcaagtctgtcaatctccaactccttggaaagcctcggggtctaaatttgctcctactgtttccgtggtgccatggtcaggtcctactgcgtcgtcgtcgccatcacctgtgccacctgtggctagttctagtagtgaagaggagacttcttgtgctggtagaaatggattcttgaaacggatgcgagagagtaatgttgatcttgattctgatgatgaaattggatgttcctcttctcataatataggaggtcgaatattgagcaatgatggagggaattctttgagttcaatgaggccatggaagcaagcaagaacaagtgttaatgttgaagaagatggt

SEQ ID NO: 8 - protein, 245 aa, Populus trichocarpa
  REGION 1..245: note = Pt_568988, domain coordinates 4-40, 89-125, ortholog of AtBBX28 (G1481), AtBBX29 (G900)
MKGCELCGSSARMFCESDEASLCWDCDEKVHSANFLVAKHCRTLLCQVCQSPTPWKASVSKFAPTVSICESCFTIPNKTKETEERMKGCELCGSSARMYCESDQASLCWDCDEKVHTANFLVAKHCRTLLCQVCQSPTPWKASGSKFAPTVSVVPWSGPTASSSPSPVPPVASSSSEEETSCAGRNGFLKRMRESNVDLDSDDEIGCSSSHNIGGRILSNDGGNSLSSMRPWKQARTSVNVEEDG

SEQ ID NO: 9 - DNA, 285 bp, Populus trichocarpa
  misc_feature 1..285: note = Pt_571834, ortholog of AtBBX28 (G1481), AtBBX29 (G900)
  source 1..285: mol_type = unassigned DNA
atgaagaagtgtgagctatgcaagaacccagcaagaacatactgtgaatcagacgaagcaaatctttgttggaactgtgatacaaaagtccatggagccaactttcttgttgctagacatgctcgtgctcttctttgtcagtcttgtcaatcgctaactccctggaaagcctctgggtctcaactagggcacactgtgtctgtatgtgagagatgtggtgccatggtcaccaacaacaccacctccaccgccatctgcaagctcttcttgttcttgtaccagttc

SEQ ID NO: 10 - protein, 95 aa, Populus trichocarpa
  REGION 1..95: note = Pt_571834, domain coordinates 4-40, ortholog of AtBBX28 (G1481), AtBBX29 (G900)
MKKCELCKNPARTYCESDEANLCWNCDTKVHGANFLVARHARALLCQSCQSLTPWKASGSQLGHTVSVCERCGAMVTNNTTSTAICKLFLFLYQF

SEQ ID NO: 11 - DNA, 1121 bp, Populus trichocarpa
  misc_feature 1..1121: note = Pt_582065, ortholog of AtBBX28 (G1481), AtBBX29 (G900)
  source 1..1121: mol_type = unassigned DNA
atgaaagggtgtgagctatgtgggagttcagcaagaatgttctgtgaatcagaccaagctagtttatgttgggattgtgatgagaaagtacactctgccaacttccttgttgcaaagcattgtagaacccttctttgtcaagtgtgtcaatctccaactccttggaaatcctcagggtctaaacttgcccctaccgtttctgtatgtgaatcatgttttgctgtccataaaaataataaaaaacaactccaagatctgaacgtcatggctagtgatcaagaaagccgagaagccggaaatgattatgacgagtctgaaaatgatcgagaattcgacgacgacgacactgatgatgatagtgatgaatatgacgaggaggaagatggagacaaccagtgaagaggagatttcctgtgctggtggaaatgggttcttgaaacggatgcgagacagtaatgttgatcttgattctgatgtaagtctctttttctttttctttcatttctttgccattttttttatattagtttttagagggcacattttaataattgaatttgtggatcttaggaaatattgggtcttcttcctcttcctttttaggttacaaactttgggtttggtgaatacatttaatgttggataaatgcaggatgaaactggatgttcctcttcgcataatttaagaggtggaagcatgagcaccgaggaagggaattctttgagttcatcaaggccatggaagcaagcaagaacaagcgttcatgtagaagaagatggtcatgatggtcaagcaatgtcaagatcatcggctatcatagactccctgaagagactgcagaaagacttggtcgcgaatggagaaaacgcgtctgctgctattcttgggatctgcaaattgagcagagatcagagcctttgatttctgcccccccccccccccctaactttttttatatgattaattagtaaccatctagtttttgaaccataactatttagtatttcattttattttgaagcttgttttagtagctggttgtgtttgtcttcatggttttgtacatgagatggtatttggttcaatggttaatgcaatatattaccttatgctatttgtt

SEQ ID NO: 12 - protein, 132 aa, Populus trichocarpa
  REGION 1..132: note = Pt_582065, domain coordinates 4-40, ortholog of AtBBX28 (G1481), AtBBX29 (G900)
MKGCELCGSSARMFCESDQASLCWDCDEKVHSANFLVAKHCRTLLCQVCQSPTPWKSSGSKLAPTVSVCESCFAVHKNNKKQLQDLNVMASDQESREAGNDYDESENDREFDDDDTDDDSDEYDEEEDGDNQ

SEQ ID NO: 13 - DNA, 420 bp, Populus trichocarpa
  misc_feature 1..420: note = Pt_758524, ortholog of AtBBX28 (G1481), AtBBX29 (G900)
  source 1..420: mol_type = unassigned DNA
atgaaaaagtgtgagctctgtgattcttttgcacaaatgcactgtgaatcggatcaagctatcctgtgttcagcctgtgatgcatatgttcacagtgcaaattttcttgctgcaaaacattcaagaacgcttctttgccatgtttgtcaatctcacacgccctggattggcaccggacccctgttaggtgctactctttcggtttgcaacagctgcataaacaactcgagttgtactgatggaaaaggcagtgagaatgatcaaatcgccaataatgatgatgagataattgcgaacatgatgaaaacgatgatggtggttaagacggcgatgaagacgatgaagaaaatcaaatgtgtttatggattagttaaacaaggcaaagatcgtcaagaagtctctacgcgtcaaatgctattt

SEQ ID NO: 14 - protein, 140 aa, Populus trichocarpa
  REGION 1..140: note = Pt_758524, domain coordinates 4-40, ortholog of AtBBX28 (G1481), AtBBX29 (G900)
MKKCELCDSFAQMHCESDQAILCSACDAYVHSANFLAAKHSRTLLCHVCQSHTPWIGTGPLLGATLSVCNSCINNSSCTDGKGSENDQIANNDDEIIANMMKTMMVVKTAMKTMKKIKCVYGLVKQGKDRQEVSTRQMLF

SEQ ID NO: 15 - DNA, 849 bp, Oryza sativa
  misc_feature 1..849: note = Os08g08120.1, ortholog of AtBBX28 (G1481), AtBBX29 (G900)
  source 1..849: mol_type = unassigned DNA
atgtcggtggcggcggaggggaaggagaagggggttggtggtgggggtggtggtgcgggtgcgggggcgtgcgagctgtgcggggcggcggcgagggtgtactgcggggcggacgaggcgacgctgtgctgggggtgcgacgcgcaggtgcacggggccaacttcctggtggcgcggcacgcgcgggcgctgctgtgccgcgggtgcgcgaggcccacgccgtggcgcgccgcggggcccaggctgggccccaccgcgtcgctctgcgagcgctgcgtccgccgcggcggtggtggccgtggtggtggaggaggtggaggcgctgctggtggtggaggccgtggtggtgggggagacgaggagatgggcggcgagggcgatgaggaggaggaggacgaggacgaggaggtggtcgtcgaggaggaggaggacgaggatgatgaagacgaggagggggagggggagggggagaaccaggtggtgccgtgggcggaggaggcggaggccacgccgccgcccgtcgcgagctccacgtcgagcagcagccgggaggccgccgccaacggcgcgaacgccgccgaccgcgtcaaggaggaccagccgtgctcgacgtcgcagccgagcttgtgccggtacgcgtcctcggctcaccatggcggtggtggccggagcgacgaggccacctcctcccggaacggcggtggcgtcggcggccgattccttgcctcccggcataggaagagatcgccctcggatttccgccggtcggggttggcgcagtcggtgagcggcgtgcaggggaggaattgctccaatgcggtcgttggtcgaaatgatgcaagcatgatg

SEQ ID NO: 16 - protein, 283 aa, Oryza sativa
  REGION 1..283: note = Os08g08120.1, domain coordinates 28-60, ortholog of AtBBX28 (G1481), AtBBX29 (G900)
MSVAAEGKEKGVGGGGGGAGAGACELCGAAARVYCGADEATLCWGCDAQVHGANFLVARHARALLCRGCARPTPWRAAGPRLGPTASLCERCVRRGGGGRGGGGGGGAAGGGGRGGGGDEEMGGEGDEEEEDEDEEVVVEEEEDEDDEDEEGEGEGENQVVPWAEEAEATPPPVASSTSSSSREAAANGANAADRVKEDQPCSTSQPSLCRYASSAHHGGGGRSDEATSSRNGGGVGGRFLASRHRKRSPSDFRRSGLAQSVSGVQGRNCSNAVVGRNDASMM

SEQ ID NO: 17 - DNA, 843 bp, Oryza sativa
  misc_feature 1..843: note = Os08g08120.2, ortholog of AtBBX28 (G1481), AtBBX29 (G900)
  source 1..843: mol_type = unassigned DNA
atgtcggtggcggcggaggggaaggagaagggggttggtggtgggggtggtggtgcgggtgcgggggcgtgcgagctgtgcggggcggcggcgagggtgtactgcggggcggacgaggcgacgctgtgctgggggtgcgacgcgcaggtgcacggggccaacttcctggtggcgcggcacgcgcgggcgctgctgtgccgcgggtgcgcgaggcccacgccgtggcgcgccgcggggcccaggctgggccccaccgcgtcgctctgcgagcgctgcgtccgccgcggcggtggtggccgtggtggtggaggaggtggaggcgctgctggtggtggaggccgtggtggtgggggagacgaggagatgggcggcgagggcgatgaggaggaggaggacgaggacgaggaggtggtcgtcgaggaggaggaggacgaggatgatgaagacgaggagggggagggggagggggagaaccaggtggtgccgtgggcggaggaggcggaggccacgccgccgcccgtcgcgagctccacgtcgagcagcagccgggaggccgccgccaacggcgcgaacgccgccgaccgcgtcaaggaggaccagccgtgctcgacgtcgcagccgagcttgtgccggtacgcgtcctcggctcaccatggcggtggtggccggagcgacgaggccacctcctcccggaacggcggtggcgtcggcggccgattccttgcctcccggcataggaagagatcgccctcggatttccgccggtcggggttggcgcagtcggtgagcggcgtgcaggggaggaattgctccaatgcggtcgttggtcgaaatgacttttcc

SEQ ID NO: 18 - protein, 281 aa, Oryza sativa
  REGION 1..281: note = Os08g08120.2, domain coordinates 28-60, ortholog of AtBBX28 (G1481), AtBBX29 (G900)
MSVAAEGKEKGVGGGGGGAGAGACELCGAAARVYCGADEATLCWGCDAQVHGANFLVARHARALLCRGCARPTPWRAAGPRLGPTASLCERCVRRGGGGRGGGGGGGAAGGGGRGGGGDEEMGGEGDEEEEDEDEEVVVEEEEDEDDEDEEGEGEGENQVVPWAEEAEATPPPVASSTSSSSREAAANGANAADRVKEDQPCSTSQPSLCRYASSAHHGGGGRSDEATSSRNGGGVGGRFLASRHRKRSPSDFRRSGLAQSVSGVQGRNCSNAVVGRNDFS

SEQ ID NO: 19 - DNA, 837 bp, Oryza sativa
  misc_feature 1..837: note = Os08g08120.3, ortholog of AtBBX28 (G1481), AtBBX29 (G900)
  source 1..837: mol_type = unassigned DNA
atgtcggtggcggcggaggggaaggagaagggggttggtggtgggggtggtggtgcgggtgcgggggcgtgcgagctgtgcggggcggcggcgagggtgtactgcggggcggacgaggcgacgctgtgctgggggtgcgacgcgcaggtgcacggggccaacttcctggtggcgcggcacgcgcgggcgctgctgtgccgcgggtgcgcgaggcccacgccgtggcgcgccgcggggcccaggctgggccccaccgcgtcgctctgcgagcgctgcgtccgccgcggcggtggtggccgtggtggtggaggaggtggaggcgctgctggtggtggaggccgtggtggtgggggagacgaggagatgggcggcgagggcgatgaggaggaggaggacgaggacgaggaggtggtcgtcgaggaggaggaggacgaggatgatgaagacgaggagggggagggggagggggagaaccaggtggtgccgtgggcggaggaggcggaggccacgccgccgcccgtcgcgagctccacgtcgagcagcagccgggaggccgccgccaacggcgcgaacgccgccgaccgcgtcaaggaggaccagccgtgctcgacgtcgcagccgagcttgtgccggtacgcgtcctcggctcaccatggcggtggtggccggagcgacgaggccacctcctcccggaacggcggtggcgtcggcggccgattccttgcctcccggcataggaagagatcgccctcggatttccgccggtcggggttggcgcagtcggtgagcggcgtgcaggggaggaattgctccaatgcggtcgttggtcgaaatgag

SEQ ID NO: 20 - protein, 279 aa, Oryza sativa
  REGION 1..279: note = Os08g08120.3, domain coordinates 28-60, ortholog of AtBBX28 (G1481), AtBBX29 (G900)
MSVAAEGKEKGVGGGGGGAGAGACELCGAAARVYCGADEATLCWGCDAQVHGANFLVARHARALLCRGCARPTPWRAAGPRLGPTASLCERCVRRGGGGRGGGGGGGAAGGGGRGGGGDEEMGGEGDEEEEDEDEEVVVEEEEDEDDEDEEGEGEGENQVVPWAEEAEATPPPVASSTSSSSREAAANGANAADRVKEDQPCSTSQPSLCRYASSAHHGGGGRSDEATSSRNGGGVGGRFLASRHRKRSPSDFRRSGLAQSVSGVQGRNCSNAVVGRNE

SEQ ID NO: 21 - DNA, 648 bp, Glycine max
  misc_feature 1..648: note = Glyma06g39810.1, ortholog of AtBBX28 (G1481), AtBBX29 (G900)
  source 1..648: mol_type = unassigned DNA
atggaggaatcatcaacaacatgcgtgttgtgcgagaagagggcaatgatgctctgcgactcggaccaggctaagctatgctgggaatgcgacgagaaagtccacagcgccaatttcttggtcgcgaaacattctagggttcttttatgtcgtttgtgtcactccccgactccgtggaaggcttcggggatgaaactcacgcccactgtgtcgttttgtaaccgctgcgttgcggaacggaacgcgaggtggaaccgattggtgaataatgaaaatgaacatcaacaacagcaacaacaacaacaacagagtgattttgtggtggatgatggtagggaatatggttctgatcatgtttttgatgatgacgatggtgattatagtgatgatagtggtgaagaagaagaagaggacgaggatgatgaagaggagaatgagaatcaagtggttccaatgtcttctggttctgccacgtcaccacctaaggttgcctgtttagcgttgaagcgattgagaaacaactctttttctgtcgattcttcacatgatgagacagcgtgctcttcatctgagatgcttagttctacattgcctagtggtgatgaatcaacctctttgagtagtgtattaaagaggcatcgggttgat

SEQ ID NO: 22 - protein, 216 aa, Glycine max
  REGION 1..216: note = Glyma06g39810.1, domain coordinates 15-44, ortholog of AtBBX28 (G1481), AtBBX29 (G900)
MEESSTTCVLCEKRAMMLCDSDQAKLCWECDEKVHSANFLVAKHSRVLLCRLCHSPTPWKASGMKLTPTVSFCNRCVAERNARWNRLVNNENEHQQQQQQQQQSDFVVDDGREYGSDHVFDDDDGDYSDDSGEEEEEDEDDEEENENQVVPMSSGSATSPPKVACLALKRLRNNSFSVDSSHDETACSSSEMLSSTLPSGDESTSLSSVLKRHRVD

SEQ ID NO: 23 - DNA, 531 bp, Glycine max
  misc_feature 1..531: note = Glyma06g45620.1, ortholog of AtBBX28 (G1481), AtBBX29 (G900)
  source 1..531: mol_type = unassigned DNA
atgaacaagtgcgaactgtgcaaagttccggcgaggatcttctgcgagtcggaccaggcgagcttatgctgggactgcgacgctaaggttcacagcgccaacttcttggtcgcgagacacgcgcgaaccttactctgccgcacgtgccgctctcccacgccgtggaaggcctccggcgcctcactcgccagcgccgtctccgtctgcgaccgctgcgccgcctccgaagaaccggaagagagtgaagccggcaatgacgacgaaccagacacggaggacgacgacaacgacgacgaggacggcgacaatcaggtcgtgccgtggtcctcctccccgccgccgcctccgccgtcgagctcctccggcagcgaatggtcggagtgcaaaaacgacgacgcttctatttccgcctcggaacaggaaacgacaacatgctcgttgaaacgacagcgcgaggacgatgatgatgcttcttccgatcgtgatcccagggtgttgagaagggttgggcgtctcaacggaggttgcgtg

SEQ ID NO: 24 - protein, 177 aa, Glycine max
  REGION 1..177: note = Glyma06g45620.1, domain coordinates 4-40, ortholog of AtBBX28 (G1481), AtBBX29 (G900)
MNKCELCKVPARIFCESDQASLCWDCDAKVHSANFLVARHARTLLCRTCRSPTPWKASGASLASAVSVCDRCAASEEPEESEAGNDDEPDTEDDDNDDEDGDNQVVPWSSSPPPPPPSSSSGSEWSECKNDDASISASEQETTTCSLKRQREDDDDASSDRDPRVLRRVGRLNGGCV

SEQ ID NO: 25 - DNA, 1236 bp, Glycine max
  misc_feature 1..1236: note = G4015, ortholog of AtBBX28 (G1481), AtBBX29 (G900)
  source 1..1236: mol_type = unassigned DNA
atggaggaatcatcaacaacatgcgtgttgtgcgagaagagggcaatgatgctctgcgactcggaccaggctaagctatgctgggaatgcgacgagaaagtccacagcgccaatttcttggtcgcgaaacattctagggttcttttatgtcgtttgtgtcactccccgactccgtggaaggcttcggggatgaaactcacgcccactgtgtcgttttgtaaccgctgcgttgcggaacggaacgcgaggtggaaccgattggtgaataatgaaaatgaacatcaacaacagcaacaacaacaacaacagagtgattttgtggtggatgatgggagggaatatggttctgatcatgtttttgatgatgacgatggtgattatagtgatgatagtggtgaagaagaagaagaggacgaggatgatgaagaggagaatgagaatcaagtggttccaatgtcttctggttctgccacgtcaccacctaaggttgcctgtttagcgttgaagcgattgagaaacaactcttttctgtcgattcttcacatgatgagacagcgtgctcttcatctgagatgcttagttctacattgcctagtggtgatgaatcaccctctttgaatggaggaatcatcaacaacatgcgtgttgtgcgagaagagggcaatgatgctctgcgactcggaccaggctaagctatgctgggaatgcgacgagaaagtccacagcgccaatttcttggtcgcgaaacattctagggttcttttatgtcgtttgtgtcactccccgactccgtggaaggcttcggggatgaaactcacgcccactgtgtcgttttgtaaccgctgcgttgcggaacggaacgcgaggtggaaccgattggtgaataatgaaaatgaacatcaacaacagcaacaacaacaacaacagagtgattttgtggtggatgatgggagggaatatggttctgatcatgtttttgatgatgacgatggtgattatagtgatgatagtggtgaagaagaagaagaggacgaggatgatgaagaggagaatgagaatcaagtggttccaatgtcttctggttctgccacgtcaccacctaaggttgcctgtttagcgttgaagcgattgagaaacaactcttttctgtcgattcttcacatgatgagacagcgtgctcttcatctgagatgcttagttctacattgcctagtggtgatgaatcaccctctttga

SEQ ID NO: 26 - protein, 205 aa, Glycine max
  REGION 1..205: note = G4015, domain coordinates 8-44, ortholog of AtBBX28 (G1481), AtBBX29 (G900)
MEESSTTCVLCEKRAMMLCDSDQAKLCWECDEKVHSANFLVAKHSRVLLCRLCHSPTPWKASGMKLTPTVSFCNRCVAERNARWNRLVNNENEHQQQQQQQQQSDFVVDDGREYGSDHVFDDDDGDYSDDSGEEEEEDEDDEEENENQVVPMSSGSATSPPKVACLALKRLRNNSFLSILHMMRQRALHLRCLVLHCLVVMNHPL

SEQ ID NO: 27 - DNA, 237 bp, Glycine max
  misc_feature 1..237: note = Glyma09g35960.1, ortholog of AtBBX28 (G1481), AtBBX29 (G900)
  source 1..237: mol_type = unassigned DNA
atgggggaatattcatgcgcgttgtgcaagaagagggcgatgatgctgtgcgactcggaccaggctaagctatgctgggagtgcgacgagaaagttcacagcgcgaatttcttggtggctaagcattcgcgtgtgcttctctgtcgtttatgccattccccgactccgtggaaggcctccggcaccaaactcatgctcacggtgtcgttttgtcagcgttgcgtcgcgcatcgacac

SEQ ID NO: 28 - protein, 79 aa, Glycine max
  REGION 1..79: note = Glyma09g35960.1, domain coordinates 13-42, ortholog of AtBBX28 (G1481), AtBBX29 (G900)
MGEYSCALCKKRAMMLCDSDQAKLCWECDEKVHSANFLVAKHSRVLLCRLCHSPTPWKASGTKLMLTVSFCQRCVAHRH

SEQ ID NO: 29 - DNA, 519 bp, Glycine max
  misc_feature 1..519: note = Glyma12g11230.1, ortholog of AtBBX28 (G1481), AtBBX29 (G900)
  source 1..519: mol_type = unassigned DNA
atgaagaagtgcgaactgtgtaaagttccggcgaggatcttctgcgagtcggaccaggctagcttatgctgggactgcgacgctaaggttcacagcgccaacttcttggtcgcgaggcacgtgcgaaccctactctgccgcacgtgtcgttctcccacgccctggaaggcctccggcgcctcactcgcccacgccgcctccgtctgcgaccgctgcgccgccgcggaagagacggaagagagcgaagccggcaatgacgacgaaccagacaccgaggacgacgacgatgtcgacaaccaggtcgtgccgtggtcctccacgccgccgccgccgccgccgtcgagctcctccggcagcgaatgctcggagtgcaaaaacaacgttgcttctttttccgcctcggaacaggaaacgacaacatcctcgttgaaacgacagcgcgaggacgacgacgatgcttcttccgatcctgatcccagggtgttgagaagggttgggcgtctcaacggaggttgcgtg

SEQ ID NO: 30 - protein, 173 aa, Glycine max
  REGION 1..173: note = Glyma12g11230.1, domain coordinates 4-40, ortholog of AtBBX28 (G1481), AtBBX29 (G900)
MKKCELCKVPARIFCESDQASLCWDCDAKVHSANFLVARHVRTLLCRTCRSPTPWKASGASLAHAASVCDRCAAAEETEESEAGNDDEPDTEDDDDVDNQVVPWSSTPPPPPPSSSSGSECSECKNNVASFSASEQETTTSSLKRQREDDDDASSDPDPRVLRRVGRLNGGCV

SEQ ID NO: 31 - DNA, 663 bp, Glycine max
  misc_feature 1..663: note = Glyma12g32470.1, ortholog of AtBBX28 (G1481), AtBBX29 (G900)
  source 1..663: mol_type = unassigned DNA
atgaagaactgcgagctatgcaagcttccagctaggaccttctgcgagtcggaccaggctagcttatgctgggactgcgacgccaaggtccacggagcgaacttcctcgtcgcgagacacacgcgcacgctcctctgccacgcgtgccagtcactcacgccgtggaaggcctccggctctgccctcggcaacaccgtctcgctgtgccagagttgcgccggcggaaccaccgaacagggcccagagagccaaggagggaatgacgacgacgacatagacaccgacgatgaggatgactacgatgacgatggtgagagcgaggagaacgaggttgcagccgacgaggaggacggagacaaccaggttgtcccttggtcctccgagccgccgccaccgtccccaagctcttccagcagcgaagagtcgattagccggtgcaacaacgtggacgaggtttcgaccacattgaaacgacgtcgcgagaacgacgattttcagggttcgaattcgaataattggaaatgtgaacggagcgaagtggacagaggatgttggttggctcggttgcggcggagaaccgccgatgatgtggcggttgagcgacggagggctagagcggcgtttccgtacggttgttgtgatgatagagcatctgaagacgtt

SEQ ID NO: 32 - protein, 221 aa, Glycine max
  REGION 1..221: note = Glyma12g32470.1, domain coordinates 10-40, ortholog of AtBBX28 (G1481), AtBBX29 (G900)
MKNCELCKLPARTFCESDQASLCWDCDAKVHGANFLVARHTRTLLCHACQSLTPWKASGSALGNTVSLCQSCAGGTTEQGPESQGGNDDDDIDTDDEDDYDDDGESEENEVAADEEDGDNQVVPWSSEPPPPSPSSSSSEESISRCNNVDEVSTTLKRRRENDDFQGSNSNNWKCERSEVDRGCWLARLRRRTADDVAVERRRARAAFPYGCCDDRASEDV

SEQ ID NO: 33 - DNA, 462 bp, Glycine max
  misc_feature 1..462: note = G4014, Glyma13g32160.1, ortholog of AtBBX28 (G1481), AtBBX29 (G900)
  source 1..462: mol_type = unassigned DNA
atgaggaagtgtgagctctgtaacagtcccgcgaagttgttctgcgaatcagatcaagccagcctctgttggaaatgcgatgctaaggttcacagtgcaaacttcctcgtcaccaaacatcccaggattcttctctgccatgtttgtcaatcactaacagcgtggcacggcacaggacccaagtttgtacccaccatgtcagtttgcaacacttgtgtcaacaacaatagtactgagacctgcagccaacagaatcatgaagacgatgatgatgatggtacgggagaggatcatgcagaaaacgacgatggtggtgtggctgaagatgatgatgatgatgatgatgaagaaaatcaagtggttccatggacatctacaccaccacccccagcttccacttcttcaaatagtgttacaactagttctaccaggttctctgatgttgaagaaggtggctccgat

SEQ ID NO: 34 - protein, 154 aa, Glycine max
  REGION 1..154: note = G4014, Glyma13g32160.1, domain coordinates 4-40, ortholog of AtBBX28 (G1481), AtBBX29 (G900)
MRKCELCNSPAKLFCESDQASLCWKCDAKVHSANFLVTKHPRILLCHVCQSLTAWHGTGPKFVPTMSVCNTCVNNNSTETCSQQNHEDDDDDGTGEDHAENDDGGVAEDDDDDDDEENQVVPWTSTPPPPASTSSNSVTTSSTRFSDVEEGGSD

SEQ ID NO: 35 - DNA, 663 bp, Glycine max
  misc_feature 1..663: note = G4016, Glyma13g37970.1, ortholog of AtBBX28 (G1481), AtBBX29 (G900)
  source 1..663: mol_type = unassigned DNA
atgaagaactgcgagttgtgcaagcttccggctcggactttctgcgagtcggaccaggctagcttatgctgggactgcgacgccaaggttcatggagccaacttcctcgtggaaagacacacgcgcacgctcctctgccacgcgtgccagtcgcccacgccgtggaaggcctccggcgccgccctcggaaacaccgtctcgctgtgcgagagatgcgccggcggaaccaccgaacaaggtcaagagagccaaggaggcaatgacgacgacatagacaccgacatagacaccgacgatgaggatgactacgatgagagcgaggacgaggttgccgccgacgaggaggacggagacaaccaggttgtcccttggtcctccgagccgccgccaccagccccgagctcttccagcagcgaagaatcggttagccggtgcaacaacgtggacgaggtttcgaccacattgaaacgccgtcgccaggaggacaatgattttcagggttggaattcgaataattggggatgtgaacggagcgaagtggagagaggaggttggttggttcggttgcggcggagaaccgccgatgatgtggcggttgagcaacggagtgctagagcggcgtctccagacggttgctgtggtgatagagcatctgaagacgtt

SEQ ID NO: 36 - protein, 221 aa, Glycine max
  REGION 1..221: note = G4016, Glyma13g37970.1, domain coordinates 10-40, ortholog of AtBBX28 (G1481), AtBBX29 (G900)
MKNCELCKLPARTFCESDQASLCWDCDAKVHGANFLVERHTRTLLCHACQSPTPWKASGAALGNTVSLCERCAGGTTEQGQESQGGNDDDIDTDIDTDDEDDYDESEDEVAADEEDGDNQVVPWSSEPPPPAPSSSSSEESVSRCNNVDEVSTTLKRRRQEDNDFQGWNSNNWGCERSEVERGGWLVRLRRRTADDVAVEQRSARAASPDGCCGDRASEDV

SEQ ID NO: 37 - DNA, 456 bp, Glycine max
  misc_feature 1..456: note = Glyma15g07140.1, ortholog of AtBBX28 (G1481), AtBBX29 (G900)
  source 1..456: mol_type = unassigned DNA
atgaggaagtgtgaactctgtaatagtcccgcaaagttgttctgtgaatcagatcaagccagcctctgttgggaatgcgatgctaaggttcacagtgcaaacttcctcgtcaccaaacatcccaggattcttctctgccatgtttgtcaatcactaacggcctggcacggcacgggacccaagtttgtacccaccatgtccgtttgcaacacttgtgtcaacaacaatagtgagacctgcagccaacagaatcatgttgaggaccacgacgacaatcatgatgatggcacgggagaggatcatgtagaaaaccatgatggtggtgtgtctgaagatgatgaagaaaatcaagttgttccatggacatctacaccaccacccccagcttccacttcttcaaatagtgttacaactagttctaccaggttctctaatgttgaagaaggtggctccgat

SEQ ID NO: 38 - protein, 152 aa, Glycine max
  REGION 1..152: note = Glyma15g07140.1, domain coordinates 4-40, ortholog of AtBBX28 (G1481), AtBBX29 (G900)
MRKCELCNSPAKLFCESDQASLCWECDAKVHSANFLVTKHPRILLCHVCQSLTAWHGTGPKFVPTMSVCNTCVNNNSETCSQQNHVEDHDDNHDDGTGEDHVENHDGGVSEDDEENQVVPWTSTPPPPASTSSNSVTTSSTRFSNVEEGGSD

SEQ ID NO: 39 - DNA, 351 bp, Arabidopsis thaliana
  misc_feature 1..351: note = AtBBX30 (G1478), AT4G15248.1, paralog of AtBBX31 (G1929)
  source 1..351: mol_type = unassigned DNA
atgtgtagagggtttgagaaagaagaagagagaagaagcgacaatggaggatgccaaagactatgcacggagagtcacaaagctccggtaagctgtgagctttgcggcgagaacgccaccgtgtattgtgaggcagacgcagctttcctttgtaggaaatgcgatcgatgggtccattctgctaattttctagctcggagacatctccggcgcgtgatctgcacgacctgtcggaagctaactcgtcgatgtcttgtcggtgataattttaatgttgttttaccggagataaggatgatagcaaggattgaagaacatagtagtgatcacaaaattccctttgtgtttctc

SEQ ID NO: 40 - protein, 117 aa, Arabidopsis thaliana
  REGION 1..117: note = AtBBX30 (G1478), AT4G15248.1, domain coordinates 39-68, paralog of AtBBX31 (G1929)
MCRGFEKEEERRSDNGGCQRLCTESHKAPVSCELCGENATVYCEADAAFLCRKCDRWVHSANFLARRHLRRVICTTCRKLTRRCLVGDNFNVVLPEIRMIARIEEHSSDHKIPFVFL

SEQ ID NO: 41 - DNA, 366 bp, Arabidopsis thaliana
  misc_feature 1..366: note = AtBBX31 (G1929), paralog of AtBBX30 (G1478)
  source 1..366: mol_type = unassigned DNA
atgtgtagaggcttgaataatgaagagagcagaagaagtgacggaggaggttgccggagtctctgcacgagaccgagtgttccggtaaggtgtgagctttgcgacggagacgcctccgtgttctgtgaagcggactcggcgttcctctgtagaaaatgtgaccggtgggttcatggagcgaattttctagcttggagacacgtaaggcgcgtgctatgcacttcttgtcagaaactcacgcgccggtgcctcgtcggagatcatgacttccacgttgttttaccgtcggtgacgacggtcggagaaaccaccgtggagaatagaagtgaacaagataatcatgaggttccgtttgtttttctctga

SEQ ID NO: 42 - protein, 121 aa, Arabidopsis thaliana
  REGION 1..121: note = AtBBX31 (G1929), paralog of AtBBX30 (G1478)
MCRGLNNEESRRSDGGGCRSLCTRPSVPVRCELCDGDASVFCEADSAFLCRKCDRWVHGANFLAWRHVRRVLCTSCQKLTRRCLVGDHDFHVVLPSVTTVGETTVENRSEQDNHEVPFVFL

SEQ ID NO: 43 - DNA, 878 bp, Populus trichocarpa
  misc_feature 1..878: note = Pt_562313, ortholog of AtBBX30 (G1478), AtBBX31 (G1929)
  source 1..878: mol_type = unassigned DNA
atgtgtaggggtattcatcaggagagaaataaccaaggcggttcttgtggcgaagaggtggtttcatctaaggcaacctcaagattggtttgttgtgagctgtgtggatcaagggcatcattatattgtcaagcagatgatgcatttttatgtcaaaaatgtgacaaatgggtgcatggagcaaatttcctagctcaaaggcatgttagatgcatgctatgcaacacatgccaaaatcttacacaaagatatctcataggggcttcaactgagttgttgcttccaactattgtgagttggagagaaagaaggcagtgcaattctaatcttgaaaagaagagctctggatcacttaaaatgccctttttgtttctttgattctgcttctctctttcaccttttctgtcccttttttcttcccttttttttagttttctggtgctgttcaaggattttttttcttgagatttattgatggagagaacacaaaactgtcacttgaagggttttgggtgcatatgagaattgttgcaagtgggtgataaaattgatcagtcaatttttctgttatatgcaccaccatatatatccaagaaaatacagagaccatagcagctgcttggaatcctggaaaaggaagagaggagaaaaacacagacagaaagatgggtgatgaaatagaggataccctggaatcattgtctccatgaaatcctctgaaagagatcaaattaaatcagaaaatgtattttttttttctctggaaattttctaattttatcacctcttttttgtccaaagtaatgtttttctggtgtgtgtacttttctttttggttataaatagtgtgtaatcatttagatcaaaaggtaagggaa

SEQ ID NO: 44 - protein, 125 aa, Populus trichocarpa
  REGION 1..125: note = Pt_562313, domain coordinates 32-68, ortholog of AtBBX30 (G1478), AtBBX31 (G1929)
MCRGIHQERNNQGGSCGEEVVSSKATSRLVCCELCGSRASLYCQADDAFLCQKCDKWVHGANFLAQRHVRCMLCNTCQNLTQRYLIGASTELLLPTIVSWRERRQCNSNLEKKSSGSLKMPFLFL

SEQ ID NO: 45 - DNA, 372 bp, Populus trichocarpa
  misc_feature 1..372: note = Pt_594447, ortholog of AtBBX30 (G1478), AtBBX31 (G1929)
  source 1..372: mol_type = unassigned DNA
atgtgtagaggtaatcaggagagaagtaaccaaggcagttcttgtaacaaggaggctgtttcacctaatgcaacctcaagattcgtttgttgtgagctatgtggctcgagggcaacattgtattgtcaagcagatcatgcatttctatgtcaaaaatgtgacggatgggtgcatggagctaatttcttagccctcaggcatgttagaaacatgttatgcaacacatgccaaaatcttacacagagatgtctcattggggcttcaactgaggtgatgctttcaactattgtgagttggagagaaagaagggatcgtaattctaatcttgaaaagaagtgctctggatcacttaaaaagcccttttcgtttctt

SEQ ID NO: 46 - protein, 124 aa, Populus trichocarpa
  REGION 1..124: note = Pt_594447, domain coordinates 31-67, ortholog of AtBBX30 (G1478), AtBBX31 (G1929)
MCRGNQERSNQGSSCNKEAVSPNATSRFVCCELCGSRATLYCQADHAFLCQKCDGWVHGANFLALRHVRNMLCNTCQNLTQRCLIGASTEVMLSTIVSWRERRDRNSNLEKKCSGSLKKPFSFL

SEQ ID NO: 47 - DNA, 372 bp, Glycine max
  misc_feature 1..372: note = Glyma12g32230.1, ortholog of AtBBX30 (G1478), AtBBX31 (G1929)
  source 1..372: mol_type = unassigned DNA
atgtgcaaaggtgcagaaggagaaaagcaacatggtttctgcagcagctttctacataaagagagtgcaaccagaagtgctatatgttgtgagctatgtgggttacaggcttcattgtattgccaagctgatgatgcctacttgtgtaaaaaatgcgataaacgggttcatgaagctaattttttggcccttaggcacattaggtgctttctgtgcaacacatgtcagaaccttacgcgaagatatctcattggagcatcactagaggtggttcttccggccaatataaactggaccattagaaatctccttaacaacagaggaatctacagaaagtgttcaagaatgcataacagcccttccctcttttta

SEQ ID NO: 48 - protein, 124 aa, Glycine max
  REGION 1..124: note = Glyma12g32230.1, domain coordinates 30-66, ortholog of AtBBX30 (G1478), AtBBX31 (G1929)
MCKGAEGEKQHGFCSSFLHKESATRSAICCELCGLQASLYCQADDAYLCKKCDKRVHEANFLALRHIRCFLCNTCQNLTRRYLIGASLEVVLPANINWTIRNLLNNRGIYRKCSRMHNSPSLFL

SEQ ID NO: 49 - DNA, 372 bp, Glycine max
  misc_feature 1..372: note = G4019, Glyma13g38240.1, ortholog of AtBBX30 (G1478), AtBBX31 (G1929)
  source 1..372: mol_type = unassigned DNA
atgtgcaaaggtgcagaaggagaaaagcaacatggtttctgcagcagctatctacataaagaatgtgcaaccagaagtgctacatgttgtgagctatgtgggttacaggcttcattgtattgtcaagctgatgatgcatacttgtgtagaaaatgtgataaaagggttcatgaagctaattttttggcccttaggcacattaggtgctttctgtgcaacacatgtcaaaaccttacgcgaagatatctcattggagcatcaatagaggtggttcttccagccaacattaactggaccattggaaatctccctagcaacagaggaatccacagaaagtgctcaagaatgcataacaacctttccctcttgtta

SEQ ID NO: 50 - protein, 124 aa, Glycine max
  REGION 1..124: note = G4019, Glyma13g38240.1, domain coordinates 30-66, ortholog of AtBBX30 (G1478), AtBBX31 (G1929)
MCKGAEGEKQHGFCSSYLHKECATRSATCCELCGLQASLYCQADDAYLCRKCDKRVHEANFLALRHIRCFLCNTCQNLTRRYLIGASIEVVLPANINWTIGNLPSNRGIHRKCSRMHNNLSLLL

SEQ ID NO: 51 - DNA, 753 bp, Arabidopsis thaliana
  misc_feature 1..753: note = AtBBX26 (G1486), AT1G60250.1, ortholog of AtBBX26 (G1486)
  source 1..753: mol_type = unassigned DNA
atggctcaagtttgtcacacatgtagacatgtaacagccgtgatccattgtgttacagaagctcttaacttctgccttacatgtgacaaccttcgccatcataataacatacatgcagaacacgtacgttaccaactttgcgataactgctcgatgtatccgtctatacttttctgttatgaggacgggatggttctttgtcaatcatgttattcgcaccattacaattgtgccactaatggccatcaaactcaagtcgtcttcgctaacatgaataatcaacaccatgatcatgcacatatgcctcatgtggttcaccataacaataacaataatcatcagcagcagcatgtgggtggtcaccaaagaagagcagagatgtttgagaggagctgccacggggataacaattgcgagagatggatgtttgcaatgagatgtgagttatgcgtagcttcgaattcaaatgctgttgtttattgtcctacacacaaccagattctatgcgatagttgcgaccggatgattcacagtcacgaggacgcggtgccgccgcattcgaggtgtaagctttgcgtaatctgcaagagaccttctcgccgttttctcattggcggctatcagtttaatttcccaccggttcatcctcctgcggcagaggggattccagttacgcccccaacagaactacctcaacaagatattaattatgattatctagatgatgttgatgatttttcttggtttggtagg

SEQ ID NO: 52 - protein, 251 aa, Arabidopsis thaliana
  REGION 1..251: note = AtBBX26 (G1486), AT1G60250.1, domain coordinates 5-36, 47-77
MAQVCHTCRHVTAVIHCVTEALNFCLTCDNLRHHNNIHAEHVRYQLCDNCSMYPSILFCYEDGMVLCQSCYSHHYNCATNGHQTQVVFANMNNQHHDHAHMPHVVHHNNNNNHQQQHVGGHQRRAEMFERSCHGDNNCERWMFAMRCELCVASNSNAVVYCPTHNQILCDSCDRMIHSHEDAVPPHSRCKLCVICKRPSRRFLIGGYQFNFPPVHPPAAEGIPVTPPTELPQQDINYDYLDDVDDFSWFGR

SEQ ID NO: 53 - DNA, 726 bp, Arabidopsis thaliana
  misc_feature 1..726: note = AtBBX20 (G1888), AT4G39070.1, paralog of AtBBX21 (G1482)
  source 1..726: mol_type = unassigned DNA
atgaagatttggtgtgctgtttgtgataaagaagaagcttcggtgttttgttgtgcggatgaagcagctctttgtaatggttgcgatcgccatgttcatttcgccaataaactagccgggaaacatctccggttctctctcacttctcctactttcaaagatgctcctctttgtgatatttgcggggagaggcgtgcattattattttgccaagaagacagagcaatactatgcagagaatgtgacattccaatacatcaagctaatgagcacactaagaaacacaatagattcctccttaccggcgttaagatctctgcctccccgtcagcctacccaagagcctccaattccaactctgctgctgcatttggtcgagccaaaacccgaccaaaatcagtatcgagcgaggtcccgagctcggcctccaatgaggtatttacgagctcttcttcgacgaccacgagcaattgctattatgggatagaagaaaactaccatcacgtgagcgattcggggtcgggatcgggttgtacaggtagtatatccgagtatttgatggagacattaccgggttggagagtggaggatttgcttgaacacccttcttgtgtctcctatgaggataacattattactaataacaataacagtgagtcttatagggtttatgatggttcttcacaattccatcatcaagggttttgggatcacaaacccttctct

SEQ ID NO: 54 - protein, 242 aa, Arabidopsis thaliana
  REGION 1..242: note = AtBBX20 (G1888), AT4G39070.1, domain coordinates 5-42, 58-95, paralog of AtBBX21 (G1482)
MKIWCAVCDKEEASVFCCADEAALCNGCDRHVHFANKLAGKHLRFSLTSPTFKDAPLCDICGERRALLFCQEDRAILCRECDIPIHQANEHTKKHNRFLLTGVKISASPSAYPRASNSNSAAAFGRAKTRPKSVSSEVPSSASNEVFTSSSSTTTSNCYYGIEENYHHVSDSGSGSGCTGSISEYLMETLPGWRVEDLLEHPSCVSYEDNIITNNNNSESYRVYDGSSQFHHQGFWDHKPFS

SEQ ID NO: 55 - DNA, 993 bp, Arabidopsis thaliana
  misc_feature 1..993: note = AtBBX21 (G1482), AT1G75540.1, paralog of AtBBX20 (G1888)
  source 1..993: mol_type = unassigned DNA
atgaagatcaggtgcgacgtctgcgataaagaagaagcgtcggtgttttgcacggccgacgaagcatctctctgcggcggctgcgaccaccaagtccaccacgctaacaaactcgcctctaaacatctccgtttctctctcctttatccttcttcttccaacacctcctctcctctctgcgacatctgtcaggataaaaaagctctgttgttctgtcaacaagatagagctattttatgcaaagattgcgattcatcgatccacgctgcgaacgaacacacaaagaaacacgataggtttcttcttacaggggttaagctctctgcaacatcgtctgtttacaaacctacttcgaaatcttcttcttcttcttcaagcaaccaagatttctctgtccctggatcatcaatctctaatcctcctcctctcaagaaacctctctcagctcctcctcagagcaacaagatccaacccttttcgaagatcaacggcggtgatgcgtcggtgaatcagtggggatccacaagcacgatttctgagtatttgatggatacgttacctggttggcacgttgaggatttcctcgattcctctcttcctacttatggtttctctaagagtggtgatgatgatggagtgttaccatatatggaaccagaagatgacaacaacactaagagaaacaacaacaacaacaacaacaacaacaacaatacagtgtcacttccatctaagaatttagggatttgggtccctcagattccacaaactcttccttcttcatacccaaatcaatacttttctcaagacaacaacatacagtttgggatgtacaacaaagaaacatcaccagaagtagtgtcttttgctccaatacaaaacatgaaacaacaaggacagaacaacaagagatggtatgatgatggtggcttcactgtcccacagatcactcctcctcctctttcttctaataaaaagtttagatctttctgg

SEQ ID NO: 56 - protein, 331 aa, Arabidopsis thaliana
  REGION 1..331: note = AtBBX21 (G1482), AT1G75540.1, domain coordinates 5-42, 60-97, paralog of AtBBX20 (G1888)
MKIRCDVCDKEEASVFCTADEASLCGGCDHQVHHANKLASKHLRFSLLYPSSSNTSSPLCDICQDKKALLFCQQDRAILCKDCDSSIHAANEHTKKHDRFLLTGVKLSATSSVYKPTSKSSSSSSSNQDFSVPGSSISNPPPLKKPLSAPPQSNKIQPFSKINGGDASVNQWGSTSTISEYLMDTLPGWHVEDFLDSSLPTYGFSKSGDDDGVLPYMEPEDDNNTKRNNNNNNNNNNNTVSLPSKNLGIWVPQIPQTLPSSYPNQYFSQDNNIQFGMYNKETSPEVVSFAPIQNMKQQGQNNKRWYDDGGFTVPQITPPPLSSNKKFRSFW

SEQ ID NO: 57 - DNA, 768 bp, Zea mays
  misc_feature 1..768: note = ACG44133.1, ortholog of AtBBX20 (G1888), AtBBX21 (G1482), AtBBX21 (G1482)
  source 1..768: mol_type = unassigned DNA
atgaaggtgcagtgcgacgtgtgcgcggccgaggcggcctccgtcttctgctgcgccgacgaggccgcgctgtgcgacgcgtgcgacgaccgcgtccaccgcgccaacaagctcgcggggaagcaccgccgcttctccctcctccacccctgttcgtcctctgcgcagaagccgccgctctgcgacatctgccaggagcggagaggcttcttgttctgcaaggaggacagggcgatcctgtgccgggagtgcgacgcgccggtgcacagcgcgaacgacatgacgaggcggcacagccggttcctgctcaccggcgtgcgcctctcgtcagcgccggtggactctgctgacccatcagaaggggaggaggaggaggagcaggagaacagcagcaggcccggcaacggcgagtcctgcagcggcggcgccggcgccggcgcgaccacggcgaccgccagcgacgggagcagcatctccgagtacctgaccaagaccctgcccgggtggcacgtcgaggacttcctcgttgacgacgcgtacgccagcgacgtcggcgcctgctcagacggcctttatcagggacaagatggacagatcagcggggtgctgcaagaagcttacatgccgtggacggggcgggagctggtgccctctgacgtcgcagacgagcgggacaactgggagcggtgggtgccgcagatgcacgcggagttcgccggcgacagcaagcggcccagagcgtcgccttcgcctccctgttcgtactgg

SEQ ID NO: 58 - protein, 256 aa, Zea mays
  REGION 1..256: note = ACG44133.1, domain coordinates 5-42, 61-98, ortholog of AtBBX20 (G1888), AtBBX21 (G1482), AtBBX21 (G1482)
MKVQCDVCAAEAASVFCCADEAALCDACDDRVHRANKLAGKHRRFSLLHPCSSSAQKPPLCDICQERRGFLFCKEDRAILCRECDAPVHSANDMTRRHSRFLLTGVRLSSAPVDSADPSEGEEEEEQENSSRPGNGESCSGGAGAGATTATASDGSSISEYLTKTLPGWHVEDFLVDDAYASDVGACSDGLYQGQDGQISGVLQEAYMPWTGRELVPSDVADERDNWERWVPQMHAEFAGDSKRPRASPSPPCSYW

SEQ ID NO: 59 - DNA, 630 bp, Zea mays
  misc_feature 1..630: note = CAW36814.1, ortholog of AtBBX20 (G1888), AtBBX21 (G1482), AtBBX21 (G1482)
  source 1..630: mol_type = unassigned DNA
atgaaggtgcagtgcgacgtgtgcgcggccgaggcggcctccgtcttctgctgtgccgacgaggccgcgctgtgcgacgcgtgcgacgaccgcgtccaccgcgccaacaagctcgcggggaagcaccgccgcttctccctcctccacccctgttcgtcctctgcgcacaagccgccgctctgcgacatctgccaggagcggagaggcttcttgttctgcaaggaggacagggcgatcctgtgccgggagtgcgacgcgccggtgcacagcgcgaacgacatgacgaggcggcacagccggttcctgctcaccggcgtgcgcctctcgtcggcgccggtggactctgctgacccatcagaaggggaggaggaggaggagcaggagaacagcagcaggcccggcaacggtgagtcctgcagcggcggcgccggcgctaccacggcgaccgctagcgacgggagcagcatctccgagtacctgaccaagaccctgcccgggtggcacgtcgaggatttcctcgttgacgacgcgtacgccagcgacgtcggcgcctgctcatcagacgggctttatcaggtcagttcagttcttccatgcgaatggaactgcaagctttcactgaggcgttcc

SEQ ID NO: 60 - protein, 210 aa, Zea mays
  REGION 1..210: note = CAW36814.1, domain coordinates 5-42, 61-98, ortholog of AtBBX20 (G1888), AtBBX21 (G1482), AtBBX21 (G1482)
MKVQCDVCAAEAASVFCCADEAALCDACDDRVHRANKLAGKHRRFSLLHPCSSSAHKPPLCDICQERRGFLFCKEDRAILCRECDAPVHSANDMTRRHSRFLLTGVRLSSAPVDSADPSEGEEEEEQENSSRPGNGESCSGGAGATTATASDGSSISEYLTKTLPGWHVEDFLVDDAYASDVGACSSDGLYQVSSVLPCEWNCKLSLRRS

SEQ ID NO: 61 - DNA, 630 bp, Zea mays; identical to SEQ ID NO: 59
  misc_feature 1..630: note = CAW39070.1, ortholog of AtBBX20 (G1888), AtBBX21 (G1482)
  source 1..630: mol_type = unassigned DNA

SEQ ID NO: 62 - protein, 210 aa, Zea mays; identical to SEQ ID NO: 60
  REGION 1..210: note = CAW39070.1, domain coordinates 5-42, 61-98, ortholog of AtBBX20 (G1888), AtBBX21 (G1482)

SEQ ID NO: 63 - DNA, 630 bp, Zea mays; identical to SEQ ID NO: 59
  misc_feature 1..630: note = CAW54447.1, ortholog of AtBBX20 (G1888), AtBBX21 (G1482)
  source 1..630: mol_type = unassigned DNA

SEQ ID NO: 64 - protein, 210 aa, Zea mays; identical to SEQ ID NO: 60
  REGION 1..210: note = CAW54447.1, domain coordinates 5-42, 61-98, ortholog of AtBBX20 (G1888), AtBBX21 (G1482)

SEQ ID NO: 65 - DNA, 630 bp, Zea mays; identical to SEQ ID NO: 59
  misc_feature 1..630: note = CAW74338.1, ortholog of AtBBX20 (G1888), AtBBX21 (G1482)
  source 1..630: mol_type = unassigned DNA

SEQ ID NO: 66 - protein, 210 aa, Zea mays; identical to SEQ ID NO: 60
  REGION 1..210: note = CAW74338.1, domain coordinates 5-42, 61-98, ortholog of AtBBX20 (G1888), AtBBX21 (G1482)

SEQ ID NO: 67 - DNA, 582 bp, Populus trichocarpa
  misc_feature 1..582: note = Pt_195637, ortholog of AtBBX20 (G1888), AtBBX21 (G1482)
  source 1..582: mol_type = unassigned DNA
atgaagatccggtgtgatgtgtgtgacaatgtagaggcgacagttttctgttgtgcggatgaagctgctctctgcgatggatgtgatcacagagttcaccatgcaaacaagttagcaagcaaacactcgaggttctctttagtccatccttctttcaaagaatcccctctctgtgatatctgtcaggagaggagagcccttctgttttgtcaagaggatagggcaattctatgcagagaatgtgaccttccaattcacaaagctaatgaacatactcagaaacacaacaggtttcttctcacaggtgtaaagctctctgcttcttcttctctgcacacagcatcatcgacctccactaataactttgactcaaacattaacactactagcaatagaaaccatcagccatacctaaagaattccaacgaaatcctcagctctccttcagttgagacagcatcagcaacaacggcatataatttcgaagaaaatcatgttagtgacaatggttcaatttcaacaagtagcatatctgagtatttagagacagtaccaggctggcgcatcgatgattttcttgat

SEQ ID NO: 68 - protein, 194 aa, Populus trichocarpa
  REGION 1..194: note = Pt_195637, domain coordinates 5-42, 58-95, ortholog of AtBBX20 (G1888), AtBBX21 (G1482)
MKIRCDVCDNVEATVFCCADEAALCDGCDHRVHHANKLASKHSRFSLVHPSFKESPLCDICQERRALLFCQEDRAILCRECDLPIHKANEHTQKHNRFLLTGVKLSASSSLHTASSTSTNNFDSNINTTSNRNHQPYLKNSNEILSSPSVETASATTAYNFEENHVSDNGSISTSSISEYLETVPGWRIDDFLD

SEQ ID NO: 69 - DNA, 930 bp, Populus trichocarpa
  misc_feature 1..930: note = Pt_550941, ortholog of AtBBX20 (G1888), AtBBX21 (G1482)
  source 1..930: mol_type = unassigned DNA
atgaagatccagtgcgatgtgtgtaacaaagaagaggcatcggtgttttgcaccgctgacgaggcagctctttgcgacacctgtgaccaccgtgttcaccatgccaacaagcttgcttcaaagcaccaacgtttttcccttctccatccttcctcaaaaaacttccccatctgtgatatctgccaggagaaacgggctttcttgttctgtcaacaagacagggcgattttatgtagagagtgtgatggtccaatacacacagcaaacgagcatacccagaagcacaacaggtttcttctcacaggagtcaagctctctgctacatctgctgtttatatatcttcttcctctgtcaccaacagtggtggtgatctcgttcctgattcaaagtctcaacagcagcagcagcagcagcaatcaatcaagaaacctgtgtttgatgctccagtgaattccaatccacctacagttcccagtaccttatctacaaacacagaagtaaacaagggtggggataatttggtaacaaatgaagggtttggttcaacaacaagtagtactatatcagagtacttgatggagactcttcctggctggcatgttgaagactttcttgattcctctactactccctttggtttctgtaagattgatgatggtctattgccgtttatggatgctcatgatcttgagagcaacatgagttctttctcatcagaaagtttggggctttgggtccctcaagcaccatctactccatacacatctcaacagtattattatccacagttggtagggcaaagtgggttcaaggagataaaagagaccacaaacatgaaagctaacagaaggttggcagatgatgtcttcactgttccacagatcagcctcccggccaatataagctctaagagatctaggcccttatgg

SEQ ID NO: 70 - protein, 310 aa, Populus trichocarpa
  REGION 1..310: note = Pt_550941, domain coordinates 5-42, 58-95, ortholog of AtBBX20 (G1888), AtBBX21 (G1482)
MKIQCDVCNKEEASVFCTADEAALCDTCDHRVHHANKLASKHQRFSLLHPSSKNFPICDICQEKRAFLFCQQDRAILCRECDGPIHTANEHTQKHNRFLLTGVKLSATSAVYISSSSVTNSGGDLVPDSKSQQQQQQQQSIKKPVFDAPVNSNPPTVPSTLSTNTEVNKGGDNLVTNEGFGSTTSSTISEYLMETLPGWHVEDFLDSSTTPFGFCKIDDGLLPFMDAHDLESNMSSFSSESLGLWVPQAPSTPYTSQQYYYPQLVGQSGFKEIKETTNMKANRRLADDVFTVPQISLPANISSKRSRPLW

SEQ ID NO: 71 - DNA, 849 bp, Populus trichocarpa
  misc_feature 1..849: note = Pt_801530, ortholog of AtBBX20 (G1888), AtBBX21 (G1482)
  source 1..849: mol_type = unassigned DNA
atgaagatccagtgtgacgtttgtagcaaagaagaggcctcggtgttttgcaccgctgacgaggcagccctttgtgacacttgtgaccaccgtgtccatcatgccaacaagcttgcttcaaagcatcagcgtttttcccttctccatccttcctcaaaaaacttccccatctgtgatatctgccaggacaaacgggctttcttgttctgtcaacaagacagggcgattttatgcagagattgtgacggtccaatacacacagcaaacgagcatacccagaagcataataggtttcttctcacaggagtcaagctctctgctacatctgctgtttatatgtcttcctcttcctccgttaccatgaattccaatccacctgcagttcccagtaccttgtcagcaaacacagtaataaacaaggacggggataatttggtaacaagtgaagggtttggttcgacaacaagtagtactatatcagagtacttgatggagactcttcctggctggcatgttgaagaatttcttgattcctcttccaccactccatttggtttcagtaagattgatgatggtctcttgccgtatatggacactcatgatcttgaacgcaatatgagttctttctcgtcagaaagtttggggctttgggtcccccaagcaccaactcctcctctatgcacatctcaacaatattactatccacaattggtagggcagagtggattcaaggagacaaaagagagcacaaacatgaaagctaacagaaggttaacagatgatgccttcactgttccacagataagccctccatcaaatattggctccaagagatctaggcccttgtgg

SEQ ID NO: 72 - protein, 283 aa, Populus trichocarpa
  REGION 1..283: note = Pt_801530, domain coordinates 5-42, 58-95, ortholog of AtBBX20 (G1888), AtBBX21 (G1482)
MKIQCDVCSKEEASVFCTADEAALCDTCDHRVHHANKLASKHQRFSLLHPSSKNFPICDICQDKRAFLFCQQDRAILCRDCDGPIHTANEHTQKHNRFLLTGVKLSATSAVYMSSSSSVTMNSNPPAVPSTLSANTVINKDGDNLVTSEGFGSTTSSTISEYLMETLPGWHVEEFLDSSSTTPFGFSKIDDGLLPYMDTHDLERNMSSFSSESLGLWVPQAPTPPLCTSQQYYYPQLVGQSGFKETKESTNMKANRRLTDDAFTVPQISPPSNIGSKRSRPLW

SEQ ID NO: 73 - DNA, 645 bp, Populus trichocarpa
  misc_feature 1..645: note = Pt_804174, ortholog of AtBBX20 (G1888), AtBBX21 (G1482)
  source 1..645: mol_type = unassigned DNA
atgaagatccggtgtgatgtgtgtgacaaggtagaggccacagttttttgttgtgcagatgaagctgctctctgtgatggatgtgatcacagagttcaccatgcaaacacgctagcaagcaaacactcaaggttctctttagtccatccttccttcaaagaatcccctctttgtgatatctgccaggagaggagagccgttctgttttgtcaagaggatagagcaattctatgcagagaatgtgaccttccaattcacaaagttaatgaacatacccagaaacacaataggtttcttctcacaggtgtaaagctctgtggtccttctctgtacgcaacatcatcctccgctagtaactgtgatgcaaacattaacactactagaaatagaaaccatcagcactacctgaagaaaccgatttcagcttccaatgaaatcttcagttctccttcagtagcgacagcgtcgccaccaacagcatatagttatgacgataatcatgttagtggcggtggttcagtttcaacaagtagcatatctgagtatttagagacggtagtaccagggtggcgcgtggatgattttcttgatccttcattcactagcaataatagtttcagcaagggttggaatgcaccctatata

SEQ ID NO: 74 - protein, 215 aa, Populus trichocarpa
  REGION 1..215: note = Pt_804174, domain coordinates 5-42, 58-95, ortholog of AtBBX20 (G1888), AtBBX21 (G1482)
MKIRCDVCDKVEATVFCCADEAALCDGCDHRVHHANTLASKHSRFSLVHPSFKESPLCDICQERRAVLFCQEDRAILCRECDLPIHKVNEHTQKHNRFLLTGVKLCGPSLYATSSSASNCDANINTTRNRNHQHYLKKPISASNEIFSSPSVATASPPTAYSYDDNHVSGGGSVSTSSISEYLETVVPGWRVDDFLDPSFTSNNSFSKGWNAPYI

SEQ ID NO: 75 - DNA, 807 bp, Oryza sativa
  misc_feature 1..807: note = Os02g43170.1, ortholog of AtBBX20 (G1888), AtBBX21 (G1482)
  source 1..807: mol_type = unassigned DNA
atgaaggtgcagtgcgacgtgtgcgcggccgaggcggcgtcggtgttctgctgcgccgacgaggccgcgctgtgcgacgcgtgcgaccaccgggtgcaccgggccaacaagctcgccgggaagcaccgccggttctcgctgctcaacccctcggcgtccggccgctcgccgacatcgacgacggcgccgctctgcgacatctgccaggagaagaggggtttcctgttctgcaaggaggaccgggcgatcctgtgccgcgagtgcgacgtgccggtgcacacggcgagcgagctcaccatgcgccacagccggtacctcctcaccggcgtgcggctctcctcggagcctgccgcgtccccggcgccgccgtcggaggaggagaacagcagcagcttctgctgcagcgccgacgacgccgtgccggccccggcggcgcccgccacgagccacggcgggagcagcggcagcagcagcatctccgagtacctcaccacgctgcccgggtggcacgtcgaggacttcctcgtcgacgacgccactgccgaggccgccgccgccgccgccgccacctcttccggcatctccgcgaacgggccgtgtcagggggtaacacggatcggagggctgcaagaatccgccggctaccctgcgtggatggcgcagcagcagctgtgctgcgacggcctcgtcgccggcgacgcgtcgccggctagccgggagcggtgggtgccgcagatgtacgcggatcagcttgccgccggcagcaagagatccaggacgtccactgcttcttcctactcctactgg

SEQ ID NO: 76 - protein, 269 aa, Oryza sativa
  REGION 1..269: note = Os02g43170.1, domain coordinates 5-42, 65-102, ortholog of AtBBX20 (G1888), AtBBX21 (G1482)
MKVQCDVCAAEAASVFCCADEAALCDACDHRVHRANKLAGKHRRFSLLNPSASGRSPTSTTAPLCDICQEKRGFLFCKEDRAILCRECDVPVHTASELTMRHSRYLLTGVRLSSEPAASPAPPSEEENSSSFCCSADDAVPAPAAPATSHGGSSGSSSISEYLTTLPGWHVEDFLVDDATAEAAAAAAATSSGISANGPCQGVTRIGGLQESAGYPAWMAQQQLCCDGLVAGDASPASRERWVPQMYADQLAAGSKRSRTSTASSYSYW

SEQ ID NO: 77 - DNA, 624 bp, Oryza sativa
  misc_feature 1..624: note = Os02g43170.2, ortholog of AtBBX20 (G1888), AtBBX21 (G1482)
  source 1..624: mol_type = unassigned DNA
atgaaggtgcagtgcgacgtgtgcgcggccgaggcggcgtcggtgttctgctgcgccgacgaggccgcgctgtgcgacgcgtgcgaccaccgggtgcaccgggccaacaagctcgccgggaagcaccgccggttctcgctgctcaacccctcggcgtccggccgctcgccgacatcgacgacggcgccgctctgcgacatctgccaggagaagaggggtttcctgttctgcaaggaggaccgggcgatcctgtgccgcgagtgcgacgtgccggtgcacacggcgagcgagctcaccatgcgccacagccggtacctcctcaccggcgtgcggctctcctcggagcctgccgcgtccccggcgccgccgtcggaggaggagaacagcagcagcttctgctgcagcgccgacgacgccgtgccggccccggcggcgcccgccacgagccacggcgggagcagcggcagcagcagcatctccgagtacctcaccacgctgcccgggtggcacgtcgaggacttcctcgtcgacgacgccactgccgaggccgccgccgccgccgccgccacctcttccggcatctccgcgaacgggccgtgtcaggttggtgccactgatcgacca

SEQ ID NO: 78 - protein, 208 aa, Oryza sativa
  REGION 1..208: note = Os02g43170.2, domain coordinates 5-42, 65-102, ortholog of AtBBX20 (G1888), AtBBX21 (G1482)
MKVQCDVCAAEAASVFCCADEAALCDACDHRVHRANKLAGKHRRFSLLNPSASGRSPTSTTAPLCDICQEKRGFLFCKEDRAILCRECDVPVHTASELTMRHSRYLLTGVRLSSEPAASPAPPSEEENSSSFCCSADDAVPAPAAPATSHGGSSGSSSISEYLTTLPGWHVEDFLVDDATAEAAAAAAATSSGISANGPCQVGATDRP

SEQ ID NO: 79 - DNA, 750 bp, Oryza sativa
  misc_feature 1..750: note = Os04g45690.1, ortholog of AtBBX20 (G1888), AtBBX21 (G1482)
  source 1..750: mol_type = unassigned DNA
atgaaggtgcagtgcgacgtgtgcgcggccgaggccgcctcggtcttctgctgcgccgacgaggccgcgctgtgcgacgcgtgcgaccgccgcgtccacagcgcgaacaagctcgccgggaagcaccgccgattctccctcctccaaccgttggcgtcgtcgtcgtccgcccagaagccaccgctctgcgacatctgtcaggagaagagggggttcttgttctgcaaggaggacagggcgatcctgtgccgggagtgcgacgtcacggtgcacaccacgagcgagctgacgaggcggcacggccggttcctcctcaccggcgtgcgcctctcgtcggcgccgatggactcccccgcgccgtcggaggaagaggaggaggaagcaggggaggactacagctgcagccccagcagcgtcgccggcaccgccgcggggagcgcgagcgacgggagcagcatctccgagtacctcaccaagacgctgcccggttggcacgtcgaggacttcctcgtcgacgaggccaccgccgcctcctcctcctcagacgggctatttcagggtgggctgctggctcagatcggtggggtgccggacggttacgcggcgtgggccggccgggagcagctgcacagtggcgtcgctgtcgccgccgacgagcgggccagccgcgagcggtgggtgccgcagatgaacgcggagtggggcgccggcagcaagcgacccagggcgtcgcctccctgcttgtactgg

SEQ ID NO: 80 - protein, 250 aa, Oryza sativa
  REGION 1..250: note = Os04g45690.1, domain coordinates 5-42, 63-100, ortholog of AtBBX20 (G1888), AtBBX21 (G1482)
MKVQCDVCAAEAASVFCCADEAALCDACDRRVHSANKLAGKHRRFSLLQPLASSSSAQKPPLCDICQEKRGFLFCKEDRAILCRECDVTVHTTSELTRRHGRFLLTGVRLSSAPMDSPAPSEEEEEEAGEDYSCSPSSVAGTAAGSASDGSSISEYLTKTLPGWHVEDFLVDEATAASSSSDGLFQGGLLAQIGGVPDGYAAWAGREQLHSGVAVAADERASRERWVPQMNAEWGAGSKRPRASPPCLYW

SEQ ID NO: 81 - DNA, 753 bp, Oryza sativa
  misc_feature 1..753: note = G5159,, ortholog of AtBBX20 (G1888), AtBBX21 (G1482)
  source 1..753: mol_type = unassigned DNA
atgaaggtgcagtgcgacgtgtgcgcggccgaggccgcctcggtcttctgctgcgccgacgaggccgcgctgtgcgacgcgtgcgaccgccgcgtccacagcgcgaacaagctcgccgggaagcaccgccgattctccctcctccaaccgttggcgtcgtcgtcgtccgcccagaagccaccgctctgcgacatctgtcaggagaagagggggttcttgttctgcaaggaggacagggcgatcctgtgccgggagtgcgacgtcacggtgcacaccacgagcgagctgacgaggcggcacggccggttcctcctcaccggcgtgcgcctctcgtcggcgccgatggactcccccgcgccgtcggaggaagaggaggaggaagcaggggaggactacagctgcagccccagcagcgtcgccggcaccgccgcggggagcgcgagcgacgggagcagcatctccgagtacctcaccaagacgctgcccggttggcacgtcgaggacttcctcgtcgacgaggccaccgccggcttctcctcctcagacgggctatttcagggtgggctgctggctcagatcggtggggtgccggacggttacgcggcgtgggccggccgggagcagctgcacagtggcgtcgctgtcgccgccgacgagcgggccagccgcgagcggtgggtgccgcagatgaacgcggagtggggcgccggcagcaagcgacccagggcgtcgcctccctgcttgtactggtga

SEQ ID NO: 82 - protein, 250 aa, Oryza sativa
  REGION 1..250: note = G5159, domain coordinates 5-42, 63-100, ortholog of AtBBX20 (G1888), AtBBX21 (G1482)
MKVQCDVCAAEAASVFCCADEAALCDACDRRVHSANKLAGKHRRFSLLQPLASSSSAQKPPLCDICQEKRGFLFCKEDRAILCRECDVTVHTTSELTRRHGRFLLTGVRLSSAPMDSPAPSEEEEEEAGEDYSCSPSSVAGTAAGSASDGSSISEYLTKTLPGWHVEDFLVDEATAGFSSSDGLFQGGLLAQIGGVPDGYAAWAGREQLHSGVAVAADERASRERWVPQMNAEWGAGSKRPRASPPCLYW

SEQ ID NO: 83 - DNA, 924 bp, Oryza sativa
  misc_feature 1..924: note = Os06g49880.1, ortholog of AtBBX20 (G1888), AtBBX21 (G1482)
  source 1..924: mol_type = unassigned DNA
atgcgggtgcagtgcgacgtctgcgccgccgagccggccgcggtgctctgctgcgccgacgaggccgcgctctgctccgcctgcgaccgccgcgtccaccgcgccaaccgcctcgccagcaagcaccgccgcctcccgctcgtccacccgtcctcctcctcctccggcgacggtggcgccgccgccgcgccgctgtgcgacgtgtgcagggagaagaggggcctcgtgttctgcgtcgaggaccgcgccatcctgtgcgccgactgcgacgagcccatccactccgccaacgacctcaccgccaagcacacccgcttcctcctcgtcggcgccaagctctcccccgccgcgctcgccgaacagccgctcccgtcctccgactgcagctccgacgacgacgccgccgccgccgccaccgaggaggagtaccactcctccgccgcatccaccggcgccgcagtgagcgcgcctcttgacgcctccagcaacggcgccggtggcggaggaggagtaggagggagcagcatctccgactacctcaccaccatctgccccggctggcgcgtcgaggacctcctccccgacgacgacgccttcgccgccgccgccgcgcaggcggggaaagagaaggacgagcgcgtgccgttcctcgacgccgacctgttcgacgtggtcgccggccggccggagaagaagggcggcgcgtgggcgccgcacgtgccgcacctgccggcgtggtgcctcgacgaggtgccggtcgtcgtcgccgcgtcggcggcgccagcggcgacacctgtaaaggcgaagcagggacacgtgcgggacagccactggagcgacagcgacgcgttcgccgtgccggagttctcgccgccgccgccgccggccaagagggcgcggcccagctcgcagttctggtgcttc

SEQ ID NO: 84 - protein, 308 aa, Oryza sativa
  REGION 1..308: note = Os06g49880.1, domain coordinates 5-42, 66-103, ortholog of AtBBX20 (G1888), AtBBX21 (G1482)
MRVQCDVCAAEPAAVLCCADEAALCSACDRRVHRANRLASKHRRLPLVHPSSSSSGDGGAAAAPLCDVCREKRGLVFCVEDRAILCADCDEPIHSANDLTAKHTRFLLVGAKLSPAALAEQPLPSSDCSSDDDAAAAATEEEYHSSAASTGAAVSAPLDASSNGAGGGGGVGGSSISDYLTTICPGWRVEDLLPDDDAFAAAAAQAGKEKDERVPFLDADLFDVVAGRPEKKGGAWAPHVPHLPAWCLDEVPVVVAASAAPAATPVKAKQGHVRDSHWSDSDAFAVPEFSPPPPPAKRARPSSQFWCF

SEQ ID NO: 85 - DNA, 921 bp, Oryza sativa
  misc_feature 1..921: note = Os06g49880.2, ortholog of AtBBX20 (G1888), AtBBX21 (G1482)
  source 1..921: mol_type = unassigned DNA
atgcgggtgcagtgcgacgtctgcgccgccgagccggccgcggtgctctgctgcgccgacgaggccgcgctctgctccgcctgcgaccgccgcgtccaccgcgccaaccgcctcgccagcaagcaccgccgcctcccgctcgtccacccgtcctcctcctcctccggcgacggtggcgccgccgccgcgccgctgtgcgacgtgtgcagggagaagaggggcctcgtgttctgcgtcgaggaccgcgccatcctgtgcgccgactgcgacgagcccatccactccgccaacgacctcaccgccaagcacacccgcttcctcctcgtcggcgccaagctctcccccgccgcgctcgccgaacagccgctcccgtcctccgactgcagctccgacgacgacgccgccgccgccgccaccgaggaggagtaccactcctccgccgcatccaccggcgccgcagtgagcgcgcctcttgacgcctccagcaacggcgccggtggcggaggaggagtaggagggagcagcatctccgactacctcaccaccatctgccccggctggcgcgtcgaggacctcctccccgacgacgacgccttcgccgccgccgccgcggcggggaaagagaaggacgagcgcgtgccgttcctcgacgccgacctgttcgacgtggtcgccggccggccggagaagaagggcggcgcgtgggcgccgcacgtgccgcacctgccggcgtggtgcctcgacgaggtgccggtcgtcgtcgccgcgtcggcggcgccagcggcgacacctgtaaaggcgaagcagggacacgtgcgggacagccactggagcgacagcgacgcgttcgccgtgccggagttctcgccgccgccgccgccggccaagagggcgcggcccagctcgcagttctggtgcttc

SEQ ID NO: 86 - protein, 307 aa, Oryza sativa
  REGION 1..307: note = Os06g49880.2, domain coordinates 5-42, 66-103, ortholog of AtBBX20 (G1888), AtBBX21 (G1482)
MRVQCDVCAAEPAAVLCCADEAALCSACDRRVHRANRLASKHRRLPLVHPSSSSSGDGGAAAAPLCDVCREKRGLVFCVEDRAILCADCDEPIHSANDLTAKHTRFLLVGAKLSPAALAEQPLPSSDCSSDDDAAAAATEEEYHSSAASTGAAVSAPLDASSNGAGGGGGVGGSSISDYLTTICPGWRVEDLLPDDDAFAAAAAAGKEKDERVPFLDADLFDVVAGRPEKKGGAWAPHVPHLPAWCLDEVPVVVAASAAPAATPVKAKQGHVRDSHWSDSDAFAVPEFSPPPPPAKRARPSSQFWCF

SEQ ID NO: 87 - DNA, 957 bp, Glycine max
  misc_feature 1..957: note = Glyma04g01120.1, ortholog of AtBBX20 (G1888), AtBBX21 (G1482)
  source 1..957: mol_type = unassigned DNA
atgaagatccagtgtgctgtgtgtgacaaagtggaagcctccgtcttctgctctgcagatgaagctgctctttgccatagttgtgaccgcaccattcaccacgccaacaagcttgcaaccaaacacccacgcttctctctccattaccccacctcccaagacttccctctttgtgatatttgccaagagaggcgtgcatatctattttgccaagaagacagggcattgctatgcagggagtgtgacgttcctatccacagggcgaatgagcatacccagaagcataacaggtttcttctcaccggagtgaagctctctggtacttctttggatcctgctgcgtcctccaccaattgcacccatggctctgaaggaagaaacaacgctcgatctagaatgaacagacccagatcatcagtttccaatgaagaaaatgcaagtaattcttcttgcaaggttgaagacaacgtggctagtgacactggttcggtttcaactagcagcatttctgagtacttgatcgaaacaatacccggttactgttttgaagaccttcttgatgcttcatttgcacctaatggtttctgtaagaaccagaactatgatcgtcataggcgtttcaggaccaagatcttcaagttattaggacgtgtttggtttgagttaaggagattccaaaatcttaacgctatcgatgatcaactgtttgtaaaaatctgttggaaaatatcttcttttgtttttgttatttgcatttctttttgggctccctgctgcattctttatgatttttttttcattctctattcccgaccactgtttcggcctcttattttagtatttttttcaattttcattgggttaattaataatattttttgggggggggtccatgcttttattgacatgaacatgaattcattgaataacgcaactgaatattatgtgccactttct

SEQ ID NO: 88 - protein, 319 aa, Glycine max
  REGION 1..319: note = Glyma04g01120.1, domain coordinates 5-42, 58-95, ortholog of AtBBX20 (G1888), AtBBX21 (G1482)
MKIQCAVCDKVEASVFCSADEAALCHSCDRTIHHANKLATKHPRFSLHYPTSQDFPLCDICQERRAYLFCQEDRALLCRECDVPIHRANEHTQKHNRFLLTGVKLSGTSLDPAASSTNCTHGSEGRNNARSRMNRPRSSVSNEENASNSSCKVEDNVASDTGSVSTSSISEYLIETIPGYCFEDLLDASFAPNGFCKNQNYDRHRRFRTKIFKLLGRVWFELRRFQNLNAIDDQLFVKICWKISSFVFVICISFWAPCCILYDFFFILYSRPLFRPLILVFFSIFIGLINNIFWGGVHAFIDMNMNSLNNATEYYVPLS

SEQ ID NO: 89 - DNA, 798 bp, Glycine max
  misc_feature 1..798: note = G5365, Glyma04g02960.1, ortholog of AtBBX20 (G1888), AtBBX21 (G1482)
  source 1..798: mol_type = unassigned DNA
atgaagatccactgcgacgtgtgtaacaagcaccaggcctctttcttctgcaccgccgacgaagctgccctatgcgacggctgcgaccaccgcgtccaccacgccaacaagctcgcctccaaacaccaacgcttctccctcacccacccctctgcgaaacattttcccctctgcgatgtttgccaggagagaagagccttcgtgttttgtcagcaagatagagcgattctgtgcaaagagtgtgacgtgcccattcattctgccaacgacctcaccaagaaccatagcaggtttcttctcactgggattaagttctctgcttctgccacgccttatgattattcatcaccaccaccaccaccaccaccaccaccacccaagagaaaccctgttctcgattctccttcaacaccgtcaccgcctaagcctgggggaaactcgctaacaaacgaagaagaacctggtttcacaggtagcagcatttcagagtacttgataaactctatcccaggcatgaagttcgaagatttcctcgattcacattctcttccctttgcttgctccaagaatagtgatgacatgttgtcgctgtttggtgagggaaacatggtttctttctcacccggtgggttctgggttcctcaagcaccaccatcttcagtacaaatggatcggcagagtgggtacagagagacaagggaaggtagcattagatcaagttttggagatgataatttcatagttccacagatgagtcctcccagcaatgtgtctaataagagatccaggcttctctgg

SEQ ID NO: 90 - protein, 266 aa, Glycine max
  REGION 1..266: note = G5365, Glyma04g02960.1, domain coordinates 5-42, 58-95, ortholog of AtBBX20 (G1888), AtBBX21 (G1482)
MKIHCDVCNKHQASFFCTADEAALCDGCDHRVHHANKLASKHQRFSLTHPSAKHFPLCDVCQERRAFVFCQQDRAILCKECDVPIHSANDLTKNHSRFLLTGIKFSASATPYDYSSPPPPPPPPPPKRNPVLDSPSTPSPPKPGGNSLTNEEEPGFTGSSISEYLINSIPGMKFEDFLDSHSLPFACSKNSDDMLSLFGEGNMVSFSPGGFWVPQAPPSSVQMDRQSGYRETREGSIRSSFGDDNFIVPQMSPPSNVSNKRSRLLW

SEQ ID NO: 91 - DNA, 582 bp, Glycine max
  misc_feature 1..582: note = Glyma04g02960.2, ortholog of AtBBX20 (G1888), AtBBX21 (G1482)
  source 1..582: mol_type = unassigned DNA
atgaagatccactgcgacgtgtgtaacaagcaccaggcctctttcttctgcaccgccgacgaagctgccctatgcgacggctgcgaccaccgcgtccaccacgccaacaagctcgcctccaaacaccaacgcttctccctcacccacccctctgcgaaacattttcccctctgcgatgtttgccaggagagaagagccttcgtgttttgtcagcaagatagagcgattctgtgcaaagagtgtgacgtgcccattcattctgccaacgacctcaccaagaaccatagcaggtttcttctcactgggattaagttctctgcttctgccacgccttatgattattcatcaccaccaccaccaccaccaccaccaccacccaagagaaaccctgttctcgattctccttcaacaccgtcaccgcctaagcctgggggaaactcgctaacaaacgaagaagaacctggtttcacaggtagcagcatttcagagtacttgataaactctatcccaggcatgaagttcgaagatttcctcgattcacattctcttccctttgcttgctccaagcaattgatcatagtt

SEQ ID NO: 92 - protein, 194 aa, Glycine max
  REGION 1..194: note = Glyma04g02960.2, domain coordinates 5-42, 58-95, ortholog of AtBBX20 (G1888), AtBBX21 (G1482)
MKIHCDVCNKHQASFFCTADEAALCDGCDHRVHHANKLASKHQRFSLTHPSAKHFPLCDVCQERRAFVFCQQDRAILCKECDVPIHSANDLTKNHSRFLLTGIKFSASATPYDYSSPPPPPPPPPPKRNPVLDSPSTPSPPKPGGNSLTNEEEPGFTGSSISEYLINSIPGMKFEDFLDSHSLPFACSKQLIIV

SEQ ID NO: 93 - DNA, 714 bp, Glycine max
  misc_feature 1..714: note = Glyma06g01140.1, ortholog of AtBBX20 (G1888), AtBBX21 (G1482)
  source 1..714: mol_type = unassigned DNA
atgaagatccagtgtgatgtgtgtgacaaagtggaagcctccgtcttctgtcctgcagatgaagctgctctttgccatagctgtgatcgcaccattcaccgcgccaacaagcttgcaaccaaacacgcccgtttctctctccactaccccacctccaaagacttccctctttgtgatatatgccaagagaggcgtgcatatctattttgccaagaagacagggcgttgttatgcagggaatgcgacgttcctatccacagagccaatgagcatacccaaaagcataacaggtttcttctcacgggtgtgaagctctctggtacttgtttagatcctgcttcgtcctccaccaattacaccaataataatagagtcactggctctgaaggaagaaacgacgctcgatctagaatgaacagacccagatcatcagtttccaatgaagaaaatgcaagtaattcttcttgcaaggttgaagacaacgtggctagtgacactggttcgatttcaactagcagcatttctgagtacttgattgaaacaatacccggttactgttttgaagacctccttgatgcttcgtttgcacctaatggtttcttaacttgtttttcttctcagaagcagaactttgatcatccctgggcgtttcaggaccaaaagcgaacaagtggagacactatggccgtgccgttcctgaaactagtcatccat

SEQ ID NO: 94 - protein, 238 aa, Glycine max
  REGION 1..238: note = Glyma06g01140.1, domain coordinates 5-42, 58-95, ortholog of AtBBX20 (G1888), AtBBX21 (G1482)
MKIQCDVCDKVEASVFCPADEAALCHSCDRTIHRANKLATKHARFSLHYPTSKDFPLCDICQERRAYLFCQEDRALLCRECDVPIHRANEHTQKHNRFLLTGVKLSGTCLDPASSSTNYTNNNRVTGSEGRNDARSRMNRPRSSVSNEENASNSSCKVEDNVASDTGSISTSSISEYLIETIPGYCFEDLLDASFAPNGFLTCFSSQKQNFDHPWAFQDQKRTSGDTMAVPFLKLVIH

SEQ ID NO: 95 - DNA, 735 bp, Glycine max
  misc_feature 1..735: note = G5367. Glyma06g02970.1, ortholog of AtBBX20 (G1888), AtBBX21 (G1482)
  source 1..735: mol_type = unassigned DNA
atgaagatccagtgcgacgtgtgtaacaaacagcaggcatcgttgttctgcaccgccgacgaagccgccctatgcgacggctgcgaccaccgcgtccaccacgccaacaagctcgcctccaaacaccaacgcttctccctcagccacccctctgcaaaacattttcctctctgcgatgtttgccaggagagaagagcctttgtgttttgtcagcaagatagagcgattctgtgcaaagagtgtgacgtgcccgttcattctgccaacgacctcaccaagaaccataacaggtttcttctcactgggattaagttttctgccctcgattctccttcaacacctcctaagcctgcaggtggaaactccctaacaaatcaacaaccacaacaacaaactggtttcacaggtagcagcatatcagaatacttgataaacactatcccaggcatggagttcgaagatttcctcgattcacattctcttccctttgcttgctccaagaatagtgatgacatgatgttgtcgatgtttggtgagggaaacatggtttcgttctcagccggagggatctgggttcctcaagcaccatcttcagtacaaatggatcagcagagtgggtacaaagacacatgggaaactagcattagatcaagttttggggatgatagtttattagttccacagatgactcctcccagcaatgtgtttaataataagagatccaggcttctatgg

SEQ ID NO: 96 - protein, 245 aa, Glycine max
  REGION 1..245: note = G5367, Glyma06g02970.1, domain coordinates 5-42, 58-95, ortholog of AtBBX20 (G1888), AtBBX21 (G1482)
MKIQCDVCNKQQASLFCTADEAALCDGCDHRVHHANKLASKHQRFSLSHPSAKHFPLCDVCQERRAFVFCQQDRAILCKECDVPVHSANDLTKNHNRFLLTGIKFSALDSPSTPPKPAGGNSLTNQQPQQQTGFTGSSISEYLINTIPGMEFEDFLDSHSLPFACSKNSDDMMLSMFGEGNMVSFSAGGIWVPQAPSSVQMDQQSGYKDTWETSIRSSFGDDSLLVPQMTPPSNVFNNKRSRLLW

SEQ ID NO: 97 - DNA, 864 bp, Glycine max
  misc_feature 1..864: note = Glyma11g12060.1, ortholog of AtBBX20 (G1888), AtBBX21 (G1482)
  source 1..864: mol_type = unassigned DNA
atgaagatccagtgtgatgtgtgtcacaatgaagttgcttcctttttctgcccctctgatgaagcctctctctgccatgcttgtgatcgcaccatccaccatgccaacaagctcgcagacaaacacaaacgcttctcccttcaccaccccacttccaaagactcccctctttgtgatatctgtcatgagaggcgtgcatatcttttttgcaaagaggacagagcaatactatgtagagaatgtgatctttctattcatggggtcaacgaacataccaagaaacacaacaggtttcttctcactggcgtaaagatcggtgctgatgcttcagacccaacatcactttcctcaaatgacactgccattgaagaaagaacaacctctagttctaaaataaacagaccaataagttctttgtcaaatgagaatatcagttcttctcccacggttggagacaacatggcttgtgacacgggttcggtttcaactagcagcatttctgagtacttgattcagaccattcccggttactgcatggaagatcttttagatgcttcatttgcatctaatggtttatctaaggattatgagcaccaatcagcgtttcagaaccaagatgttcaagtcagcatgtgttcgtttccactgcaaagttgggtaccacaatctcaagctggatcccctcaactaattagcactacctcgaatcttcatccccagattaattctttagttggagtaatggaaatgcctaaagccaaagctagtgaaggatactccaattgcatatataattatgactactcagcttacaaagttccttcaatttgtcctccattgatcaaaaaatgtaaacgctctcgt

SEQ ID NO: 98 - protein, 288 aa, Glycine max
  REGION 1..288: note = Glyma11g12060.1, domain coordinates 5-42, 58-95, ortholog of AtBBX20 (G1888), AtBBX21 (G1482)
MKIQCDVCHNEVASFFCPSDEASLCHACDRTIHHANKLADKHKRFSLHHPTSKDSPLCDICHERRAYLFCKEDRAILCRECDLSIHGVNEHTKKHNRFLLTGVKIGADASDPTSLSSNDTAIEERTTSSSKINRPISSLSNENISSSPTVGDNMACDTGSVSTSSISEYLIQTIPGYCMEDLLDASFASNGLSKDYEHQSAFQNQDVQVSMCSFPLQSWVPQSQAGSPQLISTTSNLHPQINSLVGVMEMPKAKASEGYSNCIYNYDYSAYKVPSICPPLIKKCKRSR

SEQ ID NO: 99 - DNA, 828 bp, Glycine max
  misc_feature 1..828: note = G5396 Glyma14g40650.1, ortholog of AtBBX20 (G1888), AtBBX21 (G1482)
  source 1..828: mol_type = unassigned DNA
atgaagatccagtgcgacgtgtgcaacaaacacgaggcctccgtcttctgcacagccgacgaagccgccctctgcgacggctgcgaccaccgtgtccaccatgccaacaaactcgcctccaaacaccaacgcttctctcttctccgcccttctcataaacaacaccctctctgcgatatttgccaggagagaagagccttcacgttctgtcagcaagacagagcgattctctgcaaagagtgtgacgtgtcaattcactctgccaacgaacacacccttaagcacgataggttccttctcactggtgttaaactcgcagcttctgccatgcttcgttcatcacaaactacctctgattcaaactcaaccccttctcttcttaacgtttcacatcaaactactccacttccatcttccaccaccaccaccaccaccaacaacaacaacaacaaggttgctgttgaaggaactggttcaacgagtgctagcagcatatcagagtatttgatagagactcttcctgggtggcaagttgaggactttctcgattcatattttgttccctttggtttctgtaagaatgatgaagtgttgccacggttggatgctgacgtggaggggcatatgggttcgttttcaaccgagaacatggggatctgggttcctcaagcgccaccacctcttgtgtgttcttcacaaatggatcgggtgatagttcaaagtgagaccaacatcaaaggtagcagcatatcgaggttgaaggatgatactttcactgttccacagattagtcctccctccaattccaagagagccagatttctatgg

SEQ ID NO: 100 - protein, 276 aa, Glycine max
  REGION 1..276: note = G5396 Glyma14g40650.1, domain coordinates 5-42, 58-95, ortholog of AtBBX20 (G1888), AtBBX21 (G1482)
MKIQCDVCNKHEASVFCTADEAALCDGCDHRVHHANKLASKHQRFSLLRPSHKQHPLCDICQERRAFTFCQQDRAILCKECDVSIHSANEHTLKHDRFLLTGVKLAASAMLRSSQTTSDSNSTPSLLNVSHQTTPLPSSTTTTTTNNNNNKVAVEGTGSTSASSISEYLIETLPGWQVEDFLDSYFVPFGFCKNDEVLPRLDADVEGHMGSFSTENMGIWVPQAPPPLVCSSQMDRVIVQSETNIKGSSISRLKDDTFTVPQISPPSNSKRARFLW

SEQ ID NO: 101 - DNA, 834 bp, Glycine max
  misc_feature 1..834: note = G5400 Glyma17g37430.1, ortholog of AtBBX20 (G1888), AtBBX21 (G1482)
  source 1..834: mol_type = unassigned DNA
atgaagatccagtgcgacgtttgcaacaaacacgaggcctccgtcttctgcaccgccgatgaagccgccctctgcgacggctgcgaccaccgtgtccaccatgccaacaaactcgcctccaaacaccaacgcttctctcttctccgcccttctcctaaacaacaccctctctgcgatatttgccaggagagaagagcctttacattctgtcagcaagacagagcgattctctgcaaagagtgtgacgtgtcaattcactctgccaacgaacacacccttaagcacgataggttccttctcaccggtgttaaactttctgcttctgctatgcttcgttcgtcagaaactacctctgattcaaactcaaacccttctcttcttaacttttcacaccaaacaacattactacctccatcttccaccaccaccaccaccaccagcaacaacaacaacaacaaggttgctgttgaaggaactggttcaactagtgctagcagcatatcggagtatttgatagagactcttcctgggtggcaagttgaagactttcttgattcatattctgttccctttggtttctgtaaggatgatgaagtgttgccacggtttgatggtgaaatggaggggcatctgagttctttctcaaccgagaacatggggatctgggttcctcaagcgccaccaactcttatgtgttcttcacaaatggatcgggtgatagttcacggtgagaccaatatcaaaggtagcagcagatcaaggttgaaggatgataatttcactgttccacagattagtcctccttccaattccaagagagccagatttctgtgg

SEQ ID NO: 102 - protein, 278 aa, Glycine max
  REGION 1..278: note = G5400 Glyma17g37430.1, domain coordinates 5-42, 58-95, ortholog of AtBBX20 (G1888), AtBBX21 (G1482)
MKIQCDVCNKHEASVFCTADEAALCDGCDHRVHHANKLASKHQRFSLLRPSPKQHPLCDICQERRAFTFCQQDRAILCKECDVSIHSANEHTLKHDRFLLTGVKLSASAMLRSSETTSDSNSNPSLLNFSHQTTLLPPSSTTTTTTSNNNNNKVAVEGTGSTSASSISEYLIETLPGWQVEDFLDSYSVPFGFCKDDEVLPRFDGEMEGHLSSFSTENMGIWVPQAPPTLMCSSQMDRVIVHGETNIKGSSRSRLKDDNFTVPQISPPSNSKRARFLW

SEQ ID NO: 103 - DNA, 1116 bp, Arabidopsis thaliana
  misc_feature 1..1116: note = AtBBX7 (G2440), AT3G07650.1, paralog of AtBBX8 (G1479)
  source 1..1116: mol_type = unassigned DNA
atgggttacatgtgtgacttctgtggtgaacaaagatctatggtgtactgtcgatccgatgcagcttgtttgtgtctctcctgtgaccggagtgttcattccgctaatgcattgtccaaacgacattctcggacacttgtctgcgagagatgcaatgctcagcctgctactgtcaggtgtgttgaagaaagggtttcactctgtcaaaactgtgactggtctggccacaacaacagcaacaacaacaattcttcgtcttcttctacttcgccgcagcagcataagaggcaaaccataagttgctattcgggttgcccttccagctcagaactcgcttccatttggtctttctgtttggacttagctggacaatctatttgtgaacaagaactgggtatgatgaatatagacgatgatggtcctaccgacaagaaaacttgtaatgaggataagaaagatgtcttggttggatcctcttcaattcctgaaaccagttctgtaccacaaggaaaatcatcttctgctaaggatgttggaatgtgtgaagatgacttctacgggaaccttggtatggatgaagttgacatggcacttgagaactatgaagaactctttggtaccgcctttaacccctctgaagagctattcgggcatggtggaatcgatagccttttccataagcaccaaacagctccagagggagggaattcggtgcagccagcaggcagtaatgattcgttcatgagttcgaaaactgagccaataatttgcttcgcatcgaagccagcacattcgaacatatccttctctggagtcactggagaaagtagtgctggagatttccaagaatgtggtgcatcatcttccatacagctctcgggggagccaccatggtatcctccaacattacaggataacaatgcctgctcacattcagtaacccgtaataacgcagttatgcgttacaaggagaagaagaaggctcgcaagtttgataagagagtgaggtatgcttcccgcaaagcaagagctgatgtgagacggcgtgtaaaggggcgatttgtcaaagctggtgaagcttatgattacgaccctctcaccccaaccagaagttat

SEQ ID NO: 104 - protein, 372 aa, Arabidopsis thaliana
  REGION 1..372: note = AtBBX7 (G2440), AT3G07650.1, domain coordinates 5-42, 48-76, 315-356, paralog of AtBBX8 (G1479)
MGYMCDFCGEQRSMVYCRSDAACLCLSCDRSVHSANALSKRHSRTLVCERCNAQPATVRCVEERVSLCQNCDWSGHNNSNNNNSSSSSTSPQQHKRQTISCYSGCPSSSELASIWSFCLDLAGQSICEQELGMMNIDDDGPTDKKTCNEDKKDVLVGSSSIPETSSVPQGKSSSAKDVGMCEDDFYGNLGMDEVDMALENYEELFGTAFNPSEELFGHGGIDSLFHKHQTAPEGGNSVQPAGSNDSFMSSKTEPIICFASKPAHSNISFSGVTGESSAGDFQECGASSSIQLSGEPPWYPPTLQDNNACSHSVTRNNAVMRYKEKKKARKFDKRVRYASRKARADVRRRVKGRFVKAGEAYDYDPLTPTRSY

SEQ ID NO: 105 - DNA, 1119 bp, Arabidopsis thaliana
  misc_feature 1..1119: note = AtBBX8 (G1479), AT5G48250.1, paralog of AtBBX7 (G2440)
  source 1..1119: mol_type = unassigned DNA
atgggttatatgtgtgatttctgtggtgaacaaagatcaatggtgtattgtcgatctgatgcagcgtgtttgtgtctttcgtgtgaccgtaatgttcattctgctaatgctctatcgaaacgtcattcgcggactttggtatgtgagaggtgtaatgcacagccagcttctgtccggtgtagtgatgagagggtttctctatgtcaaaactgtgattggtcaggccatgacggcaagaattctactactacttcacatcataaaaggcaaaccatcaattgttattctggttgtccctcgagtgccgaattatcctcgatatggtctttctgtatggatttgaatatctcttctgctgaagaatccgcttgtgagcaaggaatgggtttgatgactatagatgaagatggcacaggagaaaaatctggtgtccaaaaaattaatgtagaacagcctgaaaccagttcggctgcacaaggtatggatcactctagtgttccagaaaattcatcaatggctaaggagcttggagtatgtgaggatgacttcaatggaaatctcatttcagatgaagtagacttggctcttgagaactatgaagaactctttggttcagccttcaactcatcaagatatctcttcgagcacggtggaattggaagtcttttcgagaaagatgaagctcatgagggctcaatgcagcagccagcattaagcaataatgcatctgcagattcgttcatgacttgtagaaccgagccaataatttgctattcatcaaagccagcacattcgaatatctccttctctggcatcactggagaaagtaatgctggagatttccaagattgtggggcgtcatcgatgaagcagctttcaagggagccacaaccatggtgccatccaacagcacaggacattattgcttcctcacatgcaacaacacgtaataacgctgttatgcgttacaaggaaaagaagaaggctcgcaagtttgacaagcgagtaaggtatgtctctaggaaagaaagggctgatgtgagacggcgtgtgaaaggacggtttgtcaagtccggtgaagcttatgattacgacccgatgagcccaacaagaagctac

SEQ ID NO: 106 - protein, 373 aa, Arabidopsis thaliana
  REGION 1..373: note = AtBBX8 (G1479), AT5G48250.1, domain coordinates 5-42, 48-76, 316-357, paralog of AtBBX7 (G2440)
MGYMCDFCGEQRSMVYCRSDAACLCLSCDRNVHSANALSKRHSRTLVCERCNAQPASVRCSDERVSLCQNCDWSGHDGKNSTTTSHHKRQTINCYSGCPSSAELSSIWSFCMDLNISSAEESACEQGMGLMTIDEDGTGEKSGVQKINVEQPETSSAAQGMDHSSVPENSSMAKELGVCEDDFNGNLISDEVDLALENYEELFGSAFNSSRYLFEHGGIGSLFEKDEAHEGSMQQPALSNNASADSFMTCRTEPIICYSSKPAHSNISFSGITGESNAGDFQDCGASSMKQLSREPQPWCHPTAQDIIASSHATTRNNAVMRYKEKKKARKFDKRVRYVSRKERADVRRRVKGRFVKSGEAYDYDPMSPTRSY

SEQ ID NO: 107 - DNA, 1218 bp, Zea mays
  misc_feature 1..1218: note = ACF80403.1, ortholog of AtBBX7 (G2440), AtBBX8 (G1479)
  source 1..1218: mol_type = unassigned DNA
atggttcctctctgtggtttctgtgggaaacaaaggtcaatgatctactgcagatcggatgcggcatcattgtgcttatcatgcgaccgtagcgttcattcagctaatgcactgtctcggcgtcatagaaggacccttctttgtgatcgttgtggtttacagcctgcatcagtccgatgtcttgaggacaacacatcactttgccaaaactgtgattggaatgggcatgatgcagcatcaggggcttctgggcataaaaggcaggccataaactgttactcagggtgcccatcatcagcagagctttcgagaatctggtcatttattatcgatatcccgactgtagctgctgagcccaactgtgaggatgggctaagcatgatgacaattgatgacagtgatgtgactaatcatcacggtgcttcagatgataaacgattgttggaaatagctaacacagcactcatgagtgatccaccttcacctgacaagcttaaacctctgataggctcttcttctggagatgggtttgatgttctgcctctagccacagatcagcctgctggaccagtttcagcgacacctaaggtaccttatgccagagatgacaataagttcaatgatggcatgtatgaagacttatgtgtggatgatgctgacctgacattcgagaattatgaagagctattcggtacctctcacattcgaatagaggaactctttgatgacgccggaattgacagttactttgaaatgaaggaaacaccgccttttgatttcaatgagcagcccaaaattgtgcagctacaatgtagcgatgtggtaccagctgattgtgcgatgtcaaacacaggggaaagggctgattccagcctttgtattcctgttaggcaggtcagatctagtatatcccatcccttatctggtttgactggtgagagcagcgctggagatcaccaagactgtggggtgtcaccaattctcctcatgggcgagccaccctggtattctcctggtccagaaggctcattagctggaggaagcagagatagtgctctcacacgatacaaggagaagaagaagaaaagaatgtttgacaagaagatcagatatgcttctcgcaaggctagggcagacgtgaggaagagggtcaagggacggtttataaaggctggcgaagcgtatgactatgatccactaagtcaaactagaagctac

SEQ ID NO: 108 - protein, 406 aa, Zea mays
  REGION 1..406: note = ACF80403.1, domain coordinates 5-42, 48-85, 349-390, ortholog of AtBBX7 (G2440), AtBBX8 (G1479)
MVPLCGFCGKQRSMIYCRSDAASLCLSCDRSVHSANALSRRHRRTLLCDRCGLQPASVRCLEDNTSLCQNCDWNGHDAASGASGHKRQAINCYSGCPSSAELSRIWSFIIDIPTVAAEPNCEDGLSMMTIDDSDVTNHHGASDDKRLLEIANTALMSDPPSPDKLKPLIGSSSGDGFDVLPLATDQPAGPVSATPKVPYARDDNKFNDGMYEDLCVDDADLTFENYEELFGTSHIRIEELFDDAGIDSYFEMKETPPFDFNEQPKIVQLQCSDVVPADCAMSNTGERADSSLCIPVRQVRSSISHPLSGLTGESSAGDHQDCGVSPILLMGEPPWYSPGPEGSLAGGSRDSALTRYKEKKKKRMFDKKIRYASRKARADVRKRVKGRFIKAGEAYDYDPLSQTRSY

SEQ ID NO: 109 - DNA, 1221 bp, Zea mays
  misc_feature 1..1221: note = ACG34885.1, ortholog of AtBBX7 (G2440), AtBBX8 (G1479)
  source 1..1221: mol_type = unassigned DNA
atgggcgctctctgtgatttctgtggggagcagaggtctatggtttactgccgatcagatgcagcatccttgtgtttatcatgtgaccgtaatgtgcattcagctaatgctctttctcggcgtcatacaaggacacttttatgtgaccggtgtgcttcacagcctgctatggtccgctgtctagcagaaaatgcctcgctttgccaaaattgtgattggaatggccacattgcaggatcctcatctgccggacacaaaaggcagactataaattgctattcaggatgtccatcgtctgcagaactttctagaatatggtccttcgtttcagatattcctaatgtagcacctgagccaaactgtgagcaaggaataagcatgatgagcatcagcgacagtggtgtcagtaatcaagataatgctgcaggggacagtattttgttagatatagctagtgcaacacttgtaagtgatattggtacttgtgacaagcttttggtaggttcctcttcaggagctggagtgaacctacttccactggttactggtcaaacagaaacagctggatctgtggattccacaccagataaggttccctatacgccagataaagatatgttcagtaaggacagcatatatgaagatttttgtgtggatgacgctgaccttgctttcgaaaattatgaagaactctttggtacctctcatattcaaacagaacaactctttgatgatgctggaatagacatttactttgaaatgaaggaagcacctgctgggaattccactgagtccaagctgaagcaacctgcaaatagcaatgcggtatctgccgactctgggatgtcgaaccctggagtgaaaggtgattccagtgtttgtacccctttgaggcaggctaggtctagtctgtctctttcctttgctggtttgactggtgagagtagtgcgggagacaaccaagattgtgtggtatcatcattgcttcttatgggtgagcctccttggcagcctcctggccccgagggctcaattgctggaggtagtagagacagtgcactcacacgatacaaggagaagaaaatgagaagaaagttcgacaagaaaatcaggtacgcttctcgtaaggctagagcagatgtgcgaaagagggtaaagggtcggtttgttaaggctggtgaagcatatgactatgatcctctctgccaaacgaggagttac

SEQ ID NO: 110 - protein, 407 aa, Zea mays
  REGION 1..407: note = ACG34885.1, domain coordinates 5-42, 48-85, 350-391, ortholog of AtBBX7 (G2440), AtBBX8 (G1479)
MGALCDFCGEQRSMVYCRSDAASLCLSCDRNVHSANALSRRHTRTLLCDRCASQPAMVRCLAENASLCQNCDWNGHIAGSSSAGHKRQTINCYSGCPSSAELSRIWSFVSDIPNVAPEPNCEQGISMMSISDSGVSNQDNAAGDSILLDIASATLVSDIGTCDKLLVGSSSGAGVNLLPLVTGQTETAGSVDSTPDKVPYTPDKDMFSKDSIYEDFCVDDADLAFENYEELFGTSHIQTEQLFDDAGIDIYFEMKEAPAGNSTESKLKQPANSNAVSADSGMSNPGVKGDSSVCTPLRQARSSLSLSFAGLTGESSAGDNQDCVVSSLLLMGEPPWQPPGPEGSIAGGSRDSALTRYKEKKMRRKFDKKIRYASRKARADVRKRVKGRFVKAGEAYDYDPLCQTRSY

SEQ ID NO: 111 - DNA, 1218 bp, Zea mays; identical to SEQ ID NO: 107
  misc_feature 1..1218: note = ACG44361.1, ortholog of AtBBX7 (G2440), AtBBX8 (G1479)
  source 1..1218: mol_type = unassigned DNA

SEQ ID NO: 112 - protein, 406 aa, Zea mays; identical to SEQ ID NO: 108
  REGION 1..406: note = ACG44361.1, domain coordinates 5-42, 48-85, 349-390, ortholog of AtBBX7 (G2440), AtBBX8 (G1479)

SEQ ID NO: 113 - DNA, 1221 bp, Zea mays
  misc_feature 1..1221: note = ACN25813.1, ortholog of AtBBX7 (G2440), AtBBX8 (G1479)
  source 1..1221: mol_type = unassigned DNA
atggcttctctctgtgatttctgtgggaaacaaaggtcaatgatctactgcagatcagatgcggcatcattgtgcttatcatgcgaccgtaatgttcattcagctaatgcactgtctcgacgtcatacaaggacccttctttgtgatcgttgtggttcacagcctgcatcagtccgatgtcttgaggacaacgcatcactttgccaaaactgtgactggaatgggcatgatgcagaatcaggggcttctggacataaaaggcaagccataaactgctactcagggtgcccatcatctgcagagctttcgagaatctggtcatttattactgatatcccgactgtagcagctgagcccgactatgaggatggactaagcatgatgacaattgatggcagtgatgttactaatcgtcacgatacttcagatgataaaagattgttggaaatagctaacacaacactcatgagcgatccaccttcagccgacaagcttaaatctccgacaggctcttcttctggagatgggtttgatgttatgactctagccacagatcagcctgctggaccagtttcagcaacgcctaaggtacctaatgccagagatgatgataagttcaatgatggcatgtacgaagacttgtgtgtagatgatgctgaccttacattcgaggattatgaagagctatttggtacctctcacattcgaacagaggaactctttgatgacgctggaattgacggttacttcgaattgaaagagacaccacctttttatttcaacgagcagcccaaagctatgcagatagaatgtggcaacgtggtatcagctgattgtgcgatgtcaaacccaggggcaagggctgattccagcctttgtattcccgtcaggcaggtcagatctagtatatcccactccttatctggtctgactggtgagagcagcgctggagatcaccacgactgtggggtgtcaccgatgctcctcatgggtgagccaccctggcattctcctggtggtccagaaggctcagtcgctggaggcagcagagatagcgctctcacacggtacaaggagaagaagaagaggagaaagtttgacaagaagatcagatatgcttctcgcaaggctagggcggacgtgaggaagcgggtcaagggacggttcataaaggccggtgaagcgtacgactatgatccgctaagccagactagaagctac

SEQ ID NO: 114 - protein, 407 aa, Zea mays
  REGION 1..407: note = ACN25813.1, domain coordinates 5-42, 48-85, 350-391, ortholog of AtBBX7 (G2440), AtBBX8 (G1479)
MASLCDFCGKQRSMIYCRSDAASLCLSCDRNVHSANALSRRHTRTLLCDRCGSQPASVRCLEDNASLCQNCDWNGHDAESGASGHKRQAINCYSGCPSSAELSRIWSFITDIPTVAAEPDYEDGLSMMTIDGSDVTNRHDTSDDKRLLEIANTTLMSDPPSADKLKSPTGSSSGDGFDVMTLATDQPAGPVSATPKVPNARDDDKFNDGMYEDLCVDDADLTFEDYEELFGTSHIRTEELFDDAGIDGYFELKETPPFYFNEQPKAMQIECGNVVSADCAMSNPGARADSSLCIPVRQVRSSISHSLSGLTGESSAGDHHDCGVSPMLLMGEPPWHSPGGPEGSVAGGSRDSALTRYKEKKKRRKFDKKIRYASRKARADVRKRVKGRFIKAGEAYDYDPLSQTRSY

SEQ ID NO: 115 - DNA, 1218 bp, Zea mays; identical to SEQ ID NO: 107
  misc_feature 1..1218: note = NP_001131822.1, ortholog of AtBBX7 (G2440), AtBBX8 (G1479)
  source 1..1218: mol_type = unassigned DNA

SEQ ID NO: 116 - protein, 406 aa, Zea mays; identical to SEQ ID NO: 108
  REGION 1..406: note = NP_001131822.1, domain coordinates 5-42, 48-85, 349-390, ortholog of AtBBX7 (G2440), AtBBX8 (G1479)

SEQ ID NO: 117 - DNA, 1221 bp, Zea mays; identical to SEQ ID NO: 109
  misc_feature 1..1221: note = NP_001149294.1, ortholog of AtBBX7 (G2440), AtBBX8 (G1479)
  source 1..1221: mol_type = unassigned DNA

SEQ ID NO: 118 - protein, 407 aa, Zea mays; identical to SEQ ID NO: 110
  REGION 1..407: note = NP_001149294.1, domain coordinates 5-42, 48-85, 350-391, ortholog of AtBBX7 (G2440), AtBBX8 (G1479)

SEQ ID NO: 119 - DNA, 1137 bp, Populus trichocarpa
  misc_feature 1..1137: note = Pt_247140, ortholog of AtBBX7 (G2440), AtBBX8 (G1479)
  source 1..1137: mol_type = unassigned DNA
atgggttacgtttgtgatttttgtggggaacaaaggtccatggtttattgccggtcagatgctgcaagcttatgtttatcatgcgatcgaaatgttcattctgctaatgctttgtccaagcgtcattcacggaccctcttatgtgaaagatgcaattcacaaccagcgttagttagatgtgctgaagaaagaatctctctatgtcaaaattgtgattggattggccatggcacctcgacatcagcttccacacatagaaggcaaacgatcaattcttactctggctgtccatcagcttctgagctttcttcaatatggtcatttgttttagacttcccttccagaggtgaatctacttgtgagcaggagttgggtttgatgagcattgctgaaaatagcaccacagactcctggggtcctacagagaatactattggccaaaatgcatctggagttgttgaagttaatgacaggcgtgaaatagctaaatcaggtgtttggcttgggtcttcttcgatacctgaatcgagctctgtgccaaacaatctagaccagacaaccagatccgcaaatacatctttgcctaaggacttcaatatggatgaaatggatttgagtctcgaaaactatgaagaactctttggtgttaccctcaataattcagaagagcttcttgagaatggtggcattgatagcttatttgggacaaaggacatgtctgtggtcaacgccgtacagccagcaagcagcaacgcagcatctgctgattctatgatgagcaataagactgaaccaatcctttgttttacggaaaagcaaggccattccagcctctcattttctggccttaatgtagagagtagtgctgcagattatcaagattgtggagcatcttcaatgcttcttatgggagagcctccctggtgccccccatgccccgagagtcccttctcatcagctaaccggagtgatgctgtaatgcgttacaaggaaaagaagaagacacggatgtttgaaaagaaagtgaggtatgcatctcgcaaagcaagggcagatgtaagaaggcgtgtgaagggtcgttttgtcaaagctggtgaagcttatgattacgacccacttagccaaacgagaagtttc

SEQ ID NO: 120 - protein, 379 aa, Populus trichocarpa
  REGION 1..379: note = Pt_247140, domain coordinates 5-42, 48-85, 322-363, ortholog of AtBBX7 (G2440), AtBBX8 (G1479)
MGYVCDFCGEQRSMVYCRSDAASLCLSCDRNVHSANALSKRHSRTLLCERCNSQPALVRCAEERISLCQNCDWIGHGTSTSASTHRRQTINSYSGCPSASELSSIWSFVLDFPSRGESTCEQELGLMSIAENSTTDSWGPTENTIGQNASGVVEVNDRREIAKSGVWLGSSSIPESSSVPNNLDQTTRSANTSLPKDFNMDEMDLSLENYEELFGVTLNNSEELLENGGIDSLFGTKDMSVVNAVQPASSNAASADSMMSNKTEPILCFTEKQGHSSLSFSGLNVESSAADYQDCGASSMLLMGEPPWCPPCPESPFSSANRSDAVMRYKEKKKTRMFEKKVRYASRKARADVRRRVKGRFVKAGEAYDYDPLSQTRSF

SEQ ID NO: 121 - DNA, 1137 bp, Populus trichocarpa
  misc_feature 1..1137: note = Pt_409151, ortholog of AtBBX7 (G2440), AtBBX8 (G1479)
  source 1..1137: mol_type = unassigned DNA
atgggttatatttgtgatttttgtggggagcaaaggtccatggtttattgccggtcagatgctgcgtgcttatgcttatcatgcgatcaaattgttcattctgctaatgccttgtccaagcgtcactcacggaccctcttatgtgaaagatgcaattcacaacctgcattagttagacgcgtggaagaaagaatctctctttgtcagaattgtgattggatgggctatggctcctcaacatcagcttccacacataaaaggcaaacgatcaattgttactttggctgtccatcagtttctgagctttcttcaaaatggccattcattttagactccccttctgggggtggatctacttgtgagcaggagttgggtttaatgagcattgctgaaaatagcaccaaaaacacctggggtccgacagagaatactatttgccataatgcatctggaattgttggggcttcttcagtacctgaatcaagctctgtgccaaaaaatttagaccagccaaccagatctccaaattcatctttgccaaaggtatgtgatgctgatctttatgaggacttcaatatggctgaaatggatttgaatcttgaaaactatgaagaactatttggtgttaccctcaataattcagaagagctttttgagaatggtggcattgatagcttatttgggacaaaggacatgcccattgttgattccaattttggactggtcaacacaatacagccaacatgcagcaacgcaccatctactgattctatgattagtagtaagactgaaccaatcctttgttttacggcaaagcaaggccattccagcctatcattttctggccttactggagagagtaatgctggagattatcaagactgtggagcatcttcaatgcttctcatgggagagccttcatggtgtcccccgtgccttgagagtcctttaccttcagctaaccgaagtgatgcggtaaagcgttacatggaaaagaaaaagacacgcaagtttgaaaagaaagtgaggtatgcgtctcgcaaagcaagggcagatgtaagaaggcgtgtgaagggtcgttttgtcaaagctggtgatgcttatgattatgacccactgggccaaacaagaagtttc

SEQ ID NO: 122 - protein, 379 aa, Populus trichocarpa
  REGION 1..379: note = Pt_409151, domain coordinates 5-42, 48-85, 322-363, ortholog of AtBBX7 (G2440), AtBBX8 (G1479)
MGYICDFCGEQRSMVYCRSDAACLCLSCDQIVHSANALSKRHSRTLLCERCNSQPALVRRVEERISLCQNCDWMGYGSSTSASTHKRQTINCYFGCPSVSELSSKWPFILDSPSGGGSTCEQELGLMSIAENSTKNTWGPTENTICHNASGIVGASSVPESSSVPKNLDQPTRSPNSSLPKVCDADLYEDFNMAEMDLNLENYEELFGVTLNNSEELFENGGIDSLFGTKDMPIVDSNFGLVNTIQPTCSNAPSTDSMISSKTEPILCFTAKQGHSSLSFSGLTGESNAGDYQDCGASSMLLMGEPSWCPPCLESPLPSANRSDAVKRYMEKKKTRKFEKKVRYASRKARADVRRRVKGRFVKAGDAYDYDPLGQTRSF

SEQ ID NO: 123 - DNA, 1221 bp, Oryza sativa
  misc_feature 1..1221: note = Os02g49230.1, ortholog of AtBBX7 (G2440), AtBBX8 (G1479)
  source 1..1221: mol_type = unassigned DNA
atggatgctctttgtgatttctgcagggagcaaaggtcaatggtctattgccgatcagatgcagcatccttgtgcttgtcgtgtgatcgtaatgtgcattcagccaatgccctttcccgacgccatacaagaacccttctttgtgatcgttgtgttggacagcctgcagcagtccgttgtcttgaagagaacacctcactttgccaaaactgtgactggaatggacatggggcagcatcctcagctgctgggcataaaaggcagaccataaactgttactcggggtgcccatcgtcggcagagctctcgagaatctggtcctttagtatggatatcccaactgtagctgcggagccaaactgtgaggaaggaataaacatgatgagcattaatgacaatgacgtcaataaccattgtggtgctccagaagatggccgcttgttggatatagctagcacagcactcatgagtgatttacctacaggagacaagttcaaacctttaataggttcttcttcaggagatgggatgaatcttctgccacttaattcagatcagccagctgagccagtttcaacgacgcctaaggcaccctgtgtcacagacaaagatatgttcaatgatgggagcgtatatggagacttctgtgtggatgatgctgacctaacatttgagaattatgaagaattattcggtacctctcacgttcaaacagaacaactctttgatgatgctggaattgacagttattttgaaatgaaggatgtgccagcagatgaatctaatgagcagcccaaacctgtgcagccagaatgtagcaatgtcgcatctgttgattctgggatgtcaaacccagctgcaagggctgattccagccattgtattcctggtaggcaggctatatccaacatatccctttccttctctggtttgactggtgaaagcagtgctggatattttcaagattgtggggtatcgtcaatgatccttatgggtgagccaccctggcatcctcctggtcctgaaagttcatctgctggaggcagcagagataatgctcttacacggtacaaggagaagaagaagagaagaaaatttgacaagaagataaggtatgcttctcgtaaggctagggcagatgtgagaaaaagggtcaaaggacggtttgtaaaggctggcgaagcatatgactacgatccgctgagccaaacaagaagctac

SEQ ID NO: 124 - protein, 407 aa, Oryza sativa
  REGION 1..407: note = Os02g49230.1, domain coordinates 5-42, 48-85, 350-391, ortholog of AtBBX7 (G2440), AtBBX8 (G1479)
MDALCDFCREQRSMVYCRSDAASLCLSCDRNVHSANALSRRHTRTLLCDRCVGQPAAVRCLEENTSLCQNCDWNGHGAASSAAGHKRQTINCYSGCPSSAELSRIWSFSMDIPTVAAEPNCEEGINMMSINDNDVNNHCGAPEDGRLLDIASTALMSDLPTGDKFKPLIGSSSGDGMNLLPLNSDQPAEPVSTTPKAPCVTDKDMFNDGSVYGDFCVDDADLTFENYEELFGTSHVQTEQLFDDAGIDSYFEMKDVPADESNEQPKPVQPECSNVASVDSGMSNPAARADSSHCIPGRQAISNISLSFSGLTGESSAGYFQDCGVSSMILMGEPPWHPPGPESSSAGGSRDNALTRYKEKKKRRKFDKKIRYASRKARADVRKRVKGRFVKAGEAYDYDPLSQTRSY

SEQ ID NO: 125 - DNA, 1440 bp, Oryza sativa
  misc_feature 1..1440: note = Os02g49230.2, ortholog of AtBBX7 (G2440), AtBBX8 (G1479)
  source 1..1440: mol_type = unassigned DNA
atgctaataagccatcccatgctgagcaattcaagtggttcatttcatggcctgtatcaatatacttatatatctccaacacttaccaaggaatggaaacaaactcatcattatcctggtaggatcccactgaattcaactgttgaacaaaaagccaggaaggtggctgttcttgtggcactcagtgctagtggaagtgctccagtactgtacaggaccatggatgctctttgtgatttctgcagggagcaaaggtcaatggtctattgccgatcagatgcagcatccttgtgcttgtcgtgtgatcgtaatgtgcattcagccaatgccctttcccgacgccatacaagaacccttctttgtgatcgttgtgttggacagcctgcagcagtccgttgtcttgaagagaacacctcactttgccaaaactgtgactggaatggacatggggcagcatcctcagctgctgggcataaaaggcagaccataaactgttactcggggtgcccatcgtcggcagagctctcgagaatctggtcctttagtatggatatcccaactgtagctgcggagccaaactgtgaggaaggaataaacatgatgagcattaatgacaatgacgtcaataaccattgtggtgctccagaagatggccgcttgttggatatagctagcacagcactcatgagtgatttacctacaggagacaagttcaaacctttaataggttcttcttcaggagatgggatgaatcttctgccacttaattcagatcagccagctgagccagtttcaacgacgcctaaggcaccctgtgtcacagacaaagatatgttcaatgatgggagcgtatatggagacttctgtgtggatgatgctgacctaacatttgagaattatgaagaattattcggtacctctcacgttcaaacagaacaactctttgatgatgctggaattgacagttattttgaaatgaaggatgtgccagcagatgaatctaatgagcagcccaaacctgtgcagccagaatgtagcaatgtcgcatctgttgattctgggatgtcaaacccagctgcaagggctgattccagccattgtattcctggtaggcaggctatatccaacatatccctttccttctctggtttgactggtgaaagcagtgctggatattttcaagattgtggggtatcgtcaatgatccttatgggtgagccaccctggcatcctcctggtcctgaaagttcatctgctggaggcagcagagataatgctcttacacggtacaaggagaagaagaagagaagaaaatttgacaagaagataaggtatgcttctcgtaaggctagggcagatgtgagaaaaagggtcaaaggacggtttgtaaaggctggcgaagcatatgactacgatccgctgagccaaacaagaagctac

SEQ ID NO: 126 - protein, 480 aa, Oryza sativa
  REGION 1..480: note = Os02g49230.2, domain coordinates 78-115, 121-158, 423-464, ortholog of AtBBX7 (G2440), AtBBX8 (G1479)
MLISHPMLSNSSGSFHGLYQYTYISPTLTKEWKQTHHYPGRIPLNSTVEQKARKVAVLVALSASGSAPVLYRTMDALCDFCREQRSMVYCRSDAASLCLSCDRNVHSANALSRRHTRTLLCDRCVGQPAAVRCLEENTSLCQNCDWNGHGAASSAAGHKRQTINCYSGCPSSAELSRIWSFSMDIPTVAAEPNCEEGINMMSINDNDVNNHCGAPEDGRLLDIASTALMSDLPTGDKFKPLIGSSSGDGMNLLPLNSDQPAEPVSTTPKAPCVTDKDMFNDGSVYGDFCVDDADLTFENYEELFGTSHVQTEQLFDDAGIDSYFEMKDVPADESNEQPKPVQPECSNVASVDSGMSNPAARADSSHCIPGRQAISNISLSFSGLTGESSAGYFQDCGVSSMILMGEPPWHPPGPESSSAGGSRDNALTRYKEKKKRRKFDKKIRYASRKARADVRKRVKGRFVKAGEAYDYDPLSQTRSY

SEQ ID NO: 127 - DNA, 1221 bp, Oryza sativa; identical to SEQ ID NO: 123
  misc_feature 1..1221: note = Os02g49230.3, ortholog of AtBBX7 (G2440), AtBBX8 (G1479)
  source 1..1221: mol_type = unassigned DNA

SEQ ID NO: 128 - protein, 407 aa, Oryza sativa; identical to SEQ ID NO: 124
  REGION 1..407: note = Os02g49230.3, domain coordinates 5-42, 48-85, 350-391, ortholog of AtBBX7 (G2440), AtBBX8 (G1479)

SEQ ID NO: 129 - DNA, 1221 bp, Oryza sativa; identical to SEQ ID NO: 123
  misc_feature 1..1221: note = Os02g49230.4, ortholog of AtBBX7 (G2440), AtBBX8 (G1479)
  source 1..1221: mol_type = unassigned DNA

SEQ ID NO: 130 - protein, 407 aa, Oryza sativa; identical to SEQ ID NO: 124
  REGION 1..407: note = Os02g49230.4, domain coordinates 5-42, 48-85, 350-391, ortholog of AtBBX7 (G2440), AtBBX8 (G1479)

SEQ ID NO: 131 - DNA, 1218 bp, Oryza sativa
  misc_feature 1..1218: note = Os02g49230.5, ortholog of AtBBX7 (G2440), AtBBX8 (G1479)
  source 1..1218: mol_type = unassigned DNA
atggatgctctttgtgatttctgcagggagcaaaggtcaatggtctattgccgatcagatgcagcatccttgtgcttgtcgtgtgatcgtaatgtgcattcagccaatgccctttcccgacgccatacaagaacccttctttgtgatcgttgtgttggacagcctgcagcagtccgttgtcttgaagagaacacctcactttgccaaaactgtgactggaatggacatggggcagcatcctcagctgctgggcataaaaggcagaccataaactgttactcggggtgcccatcgtcggcagagctctcgagaatctggtcctttagtatggatatcccaactgtagctgcggagccaaactgtgaggaaggaataaacatgatgagcattaatgacaatgacgtcaataaccattgtggtgctccagaagatggccgcttgttggatatagctagcacagcactcatgagtgatttacctacaggagacaagttcaaacctttaataggttcttcttcaggagatgggatgaatcttctgccacttaattcagatcagccagctgagccagtttcaacgacgcctaaggcaccctgtgtcacagacaaagatatgttcaatgatgggagcgtatatggagacttctgtgtggatgatgctgacctaacatttgagaattatgaagaattattcggtacctctcacgttcaaacagaacaactctttgatgatgctggaattgacagttattttgaaatgaaggatgtgccagcagatgaatctaatgagcccaaacctgtgcagccagaatgtagcaatgtcgcatctgttgattctgggatgtcaaacccagctgcaagggctgattccagccattgtattcctggtaggcaggctatatccaacatatccctttccttctctggtttgactggtgaaagcagtgctggatattttcaagattgtggggtatcgtcaatgatccttatgggtgagccaccctggcatcctcctggtcctgaaagttcatctgctggaggcagcagagataatgctcttacacggtacaaggagaagaagaagagaagaaaatttgacaagaagataaggtatgcttctcgtaaggctagggcagatgtgagaaaaagggtcaaaggacggtttgtaaaggctggcgaagcatatgactacgatccgctgagccaaacaagaagctac

SEQ ID NO: 132 - protein, 406 aa, Oryza sativa
  REGION 1..406: note = Os02g49230.5, domain coordinates 5-42, 48-85, 349-390, ortholog of AtBBX7 (G2440), AtBBX8 (G1479)
MDALCDFCREQRSMVYCRSDAASLCLSCDRNVHSANALSRRHTRTLLCDRCVGQPAAVRCLEENTSLCQNCDWNGHGAASSAAGHKRQTINCYSGCPSSAELSRIWSFSMDIPTVAAEPNCEEGINMMSINDNDVNNHCGAPEDGRLLDIASTALMSDLPTGDKFKPLIGSSSGDGMNLLPLNSDQPAEPVSTTPKAPCVTDKDMFNDGSVYGDFCVDDADLTFENYEELFGTSHVQTEQLFDDAGIDSYFEMKDVPADESNEPKPVQPECSNVASVDSGMSNPAARADSSHCIPGRQAISNISLSFSGLTGESSAGYFQDCGVSSMILMGEPPWHPPGPESSSAGGSRDNALTRYKEKKKRRKFDKKIRYASRKARADVRKRVKGRFVKAGEAYDYDPLSQTRSY

SEQ ID NO: 133 - DNA, 1221 bp, Oryza sativa; identical to SEQ ID NO: 123
  misc_feature 1..1221: note = Os02g49230.6, ortholog of AtBBX7 (G2440), AtBBX8 (G1479)
  source 1..1221: mol_type = unassigned DNA

SEQ ID NO: 134 - protein, 407 aa, Oryza sativa; identical to SEQ ID NO: 124
  REGION 1..407: note = Os02g49230.6, domain coordinates 5-42, 48-85, 350-391, ortholog of AtBBX7 (G2440), AtBBX8 (G1479)

SEQ ID NO: 135 - DNA, 1224 bp, Oryza sativa
  misc_feature 1..1224: note = Os06g19444.1, ortholog of AtBBX7 (G2440), AtBBX8 (G1479)
  source 1..1224: mol_type = unassigned DNA
atgggcgctctttgtgatttctgcggggagcagaggtctatggtttattgccgatcagatgcagcatccttgtgcttatcgtgtgaccgtaatgtgcattcagctaatgctctttctcggcgccatacaaggacacttctgtgtgatagatgtgcttcacagcctgccatggtccgctgccttgtagaaaatgcctcgctttgccaaaattgtgattggaatgggcatagtgctgggtcttcagctgctggtcataagaggcagaccataaactgctactcagggtgcccatcatcttcagaactttcaaaaatctggacatttgtttcggatattcctaatgtagctcctgagccgaattgcgagcaagggataagcatgatgagcatcagcgacagtggtgtcagtaatcaagacaatgctgcaggggacagcagtttgttagatattgctagtgcaacccttatgagtgatcttggcactgctggcaagccaaagtctttgataggctcctcatctgaagctggcgtgaaccttcttccacttgctactgatcagatggctggatccgtggattcgacatcagctaaggtgccctatacggcagaccaagatatgttcagcaaagacagtatatatgaagatttctgtgtggacgatgttgatcttagtttcgaaaattatgaggaactatttggcacctcccatattcaaacagagcaactgtttgatgatgctgggatcgacagttactttgaatcgaaggaaataccttctggaaactctgatgagcagcccaagctgatgcagccagtgaccagcaatgcagtatctgctgattcagggatgtcgatcccaggagccaaaggtgattccagtctttgtatacctgtgaggcaagctaggtccagtatctctctttcattttctggtttgactggtgagagcagtgctggagattaccaagattgtggggtatcaccagtgctccttatgggtgagcctccttggcatcctcctggccctgaggggtcatttgctggagctaccagagacgatgctatcacacgatacaaggagaagaagaagagaagaaaatttgacaagaaaatcaggtatgcatctcgcaaggctagggcagatgtgagaaaaagagtcaagggccggtttgttaaggctggtgaagcatacgactacgatccactctgtgaaacgaggagctac

SEQ ID NO: 136 - protein, 408 aa, Oryza sativa
  REGION 1..408: note = Os06g19444.1, domain coordinates 5-42, 48-85, 351-392, ortholog of AtBBX7 (G2440), AtBBX8 (G1479)
MGALCDFCGEQRSMVYCRSDAASLCLSCDRNVHSANALSRRHTRTLLCDRCASQPAMVRCLVENASLCQNCDWNGHSAGSSAAGHKRQTINCYSGCPSSSELSKIWTFVSDIPNVAPEPNCEQGISMMSISDSGVSNQDNAAGDSSLLDIASATLMSDLGTAGKPKSLIGSSSEAGVNLLPLATDQMAGSVDSTSAKVPYTADQDMFSKDSIYEDFCVDDVDLSFENYEELFGTSHIQTEQLFDDAGIDSYFESKEIPSGNSDEQPKLMQPVTSNAVSADSGMSIPGAKGDSSLCIPVRQARSSISLSFSGLTGESSAGDYQDCGVSPVLLMGEPPWHPPGPEGSFAGATRDDAITRYKEKKKRRKFDKKIRYASRKARADVRKRVKGRFVKAGEAYDYDPLCETRSY

SEQ ID NO: 137 - DNA, 1221 bp, Oryza sativa
  misc_feature 1..1221: note = Os06g19444.2, ortholog of AtBBX7 (G2440), AtBBX8 (G1479)
  source 1..1221: mol_type = unassigned DNA
atgggcgctctttgtgatttctgcggggagcagaggtctatggtttattgccgatcagatgcagcatccttgtgcttatcgtgtgaccgtaatgtgcattcagctaatgctctttctcggcgccatacaaggacacttctgtgtgatagatgtgcttcacagcctgccatggtccgctgccttgtagaaaatgcctcgctttgccaaaattgtgattggaatgggcatagtgctgggtcttcagctgctggtcataagaggcagaccataaactgctactcagggtgcccatcatcttcagaactttcaaaaatctggacatttgtttcggatattcctaatgtagctcctgagccgaattgcgagcaagggataagcatgatgagcatcagcgacagtggtgtcagtaatcaagacaatgctgcaggggacagcagtttgttagatattgctagtgcaacccttatgagtgatcttggcactgctggcaagccaaagtctttgataggctcctcatctgaagctggcgtgaaccttcttccacttgctactgatcagatggctggatccgtggattcgacatcagctaaggtgccctatacggcagaccaagatatgttcagcaaagacagtatatatgaagatttctgtgtggacgatgttgatcttagtttcgaaaattatgaggaactatttggcacctcccatattcaaacagagcaactgtttgatgatgctgggatcgacagttactttgaatcgaaggaaataccttctggaaactctgatgagcccaagctgatgcagccagtgaccagcaatgcagtatctgctgattcagggatgtcgatcccaggagccaaaggtgattccagtctttgtatacctgtgaggcaagctaggtccagtatctctctttcattttctggtttgactggtgagagcagtgctggagattaccaagattgtggggtatcaccagtgctccttatgggtgagcctccttggcatcctcctggccctgaggggtcatttgctggagctaccagagacgatgctatcacacgatacaaggagaagaagaagagaagaaaatttgacaagaaaatcaggtatgcatctcgcaaggctagggcagatgtgagaaaaagagtcaagggccggtttgttaaggctggtgaagcatacgactacgatccactctgtgaaacgaggagctac

SEQ ID NO: 138 - protein, 407 aa, Oryza sativa
  REGION 1..407: note = Os06g19444.2, domain coordinates 5-42, 48-85, 350-391, ortholog of AtBBX7 (G2440), AtBBX8 (G1479)
MGALCDFCGEQRSMVYCRSDAASLCLSCDRNVHSANALSRRHTRTLLCDRCASQPAMVRCLVENASLCQNCDWNGHSAGSSAAGHKRQTINCYSGCPSSSELSKIWTFVSDIPNVAPEPNCEQGISMMSISDSGVSNQDNAAGDSSLLDIASATLMSDLGTAGKPKSLIGSSSEAGVNLLPLATDQMAGSVDSTSAKVPYTADQDMFSKDSIYEDFCVDDVDLSFENYEELFGTSHIQTEQLFDDAGIDSYFESKEIPSGNSDEPKLMQPVTSNAVSADSGMSIPGAKGDSSLCIPVRQARSSISLSFSGLTGESSAGDYQDCGVSPVLLMGEPPWHPPGPEGSFAGATRDDAITRYKEKKKRRKFDKKIRYASRKARADVRKRVKGRFVKAGEAYDYDPLCETRSY

SEQ ID NO: 139 - DNA, 1215 bp, Glycine max
  misc_feature 1..1215: note = Glyma02g38870.1, ortholog of AtBBX7 (G2440), AtBBX8 (G1479)
  source 1..1215: mol_type = unassigned DNA
atgggttacttatgtgatttttgtggagaccagaggtccttggtgtactgccgctctgattccgcgtgcttatgtttgtcatgcgatcgaaatgttcattctgccaatgcactttctaggcgtcattcgagaacccttgtatgcgagagatgcaattcacaacctgcatttgtgaggtctgttgaagagaaaatttcactttgtcagaactgtgattggttgggtcatggtacctcaccgtcttcttcgatgcacaagaggcaatcaatcaattgctactctggctgcccttcagcggcagaattttcttcaatatggtcctttttcttagacatcccttctttgggtgaagcatgtgagcaggaactaggtttgatgagcattaatgagaacattcctccagaagggcagaatgtgtctggttcaactgaagtgactgatctacccagcaagggtaaatcttgggctggcacaccttcaatacctgagtcaagctcagaacctcgtattctggaccagccgcctggacctgccaatgaatgtgtgcccaagttgtactgtcctgggaagaaagcttctggaatatgtgaagatgatgatctatatgatgatttcatcatggatgaagttgatctagaacttgagaactatgaggaactttttgggatggctctcagtcactctgaagagctttttgaaaatggtggaattaatagcttatttgaggcaaaggacatgtctgctagtgctggtgattcccattgtcagggtgctgttgctgctgagggatcgtctgctgggttggtcaatcctatccaaccagcatgcagcaatgcagcatctgcagattcagtgatgagtacaaaaactgaaccaattgtttgttttacggcaaggcaatctcaatcaaacatttccttttctggtgttaccaaagatagtgctggtgactatcaagactgtggtgcttcttcaatgcttctcatgggagaacctccctggtgtcctccttgccctgagagttctctgcattctgctaaccgaagcaatgctgtcatgcgctacaaggaaaaaaagaagacccggaagttcgagaagaaagtaagatatgcctctcgcaaggctagggctgatgtcagaaagcgtgtgaagggccgatttgtcaaagctggtgatgtctatgactatgaccctttgaaccaaaccagaagctac

SEQ ID NO: 140 - protein, 405 aa, Glycine max
  REGION 1..405: note = Glyma02g38870.1, domain coordinates 5-42, 48-85, 348-389, ortholog of AtBBX7 (G2440), AtBBX8 (G1479)
MGYLCDFCGDQRSLVYCRSDSACLCLSCDRNVHSANALSRRHSRTLVCERCNSQPAFVRSVEEKISLCQNCDWLGHGTSPSSSMHKRQSINCYSGCPSAAEFSSIWSFFLDIPSLGEACEQELGLMSINENIPPEGQNVSGSTEVTDLPSKGKSWAGTPSIPESSSEPRILDQPPGPANECVPKLYCPGKKASGICEDDDLYDDFIMDEVDLELENYEELFGMALSHSEELFENGGINSLFEAKDMSASAGDSHCQGAVAAEGSSAGLVNPIQPACSNAASADSVMSTKTEPIVCFTARQSQSNISFSGVTKDSAGDYQDCGASSMLLMGEPPWCPPCPESSLHSANRSNAVMRYKEKKKTRKFEKKVRYASRKARADVRKRVKGRFVKAGDVYDYDPLNQTRSY

SEQ ID NO: 141 - DNA, 843 bp, Glycine max
  misc_feature 1..843: note = Glyma13g11590.1, ortholog of AtBBX7 (G2440), AtBBX8 (G1479)
  source 1..843: mol_type = unassigned DNA
atgagtattaatgagaacaagagtgttggggttcctccagagggccagaatgtgtctggttcagatgaagttactgatcaacctgctttggacaagtctttggttggtacatcttcaatgcctgaatcaagctccaaacctcgtattctggaccagccagctagacctgccaatgaatgtctttcaaagttatactgtcctgctacaaaattccccgcattatgtgaaaatgataatctgtatgatgatttcaacatggatgaagtggatctaaatcttgagaactatgaagaactttttggtatggccctcagtcattctgaagagctatttgaaaatggtggaattgatagcttgtttgggacaaaagacatgtctgctggagatttcagttgtgaggatgctattgctgctgaggcatcacaaattccaactatgttggcacctcagcctcacaatgcagcatctgcagattccattttgagtactaaaactgaaccaattctttgttttacgggaaggcaagctcaatcaaacctttctttttctggcgttaccggagacagtagtgctggagactatcaagactgtggggcttcttcaatgcttctcatgggagaacctccttggtttgctccttgtcctgagaattccctacaatcagccaaccgtagtaatgctgtcatgcggtacaaggaaaagaagaagacacggatgtttgacaaaaaagtgaggtatgcctctcgcaaggcaagggctgatgtcagaaggcgcgtgaaaggccggtttgtcaaagctggtgacgtctacgattatgatcctttgagcacaaccagaagcttc

SEQ ID NO: 142 - protein, 281 aa, Glycine max
  REGION 1..281: note = Glyma13g11590.1, domain coordinates 224-265, ortholog of AtBBX7 (G2440), AtBBX8 (G1479)
MSINENKSVGVPPEGQNVSGSDEVTDQPALDKSLVGTSSMPESSSKPRILDQPARPANECLSKLYCPATKFPALCENDNLYDDFNMDEVDLNLENYEELFGMALSHSEELFENGGIDSLFGTKDMSAGDFSCEDAIAAEASQIPTMLAPQPHNAASADSILSTKTEPILCFTGRQAQSNLSFSGVTGDSSAGDYQDCGASSMLLMGEPPWFAPCPENSLQSANRSNAVMRYKEKKKTRMFDKKVRYASRKARADVRRRVKGRFVKAGDVYDYDPLSTTRSF

SEQ ID NO: 143 - DNA, 1233 bp, Glycine max
  misc_feature 1..1233: note = Glyma14g36930.1, ortholog of AtBBX7 (G2440), AtBBX8 (G1479)
  source 1..1233: mol_type = unassigned DNA
atgggttacttatgtgatttttgtggagaccagaggtccctggtgtactgccgctctgatgctgcgtgcttatgtttgtcgtgtgatcgaaatgttcattctgctaatgccctttctaggcgtcattcgagaacccttgtatgcgagagatgcaattcgcaacctgcatttgtgaggtgtgttgatgagaagatttcactttgtcagaactgtgattggttgggtcatggtacctcaccatcttcttcgacacacaagaggcaatcaatcaattgctactctggctgcccttcagctgcagaattttcttcaatatggtcctttttcttagacatcccttctatgggtgaagcatgtgagcaggaactaggtttgatgagcattaatgagaacagcaataagaatgcttgggcttctccagaagggcagaatgtgtctggttcagctgaagtgactgatctacccagcaagggtaaatcttgggctggcacatcttcagtacctgagtcaagctcagaacctcgtattctagaccagccgcctggacctgccaatgaatgtatgcccaagttgtactgtcctgggaagaaagtttctggaatatatgaagatgatgatctatatgatgatttcatcatggatgaagtggatctacaacttgagaactacgaggaactttttgggatggctctcagtcactctgaagagctttttgaaaatggtggaattaatagcttatttgagacaaaggacatgtctgctagtgctggtgattcccattgtcagggtgctgttgctgctgagggatcgtctgctgagttggtcaatgctatccaaccagcatgcagcaatgcagcatctgcagactcaatgatgagtacaaaaactgaaccaattgtttgttttacggcaaggcaatctctatcaaacatttctttctctggtgttaccaaagatagtgttggtgactatcaagattgtggtgcttcttcaatgcttctcatgggagagcctccctggtgtcctccttgccctgagagttctctgcattctgctaaccgcagcaatgctgtcatgcgctataaggaaaagaagaagacccggaagttcgagaagaaagtaaggtatgcctctcgcaaggctagggctgatgtcagaaagcgtgtgaagggtcgatttgtcaaagccggtgatgtctatgactatgaccctttgaaccaaaccagaagctgc

SEQ ID NO: 144 - protein, 411 aa, Glycine max
  REGION 1..411: note = Glyma14g36930.1, domain coordinates 5-42, 48-85, 354-395, ortholog of AtBBX7 (G2440), AtBBX8 (G1479)
MGYLCDFCGDQRSLVYCRSDAACLCLSCDRNVHSANALSRRHSRTLVCERCNSQPAFVRCVDEKISLCQNCDWLGHGTSPSSSTHKRQSINCYSGCPSAAEFSSIWSFFLDIPSMGEACEQELGLMSINENSNKNAWASPEGQNVSGSAEVTDLPSKGKSWAGTSSVPESSSEPRILDQPPGPANECMPKLYCPGKKVSGIYEDDDLYDDFIMDEVDLQLENYEELFGMALSHSEELFENGGINSLFETKDMSASAGDSHCQGAVAAEGSSAELVNAIQPACSNAASADSMMSTKTEPIVCFTARQSLSNISFSGVTKDSVGDYQDCGASSMLLMGEPPWCPPCPESSLHSANRSNAVMRYKEKKKTRKFEKKVRYASRKARADVRKRVKGRFVKAGDVYDYDPLNQTRSC

SEQ ID NO: 145 - DNA, 1233 bp, Glycine max; identical to SEQ ID NO: 143
  misc_feature 1..1233: note = Glyma14g36930.2, ortholog of AtBBX7 (G2440), AtBBX8 (G1479)
  source 1..1233: mol_type = unassigned DNA

SEQ ID NO: 146 - protein, 411 aa, Glycine max; identical to SEQ ID NO: 144
  REGION 1..411: note = Glyma14g36930.2, domain coordinates 5-42, 48-85, 354-395, ortholog of AtBBX7 (G2440), AtBBX8 (G1479)

SEQ ID NO: 147 - DNA, 849 bp, Glycine max
  misc_feature 1..849: note = Glyma20g07050.1, ortholog of AtBBX7 (G2440), AtBBX8 (G1479)
  source 1..849: mol_type = unassigned DNA
atgagtattaatgagaacaagtgtgttggtgttcctccggaagaccagaatgtgtctggttcagatgaagttaccgatctacctgctttgaacaagtctttggttggtgcatctttgatgcctgaatcaagctccgaaccttgtattctggaccagccagctggacctaccaatgaatgtctgccaaagttatactgtcctgctacaaaatgccctgccttatctgaagatgataatctgtatgatgatttcaacatggatgaagtggatctagatcttgagaactatgaagaactttttggtatggccctcagtcattctgaggagctttttgaaaatggtggaattgatagcttgtttgggacaaaaggcatgtctgctggagattccaattgtcaggaagctattgctgctgaggggtcgtcggttggacaggtcaatgcaatgcaaccagcttgcagcaatgcagcgtctgcagattccattttgagtactaaaactgaaccaattctttgttttactggaaggcaaactcaatcaaacctttctttttctggtgttactggagagagtagtgctggagactatcaagactgtggggcttcttcaatgcttctcatgggagaacctccttggtttgctccttgtccggagaattccctacaatccgccaaccgtagtaatgctgtcatgcggtacaaggaaaagaagaagacacggatgtttgacaaaaaagttaggtatgcctctcgcaaggcaagggctgatgtcagaaggcgtgtgaaagggcggtttgtcaaagccggtgacgtctatgattatgaccctttgagcacaaccagaagctgc

SEQ ID NO: 148 - protein, 283 aa, Glycine max
  REGION 1..283: note = Glyma20g07050.1, domain coordinates 226-267, ortholog of AtBBX7 (G2440), AtBBX8 (G1479)
MSINENKCVGVPPEDQNVSGSDEVTDLPALNKSLVGASLMPESSSEPCILDQPAGPTNECLPKLYCPATKCPALSEDDNLYDDFNMDEVDLDLENYEELFGMALSHSEELFENGGIDSLFGTKGMSAGDSNCQEAIAAEGSSVGQVNAMQPACSNAASADSILSTKTEPILCFTGRQTQSNLSFSGVTGESSAGDYQDCGASSMLLMGEPPWFAPCPENSLQSANRSNAVMRYKEKKKTRMFDKKVRYASRKARADVRRRVKGRFVKAGDVYDYDPLSTTRSC

SEQ ID NO: 149 - DNA, 849 bp, Glycine max; identical to SEQ ID NO: 147
  misc_feature 1..849: note = Glyma20g07050.2, ortholog of AtBBX7 (G2440), AtBBX8 (G1479)
  source 1..849: mol_type = unassigned DNA

SEQ ID NO: 150 - protein, 283 aa, Glycine max; identical to SEQ ID NO: 148
  REGION 1..283: note = Glyma20g07050.2, domain coordinates 226-267, ortholog of AtBBX7 (G2440), AtBBX8 (G1479)

SEQ ID NO: 151 - DNA, 744 bp, Arabidopsis thaliana
  misc_feature 1..744: note = AtBBX24 (G329), AT1G06040.1, paralog of AtBBX25 (G1894)
  source 1..744: mol_type = unassigned DNA
atgaagatacagtgtgatgtgtgtgagaaagctccggcgacggtgatttgttgcgccgacgaagctgctctctgtcctcaatgcgacatcgagattcacgccgctaacaaactcgctagcaagcaccaacgtcttcatcttaattccctctccaccaaattccctcgttgcgatatctgccaagagaaggcagctttcattttctgtgtagaggatagagctctgctttgcagggactgcgatgaatccatccacgtggctaattctcgatctgctaatcaccagaggttcttagccactgggatcaaagtagctctgacctcaactatatgtagtaaagaaattgagaagaatcaacctgagccttccaacaaccaacagaaggctaatcagattcctgctaaatccacaagccagcagcaacaacaaccttcttctgctactccacttccctgggctgttgacgatttctttcacttctctgatattgaatccaccgacaagaaaggacagcttgatcttggggcaggggagttggattggttttcagacatgggattcttcggtgatcagattaatgacaaggctcttcctgcagctgaagttcctgagctttctgtttcgcatttaggtcatgttcattcatacaaacctatgaagtcaaatgtttcacacaagaagccgaggtttgagaccagatatgatgatgatgatgaggaacacttcattgtccctgatcttggc

SEQ ID NO: 152 - protein, 248 aa, Arabidopsis thaliana
  REGION 1..248: note = AtBBX24 (G329), AT1G06040.1, domain coordinates 5-42, 57-94, paralog of AtBBX25 (G1894)
MKIQCDVCEKAPATVICCADEAALCPQCDIEIHAANKLASKHQRLHLNSLSTKFPRCDICQEKAAFIFCVEDRALLCRDCDESIHVANSRSANHQRFLATGIKVALTSTICSKEIEKNQPEPSNNQQKANQIPAKSTSQQQQQPSSATPLPWAVDDFFHFSDIESTDKKGQLDLGAGELDWFSDMGFFGDQINDKALPAAEVPELSVSHLGHVHSYKPMKSNVSHKKPRFETRYDDDDEEHFIVPDLG

SEQ ID NO: 153 - DNA, 714 bp, Arabidopsis thaliana
  misc_feature 1..714: note = AtBBX25 (G1894), AT2G31380.1, paralog of AtBBX24 (G329)
  source 1..714: mol_type = unassigned DNA
atgaagatacaatgtgatgtgtgtgagaaagctccggccacgcttatatgttgtgctgatgaagctgctctctgcgctaaatgtgacgttgaggttcatgctgctaataaactcgctagcaaacaccaacgcctttttcttgactctctctcaactaaattccctccctgcgacatctgccttgagaaggcagctttcatattctgtgtagaggatagggctctgctctgcagagattgcgatgaggcgacccatgcgccaaatactcgctctgctaatcaccagaggttcttagccactggaatccgagttgctcttagttccactagttgcaatcaagaagtggaaaagaatcactttgacccatctaatcagcagagtctctctaaaccgccaactcagcaacccgctgctccatctcctttgtgggctaccgatgaattcttcagctactctgatcttgactgcagtaataaggagaaagagcaactcgatctcggggagctggattggcttgcagagatgggtctgtttggtgaccagcctgatcaagaggctctaccggtagccgaagttcccgagctttccttttcacatttggctcatgctcattcctacaacagacctatgaagtccaatgtacccaacaagaagcagaggcttgagtaccggtatgatgatgaagaagagcacttcctagtccccgacctaggc

SEQ ID NO: 154 - protein, 238 aa, Arabidopsis thaliana
  REGION 1..238: note = AtBBX25 (G1894), AT2G31380.1, domain coordinates 5-42, 57-94, paralog of AtBBX24 (G329)
MKIQCDVCEKAPATLICCADEAALCAKCDVEVHAANKLASKHQRLFLDSLSTKFPPCDICLEKAAFIFCVEDRALLCRDCDEATHAPNTRSANHQRFLATGIRVALSSTSCNQEVEKNHFDPSNQQSLSKPPTQQPAAPSPLWATDEFFSYSDLDCSNKEKEQLDLGELDWLAEMGLFGDQPDQEALPVAEVPELSFSHLAHAHSYNRPMKSNVPNKKQRLEYRYDDEEEHFLVPDLG

SEQ ID NO: 155 - DNA, 774 bp, Zea mays
  misc_feature 1..774: note = ACF84591.1, ortholog of AtBBX24 (G329), AtBBX25 (G1894)
  source 1..774: mol_type = unassigned DNA
atgaagatccagtgcgacgcgtgcgagggcgcggctgccacggtggtgtgctgcgccgacgaggccgcgctgtgcgcgcgctgcgacgtcgagatccacgccgccaacaagctcgcgagcaagcaccagcgcctcccgctcgaggcgctctccgccaagctcccgcgctgcgacgtctgccaggagaaagcggcgttcatcttctgcgtggaggaccgggcgctcttctgccaggactgcgacgagcccatccacgtcccgggcacgctctccgggaaccaccagcgctacctggccaccggcatccgcgtcggcctcgcctcggcttccgcctgcagcgacgcctgcgacgcccacgactccgaccaccacgccccgcccaaggccaccatcgagccaccgcacgccgccgtctccgccgcggtgcagcaggtgccctcgccgccgcagttcctgccgcagggctgggccgtcgacgagctcctgcagttctccgactacgagtccagcgacaagctgcacaaggagccgacgctcgggttcaaggagctggagtggttcgccgacatcgacctcttccacgagcaggcgcccaaagccagcaggacgttggcggaggtgccggagcttttcggataccaggcggcgaacgacgcggcgtactacaggccggccaaagccgccgccggcggcggcgctggggttcgccagagcaagaaggcccggatcgaggtcaccgacgacgaggattacctcatagtccctgatcttggt

SEQ ID NO: 156 - protein, 258 aa, Zea mays
  REGION 1..258: note = ACF84591.1, domain coordinates 5-42, 57-94, ortholog of AtBBX24 (G329), AtBBX25 (G1894)
MKIQCDACEGAAATVVCCADEAALCARCDVEIHAANKLASKHQRLPLEALSAKLPRCDVCQEKAAFIFCVEDRALFCQDCDEPIHVPGTLSGNHQRYLATGIRVGLASASACSDACDAHDSDHHAPPKATIEPPHAAVSAAVQQVPSPPQFLPQGWAVDELLQFSDYESSDKLHKEPTLGFKELEWFADIDLFHEQAPKASRTLAEVPELFGYQAANDAAYYRPAKAAAGGGAGVRQSKKARIEVTDDEDYLIVPDLG

SEQ ID NO: 157 - DNA, 759 bp, Zea mays
  misc_feature 1..759: note = ACF86823.1, ortholog of AtBBX24 (G329), AtBBX25 (G1894)
  source 1..759: mol_type = unassigned DNA
atgaagatccagtgcgacgcgtgcgagggcgcggctgccaccgtggtgtgctgcgccgacgaggccgcgctgtgcgcgcgctgcgacgtcgagatccacgccgccaacaagctcgccagcaagcaccagcgcctcccgctcgaggctctctccgccagcctcccgcgctgcgacgtctgccaggagaaagcggcgttcatcttctgcgtggaggaccgggcgctcttctgccgggactgcgacgagcccatccacgtcccgggcacgctctccgggaaccaccagcgctacctcgccaccgacatccgcgtcggcttcgcctccgcttcgtcagcgtgcagtgacgcctgcgacgcccacgacgactccgaccaccacgccccgcccaaggccgccgtctcctccgcggcgcagcaggtgccctcgccgccgcagttcctgccacagggctgggccgtcgacgagctcctgcagttctccgactgcgagtccagcgacaagctgcacaaggagtccccgctcgggttcaaggagctggagtggttcaccgacatcgacctcttccacgagcagacgccgaaagccggcaggagattggctgaggtaccggagctttccggaacccaggccgcgaacgacgcggcgtactacaggccagccaaagccactgccactgccggcgccggggtgcgccagagcaagaaggcccggaccgaggtcaccgacgacgaggaccacctcatcgtccctgatctcggc

SEQ ID NO: 158 - protein, 253 aa, Zea mays
  REGION 1..253: note = ACF86823.1, domain coordinates 5-42, 57-94, ortholog of AtBBX24 (G329), AtBBX25 (G1894)
MKIQCDACEGAAATVVCCADEAALCARCDVEIHAANKLASKHQRLPLEALSASLPRCDVCQEKAAFIFCVEDRALFCRDCDEPIHVPGTLSGNHQRYLATDIRVGFASASSACSDACDAHDDSDHHAPPKAAVSSAAQQVPSPPQFLPQGWAVDELLQFSDCESSDKLHKESPLGFKELEWFTDIDLFHEQTPKAGRRLAEVPELSGTQAANDAAYYRPAKATATAGAGVRQSKKARTEVTDDEDHLIVPDLG

SEQ ID NO: 159 - DNA, 759 bp, Zea mays; identical to SEQ ID NO: 157
  misc_feature 1..759: note = ACG28309.1, ortholog of AtBBX24 (G329), AtBBX25 (G1894)
  source 1..759: mol_type = unassigned DNA

SEQ ID NO: 160 - protein, 253 aa, Zea mays; identical to SEQ ID NO: 158
  REGION 1..253: note = ACG28309.1, domain coordinates 5-42, 57-94, ortholog of AtBBX24 (G329), AtBBX25 (G1894)

SEQ ID NO: 161 - DNA, 759 bp, Zea mays
  misc_feature 1..759: note = CAW36812.1, ortholog of AtBBX24 (G329), AtBBX25 (G1894)
  source 1..759: mol_type = unassigned DNA
atgaagatccagtgcgacgcgtgcgagggcgcggctgccaccgtggtgtgctgcgccgacgaggccgcgctgtgcgcgcgctgcgacgtcgagatccacgccgccaacaagctcgccagcaagcaccagcgcctcccgctcgaggctctctccgccagcctcccgcgctgcgacgtctgccaggagaaagcggcgttcatcttctgcgtggaggaccgggcgctcttctgccgggactgcgacgagcccatccacgtcccgggcacgctctccgggaaccaccagcgctacctcgccaccggcatccgcgtcggcttcgcctccgcttcgtcagcgtgcagtgacgcctgcgacgcccacgacgactccgaccaccacgccccgcccaaggccgccgtctcctccgcggcgcagcaggtgccctcgccgccgcagttcctgccacagggctgggccgtcgacgagctcctgcagttctccgactgcgagtccagcgacaagctgcacaaggagtccccgctcgggttcaaggagctggagtggttcaccgacatcgacctcttccacgagcagacgccgaaagccggcaggagattggccgaggtaccggagctttccggaacccaggccgcgaacgacgcggcgtactacaggccagccaaagccactgccactgccgtcgccggggtgcgccagagcaagaagggccggaccgaggtcaccgacgacgaggaccacctcatcgtccctgatctcgtt

SEQ ID NO: 162 - protein, 253 aa, Zea mays
  REGION 1..253: note = CAW36812.1, domain coordinates 5-42, 57-94, ortholog of AtBBX24 (G329), AtBBX25 (G1894)
MKIQCDACEGAAATVVCCADEAALCARCDVEIHAANKLASKHQRLPLEALSASLPRCDVCQEKAAFIFCVEDRALFCRDCDEPIHVPGTLSGNHQRYLATGIRVGFASASSACSDACDAHDDSDHHAPPKAAVSSAAQQVPSPPQFLPQGWAVDELLQFSDCESSDKLHKESPLGFKELEWFTDIDLFHEQTPKAGRRLAEVPELSGTQAANDAAYYRPAKATATAVAGVRQSKKGRTEVTDDEDHLIVPDLV

SEQ ID NO: 163 - DNA, 759 bp, Zea mays; identical to SEQ ID NO: 161
  misc_feature 1..759: note = CAW39068.1, ortholog of AtBBX24 (G329), AtBBX25 (G1894)
  source 1..759: mol_type = unassigned DNA

SEQ ID NO: 164 - protein, 253 aa, Zea mays; identical to SEQ ID NO: 162
  REGION 1..253: note = CAW39068.1, domain coordinates 5-42, 57-94, ortholog of AtBBX24 (G329), AtBBX25 (G1894)

SEQ ID NO: 165 - DNA, 759 bp, Zea mays; identical to SEQ ID NO: 161
  misc_feature 1..759: note = CAW54445.1, ortholog of AtBBX24 (G329), AtBBX25 (G1894)
  source 1..759: mol_type = unassigned DNA

SEQ ID NO: 166 - protein, 253 aa, Zea mays; identical to SEQ ID NO: 162
  REGION 1..253: note = CAW54445.1, domain coordinates 5-42, 57-94, ortholog of AtBBX24 (G329), AtBBX25 (G1894)

SEQ ID NO: 167 - DNA, 759 bp, Zea mays; identical to SEQ ID NO: 161
  misc_feature 1..759: note = CAW74336.1, ortholog of AtBBX24 (G329), AtBBX25 (G1894)
  source 1..759: mol_type = unassigned DNA

SEQ ID NO: 168 - protein, 253 aa, Zea mays; identical to SEQ ID NO: 162
  REGION 1..253: note = CAW74336.1, domain coordinates 5-42, 57-94, ortholog of AtBBX24 (G329), AtBBX25 (G1894)

SEQ ID NO: 169 - DNA, 759 bp, Zea mays; identical to SEQ ID NO: 161
  misc_feature 1..759: note = CAW80388.1, ortholog of AtBBX24 (G329), AtBBX25 (G1894)
  source 1..759: mol_type = unassigned DNA

SEQ ID NO: 170 - protein, 253 aa, Zea mays; identical to SEQ ID NO: 162
  REGION 1..253: note = CAW80388.1, domain coordinates 5-42, 57-94, ortholog of AtBBX24 (G329), AtBBX25 (G1894)

SEQ ID NO: 171 - DNA, 759 bp, Zea mays; identical to SEQ ID NO: 161
  misc_feature 1..759: note = CAW82896.1, ortholog of AtBBX24 (G329), AtBBX25 (G1894)
  source 1..759: mol_type = unassigned DNA

SEQ ID NO: 172 - protein, 253 aa, Zea mays; identical to SEQ ID NO: 162
  REGION 1..253: note = CAW82896.1, domain coordinates 5-42, 57-94, ortholog of AtBBX24 (G329), AtBBX25 (G1894)

SEQ ID NO: 173 - DNA, 759 bp, Zea mays; identical to SEQ ID NO: 157
  misc_feature 1..759: note = NP_001141667.1, ortholog of AtBBX24 (G329), AtBBX25 (G1894)
  source 1..759: mol_type = unassigned DNA

SEQ ID NO: 174 - protein, 253 aa, Zea mays; identical to SEQ ID NO: 158
  REGION 1..253: note = NP_001141667.1, domain coordinates 5-42, 57-94, ortholog of AtBBX24 (G329), AtBBX25 (G1894)

SEQ ID NO: 175 - DNA, 489 bp, Populus trichocarpa
  misc_feature 1..489: note = Pt_216458, ortholog of AtBBX24 (G329), AtBBX25 (G1894)
  source 1..489: mol_type = unassigned DNA
atgaaaattcagtgtgatgtgtgtgagaaagcaccagccacagtgatttgttgtgctgatgaggctgccctttgtgaaaaatgtgacatagaagttcatgcagcaaacaagcttgctagcaaacaccaaagacttcttctccagtgcctctccaacaagctgcctccatgcgatatatgccaagaaaaggctgctttcattttctgtgttgaagacagagccctcttttgccgggattgtgatgaaccaatccattcagctggtagcctttcagcaaatcaccagcggtttctggccactggaatccgagtggctttaagctctagttgctccaaggacactcagaagagttctttagagccaccaaatcagagtgaacaacaaacttccaagttgccttggcagcatgcatcgagttttggctcttcttgggctgttgatgattttctacagttctctgacattgaagaatccactgacaaggtaata

SEQ ID NO: 176 - protein, 163 aa, Populus trichocarpa
  REGION 1..163: note = Pt_216458, domain coordinates 5-42, 57-94, ortholog of AtBBX24 (G329), AtBBX25 (G1894)
MKIQCDVCEKAPATVICCADEAALCEKCDIEVHAANKLASKHQRLLLQCLSNKLPPCDICQEKAAFIFCVEDRALFCRDCDEPIHSAGSLSANHQRFLATGIRVALSSSCSKDTQKSSLEPPNQSEQQTSKLPWQHASSFGSSWAVDDFLQFSDIEESTDKVI

SEQ ID NO: 177 - DNA, 1475 bp, Populus trichocarpa
  misc_feature 1..1475: note = Pt_827145, ortholog of AtBBX24 (G329), AtBBX25 (G1894)
  source 1..1475: mol_type = unassigned DNA
ctctgagccttatctgcacacactcagcttcttggtttctccaccaaacactcctctttctgtatcttccacagattcttgcttgcattatacagtacttcacaaagctcccatctcccatttctattattataccttctcatagtttttcctctcttcttctgttctttcctttttcttcgtggccaaactctttctttctcttgttgtttcgccagtagtttcttggggaaaagattattatcatttcaggctgttttttgagctactttgaaacatatgtttcactgtctcatttactgtatgctgagttcacttaaaaggtagagagaaactggagaaggtgtaaccaagtggggatctgctaaaagagaggaggaggtttggtctgttttaagcaagatgaagattcagtgtgatgtgtgtgagaaagcacccgccacagtgatttgttgtgctgatgaggctgcactttgtgctaaatgtgacatagaagttcatgcagcaaacaagcttgctagcaagcaccaaaggcttcttctccagtgcctctccaacaagctgcctccatgtgatatatgccaagagaaggcagctttcattttctgtgttgaagacagggccctcttttgccgggattgtgatgaaccaatccattcagctggtagcctttctgcgaatcaccagcggtttctggccactggaatccgagtggctttaagctcgagttgctccaaggacacgcagacgaattcttcagggccaccaaatcagagcgcacagcaaactcccatgaaaatccctgcacagcaaacatcgagcttcgccacttcatgggcagttgatgacttgctacagttttccgagtttgaatcatccactgacaagaaagagcaacttgagttaggagagtttgagtggctagctgacatgggtcttttcggtgagcaacttcctcaagaagctctagcagcagctgaagttcctcaactgccaatctcaccaccaaccaatgtgaactcatgcagacccaccaaatccagcatgccccataagaagcctagaattgaaatctcagacgatgacgacgagtacttaactgtgcctgatcttggctgaacttggggtgttgaactcagcacttttgagttcaacttttaaggaactatacatgctttctctaagttcccgtaggcattgatgtatgtatatactatatagtatgtatttgcatacacgggcatgaaactctgcattttaaggactagagagtagcatcacagtcagctctggggattcattttccatgttatcctatatattgttatttttgttagttcttttttccatttgaaagagcaatcacatcaatcagtgctcttgattgtaatagctcaatgtatgattctcagctattgcatgtatatctgtgtgccaggatattactagctgcaagtatatggattgtttctcgc

SEQ ID NO: 178 - protein, 238 aa, Populus trichocarpa
  REGION 1..238: note = Pt_827145, domain coordinates 5-42, 57-94, ortholog of AtBBX24 (G329), AtBBX25 (G1894)
MKIQCDVCEKAPATVICCADEAALCAKCDIEVHAANKLASKHQRLLLQCLSNKLPPCDICQEKAAFIFCVEDRALFCRDCDEPIHSAGSLSANHQRFLATGIRVALSSSCSKDTQTNSSGPPNQSAQQTPMKIPAQQTSSFATSWAVDDLLQFSEFESSTDKKEQLELGEFEWLADMGLFGEQLPQEALAAAEVPQLPISPPTNVNSCRPTKSSMPHKKPRIEISDDDDEYLTVPDLG

SEQ ID NO: 179 - DNA, 813 bp, Oryza sativa
  misc_feature 1..813: note = Os02g39360.1, ortholog of AtBBX24 (G329), AtBBX25 (G1894)
  source 1..813: mol_type = unassigned DNA
atgaagatccagtgcgacgcgtgcgagagcgcggcggcggcggtggtgtgctgcgcggacgaggcggcgctgtgcgcggcgtgcgacgtggaggtgcacgcggcgaacaagctggccgggaagcaccagcggctgccgctggaggcgctctcggcgaggctcccgcgctgcgacgtgtgccaggagaaggcggcgttcatcttctgcgtggaggaccgcgcgctcttctgccgcgactgcgacgagcccatccacgtccccggcacgctctccggcaaccaccagcgctacctcgccaccggcatccgcgtcggcttcgcctccgcctcgccctgcgacggcggcagcgacgcccatgactccgaccaccacgccccgcccatgggctcctccgagcatcatcaccatcatcagcagccggccccgaccgtcgccgtcgacacgccctcgccgcagttcctgccgcagggctgggccgtcgacgagctcctccagttctccgactacgagaccggcgacaagctgcagaaggagtcgtcgccgccgctcgggttccaggagctggagtggttcgccgacatcgacctgttccacaaccaggcgcccaagggcggcgccgccgccggccggacgacggcggaggtccccgagctcttcgcttcgcaggcggccaacgacgtggcgtactacaggccgccgaccaggaccgccgccgccgccttcaccgcggccaccggcttccgccagagcaagaaggcccgcgtcgagctccccgacgacgaggaggattacctcatcgtccctgatcttggt

SEQ ID NO: 180 - protein, 271 aa, Oryza sativa
  REGION 1..271: note = Os02g39360.1, domain coordinates 5-42, 57-94, ortholog of AtBBX24 (G329), AtBBX25 (G1894)
MKIQCDACESAAAAVVCCADEAALCAACDVEVHAANKLAGKHQRLPLEALSARLPRCDVCQEKAAFIFCVEDRALFCRDCDEPIHVPGTLSGNHQRYLATGIRVGFASASPCDGGSDAHDSDHHAPPMGSSEHHHHHQQPAPTVAVDTPSPQFLPQGWAVDELLQFSDYETGDKLQKESSPPLGFQELEWFADIDLFHNQAPKGGAAAGRTTAEVPELFASQAANDVAYYRPPTRTAAAAFTAATGFRQSKKARVELPDDEEDYLIVPDLG

SEQ ID NO: 181 - DNA, 771 bp, Oryza sativa
  misc_feature 1..771: note = Os04g41560.2, ortholog of AtBBX24 (G329), AtBBX25 (G1894)
  source 1..771: mol_type = unassigned DNA
atgaggatccagtgcgacgcgtgcgaggccgcggcggccacggtggtgtgctgcgcggacgaggcggcgctgtgcgcgcgctgcgacgtcgagatccacgccgccaacaagctcgccagcaagcaccagcgcctcccgctcgacgccgcgctccccgccgccctcccgcgctgcgacgtctgccaggagaaggcggcgttcatcttctgcgtggaggacagggcgctcttctgccgggactgcgacgagcccatccacgtcccggggacgctctccggcaaccaccagcgctacctcaccaccggcatccgcgtcgggttcagctccgtctgtagcgccaacgccgaccacctcccgccgccagcgcccaaggggaactccaagccgccggcaagcggcatcgctgctgctgctgctcccaagccggccgtgtccgcggcggcgcaggaggtgccgtcgtcaccgttcttgccgccgtcgggctgggccgtcgaggatctcctgcagctctccgactacgagtccagcgacaagaagggctctcctattgggttcaaggatctggagtggctcgatgacatcgacctgttccatgtccagtcgccggccaagggaggcagcacggcggcggaggtgcctgagctcttcgcctcgccgcagccagcgagcaacatggggctctacaaggcgagcggtgcacgccaaagcaagaagccacgggtggagatacccgatgacgacgaggacttcttcatcgttcctgatcttgga

SEQ ID NO: 182 - protein, 257 aa, Oryza sativa
  REGION 1..257: note = Os04g41560.2, domain coordinates 5-42, 58-95, ortholog of AtBBX24 (G329), AtBBX25 (G1894)
MRIQCDACEAAAATVVCCADEAALCARCDVEIHAANKLASKHQRLPLDAALPAALPRCDVCQEKAAFIFCVEDRALFCRDCDEPIHVPGTLSGNHQRYLTTGIRVGFSSVCSANADHLPPPAPKGNSKPPASGIAAAAAPKPAVSAAAQEVPSSPFLPPSGWAVEDLLQLSDYESSDKKGSPIGFKDLEWLDDIDLFHVQSPAKGGSTAAEVPELFASPQPASNMGLYKASGARQSKKPRVEIPDDDEDFFIVPDLG

SEQ ID NO: 183 - DNA, 543 bp, Oryza sativa
  misc_feature 1..543: note = Os04g41560.4, ortholog of AtBBX24 (G329), AtBBX25 (G1894)
  source 1..543: mol_type = unassigned DNA
atgaggatccagtgcgacgcgtgcgaggccgcggcggccacggtggtgtgctgcgcggacgaggcggcgctgtgcgcgcgctgcgacgtcgagatccacgccgccaacaagctcgccagcaagcaccagcgcctcccgctcgacgccgcgctccccgccgccctcccgcgctgcgacgtctgccaggagaaggcggcgttcatcttctgcgtggaggacagggcgctcttctgccgggactgcgacgagcccatccacgtcccggggacgctctccggcaaccaccagcgctacctcaccaccggcatccgcgtcgggttcagctccgtctgtagcgccaacgccgaccacctcccgccgccagcgcccaaggggaactccaagccgccggcaagcggcatcgctgctgctgctgctcccaagccggccgtgtccgcggcggcgcaggaggtgccgtcgtcaccgttcttgccgccgtcgggctgggccgtcgaggatctcctgcagctctccgactacgagtccagcgacaaggcaaggacc

SEQ ID NO: 184 - protein, 181 aa, Oryza sativa
  REGION 1..181: note = Os04g41560.4, domain coordinates 5-42, 58-95, ortholog of AtBBX24 (G329), AtBBX25 (G1894)
MRIQCDACEAAAATVVCCADEAALCARCDVEIHAANKLASKHQRLPLDAALPAALPRCDVCQEKAAFIFCVEDRALFCRDCDEPIHVPGTLSGNHQRYLTTGIRVGFSSVCSANADHLPPPAPKGNSKPPASGIAAAAAPKPAVSAAAQEVPSSPFLPPSGWAVEDLLQLSDYESSDKART

SEQ ID NO: 185 - DNA, 714 bp, Glycine max
  misc_feature 1..714: note = Glyma11g13570.1, ortholog of AtBBX24 (G329), AtBBX25 (G1894)
  source 1..714: mol_type = unassigned DNA
atgaagattcagtgtgatgtgtgtgagaaagcccctgcaaccgtgatttgttgcgcagatgaggcagctttgtgtgccaaatgtgacgttgaagttcatgctgcaaacaagcttgcaagcaagcaccagaggcttctccttcaatgtctatcaaacaagcttcccagatgtgacatatgccaagataagccagctttcatattttgtgttgaggacagagcactcttctgtaaagactgtgatgagccaattcatttagccagtagcctttctgcaaaccaccagcgcttcctcgctactggtatcagggtggctttgggttctaattgcaccaaaggccatgaaaaaggtaacatggaaccatctaatccaaatgcacaagaagttcctgtgaaaactccttctcagcaagtgcctagcttcacatcctcttgggcagttgatgacttgttggaattaacagactttgaatcgccagacaaaaagcaatccctggaatttggagaacttgaatggctagcagatgcaggccttttcagtgaacagtttcctcatgaagctttagtcgcagctgaagttcctcagcttccagtgccacacactagcagtgttgcctcacataaagcccccaaatccttggtgtcttacaaaaagcctaggattgaagtcctggatgaagatgatgatgaacactgcactgtcccggatctcgga

SEQ ID NO: 186 - protein, 238 aa, Glycine max
  REGION 1..238: note = Glyma11g13570.1, domain coordinates 5-42, 57-94, ortholog of AtBBX24 (G329), AtBBX25 (G1894)
MKIQCDVCEKAPATVICCADEAALCAKCDVEVHAANKLASKHQRLLLQCLSNKLPRCDICQDKPAFIFCVEDRALFCKDCDEPIHLASSLSANHQRFLATGIRVALGSNCTKGHEKGNMEPSNPNAQEVPVKTPSQQVPSFTSSWAVDDLLELTDFESPDKKQSLEFGELEWLADAGLFSEQFPHEALVAAEVPQLPVPHTSSVASHKAPKSLVSYKKPRIEVLDEDDDEHCTVPDLG

SEQ ID NO: 187 - DNA, 714 bp, Glycine max
  misc_feature 1..714: note = Glyma12g05570.1, ortholog of AtBBX24 (G329), AtBBX25 (G1894)
  source 1..714: mol_type = unassigned DNA
atgaaaattcagtgcgatgtgtgtgagaaagccccggcaaccgtgatttgctgcgcagatgaggcagctttgtgtgccaaatgtgacgttgaagttcatgctgcaaacaagcttgcaagcaagcaccagaggcttctccttcaatctttatctaacaagcttcccagatgtgacatatgtcaagataagccagctttcatattttgtgttgaggacagagcactcttctgtaaagactgtgatgaacctattcatttagccagtagcctttctgcaaaccaccagcgcttccttgctactggtatccgggtggctttgggttctaattgcaccaaaggcaatgaaaaaggtcacgtggaaccatctaaaccaaaagcacaagaagttcctgcgaaaattccttctcagcaagtgcctagcttcacatcctcttgggcagttgatgacttgttggaattaacagactttgaatcaccagacaaagttcagaagcaatcccttgagtttggagaacttgaatggctagcagatgtaggcctttttggtgaacagtttcctcatgaagctttagcggcggctgaagttcctcagcttccaatgactagcagtgttggctcacacaaagcccccaaatccttgttgtcttacaaaaagcctaggattgaagtcctagatgaagatgatgatgagcacttcaccgtaccagatctcgga

SEQ ID NO: 188 - protein, 238 aa, Glycine max
  REGION 1..238: note = Glyma12g05570.1, domain coordinates 5-42, 57-94, ortholog of AtBBX24 (G329), AtBBX25 (G1894)
MKIQCDVCEKAPATVICCADEAALCAKCDVEVHAANKLASKHQRLLLQSLSNKLPRCDICQDKPAFIFCVEDRALFCKDCDEPIHLASSLSANHQRFLATGIRVALGSNCTKGNEKGHVEPSKPKAQEVPAKIPSQQVPSFTSSWAVDDLLELTDFESPDKVQKQSLEFGELEWLADVGLFGEQFPHEALAAAEVPQLPMTSSVGSHKAPKSLLSYKKPRIEVLDEDDDEHFTVPDLG

SEQ ID NO: 189 - DNA, 717 bp, Glycine max
  misc_feature 1..717: note = Glyma13g41980.1, ortholog of AtBBX24 (G329), AtBBX25 (G1894)
  source 1..717: mol_type = unassigned DNA
atgaaaattcagtgtgatgtgtgtgagagagctccggcaacagtgatttgttgtgcggatgaggcagctttgtgtgccaaatgtgacgttgaagttcatgctgcaaacaagcttgcaagcaagcaccaaaggcttcttcttcaatgtctatcaagcaagcttccaagatgtgacatttgccaagacaagccagcattcatattttgtgttgaagacagagcactcttctgtcaggattgtgatgaaccaattcattcagccggaagcctttctgcgaaccaccagcgcttccttgctactggtatccgagtggcatcgagttctaattgcaccaaggacaatgaaaaaagtcacttggagcctcctactaggaatgcacaacaagtttctgcgaaaattcctcctcagcaagtgcctagcttcacttcctcttgggctgttgatgacttattggaactaacagcctttgaatcacctgagaaaaaggaatctctgcagtttggagagctggaatggcttacagatgtgggtatttttggtgaacaatttgctcaggaagccttggctgcagctgaagtacctcagcttccagtaactcataacagtagcagtgttgcttcatacagaacttccaaatcctacatgtcccacaaaaagcctaggattgaagtcctaaatgatgatgatgatgagtattttactgtgcctgatcttggc

SEQ ID NO: 190 - protein, 239 aa, Glycine max
  REGION 1..239: note = Glyma13g41980.1, domain coordinates 5-42, 57-94, ortholog of AtBBX24 (G329), AtBBX25 (G1894)
MKIQCDVCERAPATVICCADEAALCAKCDVEVHAANKLASKHQRLLLQCLSSKLPRCDICQDKPAFIFCVEDRALFCQDCDEPIHSAGSLSANHQRFLATGIRVASSSNCTKDNEKSHLEPPTRNAQQVSAKIPPQQVPSFTSSWAVDDLLELTAFESPEKKESLQFGELEWLTDVGIFGEQFAQEALAAAEVPQLPVTHNSSSVASYRTSKSYMSHKKPRIEVLNDDDDEYFTVPDLG

SEQ ID NO: 191 - DNA, 606 bp, Glycine max
  misc_feature 1..606: note = Glyma13g41980.2, ortholog of AtBBX24 (G329), AtBBX25 (G1894)
  source 1..606: mol_type = unassigned DNA
atggatattgtaataagtttcttagaagtattgctcattttgttctttcctttctcctacatataccaacaggacaagccagcattcatattttgtgttgaagacagagcactcttctgtcaggattgtgatgaaccaattcattcagccggaagcctttctgcgaaccaccagcgcttccttgctactggtatccgagtggcatcgagttctaattgcaccaaggacaatgaaaaaagtcacttggagcctcctactaggaatgcacaacaagtttctgcgaaaattcctcctcagcaagtgcctagcttcacttcctcttgggctgttgatgacttattggaactaacagcctttgaatcacctgagaaaaaggaatctctgcagtttggagagctggaatggcttacagatgtgggtatttttggtgaacaatttgctcaggaagccttggctgcagctgaagtacctcagcttccagtaactcataacagtagcagtgttgcttcatacagaacttccaaatcctacatgtcccacaaaaagcctaggattgaagtcctaaatgatgatgatgatgagtattttactgtgcctgatcttggc

SEQ ID NO: 192 - protein, 202 aa, Glycine max
  REGION 1..202: note = Glyma13g41980.2, domain coordinates 24-57, ortholog of AtBBX24 (G329), AtBBX25 (G1894)
MDIVISFLEVLLILFFPFSYIYQQDKPAFIFCVEDRALFCQDCDEPIHSAGSLSANHQRFLATGIRVASSSNCTKDNEKSHLEPPTRNAQQVSAKIPPQQVPSFTSSWAVDDLLELTAFESPEKKESLQFGELEWLTDVGIFGEQFAQEALAAAEVPQLPVTHNSSSVASYRTSKSYMSHKKPRIEVLNDDDDEYFTVPDLG

SEQ ID NO: 193 - DNA, 603 bp, Glycine max
  misc_feature 1..603: note = Glyma13g41980.3, ortholog of AtBBX24 (G329), AtBBX25 (G1894)
  source 1..603: mol_type = unassigned DNA
atggatattgtaataagtttcttagaagtattgctcattttgttctttcctttctcctacatataccaacaggacaagccagcattcatattttgtgttgaagacagagcactcttctgtcaggattgtgatgaaccaattcattcagccggaagcctttctgcgaaccaccagcgcttccttgctactggtatccgagtggcatcgagttctaattgcaccaaggacaatgaaaaaagtcacttggagcctcctactaggaatgcacaacaagtttctgcgaaaattcctcctcagcaagtgcctagcttcacttcctcttgggctgttgatgacttattggaactaacagcctttgaatcacctgagaaagaatctctgcagtttggagagctggaatggcttacagatgtgggtatttttggtgaacaatttgctcaggaagccttggctgcagctgaagtacctcagcttccagtaactcataacagtagcagtgttgcttcatacagaacttccaaatcctacatgtcccacaaaaagcctaggattgaagtcctaaatgatgatgatgatgagtattttactgtgcctgatcttggc

SEQ ID NO: 194 - protein, 201 aa, Glycine max
  REGION 1..201: note = Glyma13g41980.3, domain coordinates 24-57, ortholog of AtBBX24 (G329), AtBBX25 (G1894)
MDIVISFLEVLLILFFPFSYIYQQDKPAFIFCVEDRALFCQDCDEPIHSAGSLSANHQRFLATGIRVASSSNCTKDNEKSHLEPPTRNAQQVSAKIPPQQVPSFTSSWAVDDLLELTAFESPEKESLQFGELEWLTDVGIFGEQFAQEALAAAEVPQLPVTHNSSSVASYRTSKSYMSHKKPRIEVLNDDDDEYFTVPDLG

SEQ ID NO: 195 - DNA, 720 bp, Glycine max
  misc_feature 1..720: note = Glyma15g03400.1, ortholog of AtBBX24 (G329), AtBBX25 (G1894)
  source 1..720: mol_type = unassigned DNA
atgaaaattcagtgtgacgtgtgtgagagagctccagcaacagtgatttgttgcgcggatgaggctgctttgtgtgccaaatgtgacgttgaagttcatgctgcaaacaagcttgcaagcaagcaccaaagacttcttcttcaatgtctatcaagcaagcttcccacatgtgacatttgccaagacaagccagcattcatattttgtgttgaagacagagcactcttctgtcaggattgtgatgaaccaattcattcagccggaagcctttccgcgaaccaccagcgcttccttgctactggtatccgagtggcctcgagttctaattgcaccaaagacaatgaaaaaagtcactcggagcctcctaataggagtgcacaacaagtttctgcgaaaattcctcctcagcaagtgcctagcttcacatcctcttgggctgttgatgacttattggaactaacaggctttgaatcacctgagaaaaaggaatctctgcagtttggagagctggaatggcttacagatgtgggtattttcggcgaacaatttgctcaggaagccttggctgcagctgaagtacctcagcttcccgaaactcataacagtagcagtgttgcttcatacaaaacctccaaatcctacatgtcccacaaaaagcctaggattgaagtcctaaatgatgatgatgatgatgagtattttactgtgcccgatctcggc

SEQ ID NO: 196 - protein, 240 aa, Glycine max
  REGION 1..240: note = Glyma15g03400.1, domain coordinates 5-42, 57-94, ortholog of AtBBX24 (G329), AtBBX25 (G1894)
MKIQCDVCERAPATVICCADEAALCAKCDVEVHAANKLASKHQRLLLQCLSSKLPTCDICQDKPAFIFCVEDRALFCQDCDEPIHSAGSLSANHQRFLATGIRVASSSNCTKDNEKSHSEPPNRSAQQVSAKIPPQQVPSFTSSWAVDDLLELTGFESPEKKESLQFGELEWLTDVGIFGEQFAQEALAAAEVPQLPETHNSSSVASYKTSKSYMSHKKPRIEVLNDDDDDEYFTVPDLG

SEQ ID NO: 197 - DNA, 699 bp, Solanum lycopersicum
  misc_feature 1..699: note = AAS67368.1, ortholog of AtBBX24 (G329), AtBBX25 (G1894)
  source 1..699: mol_type = unassigned DNA
atgaagatacagtgtgatgtgtgtgagaaagctcaagctactgtgatttgctgtgctgatgaggctgctctgtgtgcaaaatgtgatattgaagttcatgctgctaataaattagcaagcaagcaccaaaggcttcatcttcagtgcctatctaacaagcttcctccttgtgatatttgccaagataaagcagccttcatcttctgtgttgaggatagagctctcttttgcaaggactgtgacgaagcaattcattcagccagcagcctcgctaagaaccaccaacgcttcttagccactggaatccgtgtagccttgagctcaagctgtaataaggaatcagtaaaaaaccaactgcagccacaaccacctcagcagaattcccaacaagttggcttgaaaatgcctccacagcagttgtcctgtataacatcaccatcttggcctgtcgatgatttactaggatttccagattatgagtcgagtgacaagaaggatctacttgagcttggtgaatttgagtggttagggggcattgatctctttggtgaacaaacagcagctgaagtgcccgagctatcagtacctcagtcgagcaatacaaatatttacaggacaaccaaatatcaaatgccttacaagaagtccagaattgaaatcccagatgatgatgagtattttactgtccccgatcttggt

SEQ ID NO: 198 - protein, 233 aa, Solanum lycopersicum
  REGION 1..233: note = AAS67368.1, domain coordinates 5-42, 57-94, ortholog of AtBBX24 (G329), AtBBX25 (G1894)
MKIQCDVCEKAQATVICCADEAALCAKCDIEVHAANKLASKHQRLHLQCLSNKLPPCDICQDKAAFIFCVEDRALFCKDCDEAIHSASSLAKNHQRFLATGIRVALSSSCNKESVKNQLQPQPPQQNSQQVGLKMPPQQLSCITSPSWPVDDLLGFPDYESSDKKDLLELGEFEWLGGIDLFGEQTAAEVPELSVPQSSNTNIYRTTKYQMPYKKSRIEIPDDDEYFTVPDLG

SEQ ID NO: 199 - DNA, 549 bp, Arabidopsis thaliana
  misc_feature 1..549: note = AtBBX19 (G902), AT4G38960.1, paralog of AtBBX18 (G1881)
  source 1..549: mol_type = unassigned DNA
atgcggattttgtgcgatgcttgcgagaacgcagccgcaatcatcttttgcgccgccgatgaagctgccctttgtcgcccctgcgatgaaaaagttcatatgtgcaacaagctagctagtcggcatgtacgtgttggtttagctgaaccaagcaatgccccatgctgtgatatatgcgaaaatgcacctgccttcttttactgtgagatagacggtagttctctttgtctgcaatgtgacatggtagtacatgttggtggcaagagaacacacggtcggtttcttttgctgagacagagaatcgagtttccaggggataagcctaaagaaaacaatacgagggacaatttgcagaaccaaagagtctctacaaatggaaatggtgaagccaatgggaagattgatgacgaaatgattgatctaaatgctaatccacaaagagtacatgagccatcatcaaataacaacgggattgatgtaaataacgagaacaatcacgagcctgcaggccttgtaccagttggaccctttaaacgagagtctgagaag

SEQ ID NO: 200 - protein, 183 aa, Arabidopsis thaliana
  REGION 1..183: note = AtBBX19 (G902), AT4G38960.1, domain coordinates 5-42, 56-84, paralog of AtBBX18 (G1881)
MRILCDACENAAAIIFCAADEAALCRPCDEKVHMCNKLASRHVRVGLAEPSNAPCCDICENAPAFFYCEIDGSSLCLQCDMVVHVGGKRTHGRFLLLRQRIEFPGDKPKENNTRDNLQNQRVSTNGNGEANGKIDDEMIDLNANPQRVHEPSSNNNGIDVNNENNHEPAGLVPVGPFKRESEK

SEQ ID NO: 201 - DNA, 516 bp, Arabidopsis thaliana
  misc_feature 1..516: note = AtBBX18 (G1881), AT2G21320.1, paralog of AtBBX19 (G902)
  source 1..516: mol_type = unassigned DNA
atgcgaattttgtgtgatgcttgtgagagcgccgccgctatcgtcttttgcgccgccgacgaagctgccctctgttgctcctgcgacgaaaaagttcataagtgcaacaagctggctagtcggcatcttcgtgtaggcttagctgatccgagtaatgcaccaagctgtgacatatgcgaaaatgcacccgcattcttttactgtgagatagatggtagttccctttgtctacaatgtgatatggtggtacatgttggtgggaagagaacacataggcggtttctattactgagacagagaattgagtttccaggcgataagcctaatcatgctgaccaactgggactacggtgtcaaaaggcttcctctggtcgtggtcaagaatcaaatgggaatggtgatcatgatcataatatgatcgatcttaactccaatcctcaaagagtacacgagcctggatcacataaccaagaggagggtattgatgtaaataacgcaaacaatcacgagcatgaa

SEQ ID NO: 202 - protein, 172 aa, Arabidopsis thaliana
  REGION 1..172: note = AtBBX18 (G1881), AT2G21320.1, domain coordinates 5-42, 56-92, paralog of AtBBX19 (G902)
MRILCDACESAAAIVFCAADEAALCCSCDEKVHKCNKLASRHLRVGLADPSNAPSCDICENAPAFFYCEIDGSSLCLQCDMVVHVGGKRTHRRFLLLRQRIEFPGDKPNHADQLGLRCQKASSGRGQESNGNGDHDHNMIDLNSNPQRVHEPGSHNQEEGIDVNNANNHEHE

SEQ ID NO: 203 - DNA, 618 bp, Zea mays
  misc_feature 1..618: note = ACF85933.1, ortholog of AtBBX19 (G902), AtBBX18 (G1881)
  source 1..618: mol_type = unassigned DNA
atgcggacgatctgcgatgtgtgcgagagcgcaccggcggtgctcttctgtgcggccgacgaggcagcgctttgccggccctgcgacgagaaggtacacatgtgtaacaagcttgctagtcggcatgtgagagttggacttgcagaccctaataaacttgtccggtgtgatatatgtgaaaattcacccgctttcttctactgtgagatagacggcacctcactttgcctgagctgtgatatgactgttcatgttggtggcaagcgaacccatggaagatacctgctcctaaggcaaagggttgaatttccaggagataaaccgggccatatggatgacgtgcctatggaaatccaagatcctgaaaaccagagagaccagaaaaagccacccaaggagcaaacggcaaaccaccataacggcgatgatccggccactgatggcaactgtgatgaccagggtaacattgattcaaaaatgatcgatcttaatatgcgcccggttcgtactcatgggcaagaatcaaattcccagactcagggggtaggtcttagtgtgaacaaccatgactctccaggggtggtgccaacaagcaattccgaaagagacaccagcaag

SEQ ID NO: 204 - protein, 206 aa, Zea mays
  REGION 1..206: note = ACF85933.1, domain coordinates 5-42, 56-84, ortholog of AtBBX19 (G902), AtBBX18 (G1881)
MRTICDVCESAPAVLFCAADEAALCRPCDEKVHMCNKLASRHVRVGLADPNKLVRCDICENSPAFFYCEIDGTSLCLSCDMTVHVGGKRTHGRYLLLRQRVEFPGDKPGHMDDVPMEIQDPENQRDQKKPPKEQTANHHNGDDPATDGNCDDQGNIDSKMIDLNMRPVRTHGQESNSQTQGVGLSVNNHDSPGVVPTSNSERDTSK

SEQ ID NO: 205 - DNA, 618 bp, Zea mays; identical to SEQ ID NO: 203
  misc_feature 1..618: note = ACG27957.1, ortholog of AtBBX19 (G902), AtBBX18 (G1881)
  source 1..618: mol_type = unassigned DNA

SEQ ID NO: 206 - protein, 206 aa, Zea mays; identical to SEQ ID NO: 204
  REGION 1..206: note = ACG27957.1, domain coordinates 5-42, 56-84, ortholog of AtBBX19 (G902), AtBBX18 (G1881)

SEQ ID NO: 207 - DNA, 615 bp, Zea mays
  misc_feature 1..615: note = ACL52378.1, ortholog of AtBBX19 (G902), AtBBX18 (G1881)
  source 1..615: mol_type = unassigned DNA
atgcggacgatctgcgatgtgtgcgagagcgcaccggcggtgctcttctgtgcggccgacgaggctgcgctttgccggccttgcgacgagaaggtacacatgtgcaacaagcttgctagtcggcatgtgagagttgggcttgcagaccctaataaactggcacgttgtgatatatgcgagaattcacctgctttcttctactgtgagatagatggcacctcactttgcctgagctgtgatatgactgttcatgttggtggcaaaagaacccatggtagatacctgctcctaaggcaaagggttgaatttccaggagataaaccgggccatatggatgacgtgcctatggagatcaaagatcctgaaaaccagagagagcaaaacacgcccaaggagcaaatggcaaaccaccataacgtcaatgatccagtctctgatggcaattgcgatggccagggtaacattgattcgaaaatgatcgatcttaatatgcgcccggctcgtactcatgggcaaggatcaaattcccagactcagggggtggatcttagtgtgaacaaccatgactctccaggggtggtgccaacaagtaattccgaaagagacgccatcaag

SEQ ID NO: 208 - protein, 205 aa, Zea mays
  REGION 1..205: note = ACL52378.1, domain coordinates 5-42, 56-88, ortholog of AtBBX19 (G902), AtBBX18 (G1881)
MRTICDVCESAPAVLFCAADEAALCRPCDEKVHMCNKLASRHVRVGLADPNKLARCDICENSPAFFYCEIDGTSLCLSCDMTVHVGGKRTHGRYLLLRQRVEFPGDKPGHMDDVPMEIKDPENQREQNTPKEQMANHHNVNDPVSDGNCDGQGNIDSKMIDLNMRPARTHGQGSNSQTQGVDLSVNNHDSPGVVPTSNSERDAIK

SEQ ID NO: 209 - DNA, 618 bp, Zea mays; identical to SEQ ID NO: 203
  misc_feature 1..618: note = NP_001141274.1, ortholog of AtBBX19 (G902), AtBBX18 (G1881)
  source 1..618: mol_type = unassigned DNA

SEQ ID NO: 210 - protein, 206 aa, Zea mays; identical to SEQ ID NO: 204
  REGION 1..206: note = NP_001141274.1, domain coordinates 5-42, 56-84, ortholog of AtBBX19 (G902), AtBBX18 (G1881)

SEQ ID NO: 211 - DNA, 1346 bp, Populus trichocarpa
  misc_feature 1..1346: note = Pt_648693, ortholog of AtBBX19 (G902), AtBBX18 (G1881)
  source 1..1346: mol_type = unassigned DNA
aacagaaggcaggcaaagcaagcgacgaatatcactccattttcctttcttcttcatcgtccccagtttcttagactcataaacaagtcctgttgtatgctctcatcccctctcctctcctctcctctccttttcaccatttaaatctccctctccagtctgtttgtcattgactgtttaacttaataataatcttaatctcatgaaatattgacaaatggtgatgagaaggagaaaggagaaataatagaaagtagagattgaaaggtgacaggacagtgacaataataaagaaagaaccaaaactgattctaaaaaacggaaatgcgaatgctttgtgatgtgtgtgaaagcgcagctgcgattcttttctgtgctgctgatgaagccgcgctttgccgctcctgcgacgagaaggttcatatgtgtaacaagctagctagtcggcatgtacgagtaggacttgcagatcctagtgatgttccacagtgtgacatatgtgaaaaggcaccagctttcttttactgcgagatagatggcagttcgctctgcttgcaatgtgatatgatagtgcatgttggtggtaaaaggacccatgggagatatctcctgttgaggcagagagttgagtttccaggggataaacctggctgtacagaggaacaaggacagcaaccgcttgatgataatgaaacaagaagggaccaaaatcagccgcctaagctcacagctagagaaaatcaacagaaccacagggcctctccagttccaatggtagagaataacactgatagtgacgggaaaatggacaacaaattgattgatcttaatgccaggcctcagcgtgtacatgggaaaaatccaactaaccaggaaaaccatgaatcctctagtttggctccttttggattctttaaaggggagccacaaaagtaagcaaggtatgatctgcatttagtaagcctaactcgtgttttcttttctccatttattgtttcataaagttgacattatggatggttgtgcgggaagcttcaagtttccattgaagtcgattttcttaaaccagctcttggatgacttctacatgcaaatatgcataggactctaaattccgctgcttgtaagatggtgaatgtaatcttcttttattcaaactgtggtttaaatatgtagctatatggatctgcaatgaagataaacaaatgttgttatggccttttaccgtgttccatgtttgtaagggcataacaaactgtggtttaaatatatagctatatggattctcaaaagaacaaaaagggcacatctgttattgagatggagcctagctgaaatttactata

SEQ ID NO: 212 - protein, 203 aa, Populus trichocarpa
  REGION 1..203: note = Pt_648693, domain coordinates 5-42, 56-88, ortholog of AtBBX19 (G902), AtBBX18 (G1881)
MRMLCDVCESAAAILFCAADEAALCRSCDEKVHMCNKLASRHVRVGLADPSDVPQCDICEKAPAFFYCEIDGSSLCLQCDMIVHVGGKRTHGRYLLLRQRVEFPGDKPGCTEEQGQQPLDDNETRRDQNQPPKLTARENQQNHRASPVPMVENNTDSDGKMDNKLIDLNARPQRVHGKNPTNQENHESSSLAPFGFFKGEPQK

SEQ ID NO: 213 - DNA, 996 bp, Populus trichocarpa
  misc_feature 1..996: note = Pt_721866, ortholog of AtBBX19 (G902), AtBBX18 (G1881)
  source 1..996: mol_type = unassigned DNA
atgcgaacgatttgtgacgtgtgtgagagcgcagccgcgattcttttttgtgcagctgatgaagccgctctttgccgttcctgtgacgagaaggttcatttgtgcaacaagctagctagtcggcacgtgcgagtaggacttgcagatcctagcgctgttccacagtgtgacatatgtgaaaatgcaccagctttcttttactgtgagatagatggtagttcgctctgcttgcaatgcgatatgatagtacatgttggtggtaaaagaacccatggtagatatctcctgttgaggcagagggttgagtttccaggggataaacctggccggatggaggagcaaggacaacaacctcttgatcataatgaaacaaggagggaccaaaaccagccacttaaactcacagctagagaaaataaacaaaaccacagagcctctccagttccaatggtagagaataacacagatagcgacggaaaaatggacaacaatttgattgatcttaatgccaggcctcagcggatacatggacaaaattcaactaaccaggaaaatcacgaatcttctagtgcggttcctgttggatccttcaaaagagagcctcaaaagtaagcaaggtgatttcggatgcatgttgagatattcagtactgcaaccagccttgtctgctcttgcacttgcagcatttcttgtggttaggctttccttcctagcgcgaagttggtgaggtgatttttagtcttttgtatcaaatatcaatagagatatagcttgtgtgaaagaaataacaagactctttgttggttagcatttggctcatgtgctggcttgagttcaccatcaccttcaggtcctgagatttataatttaataaagcacgattcattctaaagatgatgatgtgaagcgaccttcggacaggagaaagtggcttctgtcgtcgttattattttgtttttttggtcagaaatataaaaaaaatccacttttaaaaaa

SEQ ID NO: 214 - protein, 203 aa, Populus trichocarpa
  REGION 1..203: note = Pt_721866, domain coordinates 5-42, 56-88, ortholog of AtBBX19 (G902), AtBBX18 (G1881)
MRTICDVCESAAAILFCAADEAALCRSCDEKVHLCNKLASRHVRVGLADPSAVPQCDICENAPAFFYCEIDGSSLCLQCDMIVHVGGKRTHGRYLLLRQRVEFPGDKPGRMEEQGQQPLDHNETRRDQNQPLKLTARENKQNHRASPVPMVENNTDSDGKMDNNLIDLNARPQRIHGQNSTNQENHESSSAVPVGSFKREPQK

SEQ ID NO: 215 - DNA, 1073 bp, Populus trichocarpa
  misc_feature 1..1073: note = Pt_741179, ortholog of AtBBX19 (G902), AtBBX18 (G1881)
  source 1..1073: mol_type = unassigned DNA
ttatgtccctctctttctatcaccaccaacaacaaaaacattaacatttgatagacagcgtaaaaatcaaatagtgaagctgtgtcttgttgtggtgtttgttatctttattaaaagaaactgtacgtaaatattttttctattttcttgattttggagaaatgagagtgagagataattaattatgcgtaccctttgtgacgcttgtgaaagtgcttttgctattgttttctgtgctgctgatgaggccgcactttgccttgcctgcgataagaaggttcatatgtgcaacaaacttgcgagcaggcatgttcgagtgggtctggcgaatcccagtgaagttcctcgttgtgatatatgtgaaaatgcacctgctttcttctactgtgagacagatggaagttccctttgcttgcaatgtgatatgactgttcatgttggaggtaaaagaacacacgggagatatcttctgttgagacaaaaaattgagtttccaggggatcaacctcagcctgaggacccagcaccgcaacctatgtatccaggggagacaagaagaggacagaatcgaccacaaaaggcaacatcaggagagaacagacagaatcgccaagcttctccagttctgatgtcggtcactaattctgatgggcatgacaaggtggataagaatatgattgatttgaatatgaagcctcatcgaattcatgaacatgcttcaaataatcaggaacaataatatttttatattaacggagcggccgcgaagctgaaagcagggttcctatgttgcctggaaagctggagaagagcagggttttttctatgcttctcttatcaagtagttggcatttcatgtttcaatatttcattttcttctcccctgttttcctttgatatggatgatgtgctttaagtcttgcagatggatttttaccaataatgtagtttagttgctagacaagctttggacggtatacacatctattgtgaaaaaacatgcagcaattatagatggatattgtaatagaagagaaactagaaataactgaagatttaatatagttcgaaaa

SEQ ID NO: 216 - protein, 184 aa, Populus trichocarpa
  REGION 1..184: note = Pt_741179, domain coordinates 5-42, 56-88, ortholog of AtBBX19 (G902), AtBBX18 (G1881)
MRTLCDACESAFAIVFCAADEAALCLACDKKVHMCNKLASRHVRVGLANPSEVPRCDICENAPAFFYCETDGSSLCLQCDMTVHVGGKRTHGRYLLLRQKIEFPGDQPQPEDPAPQPMYPGETRRGQNRPQKATSGENRQNRQASPVLMSVTNSDGHDKVDKNMIDLNMKPHRIHEHASNNQEQ

SEQ ID NO: 217 - DNA, 1347 bp, Populus trichocarpa
  misc_feature 1..1347: note = Pt_820080, ortholog of AtBBX19 (G902), AtBBX18 (G1881)
  source 1..1347: mol_type = unassigned DNA
gctgtgccttgttgcgatgtttgatatctttattaaaggaaactgtacttgaatcctttttcttgttgcttgattttggagaaatgagagtgagagaaaattatgcgtaccctttgcgacgcctgtgagagtgctgctgctattgtcttctgtgctgctgatgaggctgcactttgccttgcctgcgatgagaaggttcatatgtgcaacaaacttgctagcaggcatgtgcgagtgggtctggcaaatcccagtgacgttccacgctgtgacatatgtgaaaatgcacctgctttcttttattgtgagacagatggaagttccctttgcttgcaatgtgatatgactgttcatgttggaggtaaaaggacacatgggagatatcttttgttgagacaaagagttgagtttccaggggataaacctcagcctgatgacctacattcgcaacctatgcatccaggggagacaaggaaaggacagaatcagccaccaaaggcaacagcagaagagaagcgacagaatcgccaggtttctccagctccaatgtcactctctaattctgatggacatgacaaagtggataaaaagatgattgatttgaatatgaagcctcaacgaactgatcacgaacaagcttctaataatcaggaactgtgacattattatgttaatggagcgatcgcaaagctgaaagcacggttcctatgccctcagagagctggacaggagcagggtttcttctatgcttctcttacgtagtacgtagttagcatgtcatgtgtcaatatttcatgttcttctcccctgtttttctttcgacatggatgatgtgcttctaaggcttggggaatgtgttttcacgaataatgtagcttagttgtttagactagcagtggacagtatccacatcttcaattgcaaagaaaaggtatagaatcaagtgcaattggttgctttgtggttgtgttcgagatatttttcttcccatgcatcccttatttccaagtgtctgatattcgcagaagtggcattgtaagaaatttctttggagtctagagaattcggtatactatttcagattactgcaagagcaagtttccagctgttctttgtcttgatttcggatggcgaacgttgtttttgaaagcagacttctgatacatgtcacaacgcttccaacgcaaatcgatagaatcgttacatttgcatgcatgggccatggatctaagctcgctggatttcaagaccagtggcatttcccatttttctgtgaacgcaacttggacacctcctgtcttctgtgtatgtgttgaagaacaataatcagtttattaaccatggttt

SEQ ID NO: 218 - protein, 185 aa, Populus trichocarpa
  REGION 1..185: note = Pt_820080, domain coordinates 5-42, 56-88, ortholog of AtBBX19 (G902), AtBBX18 (G1881)
MRTLCDACESAAAIVFCAADEAALCLACDEKVHMCNKLASRHVRVGLANPSDVPRCDICENAPAFFYCETDGSSLCLQCDMTVHVGGKRTHGRYLLLRQRVEFPGDKPQPDDLHSQPMHPGETRKGQNQPPKATAEEKRQNRQVSPAPMSLSNSDGHDKVDKKMIDLNMKPQRTDHEQASNNQEL

SEQ ID NO: 219 - DNA, 633 bp, Oryza sativa
  misc_feature 1..633: note = Os09g35880.1, ortholog of AtBBX19 (G902), AtBBX18 (G1881)
  source 1..633: mol_type = unassigned DNA
atgcggacgatctgcgacgtgtgcgagagcgcgccggcggtgctcttctgcgtggccgacgaggccgcgctctgccggtcctgcgacgagaaggtgcatatgtgtaacaagcttgctaggcggcacgtgagagttgggcttgcagaccctaataaagttcaacgctgtgatatatgtgaaaatgcccccgccttcttctattgcgagatagatggtacatcactttgccttagttgtgatatgactgttcatgttggtgggaaacgaacccatggaagatacctgctcctaaggcaacgggttgaatttccaggagataaaccaggtcatatggatgatgttgctatgcaacagaaagatcctgaaaaccggacggatcaaaagaaggcccctcactcagtaacaaaggagcaaatggcaaaccatcataatgtgtctgatgatccagcctcagatggcaactgcgatgaccagggtaacatcgattccaaaatgattgatcttaatatgagacccgtccgtactcatggacaaggttcaaactcacagactcagggcgtggatgttagcgtcaacaatcatgattctccaggagtggtgccaacatgtaatttcgaacgagaagccaacaaa

SEQ ID NO: 220 - protein, 211 aa, Oryza sativa
  REGION 1..211: note = Os09g35880.1, domain coordinates 5-42, 56-88, ortholog of AtBBX19 (G902), AtBBX18 (G1881)
MRTICDVCESAPAVLFCVADEAALCRSCDEKVHMCNKLARRHVRVGLADPNKVQRCDICENAPAFFYCEIDGTSLCLSCDMTVHVGGKRTHGRYLLLRQRVEFPGDKPGHMDDVAMQQKDPENRTDQKKAPHSVTKEQMANHHNVSDDPASDGNCDDQGNIDSKMIDLNMRPVRTHGQGSNSQTQGVDVSVNNHDSPGVVPTCNFEREANK

SEQ ID NO: 221 - DNA, 552 bp, Glycine max
  misc_feature 1..552: note = Glyma01g37370.1, ortholog of AtBBX19 (G902), AtBBX18 (G1881)
  source 1..552: mol_type = unassigned DNA
atgcgaacgctttgtgatgcttgtgagagcgccgcggctatagttttctgcgccgctgatgaggctgcactttgtcgtgcctgtgatgagaaggttcacatgtgcaacaagctagctagtaggcatgtgagggttggtcttgctagtccaagtgatgtaccacggtgtgacatatgtgagaatgcacctgctttcttctattgcgagacagatggaagttccctttgtttgcagtgtgatatgattgttcatgttggaggtaaaagaacgcatggaagatatcttctgttcaggcaaagagttgagtttccaggagataaatctagtcatgctgaaaatccggcttcacaaccattggaaccaggtgaggctaaaagaggacaaaatccacttcccaaactaaaaatgggagagaagcaacaaaatcacaagatgcctatggttccaacgccaggacctgatgctgatgggcatgccaagatggaaagtaaaatgattgatttgaacatgaagcctaatagaatacatgaacaagcatcaaataatcagcca

SEQ ID NO: 222 - protein, 184 aa, Glycine max
  REGION 1..184: note = Glyma01g37370.1, domain coordinates 5-42, 56-88, ortholog of AtBBX19 (G902), AtBBX18 (G1881)
MRTLCDACESAAAIVFCAADEAALCRACDEKVHMCNKLASRHVRVGLASPSDVPRCDICENAPAFFYCETDGSSLCLQCDMIVHVGGKRTHGRYLLFRQRVEFPGDKSSHAENPASQPLEPGEAKRGQNPLPKLKMGEKQQNHKMPMVPTPGPDADGHAKMESKMIDLNMKPNRIHEQASNNQP

SEQ ID NO: 223 - DNA, 579 bp, Glycine max
  misc_feature 1..579: note = Glyma11g07930.1, ortholog of AtBBX19 (G902), AtBBX18 (G1881)
  source 1..579: mol_type = unassigned DNA
atgcgaacgctttgtgatgcttgtgagagtgcggcggctatagttttctgcgccgctgatgaggctgcactttgtcgtgcctgtgatgagaaggttcacatgtgcaataagctagctagtaggcatgtgagggttggtcttgcaagtccaagtgatgtgccacggtgtgacatatgtgagaatgcaccagctttcttctattgcgagacagatggaagttccctttgtttgcagtgtgatatgattgttcatgttggtggtaaaagaacgcatggaagatatcttctgttcaggcaaagagttgagtttccaggagataaatctagtcatgctgaaaatccggcttcacaagcattggaaccaggtgaggctaaaagaggacaaaatccacttcccaagctaaaaatgggagagaaacaacaaaatcacaggatgcctatggttccaacaccaggacctgatgctgatgggcaaaccaagatggaaactaaaatgattgatttgaacatgaagcctaatagaatacatgaacaagcatcaaataaccagtgcagttggatgaaagtagaaccctttcca

SEQ ID NO: 224 - protein, 193 aa, Glycine max
  REGION 1..193: note = Glyma11g07930.1, domain coordinates 5-42, 56-88, ortholog of AtBBX19 (G902), AtBBX18 (G1881)
MRTLCDACESAAAIVFCAADEAALCRACDEKVHMCNKLASRHVRVGLASPSDVPRCDICENAPAFFYCETDGSSLCLQCDMIVHVGGKRTHGRYLLFRQRVEFPGDKSSHAENPASQALEPGEAKRGQNPLPKLKMGEKQQNHRMPMVPTPGPDADGQTKMETKMIDLNMKPNRIHEQASNNQCSWMKVEPFP

SEQ ID NO: 225 - DNA, 552 bp, Glycine max
  misc_feature 1..552: note = Glyma11g07930.2, ortholog of AtBBX19 (G902), AtBBX18 (G1881)
  source 1..552: mol_type = unassigned DNA
atgcgaacgctttgtgatgcttgtgagagtgcggcggctatagttttctgcgccgctgatgaggctgcactttgtcgtgcctgtgatgagaaggttcacatgtgcaataagctagctagtaggcatgtgagggttggtcttgcaagtccaagtgatgtgccacggtgtgacatatgtgagaatgcaccagctttcttctattgcgagacagatggaagttccctttgtttgcagtgtgatatgattgttcatgttggtggtaaaagaacgcatggaagatatcttctgttcaggcaaagagttgagtttccaggagataaatctagtcatgctgaaaatccggcttcacaagcattggaaccaggtgaggctaaaagaggacaaaatccacttcccaagctaaaaatgggagagaaacaacaaaatcacaggatgcctatggttccaacaccaggacctgatgctgatgggcaaaccaagatggaaactaaaatgattgatttgaacatgaagcctaatagaatacatgaacaagcatcaaataaccagcca

SEQ ID NO: 226 - protein, 184 aa, Glycine max
  REGION 1..184: note = Glyma11g07930.2, domain coordinates 5-42, 56-88, ortholog of AtBBX19 (G902), AtBBX18 (G1881)
MRTLCDACESAAAIVFCAADEAALCRACDEKVHMCNKLASRHVRVGLASPSDVPRCDICENAPAFFYCETDGSSLCLQCDMIVHVGGKRTHGRYLLFRQRVEFPGDKSSHAENPASQALEPGEAKRGQNPLPKLKMGEKQQNHRMPMVPTPGPDADGQTKMETKMIDLNMKPNRIHEQASNNQP

SEQ ID NO: 227 - DNA, 552 bp, Glycine max; identical to SEQ ID NO: 225
  misc_feature 1..552: note = Glyma11g07930.3, ortholog of AtBBX19 (G902), AtBBX18 (G1881)
  source 1..552: mol_type = unassigned DNA

SEQ ID NO: 228 - protein, 184 aa, Glycine max; identical to SEQ ID NO: 226
  REGION 1..184: note = Glyma11g07930.3, domain coordinates 5-42, 56-88, ortholog of AtBBX19 (G902), AtBBX18 (G1881)

SEQ ID NO: 229 - DNA, 567 bp, Glycine max
  misc_feature 1..567: note = Glyma11g07930.4, ortholog of AtBBX19 (G902), AtBBX18 (G1881)
  source 1..567: mol_type = unassigned DNA
atgcgaacgctttgtgatgcttgtgagagtgcggcggctatagttttctgcgccgctgatgaggctgcactttgtcgtgcctgtgatgagaaggttcacatgtgcaataagctagctagtaggcatgtgagggttggtcttgcaagtccaagtgatgtgccacggtgtgacatatgtgagaatgcaccagctttcttctattgcgagacagatggaagttccctttgtttgcagtgtgatatgattgttcatgttggtggtaaaagaacgcatggaagatatcttctgttcaggcaaagagttgagtttccaggagataaatctagtcatgctgaaaatccggcttcacaagcattggaaccaggtgaggctaaaagaggacaaaatccacttcccaagctaaaaatgggagagaaacaacaaaatcacaggatgcctatggttccaacaccaggacctgatgctgatgggcaaaccaagatggaaactaaaatgattgatttgaacatgaagcctaatagaatacatgaacaagcatcaaataaccaggttagagttcatgggaag

SEQ ID NO: 230 - protein, 189 aa, Glycine max
  REGION 1..189: note = Glyma11g07930.4, domain coordinates 5-42, 56-88, ortholog of AtBBX19 (G902), AtBBX18 (G1881)
MRTLCDACESAAAIVFCAADEAALCRACDEKVHMCNKLASRHVRVGLASPSDVPRCDICENAPAFFYCETDGSSLCLQCDMIVHVGGKRTHGRYLLFRQRVEFPGDKSSHAENPASQALEPGEAKRGQNPLPKLKMGEKQQNHRMPMVPTPGPDADGQTKMETKMIDLNMKPNRIHEQASNNQVRVHGK

SEQ ID NO: 231 - DNA, 636 bp, Glycine max
  misc_feature 1..636: note = Glyma11g11850.1, ortholog of AtBBX19 (G902), AtBBX18 (G1881)
  source 1..636: mol_type = unassigned DNA
atgcgaaccctttgcgacgtatgcgagagcgccgctgccattctcttctgcgccgctgatgaggccgctctctgctccgcttgtgaccacaagatccatatgtgtaacaaacttgctagtaggcatgttcgagttggccttgctgaccccactgatgttccacgctgtgacatatgtgaaaatgcacctgctttcttttactgtgagatagatggtagttcgctgtgtctgcaatgtgatatgattgtgcatgttggtggtaaaagaacccatggaagatatcttctattgaggcaaagagctcagtttccaggtgataaacctgcccaaatggaagaactagagctgcaacctatggatcaaaatgaatcgagaagggatgaaagtcagtcactcaagctaaaaacaagagatagtcagcagaatcacagtgtctcaccttttccaaggcaagaaaataatattgatggtcatggaaaaatggataaaaagttaattgatcttaataccaggccactgcggttaaatggatcagcaccaaacaaccaggaacaatgcatggatattttaagaggcaataatcatgaatctgccagtgtgccaccagtagaatccttcaaacaagagtctgagaag

SEQ ID NO: 232 - protein, 212 aa, Glycine max
  REGION 1..212: note = Glyma11g11850.1, domain coordinates 5-42, 56-88, ortholog of AtBBX19 (G902), AtBBX18 (G1881)
MRTLCDVCESAAAILFCAADEAALCSACDHKIHMCNKLASRHVRVGLADPTDVPRCDICENAPAFFYCEIDGSSLCLQCDMIVHVGGKRTHGRYLLLRQRAQFPGDKPAQMEELELQPMDQNESRRDESQSLKLKTRDSQQNHSVSPFPRQENNIDGHGKMDKKLIDLNTRPLRLNGSAPNNQEQCMDILRGNNHESASVPPVESFKQESEK

SEQ ID NO: 233 - DNA, 537 bp, Glycine max
  misc_feature 1..537: note = Glyma12g04130.1, ortholog of AtBBX19 (G902), AtBBX18 (G1881)
  source 1..537: mol_type = unassigned DNA
atgcgaaccctttgcgacgtatgcgagagcgccgctgccattgtcttctgcgccgctgatgaggctgctctctgctccgcttgtgaccacaagatccatatgtgtaacaaacttgctagtagacacgttcgggttggccttgctgaccccactgatgttccacgctgtgacatatgtgaaaacgcacctgctttcttttactgtgagatagatggtagttcgctgtgtctgcaatgtgatatgattgtgcatgttggtggtaaaagaactcatggaagatatcttctattgaggcaaagagttcagtttccatatagtcagcagaatcacagtgtctcacctgttccaagacaagaaaataatattgatggtcatggaaaaatggataaaaagttaattgatcttaatacgaggccactgcggttaaatggagcagcaccaaacaaccaggaacgaggtatggatattttaagaggcaataatcataaatctgcgagtgtgccaccagtagaatccttcaaacaagagtctgagaag

SEQ ID NO: 234 - protein, 179 aa, Glycine max
  REGION 1..179: note = Glyma12g04130.1, domain coordinates 5-42, 56-88, ortholog of AtBBX19 (G902), AtBBX18 (G1881)
MRTLCDVCESAAAIVFCAADEAALCSACDHKIHMCNKLASRHVRVGLADPTDVPRCDICENAPAFFYCEIDGSSLCLQCDMIVHVGGKRTHGRYLLLRQRVQFPYSQQNHSVSPVPRQENNIDGHGKMDKKLIDLNTRPLRLNGAAPNNQERGMDILRGNNHKSASVPPVESFKQESEK

SEQ ID NO: 235 - protein, 37 aa, Arabidopsis thaliana
  REGION 1..37: note = conserved domain of AtBBX28 (G1481)
CDLCNGVARMYCESDQASLCWDCDGKVHGANFLVAKH

SEQ ID NO: 236 - protein, 37 aa, Arabidopsis thaliana
  REGION 1..37: note = conserved domain of AtBBX29 (G900)
CELCCGVARMYCESDQASLCWDCDGKVHGANFLVAKH

SEQ ID NO: 237 - protein, 37 aa, Zea mays
  REGION 1..37: note = conserved domain of ACN33981.1
CELCGAAARVYCGADEATLCWGCDAQVHGANFLVARH

SEQ ID NO: 238 - protein, 37 aa, Populus trichocarpa
  REGION 1..37: note = conserved domain of Pt_568988
CELCGSSARMYCESDQASLCWDCDEKVHTANFLVAKH

SEQ ID NO: 239 - protein, 37 aa, Populus trichocarpa
  REGION 1..37: note = conserved domain of Pt_571834
CELCKNPARTYCESDEANLCWNCDTKVHGANFLVARH

SEQ ID NO: 240 - protein, 37 aa, Populus trichocarpa
  REGION 1..37: note = conserved domain of Pt_582065
CELCGSSARMFCESDQASLCWDCDEKVHSANFLVAKH

SEQ ID NO: 241 - protein, 37 aa, Populus trichocarpa
  REGION 1..37: note = conserved domain of Pt_758524
CELCDSFAQMHCESDQAILCSACDAYVHSANFLAAKH

SEQ ID NO: 242 - protein, 37 aa, Oryza sativa; identical to SEQ ID NO: 237
  REGION 1..37: note = conserved domain of Os08g08120.1

SEQ ID NO: 243 - protein, 37 aa, Oryza sativa; identical to SEQ ID NO: 237
  REGION 1..37: note = conserved domain of Os08g08120.2

SEQ ID NO: 244 - protein, 37 aa, Oryza sativa; identical to SEQ ID NO: 237
  REGION 1..37: note = conserved domain of Os08g08120.3

SEQ ID NO: 245 - protein, 37 aa, Glycine max
  REGION 1..37: note = conserved domain of Glyma06g39810.1
CVLCEKRAMMLCDSDQAKLCWECDEKVHSANFLVAKH

SEQ ID NO: 246 - protein, 37 aa, Glycine max
  REGION 1..37: note = conserved domain of Glyma06g45620.1
CELCKVPARIFCESDQASLCWDCDAKVHSANFLVARH

SEQ ID NO: 247 - protein, 37 aa, Glycine max
  REGION 1..37: note = conserved domain of G4015
CELCHQLASLYCPSDSAFLCFHCDAAVHAANFLVARH

SEQ ID NO: 248 - protein, 37 aa, Glycine max
  REGION 1..37: note = conserved domain of Glyma09g35960.1
CALCKKRAMMLCDSDQAKLCWECDEKVHSANFLVAKH

SEQ ID NO: 249 - protein, 37 aa, Glycine max; identical to SEQ ID NO: 246
  REGION 1..37: note = conserved domain of Glyma12g11230.1

SEQ ID NO: 250 - protein, 37 aa, Glycine max
  REGION 1..37: note = conserved domain of Glyma12g32470.1
CELCKLPARTFCESDQASLCWDCDAKVHGANFLVARH

SEQ ID NO: 251 - protein, 37 aa, Glycine max
  REGION 1..37: note = conserved domain of G4014
CELCNSPAKLFCESDQASLCWKCDAKVHSANFLVTKH

SEQ ID NO: 252 - protein, 37 aa, Glycine max
  REGION 1..37: note = conserved domain of G4016
CELCKLPARTFCESDQASLCWDCDAKVHGANFLVERH

SEQ ID NO: 253 - protein, 37 aa, Glycine max
  REGION 1..37: note = conserved domain of Glyma15g07140.1
CELCNSPAKLFCESDQASLCWECDAKVHSANFLVTKH

SEQ ID NO: 254 - protein, 38 aa, Arabidopsis thaliana
  REGION 1..38: note = conserved domain of AtBBX24 (G329)
CDVCEKAPATVICCADEAALCPQCDIEIHAANKLASKH

SEQ ID NO: 255 - protein, 38 aa, Arabidopsis thaliana
  REGION 1..38: note = conserved domain of AtBBX24 (G329)
CDICQEKAAFIFCVEDRALLCRDCDESIHVANSRSANH

SEQ ID NO: 256 - protein, 38 aa, Arabidopsis thaliana
  REGION 1..38: note = conserved domain of AtBBX25 (G1894)
CDVCEKAPATLICCADEAALCAKCDVEVHAANKLASKH

SEQ ID NO: 257 - protein, 38 aa, Arabidopsis thaliana
  REGION 1..38: note = conserved domain of AtBBX25 (G1894)
CDICLEKAAFIFCVEDRALLCRDCDEATHAPNTRSANH

SEQ ID NO: 258 - protein, 38 aa, Zea mays
  REGION 1..38: note = conserved domain of ACF84591.1
CDACEGAAATVVCCADEAALCARCDVEIHAANKLASKH

SEQ ID NO: 259 - protein, 38 aa, Zea mays
  REGION 1..38: note = conserved domain of ACF84591.1
CDVCQEKAAFIFCVEDRALFCQDCDEPIHVPGTLSGNH

SEQ ID NO: 260 - protein, 38 aa, Zea mays; identical to SEQ ID NO: 258
  REGION 1..38: note = conserved domain of ACF86823.1

SEQ ID NO: 261 - protein, 38 aa, Zea mays
  REGION 1..38: note = conserved domain of ACF86823.1
CDVCQEKAAFIFCVEDRALFCRDCDEPIHVPGTLSGNH

SEQ ID NO: 262 - protein, 38 aa, Zea mays; identical to SEQ ID NO: 258
  REGION 1..38: note = conserved domain of CAW36812.1

SEQ ID NO: 263 - protein, 38 aa, Zea mays; identical to SEQ ID NO: 261
  REGION 1..38: note = conserved domain of CAW36812.1

SEQ ID NO: 264 - protein, 38 aa, Populus trichocarpa
  REGION 1..38: note = conserved domain of Pt_216458
CDVCEKAPATVICCADEAALCEKCDIEVHAANKLASKH

SEQ ID NO: 265 - protein, 38 aa, Populus trichocarpa
  REGION 1..38: note = conserved domain of Pt_216458
CDICQEKAAFIFCVEDRALFCRDCDEPIHSAGSLSANH

SEQ ID NO: 266 - protein, 38 aa, Populus trichocarpa
  REGION 1..38: note = conserved domain of Pt_827145
CDVCEKAPATVICCADEAALCAKCDIEVHAANKLASKH

SEQ ID NO: 267 - protein, 38 aa, Populus trichocarpa; identical to SEQ ID NO: 265
  REGION 1..38: note = conserved domain of Pt_827145

SEQ ID NO: 268 - protein, 38 aa, Oryza sativa
  REGION 1..38: note = conserved domain of Os02g39360.1
CDACESAAAAVVCCADEAALCAACDVEVHAANKLAGKH

SEQ ID NO: 269 - protein, 38 aa, Oryza sativa; identical to SEQ ID NO: 261
  REGION 1..38: note = conserved domain of Os02g39360.1

SEQ ID NO: 270 - protein, 38 aa, Oryza sativa
  REGION 1..38: note = conserved domain of Os04g41560.2
CDACEAAAATVVCCADEAALCARCDVEIHAANKLASKH

SEQ ID NO: 271 - protein, 38 aa, Oryza sativa; identical to SEQ ID NO: 261
  REGION 1..38: note = conserved domain of Os04g41560.2

SEQ ID NO: 272 - protein, 38 aa, Oryza sativa; identical to SEQ ID NO: 270
  REGION 1..38: note = conserved domain of Os04g41560.4

SEQ ID NO: 273 - protein, 38 aa, Oryza sativa; identical to SEQ ID NO: 261
  REGION 1..38: note = conserved domain of Os04g41560.4

SEQ ID NO: 274 - protein, 38 aa, Glycine max
  REGION 1..38: note = conserved domain of Glyma11g13570
CDVCEKAPATVICCADEAALCAKCDVEVHAANKLASKH

SEQ ID NO: 275 - protein, 38 aa, Glycine max
  REGION 1..38: note = conserved domain of Glyma11g13570.1
CDICQDKPAFIFCVEDRALFCKDCDEPIHLASSLSANH

SEQ ID NO: 276 - protein, 38 aa, Glycine max; identical to SEQ ID NO: 274
  REGION 1..38: note = conserved domain of Glyma12g05570.1

SEQ ID NO: 277 - protein, 38 aa, Glycine max; identical to SEQ ID NO: 275
  REGION 1..38: note = conserved domain of Glyma12g05570.1

SEQ ID NO: 278 - protein, 38 aa, Glycine max
  REGION 1..38: note = conserved domain of Glyma13g41980.1
CDVCERAPATVICCADEAALCAKCDVEVHAANKLASKH

SEQ ID NO: 279 - protein, 38 aa, Glycine max
  REGION 1..38: note = conserved domain of Glyma13g41980.1
CDICQDKPAFIFCVEDRALFCQDCDEPIHSAGSLSANH

SEQ ID NO: 280 - protein, 34 aa, Glycine max
  REGION 1..34: note = conserved domain of Glyma13g41980.2
QDKPAFIFCVEDRALFCQDCDEPIHSAGSLSANH

SEQ ID NO: 281 - protein, 33 aa, Glycine max
  REGION 1..33: note = conserved domain of Glyma13g41980.2
DKPAFIFCVEDRALFCQDCDEPIHSAGSLSANH

SEQ ID NO: 282 - protein, 34 aa, Glycine max; identical to SEQ ID NO: 280
  REGION 1..34: note = conserved domain of Glyma13g41980.3

SEQ ID NO: 283 - protein, 33 aa, Glycine max; identical to SEQ ID NO: 281
  REGION 1..33: note = conserved domain of Glyma13g41980.3

SEQ ID NO: 284 - protein, 38 aa, Glycine max; identical to SEQ ID NO: 278
  REGION 1..38: note = conserved domain of Glyma15g03400.1

SEQ ID NO: 285 - protein, 38 aa, Glycine max; identical to SEQ ID NO: 279
  REGION 1..38: note = conserved domain of Glyma15g03400.1

SEQ ID NO: 286 - protein, 38 aa, Solanum lycopersicum
  REGION 1..38: note = conserved domain of AAS67368
CDVCEKAQATVICCADEAALCAKCDIEVHAANKLASKH

SEQ ID NO: 287 - protein, 38 aa, Solanum lycopersicum
  REGION 1..38: note = conserved domain of AAS67368
CDICQDKAAFIFCVEDRALFCKDCDEAIHSASSLAKNH

SEQ ID NO: 288 - protein, 38 aa, Arabidopsis thaliana
  REGION 1..38: note = conserved domain of AtBBX20 (G1888)
CAVCDKEEASVFCCADEAALCNGCDRHVHFANKLAGKH

SEQ ID NO: 289 - protein, 38 aa, Arabidopsis thaliana
  REGION 1..38: note = conserved domain of AtBBX20 (G1888)
CDICGERRALLFCQEDRAILCRECDIPIHQANEHTKKH

SEQ ID NO: 290 - protein, 38 aa, Arabidopsis thaliana
  REGION 1..38: note = conserved domain of AtBBX21 (G1482)
CDVCDKEEASVFCTADEASLCGGCDHQVHHANKLASKH

SEQ ID NO: 291 - protein, 38 aa, Arabidopsis thaliana
  REGION 1..38: note = conserved domain of AtBBX21 (G1482)
CDICQDKKALLFCQQDRAILCKDCDSSIHAANEHTKKH

SEQ ID NO: 292 - protein, 38 aa, Zea mays
  REGION 1..38: note = conserved domain of ACG44133.1
CDVCAAEAASVFCCADEAALCDACDDRVHRANKLAGKH

SEQ ID NO: 293 - protein, 38 aa, Zea mays
  REGION 1..38: note = conserved domain of ACG44133.1
CDICQERRGFLFCKEDRAILCRECDAPVHSANDMTRRH

SEQ ID NO: 294 - protein, 38 aa, Zea mays; identical to SEQ ID NO: 292
  REGION 1..38: note = conserved domain of CAW36814.1

SEQ ID NO: 295 - protein, 38 aa, Zea mays; identical to SEQ ID NO: 293
  REGION 1..38: note = conserved domain of CAW36814.1

SEQ ID NO: 296 - protein, 38 aa, Populus trichocarpa
  REGION 1..38: note = conserved domain of Pt_195637
CDVCDNVEATVFCCADEAALCDGCDHRVHHANKLASKH

SEQ ID NO: 297 - protein, 38 aa, Populus trichocarpa
  REGION 1..38: note = conserved domain of Pt_195637
CDICQERRALLFCQEDRAILCRECDLPIHKANEHTQKH

SEQ ID NO: 298 - protein, 38 aa, Populus trichocarpa
  REGION 1..38: note = conserved domain of Pt_550941
CDVCNKEEASVFCTADEAALCDTCDHRVHHANKLASKH

SEQ ID NO: 299 - protein, 38 aa, Populus trichocarpa
  REGION 1..38: note = conserved domain of Pt_550941
CDICQEKRAFLFCQQDRAILCRECDGPIHTANEHTQKH

SEQ ID NO: 300 - protein, 38 aa, Populus trichocarpa
  REGION 1..38: note = conserved domain of Pt_801530
CDVCSKEEASVFCTADEAALCDTCDHRVHHANKLASKH

SEQ ID NO: 301 - protein, 38 aa, Populus trichocarpa
  REGION 1..38: note = conserved domain of Pt_801530
CDICQDKRAFLFCQQDRAILCRDCDGPIHTANEHTQKH

SEQ ID NO: 302 - protein, 38 aa, Populus trichocarpa
  REGION 1..38: note = conserved domain of Pt_804174
CDVCDKVEATVFCCADEAALCDGCDHRVHHANTLASKH

SEQ ID NO: 303 - protein, 38 aa, Populus trichocarpa
  REGION 1..38: note = conserved domain of Pt_804174
CDICQERRAVLFCQEDRAILCRECDLPIHKVNEHTQKH

SEQ ID NO: 304 - protein, 38 aa, Oryza sativa
  REGION 1..38: note = conserved domain of Os02g43170.1
CDVCAAEAASVFCCADEAALCDACDHRVHRANKLAGKH

SEQ ID NO: 305 - protein, 38 aa, Oryza sativa
  REGION 1..38: note = conserved domain of Os02g43170.1
CDICQEKRGFLFCKEDRAILCRECDVPVHTASELTMRH

SEQ ID NO: 306 - protein, 38 aa, Oryza sativa; identical to SEQ ID NO: 304
  REGION 1..38: note = conserved domain of Os02g43170.2

SEQ ID NO: 307 - protein, 38 aa, Oryza sativa; identical to SEQ ID NO: 305
  REGION 1..38: note = conserved domain of Os02g43170.2

SEQ ID NO: 308 - protein, 38 aa, Oryza sativa
  REGION 1..38: note = conserved domain of Os04g45690.1
CDVCAAEAASVFCCADEAALCDACDRRVHSANKLAGKH

SEQ ID NO: 309 - protein, 38 aa, Oryza sativa
  REGION 1..38: note = conserved domain of Os04g45690.1
CDICQEKRGFLFCKEDRAILCRECDVTVHTTSELTRRH

SEQ ID NO: 310 - protein, 38 aa, Oryza sativa; identical to SEQ ID NO: 308
  REGION 1..38: note = conserved domain of G5159

SEQ ID NO: 311 - protein, 38 aa, Oryza sativa; identical to SEQ ID NO: 309
  REGION 1..38: note = conserved domain of G5159

SEQ ID NO: 312 - protein, 38 aa, Oryza sativa
  REGION 1..38: note = conserved domain of Os06g49880.1
CDVCAAEPAAVLCCADEAALCSACDRRVHRANRLASKH

SEQ ID NO: 313 - protein, 38 aa, Oryza sativa
  REGION 1..38: note = conserved domain of Os06g49880.1
CDVCREKRGLVFCVEDRAILCADCDEPIHSANDLTAKH

SEQ ID NO: 314 - protein, 38 aa, Oryza sativa; identical to SEQ ID NO: 312
  REGION 1..38: note = conserved domain of Os06g49880.2

SEQ ID NO: 315 - protein, 38 aa, Oryza sativa; identical to SEQ ID NO: 313
  REGION 1..38: note = conserved domain of Os06g49880.2

SEQ ID NO: 316 - protein, 38 aa, Glycine max
  REGION 1..38: note = conserved domain of Glyma04g01120.1
CAVCDKVEASVFCSADEAALCHSCDRTIHHANKLATKH

SEQ ID NO: 317 - protein, 38 aa, Glycine max
  REGION 1..38: note = conserved domain of Glyma04g01120.1
CDICQERRAYLFCQEDRALLCRECDVPIHRANEHTQKH

SEQ ID NO: 318 - protein, 38 aa, Glycine max
  REGION 1..38: note = conserved domain of G5365
CDVCNKHQASFFCTADEAALCDGCDHRVHHANKLASKH

SEQ ID NO: 319 - protein, 38 aa, Glycine max
  REGION 1..38: note = conserved domain of G5365
CDVCQERRAFVFCQQDRAILCKECDVPIHSANDLTKNH

SEQ ID NO: 320 - protein, 38 aa, Glycine max; identical to SEQ ID NO: 318
  REGION 1..38: note = conserved domain of Glyma04g02960.2

SEQ ID NO: 321 - protein, 38 aa, Glycine max; identical to SEQ ID NO: 319
  REGION 1..38: note = conserved domain of Glyma04g02960.2

SEQ ID NO: 322 - protein, 38 aa, Glycine max
  REGION 1..38: note = conserved domain of Glyma06g01140.1
CDVCDKVEASVFCPADEAALCHSCDRTIHRANKLATKH

SEQ ID NO: 323 - protein, 38 aa, Glycine max; identical to SEQ ID NO: 317
  REGION 1..38: note = conserved domain of Glyma06g01140.1

SEQ ID NO: 324 - protein, 38 aa, Glycine max
  REGION 1..38: note = conserved domain of G5367
CDVCNKQQASLFCTADEAALCDGCDHRVHHANKLASKH

SEQ ID NO: 325 - protein, 38 aa, Glycine max
  REGION 1..38: note = conserved domain of G5367
CDVCQERRAFVFCQQDRAILCKECDVPVHSANDLTKNH

SEQ ID NO: 326 - protein, 38 aa, Glycine max
  REGION 1..38: note = conserved domain of Glyma11g12060.1
CDVCHNEVASFFCPSDEASLCHACDRTIHHANKLADKH

SEQ ID NO: 327 - protein, 38 aa, Glycine max
  REGION 1..38: note = conserved domain of Glyma11g12060.1
CDICHERRAYLFCKEDRAILCRECDLSIHGVNEHTKKH

SEQ ID NO: 328 - protein, 38 aa, Glycine max
  REGION 1..38: note = conserved domain of G5396
CDVCNKHEASVFCTADEAALCDGCDHRVHHANKLASKH

SEQ ID NO: 329 - protein, 38 aa, Glycine max
  REGION 1..38: note = conserved domain of G5396
CDICQERRAFTFCQQDRAILCKECDVSIHSANEHTLKH

SEQ ID NO: 330 - protein, 38 aa, Glycine max; identical to SEQ ID NO: 328
  REGION 1..38: note = conserved domain of G5400

SEQ ID NO: 331 - protein, 38 aa, Glycine max; identical to SEQ ID NO: 329
  REGION 1..38: note = conserved domain of G5400

SEQ ID NO: 332 - protein, 37 aa, Arabidopsis thaliana
  REGION 1..37: note = conserved domain of AtBBX26 (G1486)
CHTCRHVTAVIHCVTEALNFCLTCDNLRHHNNIHAEH

SEQ ID NO: 333 - protein, 37 aa, Arabidopsis thaliana
  REGION 1..37: note = conserved domain of AtBBX30 (G1478)
CELCGENATVYCEADAAFLCRKCDRWVHSANFLARRH

SEQ ID NO: 334 - protein, 37 aa, Arabidopsis thaliana
  REGION 1..37: note = conserved domain of AtBBX31 (G1929)
CELCDGDASVFCEADSAFLCRKCDRWVHGANFLAWRH

SEQ ID NO: 335 - protein, 37 aa, Populus trichocarpa
  REGION 1..37: note = conserved domain of Pt_562313
CELCGSRASLYCQADDAFLCQKCDKWVHGANFLAQRH

SEQ ID NO: 336 - protein, 37 aa, Populus trichocarpa
  REGION 1..37: note = conserved domain of Pt_594447
CELCGSRATLYCQADHAFLCQKCDGWVHGANFLALRH

SEQ ID NO: 337 - protein, 37 aa, Glycine max
  REGION 1..37: note = conserved domain of Glyma12g32230.1
CELCGLQASLYCQADDAYLCKKCDKRVHEANFLALRH

SEQ ID NO: 338 - protein, 37 aa, Glycine max
  REGION 1..37: note = conserved domain of G4019
CELCGLQASLYCQADDAYLCRKCDKRVHEANFLALRH

SEQ ID NO: 339 - protein, 38 aa, Arabidopsis thaliana
  REGION 1..38: note = conserved domain of AtBBX7 (G2440)
CDFCGEQRSMVYCRSDAACLCLSCDRSVHSANALSKRH

SEQ ID NO: 340 - protein, 47 aa, Arabidopsis thaliana
  REGION 1..47: note = conserved domain of AtBBX7 (G2440)
CERCNAQPATVRCVEERVSLCQNCDWSGHNNSNNNNSSSSSTSPQQH

SEQ ID NO: 341 - protein, 42 aa, Arabidopsis thaliana
  REGION 1..42: note = conserved domain of AtBBX7 (G2440)
RNNAVMRYKEKKKARKFDKRVRYASRKARADVRRRVKGRFVK

SEQ ID NO: 342 - protein, 38 aa, Arabidopsis thaliana
  REGION 1..38: note = conserved domain of AtBBX8 (G1479)
CDFCGEQRSMVYCRSDAACLCLSCDRNVHSANALSKRH

SEQ ID NO: 343 - protein, 40 aa, Arabidopsis thaliana
  REGION 1..40: note = conserved domain of AtBBX8 (G1479)
CERCNAQPASVRCSDERVSLCQNCDWSGHDGKNSTTTSHH

SEQ ID NO: 344 - protein, 42 aa, Arabidopsis thaliana
  REGION 1..42: note = conserved domain of AtBBX8 (G1479)
RNNAVMRYKEKKKARKFDKRVRYVSRKERADVRRRVKGRFVK

SEQ ID NO: 345 - protein, 38 aa, Zea mays
  REGION 1..38: note = conserved domain of ACF80403.1
CGFCGKQRSMIYCRSDAASLCLSCDRSVHSANALSRRH

SEQ ID NO: 346 - protein, 38 aa, Zea mays
  REGION 1..38: note = conserved domain of ACF80403.1
CDRCGLQPASVRCLEDNTSLCQNCDWNGHDAASGASGH

SEQ ID NO: 347 - protein, 42 aa, Zea mays
  REGION 1..42: note = conserved domain of ACF80403.1
RDSALTRYKEKKKKRMFDKKIRYASRKARADVRKRVKGRFIK

SEQ ID NO: 348 - protein, 38 aa, Zea mays
  REGION 1..38: note = conserved domain of ACG34885.1
CDFCGEQRSMVYCRSDAASLCLSCDRNVHSANALSRRH

SEQ ID NO: 349 - protein, 38 aa, Zea mays
  REGION 1..38: note = conserved domain of ACG34885.1
CDRCASQPAMVRCLAENASLCQNCDWNGHIAGSSSAGH

SEQ ID NO: 350 - protein, 42 aa, Zea mays
  REGION 1..42: note = conserved domain of ACG34885.1
RDSALTRYKEKKMRRKFDKKIRYASRKARADVRKRVKGRFVK

SEQ ID NO: 351 - protein, 38 aa, Zea mays
  REGION 1..38: note = conserved domain of ACN25813.1
CDFCGKQRSMIYCRSDAASLCLSCDRNVHSANALSRRH

SEQ ID NO: 352 - protein, 38 aa, Zea mays
  REGION 1..38: note = conserved domain of ACN25813.1
CDRCGSQPASVRCLEDNASLCQNCDWNGHDAESGASGH

SEQ ID NO: 353 - protein, 20 aa, Zea mays
  REGION 1..20: note = conserved domain of ACN25813.1
YASRKARADVRKRVKGRFIK

SEQ ID NO: 354 - protein, 38 aa, Populus trichocarpa
  REGION 1..38: note = conserved domain of Pt_247140
CDFCGEQRSMVYCRSDAASLCLSCDRNVHSANALSKRH

SEQ ID NO: 355 - protein, 38 aa, Populus trichocarpa
  REGION 1..38: note = conserved domain of Pt_247140
CERCNSQPALVRCAEERISLCQNCDWIGHGTSTSASTH

SEQ ID NO: 356 - protein, 42 aa, Populus trichocarpa
  REGION 1..42: note = conserved domain of Pt_247140
RSDAVMRYKEKKKTRMFEKKVRYASRKARADVRRRVKGRFVK

SEQ ID NO: 357 - protein, 38 aa, Populus trichocarpa
  REGION 1..38: note = conserved domain of Pt_409151
CDFCGEQRSMVYCRSDAACLCLSCDQIVHSANALSKRH

SEQ ID NO: 358 - protein, 38 aa, Populus trichocarpa
  REGION 1..38: note = conserved domain of Pt_409151
CERCNSQPALVRRVEERISLCQNCDWMGYGSSTSASTH

SEQ ID NO: 359 - protein, 42 aa, Populus trichocarpa
  REGION 1..42: note = conserved domain of Pt_409151
RSDAVKRYMEKKKTRKFEKKVRYASRKARADVRRRVKGRFVK

SEQ ID NO: 360 - protein, 38 aa, Oryza sativa
  REGION 1..38: note = conserved domain of Os02g49230.1
CDFCREQRSMVYCRSDAASLCLSCDRNVHSANALSRRH

SEQ ID NO: 361 - protein, 38 aa, Oryza sativa
  REGION 1..38: note = conserved domain of Os02g49230.1
CDRCVGQPAAVRCLEENTSLCQNCDWNGHGAASSAAGH

SEQ ID NO: 362 - protein, 42 aa, Oryza sativa
  REGION 1..42: note = conserved domain of Os02g49230.1
RDNALTRYKEKKKRRKFDKKIRYASRKARADVRKRVKGRFVK

SEQ ID NO: 363 - protein, 38 aa, Oryza sativa; identical to SEQ ID NO: 360
  REGION 1..38: note = conserved domain of Os02g49230.2

SEQ ID NO: 364 - protein, 38 aa, Oryza sativa; identical to SEQ ID NO: 361
  REGION 1..38: note = conserved domain of Os02g49230.2

SEQ ID NO: 365 - protein, 42 aa, Oryza sativa; identical to SEQ ID NO: 362
  REGION 1..42: note = conserved domain of Os02g49230.2

SEQ ID NO: 366 - protein, 38 aa, Oryza sativa; identical to SEQ ID NO: 360
  REGION 1..38: note = conserved domain of Os02g49230.3

SEQ ID NO: 367 - protein, 38 aa, Oryza sativa; identical to SEQ ID NO: 361
  REGION 1..38: note = conserved domain of Os02g49230.3

SEQ ID NO: 368 - protein, 42 aa, Oryza sativa; identical to SEQ ID NO: 362
  REGION 1..42: note = conserved domain of Os02g49230.3

SEQ ID NO: 369 - protein, 38 aa, Oryza sativa; identical to SEQ ID NO: 360
  REGION 1..38: note = conserved domain of Os02g49230.4

SEQ ID NO: 370 - protein, 38 aa, Oryza sativa; identical to SEQ ID NO: 361
  REGION 1..38: note = conserved domain of Os02g49230.4

SEQ ID NO: 371 - protein, 42 aa, Oryza sativa; identical to SEQ ID NO: 362
  REGION 1..42: note = conserved domain of Os02g49230.4

SEQ ID NO: 372 - protein, 38 aa, Oryza sativa; identical to SEQ ID NO: 360
  REGION 1..38: note = conserved domain of Os02g49230.5

SEQ ID NO: 373 - protein, 38 aa, Oryza sativa; identical to SEQ ID NO: 361
  REGION 1..38: note = conserved domain of Os02g49230.5

SEQ ID NO: 374 - protein, 42 aa, Oryza sativa; identical to SEQ ID NO: 362
  REGION 1..42: note = conserved domain of Os02g49230.5

SEQ ID NO: 375 - protein, 38 aa, Oryza sativa; identical to SEQ ID NO: 360
  REGION 1..38: note = conserved domain of Os02g49230.6

SEQ ID NO: 376 - protein, 38 aa, Oryza sativa; identical to SEQ ID NO: 361
  REGION 1..38: note = conserved domain of Os02g49230.6

SEQ ID NO: 377 - protein, 42 aa, Oryza sativa; identical to SEQ ID NO: 362
  REGION 1..42: note = conserved domain of Os02g49230.6

SEQ ID NO: 378 - protein, 38 aa, Oryza sativa; identical to SEQ ID NO: 348
  REGION 1..38: note = conserved domain of Os06g19444.1

SEQ ID NO: 379 - protein, 38 aa, Oryza sativa
  REGION 1..38: note = conserved domain of Os06g19444.1
CDRCASQPAMVRCLVENASLCQNCDWNGHSAGSSAAGH

SEQ ID NO: 380 - protein, 20 aa, Oryza sativa
  REGION 1..20: note = conserved domain of Os06g19444.1
YASRKARADVRKRVKGRFVK

SEQ ID NO: 381 - protein, 38 aa, Oryza sativa; identical to SEQ ID NO: 348
  REGION 1..38: note = conserved domain of Os06g19444.2

SEQ ID NO: 382 - protein, 38 aa, Oryza sativa; identical to SEQ ID NO: 379
  REGION 1..38: note = conserved domain of Os06g19444.2

SEQ ID NO: 383 - protein, 20 aa, Oryza sativa
  REGION 1..20: note = conserved domain of Os06g19444.2
YASRKARADVRKRVKGRFVK

SEQ ID NO: 384 - protein, 38 aa, Glycine max
  REGION 1..38: note = conserved domain of Glyma02g38870.1
CDFCGDQRSLVYCRSDSACLCLSCDRNVHSANALSRRH

SEQ ID NO: 385 - protein, 38 aa, Glycine max
  REGION 1..38: note = conserved domain of Glyma02g38870.1
CERCNSQPAFVRSVEEKISLCQNCDWLGHGTSPSSSMH

SEQ ID NO: 386 - protein, 42 aa, Glycine max
  REGION 1..42: note = conserved domain of Glyma02g38870.1
RSNAVMRYKEKKKTRKFEKKVRYASRKARADVRKRVKGRFVK

SEQ ID NO: 387 - protein, 42 aa, Glycine max
  REGION 1..42: note = conserved domain of Glyma13g11590.1
RSNAVMRYKEKKKTRMFDKKVRYASRKARADVRRRVKGRFVK

SEQ ID NO: 388 - protein, 38 aa, Glycine max
  REGION 1..38: note = conserved domain of Glyma14g36930.1
CDFCGDQRSLVYCRSDAACLCLSCDRNVHSANALSRRH

SEQ ID NO: 389 - protein, 38 aa, Glycine max
  REGION 1..38: note = conserved domain of Glyma14g36930.1
CERCNSQPAFVRCVDEKISLCQNCDWLGHGTSPSSSTH

SEQ ID NO: 390 - protein, 42 aa, Glycine max; identical to SEQ ID NO: 386
  REGION 1..42: note = conserved domain of Glyma14g36930.1

SEQ ID NO: 391 - protein, 38 aa, Glycine max; identical to SEQ ID NO: 388
  REGION 1..38: note = conserved domain of Glyma14g36930.2

SEQ ID NO: 392 - protein, 38 aa, Glycine max; identical to SEQ ID NO: 389
  REGION 1..38: note = conserved domain of Glyma14g36930.2

SEQ ID NO: 393 - protein, 42 aa, Glycine max; identical to SEQ ID NO: 386
  REGION 1..42: note = conserved domain of Glyma14g36930.2

SEQ ID NO: 394 - protein, 42 aa, Glycine max; identical to SEQ ID NO: 387
  REGION 1..42: note = conserved domain of Glyma20g07050.1

SEQ ID NO: 395 - protein, 42 aa, Glycine max; identical to SEQ ID NO: 387
  REGION 1..42: note = conserved domain of Glyma20g07050.2

SEQ ID NO: 396 - protein, 38 aa, Arabidopsis thaliana
  REGION 1..38: note = conserved domain of AtBBX19 (G902)
CDACENAAAIIFCAADEAALCRPCDEKVHMCNKLASRH

SEQ ID NO: 397 - protein, 36 aa, Arabidopsis thaliana
  REGION 1..36: note = conserved domain of AtBBX19 (G902)
CDICENAPAFFYCEIDGSSLCLQCDMVVHVGGKRTH

SEQ ID NO: 398 - protein, 38 aa, Arabidopsis thaliana
  REGION 1..38: note = conserved domain of AtBBX18 (G1881)
CDACESAAAIVFCAADEAALCCSCDEKVHKCNKLASRH

SEQ ID NO: 399 - protein, 36 aa, Arabidopsis thaliana; identical to SEQ ID NO: 397
  REGION 1..36: note = conserved domain of AtBBX18 (G1881)

SEQ ID NO: 400 - protein, 38 aa, Zea mays
  REGION 1..38: note = conserved domain of ACF85933.1
CDVCESAPAVLFCAADEAALCRPCDEKVHMCNKLASRH

SEQ ID NO: 401 - protein, 36 aa, Zea mays
  REGION 1..36: note = conserved domain of ACF85933.1
CDICENSPAFFYCEIDGTSLCLSCDMTVHVGGKRTH

SEQ ID NO: 402 - protein, 38 aa, Zea mays; identical to SEQ ID NO: 400
  REGION 1..38: note = conserved domain of ACL52378.1

SEQ ID NO: 403 - protein, 36 aa, Zea mays; identical to SEQ ID NO: 401
  REGION 1..36: note = conserved domain of ACL52378.1

SEQ ID NO: 404 - protein, 38 aa, Populus trichocarpa
  REGION 1..38: note = conserved domain of Pt_648693
CDVCESAAAILFCAADEAALCRSCDEKVHMCNKLASRH

SEQ ID NO: 405 - protein, 36 aa, Populus trichocarpa
  REGION 1..36: note = conserved domain of Pt_648693
CDICEKAPAFFYCEIDGSSLCLQCDMIVHVGGKRTH

SEQ ID NO: 406 - protein, 38 aa, Populus trichocarpa
  REGION 1..38: note = conserved domain of Pt_721866
CDVCESAAAILFCAADEAALCRSCDEKVHLCNKLASRH

SEQ ID NO: 407 - protein, 36 aa, Populus trichocarpa
  REGION 1..36: note = conserved domain of Pt_721866
CDICENAPAFFYCEIDGSSLCLQCDMIVHVGGKRTH

SEQ ID NO: 408 - protein, 38 aa, Populus trichocarpa
  REGION 1..38: note = conserved domain of Pt_741179
CDACESAFAIVFCAADEAALCLACDKKVHMCNKLASRH

SEQ ID NO: 409 - protein, 36 aa, Populus trichocarpa
  REGION 1..36: note = conserved domain of Pt_741179
CDICENAPAFFYCETDGSSLCLQCDMTVHVGGKRTH

SEQ ID NO: 410 - protein, 38 aa, Populus trichocarpa
  REGION 1..38: note = conserved domain of Pt_820080
CDACESAAAIVFCAADEAALCLACDEKVHMCNKLASRH

SEQ ID NO: 411 - protein, 36 aa, Populus trichocarpa; identical to SEQ ID NO: 409
  REGION 1..36: note = conserved domain of Pt_820080

SEQ ID NO: 412 - protein, 38 aa, Oryza sativa
  REGION 1..38: note = conserved domain of Os09g35880.1
CDVCESAPAVLFCVADEAALCRSCDEKVHMCNKLARRH

SEQ ID NO: 413 - protein, 36 aa, Oryza sativa
  REGION 1..36: note = conserved domain of Os09g35880.1
CDICENAPAFFYCEIDGTSLCLSCDMTVHVGGKRTH

SEQ ID NO: 414 - protein, 38 aa, Glycine max
  REGION 1..38: note = conserved domain of Glyma01g37370.1
CDACESAAAIVFCAADEAALCRACDEKVHMCNKLASRH

SEQ ID NO: 415 - protein, 36 aa, Glycine max
  REGION 1..36: note = conserved domain of Glyma01g37370.1
CDICENAPAFFYCETDGSSLCLQCDMIVHVGGKRTH

SEQ ID NO: 416 - protein, 38 aa, Glycine max; identical to SEQ ID NO: 414
  REGION 1..38: note = conserved domain of Glyma11g07930.1

SEQ ID NO: 417 - protein, 36 aa, Glycine max; identical to SEQ ID NO: 415
  REGION 1..36: note = conserved domain of Glyma11g07930.1

SEQ ID NO: 418 - protein, 38 aa, Glycine max; identical to SEQ ID NO: 414
  REGION 1..38: note = conserved domain of Glyma11g07930.2

SEQ ID NO: 419 - protein, 36 aa, Glycine max; identical to SEQ ID NO: 415
  REGION 1..36: note = conserved domain of Glyma11g07930.2

SEQ ID NO: 420 - protein, 38 aa, Glycine max; identical to SEQ ID NO: 414
  REGION 1..38: note = conserved domain of Glyma11g07930.3

SEQ ID NO: 421 - protein, 36 aa, Glycine max; identical to SEQ ID NO: 415
  REGION 1..36: note = conserved domain of Glyma11g07930.3

SEQ ID NO: 422 - protein, 38 aa, Glycine max; identical to SEQ ID NO: 414
  REGION 1..38: note = conserved domain of Glyma11g07930.4

SEQ ID NO: 423 - protein, 36 aa, Glycine max; identical to SEQ ID NO: 415
  REGION 1..36: note = conserved domain of Glyma11g07930.4

SEQ ID NO: 424 - protein, 38 aa, Glycine max
  REGION 1..38: note = conserved domain of Glyma11g11850.1
CDVCESAAAILFCAADEAALCSACDHKIHMCNKLASRH

SEQ ID NO: 425 - protein, 36 aa, Glycine max; identical to SEQ ID NO: 407
  REGION 1..36: note = conserved domain of Glyma11g11850.1

SEQ ID NO: 426 - protein, 38 aa, Glycine max
  REGION 1..38: note = conserved domain of Glyma12g04130.1
CDVCESAAAIVFCAADEAALCSACDHKIHMCNKLASRH

SEQ ID NO: 427 - protein, 36 aa, Glycine max; identical to SEQ ID NO: 407
  REGION 1..36: note = conserved domain of Glyma12g04130.1

SEQ ID NO: 428 - DNA, 3157 bp, synthetic construct
  misc_feature 1..3157: note = pMEN65 vector (35S::MCS::E9)
  source 1..3157: mol_type = other DNA
aagcttnnnnctgcagnnnnnnnnnnnnnnnnnnnnnnnnnnnnnnnnnnnntcggattccattgcccagctatctgtcactttattgtgaagatagtgaaaaagaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgacaagctgactctagcagatctggtaccgtcgacggtgagctccgcggccgctctagacaggcctcgtaccggatcctctagctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcgacctgcagccactcgaagcggccggccgccactcgagatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggcccggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggctcgag

SEQ ID NO: 429 - DNA, 906 bp, Arabidopsis thaliana
  misc_feature 1..906: note = BBX32 (G1988)
  source 1..906: mol_type = unassigned DNA
tgctactctcatcaaccatgaaccataaaaactccaccgctctttctctccctcaatcatttacatctcttccttaaatctctcttcccaccatcatcattccaaaccaattctctctcacttctttctggtgatcagagagatcgactcaatggtgagcttttgcgagctttgtggtgccgaagctgatctccattgtgccgcggactctgccttcctctgccgttcttgtgacgctaagttccatgcctcaaattttctcttcgctcgtcatttccggcgtgtcatctgcccaaattgcaaatctcttactcaaaatttcgtttctggtcctcttcttccttggcctccacgaacaacatgttgttcagaatcgtcgtcttcttcttgctgctcgtctcttgactgtgtctcaagctccgagctatcgtcaacgacgcgtgacgtaaacagagcgcgagggagggaaaacagagtgaatgccaaggccgttgcggttacggtggcggatggcatttttgtaaattggtgtggtaagttaggactaaacagggatttaacaaacgctgtcgtttcatatgcgtctttggctttggctgtggagacgaggccaagagcgacgaagagagtgttcttagcggcggcgttttggttcggcgttaagaacacgacgacgtggcagaatttaaagaaagtagaagatgtgactggagtttcagctgggatgattcgagcggttgaaagcaaattggcgcgtgcaatgacgcagcagcttagacggtggcgcgtggattcggaggaaggatgggctgaaaacgacaacgtttgagaaatattattgacatgggtcccgcattatgcaaattaggacatttagtgtttagtgcattaattatagtttgtgtc

SEQ ID NO: 430 - protein, 225 aa, Arabidopsis thaliana
  REGION 1..225: note = G1988 polypeptide (BBX32, domain in aa coordinates: 5-50)
MVSFCELCGAEADLHCAADSAFLCRSCDAKFHASNFLFARHFRRVICPNCKSLTQNFVSGPLLPWPPRTTCCSESSSSSCCSSLDCVSSSELSSTTRDVNRARGRENRVNAKAVAVTVADGIFVNWCGKLGLNRDLTNAVVSYASLALAVETRPRATKRVFLAAAFWFGVKNTTTWQNLKKVEDVTGVSAGMIRAVESKLARAMTQQLRRWRVDSEEGWAENDNV

SEQ ID NO: 431 - DNA, 732 bp, Glycine max
  misc_feature 1..732: note = G4004
  source 1..732: mol_type = unassigned DNA
atgaagcccaagacttgcgagctttgtcatcaactagcttctctctattgtccctccgattccgcatttctctgcttccactgcgacgccgccgtccacgccgccaacttcctcgtagctcgccacctccgccgcctcctctgctccaaatgcaaccgtttcgccgcaattcacatctccggtgctatatcccgccacctctcctccacctgcacctcttgctccctggagattccttccgccgactccgattctctcccttcctcttctacctgcgtctccagttccgagtcttgctctacgaatcagattaaggcggagaagaagaggaggaggaggaggaggagtttctcgagttcctccgtgaccgacgacgcatctccggcggcgaagaagcggcggagaaatggcggatcggtggcggaggtgtttgagaaatggagcagagagatagggttagggttaggggtgaacggaaatcgcgtggcgtcgaacgctctgagtgtgtgcctcggaaagtggaggtcgcttccgttcagggtggctgctgcgacgtcgttttggttggggctgagattttgtggggacagaggcctcgccacgtgtcagaatctggcgaggttggaggcaatatctggagtgccagcaaagctgattctgggcgcacatgccaacctcgcacgtgtcttcacgcaccgccgcgaattgcaggaaggatggggcgagtcctag

SEQ ID NO: 432 - protein, 243 aa, Glycine max
  REGION 1..243: note = G4004 polypeptide
MKPKTCELCHQLASLYCPSDSAFLCFHCDAAVHAANFLVARHLRRLLCSKCNRFAAIHISGAISRHLSSTCTSCSLEIPSADSDSLPSSSTCVSSSESCSTNQIKAEKKRRRRRRSFSSSSVTDDASPAAKKRRRNGGSVAEVFEKWSREIGLGLGVNGNRVASNALSVCLGKWRSLPFRVAAATSFWLGLRFCGDRGLATCQNLARLEAISGVPAKLILGAHANLARVFTHRRELQEGWGES

SEQ ID NO: 433 - DNA, 726 bp, Glycine max
  misc_feature 1..726: note = G4005
  source 1..726: mol_type = unassigned DNA
atgaagggtaagacttgcgagctttgtgatcaacaagcttctctctattgtccctccgattccgcatttctctgctccgactgcgacgccgccgtgcacgccgccaactttctcgtagctcgtcacctccgtcgcctcctctgctccaaatgcaaccgtttcgccggatttcacatctcctccggcgctatatcccgccacctctcgtccacctgcagctcttgctccccggagaatccttccgctgactactccgattctctcccttcctcttctacctgcgtctccagttccgagtcttgctccacgaagcagattaaggcggagaagaagaggagttggtcgggttcctccgtgaccgacgacgcatctccggcggcgaagaagcggcagaggagtggaggatcggaggaggtgtttgagaaatggagcagagagatagggttagggttagggttaggggtaaacggaaatcgcgtggcgtcgaacgctctgagtgtgtgcctgggaaagtggaggtggcttccgttcagggtggctgctgcgacgtcgttttggttggggctgagattttgtggggacagagggctggcctcgtgtcagaatctggcgaggttggaggcaatatccggagtgccagttaagctgattctggccgcacatggcgacctggcacgtgtcttcacgcaccgccgcgaattgcaggaaggatggggcgagtcctag

SEQ ID NO: 434 - protein, 241 aa, Glycine max
  REGION 1..241: note = G4005 polypeptide
MKGKTCELCDQQASLYCPSDSAFLCSDCDAAVHAANFLVARHLRRLLCSKCNRFAGFHISSGAISRHLSSTCSSCSPENPSADYSDSLPSSSTCVSSSESCSTKQIKAEKKRSWSGSSVTDDASPAAKKRQRSGGSEEVFEKWSREIGLGLGLGVNGNRVASNALSVCLGKWRWLPFRVAAATSFWLGLRFCGDRGLASCQNLARLEAISGVPVKLILAAHGDLARVFTHRRELQEGWGES

SEQ ID NO: 435 - DNA, 1047 bp, Glycine max
  misc_feature 1..1047: note = GmBBX1
  source 1..1047: mol_type = unassigned DNA
atgttggatggagaagcaacaatgggcacgtgggctcgcatgtgcgacacgtgccgttcggccccctcctccgtgttctgccgcgcccacaccgccttcctctgcgccacgtgcgacgcgcgcctccacgcctcgctgacgtggcacgagcgcgtgtgggtgtgcgaggcctgcgagcgcgcccctgcggccttcctctgcaaggctgacgccgcctccctctgcgcctcctgcgacgccgacatccacgccgccaaccccctcgccagccgccaccaccgtgtccccatcctccccatcgccgccgcccccggcaacaacgacaacgacaacgtcgacgatgctgacttggacgacgatgacgaaaccgcttcatggctcttgctcaaccctgtcaaaagcgctagtgtccctaacaacaataacactaataatgggttctcgtataatggtgaggttgatgagtatttggaccttgttgatgactgcgacaaccaccactttgcttctgttgctactactacggaccattactctcatcagcaccaacatttcggtgttgtttctcacaagagttatgctggggacagtgttgttccggttcagcaccaccagcattttcagcttggcttggagtttgacaactccaaagctgccttcagttacaatgcttctgttaatcaaagtgtttcagtttcatcaatggatattggtgttgtacctgaatcaccgatgagggatgtctcaattggccatacaagaacccccaaagggacaattgacctattttctggacctcccattcaggtgccttcccatttttctccaatggacagggaggccagagtcctaaggtacagggagaaaaagaagacaagaaaatttgagaagacaatcaggtatgcctcaaggaaggcctatgcagagactagaccccgtataaaaggtcgatttgccaagagaacagatgtagaagctgaagtggatcagatgttctccacaacactaattacagaagttggatatggcattgttccctctttctga

SEQ ID NO: 436 - protein, 348 aa, Glycine max
  REGION 1..348: note = GmBBX1 soybean B-box polypeptide
MLDGEATMGTWARMCDTCRSAPSSVFCRAHTAFLCATCDARLHASLTWHERVWVCEACERAPAAFLCKADAASLCASCDADIHAANPLASRHHRVPILPIAAAPGNNDNDNVDDADLDDDDETASWLLLNPVKSASVPNNNNTNNGFSYNGEVDEYLDLVDDCDNHHFASVATTTDHYSHQHQHFGVVSHKSYAGDSVVPVQHHQHFQLGLEFDNSKAAFSYNASVNQSVSVSSMDIGVVPESPMRDVSIGHTRTPKGTIDLFSGPPIQVPSHFSPMDREARVLRYREKKKTRKFEKTIRYASRKAYAETRPRIKGRFAKRTDVEAEVDQMFSTTLITEVGYGIVPSF

SEQ ID NO: 437 - DNA, 1059 bp, Glycine max
  misc_feature 1..1059: note = GmBBX2
  source 1..1059: mol_type = unassigned DNA
atgttggagggccaagcaacaacgcccacgtggccgcgcatgtgcgacacgtgccgctccgtcccctccaccgtcttctgccgctcccacaccgcctttctctgcgccacgtgcgacactcgcctccacgtctctctcacgtggcacgagcgcgtgtgggtgtgcgaggcgtgcgagcgcgccccggcggcgttcctctgcaaggccgacgccgcctccctctgcgcctcctgcgacgccgacatccacgccgccaaccccctcgccagccgccaccaccgcgtccccatcctccccatcgccgccgccaacaacaacaacaacgacgacgacgacgttgctgacgttgacgacgaagacgaaaccgcttcatggctcttgctcaaccctatcaagagtgctactgtccctaacaccaataacaataacaataataacgggttcttgtataacggtgaggttgatgagtatttggaccttgttgataattgtaactcttgtggtgataataatcacttcgcttcagctgctgctactactgatcattacgctcagcaccaacatttcgctggtgtttctcagaagagttatgctggggacagtgttgttccggttcagcaacaccagcattttcagcttgggttggagtttgacaactccaaacctgccttcagttacaatggttctgttagtcaaagtgtttcagtttcatcaatggatattggtgttgtacctgaatcaccaatgagggatgtctcaattgcccatacaagaccccccaaagggacaattgacctattttctggacctccaattcaggtgccttcccatttttctccaatggacagggaggccagagtcctaaggtacagggagaaaaagaagatgagaaaatttgagaagacaatcaggtatgcctcaaggaaggcctatgcagagactagacctcgtataaaaggtcgatttgccaagagaacagatgtagaagctgaagtggatcagatgttctcgacaacactaattacagaagttggatatggaattgttccctctttctga

SEQ ID NO: 438 - protein, 352 aa, Glycine max
  REGION 1..352: note = GmBBX2 soybean B-box polypeptide
MLEGQATTPTWPRMCDTCRSVPSTVFCRSHTAFLCATCDTRLHVSLTWHERVWVCEACERAPAAFLCKADAASLCASCDADIHAANPLASRHHRVPILPIAAANNNNNDDDDVADVDDEDETASWLLLNPIKSATVPNTNNNNNNNGFLYNGEVDEYLDLVDNCNSCGDNNHFASAAATTDHYAQHQHFAGVSQKSYAGDSVVPVQQHQHFQLGLEFDNSKPAFSYNGSVSQSVSVSSMDIGVVPESPMRDVSIAHTRPPKGTIDLFSGPPIQVPSHFSPMDREARVLRYREKKKMRKFEKTIRYASRKAYAETRPRIKGRFAKRTDVEAEVDQMFSTTLITEVGYGIVPSF

SEQ ID NO: 439 - DNA, 1086 bp, Glycine max
  misc_feature 1..1086: note = GmBBX3
  source 1..1086: mol_type = unassigned DNA
atgttgaaggaaggcaccaacaacgttggtggcagcaccggcacctggtcacatgtctgcgacacgtgccggtcagcgccatgcgtactgtactgccatgcagactcagcatacctttgctcatcctgcgatgctcgtgtccatgcggctaaccgtgtggcctcaagacacgagcgtgtgtgggtgtgcgaagcgtgtgaacgtgctcccgcagcgtttctatgcaaagccgacgcagcttctctttgttcttcctgtgatgctgacattcactcagcaaaccctctcgctagccgccaccaccgcgtgcccattctcccgatctctggttccctcttcggggaaccagagcatgaacgcgtgtacgcgttcgtgaatgaagtggaagcggaggaggaagaggaagaggtttttgatgagtatgatgaggttgaagcagcttcgtggttgttgccacatcctatgaaaaatgataaaattgacgagaatggtggtgataagggttttttgtttggtgatgagtatttcgacaaccttgttgattgtaactcatgtggtcacaataacaaccagtttagcaacgtttatgatcagcaccagcagaattacagcaacactgtccctcagaactatgcagtggttccagttcaggtgccgcagcattttcaaccgggtttggactttgactcatcaaaagctgggttcagttacgatggttctcttagtcaaagtgtttcggtttcatcgatggatgttggtgttgtactcgaatcaacaataagtgacatctcaatgtcccactcaaagtcgccaatagggacaactgacctatttcctccccttcccatgccttcacatctcacaccaatggacagagaggcaagagtcctaagatacagggagaaaaagaagacaagaaaatttgagaagaaaataaggtatgcctcaaggaaggcctatgcagagactagaccccgcataaagggtcgttttgcaaagagaaccgatgtagaagctgaagtggaccagatgttctccacaacactattcactgaagttggaggtagcatttttcccactttctag

SEQ ID NO: 440 - protein, 361 aa, Glycine max
  REGION 1..361: note = GmBBX3 soybean B-box polypeptide
MLKEGTNNVGGSTGTWSHVCDTCRSAPCVLYCHADSAYLCSSCDARVHAANRVASRHERVWVCEACERAPAAFLCKADAASLCSSCDADIHSANPLASRHHRVPILPISGSLFGEPEHERVYAFVNEVEAEEEEEEVFDEYDEVEAASWLLPHPMKNDKIDENGGDKGFLFGDEYFDNLVDCNSCGHNNNQFSNVYDQHQQNYSNTVPQNYAVVPVQVPQHFQPGLDFDSSKAGFSYDGSLSQSVSVSSMDVGVVLESTISDISMSHSKSPIGTTDLFPPLPMPSHLTPMDREARVLRYREKKKTRKFEKKIRYASRKAYAETRPRIKGRFAKRTDVEAEVDQMFSTTLFTEVGGSIFPTF

SEQ ID NO: 441 - DNA, 1101 bp, Glycine max
  misc_feature 1..1101: note = GmBBX4
  source 1..1101: mol_type = unassigned DNA
atgttgaaggaaggcaccaacaacgttggtggcagcaacactggcaccacctggtcacgtgtctgtgacacgtgcctgtctgcgccatgcgtgctgtactgccatgcagactcagcatatctttgctcttcctgtgatgctcgtgtgcatgcagctaaccgtgtggcctcgagacacaagcgtgtgtgggtgtgcgaagcgtgtgagcgtgctccggcggcgtttctatgcaaagctgacgcagcttctctttgttcttcctgtgatgctgacattcactcagcaaaccctctcgctagccgccacaaccgtgttcccattctcccgatctcaggttccctcttcagggaaccagagcacaatcacaaacgcgtggaacacgcgttcgtgaatgaggttgaggaggaagaggaaggggtttttgatgagtatgaagacgaggttgaagctgcttcgtggttgttgccacatcctatgaaaaataatgatgaaattgaggagaatgattgtggtgacgagggttttttgtttgttgatgagtatttggacaatcttgttgattgttgtaactcatgtggtcacaatgacaaccagtttagcaacgtttatcagcaccagcagaattacaacactgtccctcagaactatgtagtggttccagttcaggtgccgcagcattttcaacccggtttggactttgactcatcaaaagctgggttcagttacgatggttctcttagtcaaagtgtttcggtttcatcaatggatgttggagttgtacccgaatcaacagtaagtggcatctcaatgtcccactcaaagtcaccaatagggacaaatgacctatttcctccccttctcatgccttcacatctcacaccaatggacagagaggcaagagtcctaagatatagggagaaaaagaagacaagaaagttcgagaagaaaataagatatgcctcaaggaaggcctatgcagagactagaccccgcataaagggtcgttttgcaaagagaactgatgtagaagctgaagtggatcagatgttctccacaaaactattcaatgaagttggaggtagcatttttcccactttctag

SEQ ID NO: 442 - protein, 366 aa, Glycine max
  REGION 1..366: note = GmBBX4 soybean B-box polypeptide
MLKEGTNNVGGSNTGTTWSRVCDTCLSAPCVLYCHADSAYLCSSCDARVHAANRVASRHKRVWVCEACERAPAAFLCKADAASLCSSCDADIHSANPLASRHNRVPILPISGSLFREPEHNHKRVEHAFVNEVEEEEEGVFDEYEDEVEAASWLLPHPMKNNDEIEENDCGDEGFLFVDEYLDNLVDCCNSCGHNDNQFSNVYQHQQNYNTVPQNYVVVPVQVPQHFQPGLDFDSSKAGFSYDGSLSQSVSVSSMDVGVVPESTVSGISMSHSKSPIGTNDLFPPLLMPSHLTPMDREARVLRYREKKKTRKFEKKIRYASRKAYAETRPRIKGRFAKRTDVEAEVDQMFSTKLFNEVGGSIFPTF

SEQ ID NO: 443 - DNA, 1098 bp, Glycine max
  misc_feature 1..1098: note = GmBBX5
  source 1..1098: mol_type = unassigned DNA
atgggaattgaaagaggaggcttgaagggattcaggagcggctggagtgtgccgccgaagaagccatgcgactcctgtaagctggcttcggcggcgcttttctgccacctcgattccgcgtttctctgcatcgcgtgcgattccaagattcactgcgcgaataaactcgcgtcgcgccacgagcgcgtgtggatgtgtgaggtgtgcgagcaggcacccgcttctgtcacgtgcaaggctgacgcggccgccctttgcgtcacgtgcgactccgacatccactccgccaacccactcgcgcaacgccacgagcgcgttcccgtggagcccttcttcgattccgcggaatccatcgtcaaagcctccgccaccgcctctttcgggttcgtcgtcccctccgacgatggcgctgcctccgacgtcttcgcccccgacgactccgactccgccgcctggctcatcccgaaccccaatttcgggtccaaactcatggacgcccccgaaatcaagtccaaggagatattcttctctgaaatggaccccttcctcgattttgattactcaaactccttccaaaaccacaacagcgccgtaaacgacagtgtcgttccagttcaaaccaaaccctctctcgcccctccccccatcaacaatcaccagcaccaccaccaatctgagacttgcttcgacatcgatttttgccgctcgaagctctcctcctttaactacccatcccagtctcttagccaaagcgtttcttcgtcgtcgcttgatgtcggagtggtgccggacgggaacacagtgtcggacatgtcgtattcgagtgggattgtggtgtcgggtggacaaggcgcgacgcagttgtgtgggatggatcgggaagcgagggtgctgcgatacagagagaaaaggaagaaccgaaaatttgagaagacgattcgatacgcatcgcgaaaggcttatgcggaaacccggcccaggataaaaggccggttcgcgaaacgaaccgaaattgactcggatgtggagcgtctctatagccctggcgctgcggcgctcatgctggacactccatacggtgtcgtaccaacgttttag

SEQ ID NO: 444 - protein, 365 aa, Glycine max
  REGION 1..365: note = GmBBX5 soybean B-box polypeptide
MGIERGGLKGFRSGWSVPPKKPCDSCKLASAALFCHLDSAFLCIACDSKIHCANKLASRHERVWMCEVCEQAPASVTCKADAAALCVTCDSDIHSANPLAQRHERVPVEPFFDSAESIVKASATASFGFVVPSDDGAASDVFAPDDSDSAAWLIPNPNFGSKLMDAPEIKSKEIFFSEMDPFLDFDYSNSFQNHNSAVNDSVVPVQTKPSLAPPPINNHQHHHQSETCFDIDFCRSKLSSFNYPSQSLSQSVSSSSLDVGVVPDGNTVSDMSYSSGIVVSGGQGATQLCGMDREARVLRYREKRKNRKFEKTIRYASRKAYAETRPRIKGRFAKRTEIDSDVERLYSPGAAALMLDTPYGVVPTF

SEQ ID NO: 445 - DNA, 1125 bp, Glycine max
  misc_feature 1..1125: note = GmBBX6
  source 1..1125: mol_type = unassigned DNA
atgggaattgaaagaggaggcttcaagggattcaggagcgcctggagcgtgccgcccaagccgtgcgattcctgcaagctggcctcggcggcgcttttctgccgccccgattccgcgtttctctgcatcgcctgcgattccaacattcactgctcgaacaaactcgcgtcgcgccacgagcgcgtgtggatgtgtgaggtgtgcgagcaggcacccgctgctgtcacgtgcaaggccgacgccgccgccctctgcgtcacgtgtgactccgacatccactccgcgaaccccctcgcgcaacgccacgagcgcgtccccgtggaacctttctttgactccgctgagtccatagtgaaagcctctgctgccgccaccttcgggttcattgtcccatccgacgacggcggcgcctccgacgctttcgcccccgatgactccgacgccgccgcgtggctcatcccgaaccctaatttcgggtccaagctcatggacgcccccgaaatcaagtccaaggagatattcttctctgaaatggaccccttcctcgattttgattactcaaactccttccaaaacaacaacagcgccggaaacgacagcgtcgttccagttcaaaaaccctctctcgcccctcccctaatcaataatcaccaccaccaccaatcagaaacttgcttcgacgtcgatttttgtcgctcgaagctctcctcctttaactacccttccaattctcttagccaaagcgtttcgtcgtcgtcgcttgatgttggagtggtgccggacgggaacacggtgtcagacatgtcgtattcttttggtcggaacagttcggattcgagtgggattgttgttgtgtcaggaaatagtgtggggcaaggtgcgacgcagttgtgtggaatggatcgggaagcgagggtactgcgatacagagagaaaaggaagaaccgaaaattcgagaagacaattcgatacgcatcgcgaaaagcatatgcggaaacccggcccaggataaaaggccgcttcgccaaacgaacagaaattgactcggatgtggagcgtctctatagccctggccctgcggtgctcatgctggacactccatacggcgtcgtaccatcgttttag

SEQ ID NO: 446 - protein, 89 aa, Glycine max
  REGION 1..89: note = GmBBX6 soybean B-box polypeptide
MGIERGGFKGFRSAWSVPPKPCDSCKLASAALFCRPDSAFLCIACDSNIHCSNKLASRHERVWMCEVCEQAPAAVTCKADAAALCVTCD

SEQ ID NO: 447 - DNA, 684 bp, Glycine max
  misc_feature 1..684: note = GmBBX7
  source 1..684: mol_type = unassigned DNA
atggtcaatcaactcttcgactcatgcaaatcagccactgctaccttgtactgccgcatcgacacgacgttcctctgcggcacctgtgactctaaggttcatgccgccaacaagctcgtgtcgcgccacccgcgcgttgcactctgcgaggtgtgcaagcaggcgtcaacgcatgtgacctgcaaggccggcgctgccgctctctgcctcacctgtgactctgagatccactccaccaaccccctcgccagccgccacgagcgcatccccatcacgctgttcttcgagtacgtacactcagtcaaggcctcttcatcgatcaacttccaccactgcttcttctccgacaccgacgctgatgtcagcaccaaggaggccgaggtcgcgtcctggctactaaataatctgaagacatatctaaattcgtctcagtacttgttctcagaaaagttccgccatcgccgatggcgtcattccagtgtagagcaacttcaagccgttcacctacgagcaccaaacatctttcttcacagctacttctctcgatctagaaccttcggagtaattaattattacatactaactggagttagatccaacataaatgacttctctctcaacttacaaaatcaagttaaaaatattattaagtctaaaattgctctcaataattttttccaataa

SEQ ID NO: 448 - protein, 227 aa, Glycine max
  REGION 1..227: note = GmBBX7 soybean B-box polypeptide
MVNQLFDSCKSATATLYCRIDTTFLCGTCDSKVHAANKLVSRHPRVALCEVCKQASTHVTCKAGAAALCLTCDSEIHSTNPLASRHERIPITLFFEYVHSVKASSSINFHHCFFSDTDADVSTKEAEVASWLLNNLKTYLNSSQYLFSEKFRHRRWRHSSVEQLQAVHLRAPNIFLHSYFSRSRTFGVINYYILTGVRSNINDFSLNLQNQVKNIIKSKIALNNFFQ

SEQ ID NO: 449 - DNA, 930 bp, Glycine max
  misc_feature 1..930: note = GmBBX11
  source 1..930: mol_type = unassigned DNA
atggcttccaagctctgcgactcatgcaaatccgccactgctaccttgtactgccgccctgacgccgcgttcctctgcggcgcctgcgattctaaggtccacgccgccaacaagctcgcgtcgcgccacccgcgcgtggcgctctgcgaggtgtgcgagcaggccccggcgcacgtaacctgcaaggctgatgccgccgcgctctgccttgcctgcgaccgcgacatccactccgccaaccccctcgccagccgccacgagcgcatccccgtctcaccgttcttcgagtccgtacactccgtcaaggcctcctcgccgatcaacttcctagatgaccaccgcttcttctccgacgccgatgctgacgtcagcaccgaggaggccgaggccgcctcctggctgcttcctaatccgaagacagatctaaattcctctcagtacttgttctccgaaaccgagccggttccttacatagatctggattatgctgccgtggatcctaaggcggagcagaaaagctccgccaccgccgacggcgtcgttcccgtgcagagcaacttcgagccattcgcctacggttacaagtacaataccactctctcacagtctcagatgagccagagtgtatcgtcgtcgtcgatggaggttggagtcgtgccggacgggaacacaatgtcggagacatcgaactgcagctacagcaaggtgcctccggtgacggtgacggtgacggcgcagttctcggcggcggatagggaggcgagagtgttgaggtacagggagaagaggaagaaccgtaagttcgagaagacgattcgttacgcatcgagaaaagcatatgcggaggcgaggccaaggatcaaagggaggtttgcgaaacgcactgacccggaccctctggctggatacggcgtcgttccgtcgtgttga

SEQ ID NO: 450 - protein, 174 aa, Glycine max
  REGION 1..174: note = GmBBX11 soybean B-box polypeptide
MASKLCDSCKSATATLYCRPDAAFLCGACDSKVHAANKLASRHPRVALCEVCEQAPAHVTCKADAAALCLACDRDIHSANPLASRHERIPVSPFFESVHSVKASSPINFLDDHRFFSDADADVSTEEAEAASWLLPNPKTDLNSSQYLFSETEPVPYIDLDYAAVDPKAEQKSS

SEQ ID NO: 451 - DNA, 933 bp, Glycine max
  misc_feature 1..933: note = GmBBX12
  source 1..933: mol_type = unassigned DNA
atggcttccaagctctgcgactcatgcaaatcagccactgctaccttgtactgccgccccgacgcggccttcctctgcggcgcctgtgactctaaggttcatgccgccaacaagctcgcatcgcgccacccgcgcgtcgtgctctgcgaggtgtgcgagcaggcgcccgcgcatgtgacctgtaaagccgacgccgccgcgctctgccttgcctgcgatcgcgatatccactccgccaaccccctcgccagccgccacgagcgcatccccgtcacgccgttcttcgagtccgtacattcagtcaaggcctcctcgccgatcaacttccaccaccgcttcttctccgacgccgacgctgatgctgacgtcagcaccgaggaggccgaggccgcgtcatggctgctacctaatccaaagacagatctaaattcgtctcagtacttgttctccgaaaccgaaccggttccttacatagatctggattacgcggccatggatccgaagacggagcagaaaagttccgccaccgccgacggcgtcgttccggtgcagagcaacttcgagccgttcacctacggttacaagtacaacactactctctcacagtcacagtctcatatgagccagagtgtatcttctccgtcgtcgatggaagttggagttgtgccggacgggaacacgatgtcggagatatcgaactgcagctacagcaaagtggcaccggtgacggtgacggcgcagttctcggcggcggatagggaggctagagtgttgaggtacagggagaagaggaagaaccgaaagttcgagaagaccattcgttacgcatcgagaaaagcgtatgccgaaacgaggccaagaatcaaaggaaggttcgcgaaacgcactgatgcggaccctctggctggatacggcgtcgttccgtcgtgttga

SEQ ID NO: 452 - protein, 310 aa, Glycine max
  REGION 1..310: note = GmBBX12 soybean B-box polypeptide
MASKLCDSCKSATATLYCRPDAAFLCGACDSKVHAANKLASRHPRVVLCEVCEQAPAHVTCKADAAALCLACDRDIHSANPLASRHERIPVTPFFESVHSVKASSPINFHHRFFSDADADADVSTEEAEAASWLLPNPKTDLNSSQYLFSETEPVPYIDLDYAAMDPKTEQKSSATADGVVPVQSNFEPFTYGYKYNTTLSQSQSHMSQSVSSPSSMEVGVVPDGNTMSEISNCSYSKVAPVTVTAQFSAADREARVLRYREKRKNRKFEKTIRYASRKAYAETRPRIKGRFAKRTDADPLAGYGVVPSC

SEQ ID NO: 453 - DNA, 1260 bp, Glycine max
  misc_feature 1..1260: note = GmBBX13
  source 1..1260: mol_type = unassigned DNA
atgagttcggctactaagaacgcggcaaacgcggttggggccaaaacggcaagagcatgtgacagctgcataaccaaacgtgcacgttggtactgtgctgccgatgatgcttttctctgccaggcctgcgactcttctgttcactcggcgaatcctttggctcgaagacacgaacgagtccgcttgaaaacggcgtcctacaagtccactgatgaacaacaacaacaacaacaacaacaacctcccacgtggcacactaagaaacctcgaacgccgaggcacgggaaacattctcgtaacaacaaccctttccatttggtccctgaagagggttcggaggaggcgaattcccatgatgagaacgaggagcagcttctttatagggtccccatattcgacccttttgttgctgagctatgtgggactaatagttctccttctcctgttacttcaactgatcaaggagtagttgctgctgctgaggttgaatacaaagggtttcaaagcaatggtttttgtagcaatagcagtgagatggaaaacttgcatgggatgcttccttctgatgcagaacttgctgagttcgctgctgatgtggagagcttgttgggtagggggcttgaaaacgagtgcgtggggatggaagaattgggactggttgatgcgaaagaggaggagtgttcggtaggtagtgggaaggtgaagatggaagatcaggaagagagccctctggtggagatggagatggatatggtcgtgggcagagatgatcaatcgtttgagttgagctttgattatgaaacgtgtgaagaggtgaaggtttgtgatttgggattggggaatgaattaggagcgaagaaagagaatgatgatgaggtgaaaaagaacaagatttcgttgcagctagactatgaggcggtaatcatcgcctgggctagccaaaaatctccgtggaccaccgctgacaaaccaaacttggaccctgatgagtgctggaaacaatgcatgggaagttgcgaaacggcatatcatcatccttgcggtgaaatgggtggatttgggattcatccagtgataatagacgggggcagagaggcgagggtgtcaagatacagagagaagcgccgaacgaggttgttctcgaagaaaataaggtacgaagttaggaaattgaatgctgagaagagacccagaatgaaggggaggtttgtcaagagggcttcctttgcaccaccaacatttcctttgctaaataaataa

SEQ ID NO: 454 - protein, 419 aa, Glycine max
  REGION 1..419: note = GmBBX13 soybean B-box polypeptide
MSSATKNAANAVGAKTARACDSCITKRARWYCAADDAFLCQACDSSVHSANPLARRHERVRLKTASYKSTDEQQQQQQQQPPTWHTKKPRTPRHGKHSRNNNPFHLVPEEGSEEANSHDENEEQLLYRVPIFDPFVAELCGTNSSPSPVTSTDQGVVAAAEVEYKGFQSNGFCSNSSEMENLHGMLPSDAELAEFAADVESLLGRGLENECVGMEELGLVDAKEEECSVGSGKVKMEDQEESPLVEMEMDMVVGRDDQSFELSFDYETCEEVKVCDLGLGNELGAKKENDDEVKKNKISLQLDYEAVIIAWASQKSPWTTADKPNLDPDECWKQCMGSCETAYHHPCGEMGGFGIHPVIIDGGREARVSRYREKRRTRLFSKKIRYEVRKLNAEKRPRMKGRFVKRASFAPPTFPLLNK

SEQ ID NO: 455 - DNA, 1257 bp, Glycine max
  misc_feature 1..1257: note = GmBBX14
  source 1..1257: mol_type = unassigned DNA
atgagttcggctactaagaacgcagcaaacgcggttgggggaaaaactgcaagagcatgtgacggctgcataacgaaacgtgcacgttggtactgtgctgccgatgatgcttttctctgccaggcttgtgactcttcggttcacttggcgaatcctttggctcgaagacacgaacgagtccgcttgaaaaccgcgtcgtacaagtccaccgatgaacgacgacaacctcccacgtggcacaccaagaaacctcgaacgcccagacatgggaaacattctcgtaacaacaaccctttccacttggtccctgaagagggttccgaagaggcgaattcccatgatgagaatgaggagcagctcgtttatagggtccccatagtcgacccttttgttgctgagctatgtgggactaatagttctccttcttctgttacttcaactgatcaaggggtagtagctgctgctgctgctgcttctgctgaggttgaatacaaagggattcaaagcaatgatttttgtagcaatagcaatgagatcgaaaacttgcatgggatgcttccttcggatgcagaacttgctgagtttgctgctgatgttgagagctttttgggtagggggcttgaaaacaagtgcgtggggatggaagaattgggactcgttgataccaaagaagaggagtgttcggtgggtagtgggaaggtgaaggtggaagaggaagagagccctctgatggagatggatatgggaagagatgatcaatcatttgagttgagctttgattatgagacgtgtgaagaggtaaaggagatgaaggttagtgatttggaattggggaatgagttaggagagatgaaagagaatgatgatgatgaggtgaaaaggaagaaggtatcgttgcagcttgattatgaggcgataatcatcgcctgggctagccaaaagtctccgtggaccactgctgacaaacaaaacttggaccctgatgagtgctggcaccaatgcatgggaagttgcggaacggcatttcatcacccttacggtgaactgggtggatttgggattcattcagtgatagtagacgggggcagagaggcaagggtgtcaagatacagagagaagcgccgaactaggttgttctcgaagaaaataaggtacgaggttaggaaattgaatgcagagaagagacccagaatgaaggggaggtttgttaagagggcttcctttgcaccaccaacatttcctttgctaaataaataa

SEQ ID NO: 456 - protein, 88 aa, Glycine max
  REGION 1..88: note = GmBBX14 soybean B-box polypeptide
MSSATKNAANAVGGKTARACDGCITKRARWYCAADDAFLCQACDSSVHLANPLARRHERVRLKTASYKSTDERRQPPTWHTKKPRTPR

SEQ ID NO: 457 - DNA, 1098 bp, Glycine max
  misc_feature 1..1098: note = GmBBX15
  source 1..1098: mol_type = unassigned DNA
atgagagacatgaaagacgcaggtgccttgggaggcaagacagcaagagcctgcgacagctgcgtaagcagaagagcaaggtggttctgtgcagctgatgatgctttcctctgccatggctgcgacactttggttcattccgcgaaccagttagcaagcaggcacgaaagggttcggcttcaaactgcttcctcaaaagtaaccaccacgcacgcatggcacagtggattcacacgcaaggcaagaaccccgcgtcacaacagcaagcactttgcgctgcaacagcgtctcaaagacgaagttctcttcaacaacaccagtgttcttcccctcgtgccggagctcggaggggaagaacaagaaccggtggttgtggataacgatgagacggaggagcagatgctgtgtcgggtgcccgtgttcgacccttttgatgtgagaaccgatgacttggatagtttttctgacatggatttcgctgagtttgctgctgatgttgaaagcctacttgataaagaagacgatgaaataagtgcgtgtgtgggagggggagagggagttcaaggtgcaatggtgaaggttaaggatgaagaagaaattgatggtgatgtagcgtgttatctggaatcggtttttgatgatgcctttcattggaataacattgagtctgtgctgtcagatgcacgggaagagaaggagggtgtggtggcgtgtgatggaggtgtcggtgatgaagaaggaggaacaaagagagacatatttttgagactaaactatgatgaggttattactgcttggtctagccaaggttcttctccatggacaacttcaaacccacctaagttcaactctgattatgacttctccttgggtttgagtggtgtaggtggggaagtaagaagcttaagggggcacctagatggtggaagagaagcaagagtgtcaaggtatagagagaagagaaggactcgtttgtttgcaaagaagataagatatgaagtaaggaagttgaacgcagagaaaaggcctagaatgaaaggtcgttttgtgaagaggacatgctttgttggagccaatgcttttcctgcttaccactga

SEQ ID NO: 458 - protein, 365 aa, Glycine max
  REGION 1..365: note = GmBBX15 soybean B-box polypeptide
MRDMKDAGALGGKTARACDSCVSRRARWFCAADDAFLCHGCDTLVHSANQLASRHERVRLQTASSKVTTTHAWHSGFTRKARTPRHNSKHFALQQRLKDEVLFNNTSVLPLVPELGGEEQEPVVVDNDETEEQMLCRVPVFDPFDVRTDDLDSFSDMDFAEFAADVESLLDKEDDEISACVGGGEGVQGAMVKVKDEEEIDGDVACYLESVFDDAFHWNNIESVLSDAREEKEGVVACDGGVGDEEGGTKRDIFLRLNYDEVITAWSSQGSSPWTTSNPPKFNSDYDFSLGLSGVGGEVRSLRGHLDGGREARVSRYREKRRTRLFAKKIRYEVRKLNAEKRPRMKGRFVKRTCFVGANAFPAYH

SEQ ID NO: 459 - DNA, 1116 bp, Glycine max
  misc_feature 1..1116: note = GmBBX16
  source 1..1116: mol_type = unassigned DNA
atgagagacatgaaagacgcaggtgccctgggaggcaagacagcacgagcctgcgatagctgcgtaagcagaagggcaaggtggttctgtgcagcagatgatgctttcctctgccatgcttgcgacactttggttcattccgcgaaccagttagcaagcagacacgaaagggttcggcttcaaactgcatcctctaaggcaaccacaaccactacgcacgcatggcacagtggattcacacgcaaggcaagaaccccgcgtcacaacaacagcaagcactttgcgctgcaacagcgtctcaaacacgaagttctcttcaacaacaccagtgttcttcccctcgtgccggagctcggaggggaagaacaagaaccggtggttgtggacaacgatgaaacggaggaacagatgctgtgtcgcgtgcccgtgttcgacccttttgatgtgagaaccgatgacttggatagcttttctgacatggatttttctgaatttgctgctgatgttgaaggccggcttgacaaagaagacgatgaaataagtgcgtatgtgggaggaggagagggagttcaaggtgtattggccaaggttaagggcgaagaagaaattgatggtgatgtagcgtgttatctggagtcagtttttgacatgatcagtgatgatgcctttcattggaataatattgagtctgtggtgtcagatgcccgggaagagaaggggtgtgtggtgccatgtgatggtggggttagtgaacaaggaggaataaagagagacatatttttgagactaaactatgatgaggttattactgcttggtctagccaaggttcttccccatggacaacttcaaacccacctaagttcaactctgattatgacttctccttgggtttgagtggtgtagatggagaaggaagaagcttaaggggacacctagatggtggaagagaagcaagagtgtcgaggtatagagagaagagaaggactcgtttgtttgcaaagaagataagatatgaagtaaggaagttgaacgcagagaaaaggcctagaatgaaaggtcgttttgtgaagaggacatgctttgttggagccaatgcttttcctgcttaccactga

SEQ ID NO: 460 - protein, 309 aa, Glycine max
  REGION 1..309: note = GmBBX16 soybean B-box polypeptide
ASSKATTTTTHAWHSGFTRKARTPRHNNSKHFALQQRLKHEVLFNNTSVLPLVPELGGEEQEPVVVDNDETEEQMLCRVPVFDPFDVRTDDLDSFSDMDFSEFAADVEGRLDKEDDEISAYVGGGEGVQGVLAKVKGEEEIDGDVACYLESVFDMISDDAFHWNNIESVVSDAREEKGCVVPCDGGVSEQGGIKRDIFLRLNYDEVITAWSSQGSSPWTTSNPPKFNSDYDFSLGLSGVDGEGRSLRGHLDGGREARVSRYREKRRTRLFAKKIRYEVRKLNAEKRPRMKGRFVKRTCFVGANAFPAYH

SEQ ID NO: 461 - DNA, 1179 bp, Glycine max
  misc_feature 1..1179: note = GmBBX17
  source 1..1179: mol_type = unassigned DNA
atgaccaacgaaatgaaagaagcaagtgccttaggagctagaacagcacgagcatgcgagagctgcctaaaagtaagagcaaggtggtattgtgcagctgatgatgctttcctttgccatggctgtgacaacatggttcactcagcaaaccaattagcaagcagacacgaaagggtgaagcttcaaactgcatcctccaaggtcaactactcagtgacccctaaggtagcatggcacagtggattcacacgcaaggcaagaacccctcgcagcaacaacaagcactcatcgctgcaacaacaacaacaaaaaccattacatgaagaacaaggagaagaagaagaacttggaagtgaagagccattgcttaatgatgagaccgaggagcaactcctgtgtcgcgtgcccgtatttgatgctgagctttcctttgacttggaaaacttttcttctgagtttctaccaacagatatggatcttgctgagttttctgctgatgtggaaagcttacttggaaacggggttgatgaggattcacctgtgaagaaaggatcagaactagtgcttcttgattgcaaagaagaaggagatgatgaaatggatgcatgtgttaatggaattggagccaacaaagatgcaatggttaaggtcaaagatgaagaagaacttgatgctgatgatgacacagcttgccatttggattcgattcttgacatgaatagcgaggcctttaattggaatattgctgagtcagagtcacctgcacaggcacaagaagatgaaaagtcattactgcttgggcaagtcaaggttctccatggactacaggaaccccacctaaacttggtaacttctcttcaaccctctttgcttatgcagcagtatcttcttgaggaaggttgtgttgcgtcttcagtaaccgatgtaaaattcgtcgatatacctgaaatcattcgtagggcggagatgtgttatgccttcgatgggggaagagaagcaagagtgtcaaggtaccgagagaagagaagaacgcgtttatttgcaaagaagataaggtatgaagtgaggaagttaaacgcggagaaaagacctagaatgaaaggtcgatttgtgaagagtgttcctttgttggagccactgctttgccagcttaattatcaatcatcaccactgacatga

SEQ ID NO: 462 - protein, 392 aa, Glycine max
  REGION 1..392: note = GmBBX17 soybean B-box polypeptide
MTNEMKEASALGARTARACESCLKVRARWYCAADDAFLCHGCDNMVHSANQLASRHERVKLQTASSKVNYSVTPKVAWHSGFTRKARTPRSNNKHSSLQQQQQKPLHEEQGEEEELGSEEPLLNDETEEQLLCRVPVFDAELSFDLENFSSEFLPTDMDLAEFSADVESLLGNGVDEDSPVKKGSELVLLDCKEEGDDEMDACVNGIGANKDAMVKVKDEEELDADDDTACHLDSILDMNSEAFNWNIAESESPAQAQEDEKSLLLGQVKVLHGLQEPHLNLVTSLQPSLLMQQYLLEEGCVASSVTDVKFVDIPEIIRRAEMCYAFDGGREARVSRYREKRRTRLFAKKIRYEVRKLNAEKRPRMKGRFVKSVPLLEPLLCQLNYQSSPLT

SEQ ID NO: 463 - DNA, 1284 bp, Glycine max
  misc_feature 1..1284: note = GmBBX18
  source 1..1284: mol_type = unassigned DNA
atgaccaacgaaatgaaagaagcaagtgccttaggagctagaacagcacgagcatgcgagagctgcctaaaagtaagagcacggtggtattgtgcagctgatgatgcattcctttgccatggctgtgacaacatggttcactcagcaaaccaattagcaagtagacacgaaagggttaagcttcaaactgcatcctctaaggtcaactactcagtgacccctaaggtagcatggcacagtggattcacacgcaaggcaagaacgccacgccacaacaacaacagacactcatcattgaaacaacaacaacaaaaaaaaccattacatgaagaacgaggagaggaagaagtgtttttcaataacactattagtcttcttccccttgtgcctgagcttggaagtgaagagccattgcttaatgatgagactgaggagcaactactgtgtcgcgtgccggtgtttgatgctgagctgtgtagcatatacaatgaagtgaaggacgaggttgtagctgctggtgaagaagccttggacttggaaaacttttcttccgagtttcttccatcagatacggaccttgctgagtttactgctgatgtgaaaagctttcttggaaacggggctgatgaggattcacctgatgatgatgagcacgtgaaagaatcagaactactgattcttgattgcaaagaagaagaaggagatgaggaaatggatgaatgtattgatggagtactaattggagccaaagatgcaatggtgaaggtcaaagatgaagaagaacttgatgctgatgatgacacagcttgccatttggattcgattcttgatatgaacggcgaggcatttaattggaatattgttgaatcagaatcacctgcacaggcacaagaagatgaagtgagtaaagtgggaactaagaaggacatatttttgagattaaactatgaagaagtcattactgcttgggcaagtcaaggttctccatggactaatgggaccccacctaagttcttcaactctgatgattgctggctagacttcttgggttcgaatggtgggaatgttcaatgctgttatggggcagtgggaagcttaagggtgcatgcagatgggggaagggaagcaagagtgtcaaggtaccgagagaagagaagaacgcgtttatttgcaaagaagataaggtatgaagtgaggaagttgaacgcggagaaaagacctagaatgaaaggtcgatttgtgaagaggacaccctttgttggagccactgcgttgccagcttaa

SEQ ID NO: 464 - protein, 171 aa, Glycine max
  REGION 1..171: note = GmBBX18 soybean B-box polypeptide
MTNEMKEASALGARTARACESCLKVRARWYCAADDAFLCHGCDNMVHSANQLASRHERVKLQTASSKVNYSVTPKVAWHSGFTRKARTPRHNNNRHSSLKQQQQKKPLHEERGEEEVFFNNTISLLPLVPELGSEEPLLNDETEEQLLCRVPVFDAELCSIYNEVKDEVVA

SEQ ID NO: 465 - DNA, 1230 bp, Glycine max
  misc_feature 1..1230: note = GmBBX19
  source 1..1230: mol_type = unassigned DNA
atgggttatatatgtgatttttgtggagaccagagatccatggtgtactgccgctctgacgctgcatgcttatgtttgtcttgtgatcggaatgttcattctgctaatgccctttctaagcgtcattcacgcacccttttatgcgagagatgcaattcacaacctgcctttgtaaggtgtgttgaagagaagatttcactttgtcagaattgcgactggttgggtcatggcacctctacatcttcttcaacacataagagacaagcaatcaattgctactctggctgcccttcagcggcagaactgtcttcgatatggtcatttgtgttagaaatcgctgccataagtgagtctacatgtgagcaagaactaggcttaatgagtattaatgagaacaagtgtgttggtgttcctccggaagaccagaatgtgtctggttcagatgaagttaccgatctacctgctttgaacaagtctttggttggtgcatctttgatgcctgaatcaagctccgaaccttgtattctggaccagccagctggacctaccaatgaatgtctgccaaagttatactgtcctgctacaaaatgccctgccttatctgaagatgataatctgtatgatgatttcaacatggatgaagtggatctagatcttgagaactatgaagaactttttggtatggccctcagtcattctgaggagctttttgaaaatggtggaattgatagcttgtttgggacaaaaggcatgtctgctggagattccaattgtcaggaagctattgctgctgaggggtcgtcggttggacaggtcaatgcaatgcaaccagcttgcagcaatgcagcgtctgcagattccattttgagtactaaaactgaaccaattctttgttttactggaaggcaaactcaatcaaacctttctttttctggtgttactggagagagtagtgctggagactatcaagactgtggggcttcttcaatgcttctcatgggagaacctccttggtttgctccttgtccggagaattccctacaatccgccaaccgtagtaatgctgtcatgcggtacaaggaaaagaagaagacacggatgtttgacaaaaaagttaggtatgcctctcgcaaggcaagggctgatgtcagaaggcgtgtgaaagggcggtttgtcaaagccggtgacgtctatgattatgaccctttgagcacaaccagaagctgctga

SEQ ID NO: 466 - protein, 409 aa, Glycine max
  REGION 1..409: note = GmBBX19 soybean B-box polypeptide
MGYICDFCGDQRSMVYCRSDAACLCLSCDRNVHSANALSKRHSRTLLCERCNSQPAFVRCVEEKISLCQNCDWLGHGTSTSSSTHKRQAINCYSGCPSAAELSSIWSFVLEIAAISESTCEQELGLMSINENKCVGVPPEDQNVSGSDEVTDLPALNKSLVGASLMPESSSEPCILDQPAGPTNECLPKLYCPATKCPALSEDDNLYDDFNMDEVDLDLENYEELFGMALSHSEELFENGGIDSLFGTKGMSAGDSNCQEAIAAEGSSVGQVNAMQPACSNAASADSILSTKTEPILCFTGRQTQSNLSFSGVTGESSAGDYQDCGASSMLLMGEPPWFAPCPENSLQSANRSNAVMRYKEKKKTRMFDKKVRYASRKARADVRRRVKGRFVKAGDVYDYDPLSTTRSC

SEQ ID NO: 467 - DNA, 1224 bp, Glycine max
  misc_feature 1..1224: note = GmBBX20
  source 1..1224: mol_type = unassigned DNA
atgggctatatatgtgatttctgtggagaccagagatccatggtgtactgccgctctgatgctgcatgcttatgtttgtcttgtgatcggaatgttcattctgctaatgccctttctaagcgtcattcacgcacccttttatgtgagagatgcaattcacaacctgcctttgtaaggtgtgttgaagagaaaatttcactttgtcagaattgtgattggttgggtcatggcacctctacatcttcttcaacacacaagagacaggcaatcaattgctactctggctgcccttcagcggctgaactgtcttcaatatggtcatttgtgttagatatcgctgccataagtgaatctacatgtgagcaagaactaggcttaatgagtattaatgagaacaagagtgttggggttcctccagagggccagaatgtgtctggttcagatgaagttactgatcaacctgctttggacaagtctttggttggtacatcttcaatgcctgaatcaagctccaaacctcgtattctggaccagccagctagacctgccaatgaatgtctttcaaagttatactgtcctgctacaaaattccccgcattatgtgaaaatgataatctgtatgatgatttcaacatggatgaagtggatctaaatcttgagaactatgaagaactttttggtatggccctcagtcattctgaagagctatttgaaaatggtggaattgatagcttgtttgggacaaaagacatgtctgctggagatttcagttgtgaggatgctattgctgctgaggcatcacaaattccaactatgttggcacctcagcctcacaatgcagcatctgcagattccattttgagtactaaaactgaaccaattctttgttttacgggaaggcaagctcaatcaaacctttctttttctggcgttaccggagacagtagtgctggagactatcaagactgtggggcttcttcaatgcttctcatgggagaacctccttggtttgctccttgtcctgagaattccctacaatcagccaaccgtagtaatgctgtcatgcggtacaaggaaaagaagaagacacggatgtttgacaaaaaagtgaggtatgcctctcgcaaggcaagggctgatgtcagaaggcgcgtgaaaggccggtttgtcaaagctggtgacgtctacgattatgatcctttgagcacaaccagaagcttctga

SEQ ID NO: 468 - protein, 407 aa, Glycine max
  REGION 1..407: note = GmBBX20 soybean B-box polypeptide
MGYICDFCGDQRSMVYCRSDAACLCLSCDRNVHSANALSKRHSRTLLCERCNSQPAFVRCVEEKISLCQNCDWLGHGTSTSSSTHKRQAINCYSGCPSAAELSSIWSFVLDIAAISESTCEQELGLMSINENKSVGVPPEGQNVSGSDEVTDQPALDKSLVGTSSMPESSSKPRILDQPARPANECLSKLYCPATKFPALCENDNLYDDFNMDEVDLNLENYEELFGMALSHSEELFENGGIDSLFGTKDMSAGDFSCEDAIAAEASQIPTMLAPQPHNAASADSILSTKTEPILCFTGRQAQSNLSFSGVTGDSSAGDYQDCGASSMLLMGEPPWFAPCPENSLQSANRSNAVMRYKEKKKTRMFDKKVRYASRKARADVRRRVKGRFVKAGDVYDYDPLSTTRSF

SEQ ID NO: 469 - DNA, 1236 bp, Glycine max
  misc_feature 1..1236: note = GmBBX21
  source 1..1236: mol_type = unassigned DNA
atgggttacttatgtgatttttgtggagaccagaggtccctggtgtactgccgctctgatgctgcgtgcttatgtttgtcgtgtgatcgaaatgttcattctgctaatgccctttctaggcgtcattcgagaacccttgtatgcgagagatgcaattcgcaacctgcatttgtgaggtgtgttgatgagaagatttcactttgtcagaactgtgattggttgggtcatggtacctcaccatcttcttcgacacacaagaggcaatcaatcaattgctactctggctgcccttcagctgcagaattttcttcaatatggtcctttttcttagacatcccttctatgggtgaagcatgtgagcaggaactaggtttgatgagcattaatgagaacagcaataagaatgcttgggcttctccagaagggcagaatgtgtctggttcagctgaagtgactgatctacccagcaagggtaaatcttgggctggcacatcttcagtacctgagtcaagctcagaacctcgtattctagaccagccgcctggacctgccaatgaatgtatgcccaagttgtactgtcctgggaagaaagtttctggaatatatgaagatgatgatctatatgatgatttcatcatggatgaagtggatctacaacttgagaactacgaggaactttttgggatggctctcagtcactctgaagagctttttgaaaatggtggaattaatagcttatttgagacaaaggacatgtctgctagtgctggtgattcccattgtcagggtgctgttgctgctgagggatcgtctgctgagttggtcaatgctatccaaccagcatgcagcaatgcagcatctgcagactcaatgatgagtacaaaaactgaaccaattgtttgttttacggcaaggcaatctctatcaaacatttctttctctggtgttaccaaagatagtgttggtgactatcaagattgtggtgcttcttcaatgcttctcatgggagagcctccctggtgtcctccttgccctgagagttctctgcattctgctaaccgcagcaatgctgtcatgcgctataaggaaaagaagaagacccggaagttcgagaagaaagtaaggtatgcctctcgcaaggctagggctgatgtcagaaagcgtgtgaagggtcgatttgtcaaagccggtgatgtctatgactatgaccctttgaaccaaaccagaagctgctga

SEQ ID NO: 470 - protein, 411 aa, Glycine max; identical to SEQ ID NO: 144
  REGION 1..411: note = GmBBX21 soybean B-box polypeptide

SEQ ID NO: 471 - DNA, 1218 bp, Glycine max
  misc_feature 1..1218: note = GmBBX22
  source 1..1218: mol_type = unassigned DNA
atgggttacttatgtgatttttgtggagaccagaggtccttggtgtactgccgctctgattccgcgtgcttatgtttgtcatgcgatcgaaatgttcattctgccaatgcactttctaggcgtcattcgagaacccttgtatgcgagagatgcaattcacaacctgcatttgtgaggtctgttgaagagaaaatttcactttgtcagaactgtgattggttgggtcatggtacctcaccgtcttcttcgatgcacaagaggcaatcaatcaattgctactctggctgcccttcagcggcagaattttcttcaatatggtcctttttcttagacatcccttctttgggtgaagcatgtgagcaggaactaggtttgatgagcattaatgagaacattcctccagaagggcagaatgtgtctggttcaactgaagtgactgatctacccagcaagggtaaatcttgggctggcacaccttcaatacctgagtcaagctcagaacctcgtattctggaccagccgcctggacctgccaatgaatgtgtgcccaagttgtactgtcctgggaagaaagcttctggaatatgtgaagatgatgatctatatgatgatttcatcatggatgaagttgatctagaacttgagaactatgaggaactttttgggatggctctcagtcactctgaagagctttttgaaaatggtggaattaatagcttatttgaggcaaaggacatgtctgctagtgctggtgattcccattgtcagggtgctgttgctgctgagggatcgtctgctgggttggtcaatcctatccaaccagcatgcagcaatgcagcatctgcagattcagtgatgagtacaaaaactgaaccaattgtttgttttacggcaaggcaatctcaatcaaacatttccttttctggtgttaccaaagatagtgctggtgactatcaagactgtggtgcttcttcaatgcttctcatgggagaacctccctggtgtcctccttgccctgagagttctctgcattctgctaaccgaagcaatgctgtcatgcgctacaaggaaaaaaagaagacccggaagttcgagaagaaagtaagatatgcctctcgcaaggctagggctgatgtcagaaagcgtgtgaagggccgatttgtcaaagctggtgatgtctatgactatgaccctttgaaccaaaccagaagctactga

SEQ ID NO: 472 - protein, 405 aa, Glycine max; identical to SEQ ID NO: 140
  REGION 1..405: note = GmBBX22 soybean B-box polypeptide

SEQ ID NO: 473 - DNA, 1155 bp, Glycine max
  misc_feature 1..1155: note = GmBBX23
  source 1..1155: mol_type = unassigned DNA
atggatcctctatgtgagttttgtggggtggtgagagctgtggtttactgcaagtcagattctgctcgtctctgcttgcattgcgatggctgtgtacactctgcaaattctttgtctcgcaggcattcgcgttccctcctctgcgataagtgcaattcccagccggcaatgatccgctgcatggatcataagttgtcattgtgccaaggttgtgactggaatccaaatgactgttctgcattgggacataggcgtgttgcattgaattgttacactggttgtccctctttggctgagttttccaggatttggtcctttgtgtttgatgctaattcctcacttggtggttgggaatcggttggtactttgccaaaaagtgagagctgcactagccagtgtttggagcaaactgatcataacggtggttcctttggtttgtctcaaatcattccatcaaatccaaattacacgccgttctgcaaagacgaagcatttttctttcctcaagactcaaaccagccaaaggaatgtcctaatctaggaattcatgatgggagtgaactttgtgaaggtttcaatgttgataaccttcaattaaactttgaaagtgctgatgaaatatttggctgttcacaagatgccacaagataccaccttgaagatggaggaatggactgtttattgatggataaaaacatatcagtcacagaatctaattcacttattgagagtgccttggaggcatcatcatctatccaacaagattgtgtggcttttcaatcatccagggcgggtgggtcagctagtgtgatgcaggttatcaacagcaatacaaattctgcactaatgaatcctagctgcactagaaacatcagtctgggatttcctcagggtgttcattcaaagatgccacttcaatttcccaacattgttggtgaaaataattctactgaatatcaagactgcaggttttcgcaagtgtttctgccgggtgaatccccctgggaatcaaatttggagggtacatgcccacaagcaagggataaagctaaaatgagatacaatgagaaaaagaaaactcgaatgtatgtacctttaatgctctcaatatattatggtaaaatgaaatggaaactgttctttaaatttgtttaa

SEQ ID NO: 474 - protein, 384 aa, Glycine max
  REGION 1..384: note = GmBBX23 soybean B-box polypeptide
MDPLCEFCGVVRAVVYCKSDSARLCLHCDGCVHSANSLSRRHSRSLLCDKCNSQPAMIRCMDHKLSLCQGCDWNPNDCSALGHRRVALNCYTGCPSLAEFSRIWSFVFDANSSLGGWESVGTLPKSESCTSQCLEQTDHNGGSFGLSQIIPSNPNYTPFCKDEAFFFPQDSNQPKECPNLGIHDGSELCEGFNVDNLQLNFESADEIFGCSQDATRYHLEDGGMDCLLMDKNISVTESNSLIESALEASSSIQQDCVAFQSSRAGGSASVMQVINSNTNSALMNPSCTRNISLGFPQGVHSKMPLQFPNIVGENNSTEYQDCRFSQVFLPGESPWESNLEGTCPQARDKAKMRYNEKKKTRMYVPLMLSIYYGKMKWKLFFKFV

SEQ ID NO: 475 - DNA, 1272 bp, Glycine max
  misc_feature 1..1272: note = GmBBX24
  source 1..1272: mol_type = unassigned DNA
atggatcctctatgtgagttttgtcgggtggtgagagctgtggtttactgtaagtcagattctgctcgtctctgcttgcattgcgatggctgtgtacactctgcaaattctttgtctcgcaggcattcgcgttccctcctatgcgataagtgcaattcccagccggcaatgatccgctgcatgaatcataagttgtcattatgtcaaggttgtgactggaatccaaatgactgttctgcattgggacataggcgccttgcattgaattgctacactggttgcccctctttggctgagttttctaggctctggtcttttgtgtttgatgctaattcctcacttggtggttgggaatcggttggtactttgccaaaaagtgagagctgcactagccagtgtttggagcaaactgatcataatggtggttattttggtttggtgagtgacaagttggatgaaattgaatcttgtgtcagatatcaacctcggatggatcagtctcaaatcattccatcaaatccaaattacacaccgtactgcaaagacgaagcatttttctttcctcaagactcaaaccagccaaagctgcaggaatgtcctaatctaggaattcatgatgggaatgaactttgtgaaggtttcaatgtggataacgttcaattaaactttgaaattgctgatgaaatattttactgttcacaagttgccacaagataccaccttgaagatggaggaatggactgtttattgatggataaaaacatagcagttacagaatctagttcacttattgagagtgccatggaggcatcatcatctatccaacaagattgtgtggcttttcaatcatcgagggctggtgggtcagctagtgtgatgcaggttatcaacagcaataccaattgtgcacttatgaatcctagctgcactaaaaacatcagtctgggatttcctcagggcctagttcattcaaacatgccacttcaatttcccaacatagttggtgaaaataattctactgaatatcaagactgcgggttatcgcgagtgggtgaatccccctgggaatcaaatttggagggtacatgcccacaagcaagggataaagctaaaatgagatacaatgagaaaaagaaaactcgaacctttggtaagcaaataagatatgcttctcgcaaagcaagggccgatacaagaaaacgtgtgaaaggtagatttgttaaagcaggtgaagcatatgattatgaccctcttgggacaagggacatctaa

SEQ ID NO: 476 - protein, 423 aa, Glycine max
  REGION 1..423: note = GmBBX24 soybean B-box polypeptide
MDPLCEFCRVVRAVVYCKSDSARLCLHCDGCVHSANSLSRRHSRSLLCDKCNSQPAMIRCMNHKLSLCQGCDWNPNDCSALGHRRLALNCYTGCPSLAEFSRLWSFVFDANSSLGGWESVGTLPKSESCTSQCLEQTDHNGGYFGLVSDKLDEIESCVRYQPRMDQSQIIPSNPNYTPYCKDEAFFFPQDSNQPKLQECPNLGIHDGNELCEGFNVDNVQLNFEIADEIFYCSQVATRYHLEDGGMDCLLMDKNIAVTESSSLIESAMEASSSIQQDCVAFQSSRAGGSASVMQVINSNTNCALMNPSCTKNISLGFPQGLVHSNMPLQFPNIVGENNSTEYQDCGLSRVGESPWESNLEGTCPQARDKAKMRYNEKKKTRTFGKQIRYASRKARADTRKRVKGRFVKAGEAYDYDPLGTRDI

SEQ ID NO: 477 - DNA, 1050 bp, Glycine max
  misc_feature 1..1050: note = GmBBX25
  source 1..1050: mol_type = unassigned DNA
atgagtggtgaagcgcgatcctgtgactactgtggccactccacagcacttctttactgcagagccgattcagcaaaactctgtttctcgtgcgatcgcgaggttcactccaccaaccagctcttctcgaaacacacgcgcacgctgctctgcgacgcatgcgatcattcccctgccaccatactctgttccaccgacacctctgttctgtgccagaactgcgactgggagaagcacaaccctgcgctctccgattcccttcacgaacgaaggcccctcgaaggcttcactgggtgcccttcagtctccgaactcttatccatcgtggggttttcggatttaagcaaaaaatctcttctttcttcgcctcagggaagtggcgctgatgggatcttcggtggagaaattgaagggctttcggatttgtttgtgtgggattctccttctttcgttacgcttgatgatttgatttcttcttctccttcttctcatagctttcaggccatggaggttcctcctctgcccaagaatcggaaggctgcttgtgggcaacacaaagaagagattcttagtcagcttcgggaattggccaagtctgagccattggaccttgaaccatatgtgtcatcggggaacttgtcctcgggttttgaacgtgaaccagaggctgatatctttccttctcatgagtggcatagagaaagcagcgagcctatgtatcaggttgttcctcctgacccattaatgagaacttatacagaagaaattccatttaaacattctacttctgctgttggggaaactcaaacctacggtgacaatggaggaaaaccatccatttctcttaaatctgaaaccctatcaactactcccaaagctgcagcatgtgagttaacaagccaagaaagagattctgcattattacggtacaagcagaagaagaaaactaggagatttgacaaacacataaggtatgaatcacgaaaagttcgggcagaaagcagggtaagagtcaagggtcgatttgccaagatggaacatgaacattga

SEQ ID NO: 478 - protein, 178 aa, Glycine max
  REGION 1..178: note = GmBBX25 soybean B-box polypeptide
MSGEARSCDYCGHSTALLYCRADSAKLCFSCDREVHSTNQLFSKHTRTLLCDACDHSPATILCSTDTSVLCQNCDWEKHNPALSDSLHERRPLEGFTGCPSVSELLSIVGFSDLSKKSLLSSPQGSGADGIFGGEIEGLSDLFVWDSPSFVTLDDLISSSPSSHSFQAMEVPPLPKNR

SEQ ID NO: 479 - DNA, 1056 bp, Glycine max
  misc_feature 1..1056: note = GmBBX26
  source 1..1056: mol_type = unassigned DNA
atgagtggtgctgaagcgcgaccttgtgactactgtggcaactccacagcacttctctattgcagagccgattcagcaaaactctgtttctcgtgcgatcgcgaagttcactccaccaaccagctcttctccaagcacacgcgcacgctgctctgcgacgcatgcgatgattcccctgcgaccatactctgttcaaccgacacctctgttctgtgccagaactgcgactgggagaaccacaaccctgcgctctccgattcccttcacgagcgaaggcccctcgaaggcttcactgggtgcccttccgtctccgaactcttgtccatcgtggggttttcggatatcagcaaaaaatctctgcttttttcgcctcagggaagtgtcgctgatgggttcttcggcgccagcgagattgaagggctttcggatatgttcgtgtgggatgcgccttcttttgttacgcttgatgatttgatttcttcctctccttcttctcatagctttcaggccatgaaggttcctcctctgcccaagaatcgaaaggctgcttgtgggcaacacaaagaagagattcttagtcagcttcgggaattggccaagtctgagcctttggaccttgaaccatatgtgcaatcgggtaacttgtccttgggttttgaacgtgacccggaggctgatatctttccttctcatgagtggcatagagaaagcagcgagcctatgcatcaagttgtgcctcctgacccatcaatgggaacttatactgaagaaattccagttaaacattctacttctgctgttggggaaaatcacacctatggtgacaatgaagggaaaccatccatttctctcaagtctgaaaccctatcaactactcccaaagctgcagcatgtgagttaacaagccaagaaagagattctgcattattacggtacaagcagaagaagaaaacaaggagatttgacaagcacataaggtatgaatcacgaaaagttcgggctgaaagcagggtaagagtcaagggccgatttgccaagatgggacatgaacattga

SEQ ID NO: 480 - protein, 351 aa, Glycine max
  REGION 1..351: note = GmBBX26 soybean B-box polypeptide
MSGAEARPCDYCGNSTALLYCRADSAKLCFSCDREVHSTNQLFSKHTRTLLCDACDDSPATILCSTDTSVLCQNCDWENHNPALSDSLHERRPLEGFTGCPSVSELLSIVGFSDISKKSLLFSPQGSVADGFFGASEIEGLSDMFVWDAPSFVTLDDLISSSPSSHSFQAMKVPPLPKNRKAACGQHKEEILSQLRELAKSEPLDLEPYVQSGNLSLGFERDPEADIFPSHEWHRESSEPMHQVVPPDPSMGTYTEEIPVKHSTSAVGENHTYGDNEGKPSISLKSETLSTTPKAAACELTSQERDSALLRYKQKKKTRRFDKHIRYESRKVRAESRVRVKGRFAKMGHEH

SEQ ID NO: 481 - DNA, 702 bp, Glycine max
  misc_feature 1..702: note = GmBBX27
  source 1..702: mol_type = unassigned DNA
atgatgaatggttctccaaattcaaaacagagaacatgtgactattgcgggagcttcacggctcttctgtactgcagagccgattctgcgaagctttgtttcttctgtgaccgtaaagttcacttcccaaaccagcttttctcgaagcacaagcgcgcgcagctctgtgatgcgtgtggagattcccctgcctcggtgctctgttctgcagaaaactctgttctgtgccaaaactgtgattgcggaaagcaaaaacatttggtgtctgaggcgcaccaacggaggccactcgaggggttctcggggtgcccttctgtgactgagttgttaacaattctgggtctcagtgaaaaatctctgctttcaaatgagggaacttcccagattgatgatgacctttcggatttgcacgtgtggagtgctccttctattaatgggttgaaacgtaagggtgcgtgcgggagacacaaagaagagattcttagtcaactccgtgagctaataaagttggagcccgacttgattcatgctgaacgacaagggcaatttggaaatttgcctactgattttgaacgagacgtggaggctaatatatttccttcatatgagtgcacaaatgggtccctaaaacgaataattcaaatagatgaaaggactatagtagcctacgtttgtaatcaatcatcaagggctaaatgcaag

SEQ ID NO: 482 - protein, 177 aa, Glycine max
  REGION 1..177: note = GmBBX27 soybean B-box polypeptide
MMNGSPNSKQRTCDYCGSFTALLYCRADSAKLCFFCDRKVHFPNQLFSKHKRAQLCDACGDSPASVLCSAENSVLCQNCDCGKQKHLVSEAHQRRPLEGFSGCPSVTELLTILGLSEKSLLSNEGTSQIDDDLSDLHVWSAPSINGLKRKGACGRHKEEILSQLRELIKLEPDLIHA

SEQ ID NO: 483 - DNA, 666 bp, Glycine max
  misc_feature 1..666: note = GmBBX28
  source 1..666: mol_type = unassigned DNA
atgatgagtggttctccttctccaaattcaaaacagagaacatgcgactattgtggggacttcacggctcttctgtattgcagtgccgattctgcgaagctatgtttcttctgtgaccgcaaagttcactccccaaaccagcttttctccaagcacacgcgagcgcagctctgtgattcgtgtggagattcccctgcttcggtgctctgttctgcagagaactctgttctttgccataactgtgactgcgaaaagcacaaacatttggcgtctgaagtgcaccaacgaaaacccctcgaggggttctcggggtgcccttctgtgactgagttgttaacgattcttggtctttctgaaaaatctctgctttcaaatgaggggacttcccagattgattacgacctttcggatttgcacgaatctctagcaagtatattctcaggtgtgaacaatttctttatttggttgatattgttattgaatcagaatcgtaagagtgcgtttgggagacacaaagaagagattcttagtcaactccgtgagctaataaagttggagcccgacttgattcatggagaagtagatgctgagcgacaagggcaatttggaaatttgcctactggctttgaacgtgacgtggaggctagtatgtttccttcatatgag

SEQ ID NO: 484 - protein, 222 aa, Glycine max
  REGION 1..222: note = GmBBX28 soybean B-box polypeptide
MMSGSPSPNSKQRTCDYCGDFTALLYCSADSAKLCFFCDRKVHSPNQLFSKHTRAQLCDSCGDSPASVLCSAENSVLCHNCDCEKHKHLASEVHQRKPLEGFSGCPSVTELLTILGLSEKSLLSNEGTSQIDYDLSDLHESLASIFSGVNNFFIWLILLLNQNRKSAFGRHKEEILSQLRELIKLEPDLIHGEVDAERQGQFGNLPTGFERDVEASMFPSYE

SEQ ID NO: 485 - DNA, 1083 bp, Glycine max
  misc_feature 1..1083: note = GmBBX29
  source 1..1083: mol_type = unassigned DNA
atgttgccatgcgactactgcgactccaaacccgccttactcttctgccgagctgattccgctaaactctgtttagtttgcgaccagcacgtgcacgccgccaacgccctctccctcaagcacgtgcgcttccagatttgcgactcctgcaagtccgacaccgccgtcctccgctgctccacccacaacctcgtcctctgtcacaactgcgacgtggacgcccacggtgccgacgcttcatccctccaccaccaccaccaccaccaccgccgccttcacggcctctccggctgtccctccgtcccggagatcgcctccgcgctgggccttgatttccgggcccaggagcccgtggtccccaccgccgcctcccgcgatgaggtctatgagcaggtgctggaaattgcgcgccagaggaacaacggcctcggcgccgaacaactcaagttcgacgactcacccggaaacgacacgccgttcacctccttgcttatgttgcagaattctgaatctgagtttgacgctagaaaaagtaacgataacaacggttacggaaccgaggcatgggatcttcactggaattataataaccccgcgtatcagcctcctcaggtgtgggattttcaattacaaaaatcaacagattgcaatgaaccaagagttgatgtgcacaacatgaattactcaacaattggtgatgatattgatattctatcaagaaataatcaatcggatcagtcatcatcaagccatgcaaagaggaaagaagagagcaacaaaaaagctagaggtgggttatcatcagagtccacattgtttgaatccataccgtatagtggcaccaacaatgtcgttgtcatggagcatcttgtgggtgggaatgagaatgttagcaccttaaaagccagggttagtttgcaagagttggctaagaatagaggggatgccatgttacgctataaggagaagaagaaaacgcgaaggtacgacaagcacattcgctatgaatcaaggaaggccagggctgatactagaaaaagggtgagagggagatttgtgaaggcaagtgatgttcaagcatga

SEQ ID NO: 486 - protein, 360 aa, Glycine max
  REGION 1..360: note = GmBBX29 soybean B-box polypeptide
MLPCDYCDSKPALLFCRADSAKLCLVCDQHVHAANALSLKHVRFQICDSCKSDTAVLRCSTHNLVLCHNCDVDAHGADASSLHHHHHHHRRLHGLSGCPSVPEIASALGLDFRAQEPVVPTAASRDEVYEQVLEIARQRNNGLGAEQLKFDDSPGNDTPFTSLLMLQNSESEFDARKSNDNNGYGTEAWDLHWNYNNPAYQPPQVWDFQLQKSTDCNEPRVDVHNMNYSTIGDDIDILSRNNQSDQSSSSHAKRKEESNKKARGGLSSESTLFESIPYSGTNNVVVMEHLVGGNENVSTLKARVSLQELAKNRGDAMLRYKEKKKTRRYDKHIRYESRKARADTRKRVRGRFVKASDVQA

SEQ ID NO: 487 - DNA, 1146 bp, Glycine max
  misc_feature 1..1146: note = GmBBX30
  source 1..1146: mol_type = unassigned DNA
atgttgccatgcgattactgccattccaaacccgccatactcttctgcagaccagattccgctaaactatgcttgctatgcgaccagcacgtgcacgccgccaacgccctctccctcaagcacgtgcgcttccagatttgcgactcctgcaagaccgacaccgctgtcctccgctgctccaccgacaacctcgtcctctgccaccactgcgatgtggaaacccacggtgctgccgcttcctcccaccaccaacgtcaccgcctccacggcctctccggctgcccctccgtcacggagatcgtctccgcgctgtgcctcgatttccgggcccaggatcccgtggtccccaccgccgcctccggcggtcgcgatgaggtgtacgagcaggtgctggaaatcgcgcggcagaggaacgacgacctcggcgccgaacaactcaagttcgacgaatcccccataaacgacgtcgttgttgttgacgagatgctgatgcagcaaacgccgttcacgtctttgcttatgttgccaaattccgaatctgaattcgactcccgaaaaagtaacgataacaacggttacggaaccgaggcaggggatcttctctggaattataaccccgcgtatcagcctcctcaggtgtgggattttcagttacaaaaatcaagagattgccatgaaccaagagttgtaacatttgatggtctagaagttccaaaattatttcaggatgagcacaatatgaagtactcaacaattggtgatgacattgatattctatcaagaaataatcaatcggatcagtcatcatcaagccatgcaaagaagaaagaagagaacaacaaaaaagctaaaggtgggttatcatcagagtccaaactgtttgaatccataccgtacaatggcaccaacaatgtcgtagtcatggagcatcttgttggtgggaatgaaaatgttggcaccttaacagccagggttagtttggaagagttggctaagaatagaggggatgccatgttgcgttacaaggagaagaagaaaactcgaaggtacgataagcacattcgctatgaatcaaggaaggccagggctgatactagaaaaagggtgagagggagatttgtgaaggcaagtgatgttcaagcgtga

SEQ ID NO: 488 - protein, 381 aa, Glycine max
  REGION 1..381: note = GmBBX30 soybean B-box polypeptide
MLPCDYCHSKPAILFCRPDSAKLCLLCDQHVHAANALSLKHVRFQICDSCKTDTAVLRCSTDNLVLCHHCDVETHGAAASSHHQRHRLHGLSGCPSVTEIVSALCLDFRAQDPVVPTAASGGRDEVYEQVLEIARQRNDDLGAEQLKFDESPINDVVVVDEMLMQQTPFTSLLMLPNSESEFDSRKSNDNNGYGTEAGDLLWNYNPAYQPPQVWDFQLQKSRDCHEPRVVTFDGLEVPKLFQDEHNMKYSTIGDDIDILSRNNQSDQSSSSHAKKKEENNKKAKGGLSSESKLFESIPYNGTNNVVVMEHLVGGNENVGTLTARVSLEELAKNRGDAMLRYKEKKKTRRYDKHIRYESRKARADTRKRVRGRFVKASDVQA

SEQ ID NO: 489 - DNA, 570 bp, Glycine max
  misc_feature 1..570: note = GmBBX31
  source 1..570: mol_type = unassigned DNA
atgcgaacgctttgtgatgcttgtgagagtgcggcggctatagttttctgcgccgctgatgaggctgcactttgtcgtgcctgtgatgagaaggttcacatgtgcaataagctagctagtaggcatgtgagggttggtcttgcaagtccaagtgatgtgccacggtgtgacatatgtgagaatgcaccagctttcttctattgcgagacagatggaagttccctttgtttgcagtgtgatatgattgttcatgttggtggtaaaagaacgcatggaagatatcttctgttcaggcaaagagttgagtttccaggagataaatctagtcatgctgaaaatccggcttcacaagcattggaaccaggtgaggctaaaagaggacaaaatccacttcccaagctaaaaatgggagagaaacaacaaaatcacaggatgcctatggttccaacaccaggacctgatgctgatgggcaaaccaagatggaaactaaaatgattgatttgaacatgaagcctaatagaatacatgaacaagcatcaaataaccaggttagagttcatgggaagtaa

SEQ ID NO: 490 - protein, 189 aa, Glycine max; identical to SEQ ID NO: 230
  REGION 1..189: note = GmBBX31 soybean B-box polypeptide

SEQ ID NO: 491 - DNA, 555 bp, Glycine max
  misc_feature 1..555: note = GmBBX33
  source 1..555: mol_type = unassigned DNA
atgcgaacgctttgtgatgcttgtgagagcgccgcggctatagttttctgcgccgctgatgaggctgcactttgtcgtgcctgtgatgagaaggttcacatgtgcaacaagctagctagtaggcatgtgagggttggtcttgctagtccaagtgatgtaccacggtgtgacatatgtgagaatgcacctgctttcttctattgcgagacagatggaagttccctttgtttgcagtgtgatatgattgttcatgttggaggtaaaagaacgcatggaagatatcttctgttcaggcaaagagttgagtttccaggagataaatctagtcatgctgaaaatccggcttcacaaccattggaaccaggtgaggctaaaagaggacaaaatccacttcccaaactaaaaatgggagagaagcaacaaaatcacaagatgcctatggttccaacgccaggacctgatgctgatgggcatgccaagatggaaagtaaaatgattgatttgaacatgaagcctaatagaatacatgaacaagcatcaaataatcagccataa

SEQ ID NO: 492 - protein, 184 aa, Glycine max; identical to SEQ ID NO: 222
  REGION 1..184: note = GmBBX33 soybean B-box polypeptide

SEQ ID NO: 493 - DNA, 639 bp, Glycine max
  misc_feature 1..639: note = GmBBX34
  source 1..639: mol_type = unassigned DNA
atgcgaaccctttgcgacgtatgcgagagcgccgctgccattgtcttctgcgccgctgatgaggctgctctctgctccgcttgtgaccacaagatccatatgtgtaacaaacttgctagtagacacgttcgggttggccttgctgaccccactgatgttccacgctgtgacatatgtgaaaacgcacctgctttcttttactgtgagatagatggtagttcgctgtgtctgcaatgtgatatgattgtgcatgttggtggtaaaagaactcatggaagatatcttctattgaggcaaagagttcagtttccatgtgataaacctgcccaaatggaagaactagggctgcaacctatggatcaaaatgaatcaaggagggatgaaagtcagtcactcaagctaaaaataagagatagtcagcagaatcacagtgtctcacctgttccaagacaagaaaataatattgatggtcatggaaaaatggataaaaagttaattgatcttaatacgaggccactgcggttaaatggagcagcaccaaacaaccaggaacgaggtatggatattttaagaggcaataatcataaatctgcgagtgtgccaccagtagaatccttcaaacaagagtctgagaagtaa

SEQ ID NO: 494 - protein, 90 aa, Glycine max
  REGION 1..90: note = GmBBX34 soybean B-box polypeptide
MRTLCDVCESAAAIVFCAADEAALCSACDHKIHMCNKLASRHVRVGLADPTDVPRCDICENAPAFFYCEIDGSSLCLQCDMIVHVGGKRT

SEQ ID NO: 495 - DNA, 639 bp, Glycine max
  misc_feature 1..639: note = GmBBX35
  source 1..639: mol_type = unassigned DNA
atgcgaaccctttgcgacgtatgcgagagcgccgctgccattctcttctgcgccgctgatgaggccgctctctgctccgcttgtgaccacaagatccatatgtgtaacaaacttgctagtaggcatgttcgagttggccttgctgaccccactgatgttccacgctgtgacatatgtgaaaatgcacctgctttcttttactgtgagatagatggtagttcgctgtgtctgcaatgtgatatgattgtgcatgttggtggtaaaagaacccatggaagatatcttctattgaggcaaagagctcagtttccaggtgataaacctgcccaaatggaagaactagagctgcaacctatggatcaaaatgaatcgagaagggatgaaagtcagtcactcaagctaaaaacaagagatagtcagcagaatcacagtgtctcaccttttccaaggcaagaaaataatattgatggtcatggaaaaatggataaaaagttaattgatcttaataccaggccactgcggttaaatggatcagcaccaaacaaccaggaacaatgcatggatattttaagaggcaataatcatgaatctgccagtgtgccaccagtagaatccttcaaacaagagtctgagaagtaa

SEQ ID NO: 496 - protein, 212 aa, Glycine max; identical to SEQ ID NO: 232
  REGION 1..212: note = GmBBX35 soybean B-box polypeptide

SEQ ID NO: 497 - DNA, 801 bp, Glycine max
  misc_feature 1..801: note = GmBBX36
  source 1..801: mol_type = unassigned DNA
atgaagatccactgcgacgtgtgtaacaagcaccaggcctctttcttctgcaccgccgacgaagctgccctatgcgacggctgcgaccaccgcgtccaccacgccaacaagctcgcctccaaacaccaacgcttctccctcacccacccctctgcgaaacattttcccctctgcgatgtttgccaggagagaagagccttcgtgttttgtcagcaagatagagcgattctgtgcaaagagtgtgacgtgcccattcattctgccaacgacctcaccaagaaccatagcaggtttcttctcactgggattaagttctctgcttctgccacgccttatgattattcatcaccaccaccaccaccaccaccaccaccacccaagagaaaccctgttctcgattctccttcaacaccgtcaccgcctaagcctgggggaaactcgctaacaaacgaagaagaacctggtttcacaggtagcagcatttcagagtacttgataaactctatcccaggcatgaagttcgaagatttcctcgattcacattctcttccctttgcttgctccaagaatagtgatgacatgttgtcgctgtttggtgagggaaacatggtttctttctcacccggtgggttctgggttcctcaagcaccaccatcttcagtacaaatggatcggcagagtgggtacagagagacaagggaaggtagcattagatcaagttttggagatgataatttcatagttccacagatgagtcctcccagcaatgtgtctaataagagatccaggcttctctggtaa

SEQ ID NO: 498 - protein, 266 aa, Glycine max; identical to SEQ ID NO: 90
  REGION 1..266: note = GmBBX36 soybean B-box polypeptide

SEQ ID NO: 499 - DNA, 738 bp, Glycine max
  misc_feature 1..738: note = GmBBX37
  source 1..738: mol_type = unassigned DNA
atgaagatccagtgcgacgtgtgtaacaaacagcaggcatcgttgttctgcaccgccgacgaagccgccctatgcgacggctgcgaccaccgcgtccaccacgccaacaagctcgcctccaaacaccaacgcttctccctcagccacccctctgcaaaacattttcctctctgcgatgtttgccaggagagaagagcctttgtgttttgtcagcaagatagagcgattctgtgcaaagagtgtgacgtgcccgttcattctgccaacgacctcaccaagaaccataacaggtttcttctcactgggattaagttttctgccctcgattctccttcaacacctcctaagcctgcaggtggaaactccctaacaaatcaacaaccacaacaacaaactggtttcacaggtagcagcatatcagaatacttgataaacactatcccaggcatggagttcgaagatttcctcgattcacattctcttccctttgcttgctccaagaatagtgatgacatgatgttgtcgatgtttggtgagggaaacatggtttcgttctcagccggagggatctgggttcctcaagcaccatcttcagtacaaatggatcagcagagtgggtacaaagacacatgggaaactagcattagatcaagttttggggatgatagtttattagttccacagatgactcctcccagcaatgtgtttaataataagagatccaggcttctatggtaa

SEQ ID NO: 500 - protein, 245 aa, Glycine max; identical to SEQ ID NO: 96
  REGION 1..245: note = GmBBX37 soybean B-box polypeptide

SEQ ID NO: 501 - DNA, 831 bp, Glycine max
  misc_feature 1..831: note = GmBBX38
  source 1..831: mol_type = unassigned DNA
atgaagatccagtgcgacgtgtgcaacaaacacgaggcctccgtcttctgcacagccgacgaagccgccctctgcgacggctgcgaccaccgtgtccaccatgccaacaaactcgcctccaaacaccaacgcttctctcttctccgcccttctcataaacaacaccctctctgcgatatttgccaggagagaagagccttcacgttctgtcagcaagacagagcgattctctgcaaagagtgtgacgtgtcaattcactctgccaacgaacacacccttaagcacgataggttccttctcactggtgttaaactcgcagcttctgccatgcttcgttcatcacaaactacctctgattcaaactcaaccccttctcttcttaacgtttcacatcaaactactccacttccatcttccaccaccaccaccaccaccaacaacaacaacaacaaggttgctgttgaaggaactggttcaacgagtgctagcagcatatcagagtatttgatagagactcttcctgggtggcaagttgaggactttctcgattcatattttgttccctttggtttctgtaagaatgatgaagtgttgccacggttggatgctgacgtggaggggcatatgggttcgttttcaaccgagaacatggggatctgggttcctcaagcgccaccacctcttgtgtgttcttcacaaatggatcgggtgatagttcaaagtgagaccaacatcaaaggtagcagcatatcgaggttgaaggatgatactttcactgttccacagattagtcctccctccaattccaagagagccagatttctatggtag

SEQ ID NO: 502 - protein, 276 aa, Glycine max; identical to SEQ ID NO: 100
  REGION 1..276: note = GmBBX38 soybean B-box polypeptide

SEQ ID NO: 503 - DNA, 837 bp, Glycine max
  misc_feature 1..837: note = GmBBX39
  source 1..837: mol_type = unassigned DNA
atgaagatccagtgcgacgtttgcaacaaacacgaggcctccgtcttctgcaccgccgatgaagccgccctctgcgacggctgcgaccaccgtgtccaccatgccaacaaactcgcctccaaacaccaacgcttctctcttctccgcccttctcctaaacaacaccctctctgcgatatttgccaggagagaagagcctttacattctgtcagcaagacagagcgattctctgcaaagagtgtgacgtgtcaattcactctgccaacgaacacacccttaagcacgataggttccttctcaccggtgttaaactttctgcttctgctatgcttcgttcgtcagaaactacctctgattcaaactcaaacccttctcttcttaacttttcacaccaaacaacattactacctccatcttccaccaccaccaccaccaccagcaacaacaacaacaacaaggttgctgttgaaggaactggttcaactagtgctagcagcatatcggagtatttgatagagactcttcctgggtggcaagttgaagactttcttgattcatattctgttccctttggtttctgtaagaatgatgaagtgttgccacggtttgatggtgaaatggaggggcatctgagttctttctcaaccgagaacatggggatctgggttcctcaagcgccaccaactcttatgtgttcttcacaaatggatcgggtgatagttcacggtgagaccaatatcaaaggtagcagcagatcaaggttgaaggatgataatttcactgttccacagattagtcctccttccaattccaagagagccagatttctgtggtag

SEQ ID NO: 504 - protein, 278 aa, Glycine max
  REGION 1..278: note = GmBBX39 soybean B-box polypeptide
MKIQCDVCNKHEASVFCTADEAALCDGCDHRVHHANKLASKHQRFSLLRPSPKQHPLCDICQERRAFTFCQQDRAILCKECDVSIHSANEHTLKHDRFLLTGVKLSASAMLRSSETTSDSNSNPSLLNFSHQTTLLPPSSTTTTTTSNNNNNKVAVEGTGSTSASSISEYLIETLPGWQVEDFLDSYSVPFGFCKNDEVLPRFDGEMEGHLSSFSTENMGIWVPQAPPTLMCSSQMDRVIVHGETNIKGSSRSRLKDDNFTVPQISPPSNSKRARFLW

SEQ ID NO: 505 - DNA, 960 bp, Glycine max
  misc_feature 1..960: note = GmBBX40
  source 1..960: mol_type = unassigned DNA
atgaagatccagtgtgctgtgtgtgacaaagtggaagcctccgtcttctgctctgcagatgaagctgctctttgccatagttgtgaccgcaccattcaccacgccaacaagcttgcaaccaaacacccacgcttctctctccattaccccacctcccaagacttccctctttgtgatatttgccaagagaggcgtgcatatctattttgccaagaagacagggcattgctatgcagggagtgtgacgttcctatccacagggcgaatgagcatacccagaagcataacaggtttcttctcaccggagtgaagctctctggtacttctttggatcctgctgcgtcctccaccaattgcacccatggctctgaaggaagaaacaacgctcgatctagaatgaacagacccagatcatcagtttccaatgaagaaaatgcaagtaattcttcttgcaaggttgaagacaacgtggctagtgacactggttcggtttcaactagcagcatttctgagtacttgatcgaaacaatacccggttactgttttgaagaccttcttgatgcttcatttgcacctaatggtttctgtaagaaccagaactatgatcgtcataggcgtttcaggaccaagatcttcaagttattaggacgtgtttggtttgagttaaggagattccaaaatcttaacgctatcgatgatcaactgtttgtaaaaatctgttggaaaatatcttcttttgtttttgttatttgcatttctttttgggctccctgctgcattctttatgatttttttttcattctctattcccgaccactgtttcggcctcttattttagtatttttttcaattttcattgggttaattaataatattttttgggggggggtccatgcttttattgacatgaacatgaattcattgaataacgcaactgaatattatgtgccactttcttaa

SEQ ID NO: 506 - protein, 319 aa, Glycine max; identical to SEQ ID NO: 88
  REGION 1..319: note = GmBBX40 soybean B-box polypeptide

SEQ ID NO: 507 - DNA, 702 bp, Glycine max
  misc_feature 1..702: note = GmBBX41
  source 1..702: mol_type = unassigned DNA
atgaagatccagtgtgatgtgtgtgacaaagtggaagcctccgtcttctgtcctgcagatgaagctgctctttgccatagctgtgatcgcaccattcaccgcgccaacaagcttgcaaccaaacacgcccgtttctctctccactaccccacctccaaagacttccctctttgtgatatatgccaagagaggcgtgcatatctattttgccaagaagacagggcgttgttatgcagggaatgcgacgttcctatccacagagccaatgagcatacccaaaagcataacaggtttcttctcacgggtgtgaagctctctggtacttgtttagatcctgcttcgtcctccaccaattacaccaataataatagagtcactggctctgaaggaagaaacgacgctcgatctagaatgaacagacccagatcatcagtttccaatgaagaaaatgcaagtaattcttcttgcaaggttgaagacaacgtggctagtgacactggttcgatttcaactagcagcatttctgagtacttgattgaaacaatacccggttactgttttgaagacctccttgatgcttcgtttgcacctaatggtttctgtaagaagcagaactttgatcatccctgggcgtttcaggaccaaaagcgaacaagtggagacactatggccgtgccgttcctgaaactagtcatccattga

SEQ ID NO: 508 - protein, 233 aa, Glycine max
  REGION 1..233: note = GmBBX41 soybean B-box polypeptide
MKIQCDVCDKVEASVFCPADEAALCHSCDRTIHRANKLATKHARFSLHYPTSKDFPLCDICQERRAYLFCQEDRALLCRECDVPIHRANEHTQKHNRFLLTGVKLSGTCLDPASSSTNYTNNNRVTGSEGRNDARSRMNRPRSSVSNEENASNSSCKVEDNVASDTGSISTSSISEYLIETIPGYCFEDLLDASFAPNGFCKKQNFDHPWAFQDQKRTSGDTMAVPFLKLVIH

SEQ ID NO: 509 - DNA, 648 bp, Glycine max
  misc_feature 1..648: note = GmBBX42
  source 1..648: mol_type = unassigned DNA
atgaagatccagtgtgatgtgtgccacaaagaagtggcctccttcttctgcccctcagatgaagcctctctctgccatgcttgtgatcgcaccatccaccatcccaacaagctctcagaaaaacacaaacgcttctctcttcaccaccccatttccaccaaagactctcctctttgtgatatctgtcataatgctcaactcattattattcctttccaggagaagcatatggcttgtgacaaggtttcggtttcaacaagcagcatttctgagtacttgattcagaccattcccggttattgcatggaagatcttctagatgcttcattcgcatccaatagtttatctaaggattatgagcaccaatcagcgtttcagaaccaagatgttcaagtcagcatgcgttcgttcccactgcaaacttgggtaccacaatctcaaggtggatcccctcaacgtagctttacttcgaatctttatccccagattgattctttagttggagtgatggaaatgcctaaagccaaagctggtgaaggatactccaattggatatataattatgactacgcagcttacaaacttccttcaatttgtcctccattgatcaaaaaaatgcaaacgctctcgttaatcaattgtatgtga

SEQ ID NO: 510 - protein, 215 aa, Glycine max
  REGION 1..215: note = GmBBX42 soybean B-box polypeptide
MKIQCDVCHKEVASFFCPSDEASLCHACDRTIHHPNKLSEKHKRFSLHHPISTKDSPLCDICHNAQLIIIPFQEKHMACDKVSVSTSSISEYLIQTIPGYCMEDLLDASFASNSLSKDYEHQSAFQNQDVQVSMRSFPLQTWVPQSQGGSPQRSFTSNLYPQIDSLVGVMEMPKAKAGEGYSNWIYNYDYAAYKLPSICPPLIKKMQTLSLINCM

SEQ ID NO: 511 - DNA, 867 bp, Glycine max
  misc_feature 1..867: note = GmBBX43
  source 1..867: mol_type = unassigned DNA
atgaagatccagtgtgatgtgtgtcacaatgaagttgcttcctttttctgcccctctgatgaagcctctctctgccatgcttgtgatcgcaccatccaccatgccaacaagctcgcagacaaacacaaacgcttctcccttcaccaccccacttccaaagactcccctctttgtgatatctgtcatgagaggcgtgcatatcttttttgcaaagaggacagagcaatactatgtagagaatgtgatctttctattcatggggtcaacgaacataccaagaaacacaacaggtttcttctcactggcgtaaagatcggtgctgatgcttcagacccaacatcactttcctcaaatgacactgccattgaagaaagaacaacctctagttctaaaataaacagaccaataagttctttgtcaaatgagaatatcagttcttctcccacggttggagacaacatggcttgtgacacgggttcggtttcaactagcagcatttctgagtacttgattcagaccattcccggttactgcatggaagatcttttagatgcttcatttgcatctaatggtttatctaaggattatgagcaccaatcagcgtttcagaaccaagatgttcaagtcagcatgtgttcgtttccactgcaaagttgggtaccacaatctcaagctggatcccctcaactaattagcactacctcgaatcttcatccccagattaattctttagttggagtaatggaaatgcctaaagccaaagctagtgaaggatactccaattgcatatataattatgactactcagcttacaaagttccttcaatttgtcctccattgatcaaaaaatgtaaacgctctcgttaa

SEQ ID NO: 512 - protein, 288 aa, Glycine max; identical to SEQ ID NO: 98
  REGION 1..288: note = GmBBX43 soybean B-box polypeptide

SEQ ID NO: 513 - DNA, 879 bp, Glycine max
  misc_feature 1..879: note = GmBBX44
  source 1..879: mol_type = unassigned DNA
atgaagattcagtgcaacgtgtgcgaggccgcggaggccaaggttctgtgctgcgcggacgaggcagcactgtgctgggaatgcgacgagaaggttcacgcagcaaacaagctcgccagcaagcaccagagggtccctctctctctctccgcctctcacatgcccaagtgtgacatatgccaggaaatggttggttatttcttctgtttagaggatcgagctttgctatgtaggaattgtgatgtatctatacatacagcaaatgcctgtgtctctgatcatcaaaggtttttgcttactggtgtgagagtaggccttgaagctactgagcgcggtgcttcctcgtcttctgtgaagtcacagtctggggagaaaatgtctgatgctaaatcttcatccatctccagaaatgtttcctcactgccccagccatcaaatgccaatgaagtgttacccctccaaatgcaaggagttgaggagtttccaccaagcaatttctcaggttatacttctggaaacgtctcacaatggcccattgaagaatttctcggattaaatgaactcagtcagtattataattacatggatggatcatctaaggctgacagtggtaaacttggggattctgattcttctgttttgaggtctggtgaagaggatatggatgatgacggcttcttgggacgtgttccagattcatcctggacagttcctcagatcccttcccctcctacagcttcgggtttatattggccgaaagtccctcaatatacatctgacagtgccatgtctgttcctgacatatgcttttctcatgtgcgacagccccaccatgcccagcataattctaatgtttccaaaaggcgtaggcaactataa

SEQ ID NO: 514 - protein, 292 aa, Glycine max
  REGION 1..292: note = GmBBX44 soybean B-box polypeptide
MKIQCNVCEAAEAKVLCCADEAALCWECDEKVHAANKLASKHQRVPLSLSASHMPKCDICQEMVGYFFCLEDRALLCRNCDVSIHTANACVSDHQRFLLTGVRVGLEATERGASSSSVKSQSGEKMSDAKSSSISRNVSSLPQPSNANEVLPLQMQGVEEFPPSNFSGYTSGNVSQWPIEEFLGLNELSQYYNYMDGSSKADSGKLGDSDSSVLRSGEEDMDDDGFLGRVPDSSWTVPQIPSPPTASGLYWPKVPQYTSDSAMSVPDICFSHVRQPHHAQHNSNVSKRRRQL

SEQ ID NO: 515 - DNA, 1125 bp, Glycine max
  misc_feature 1..1125: note = GmBBX45
  source 1..1125: mol_type = unassigned DNA
atgaacctcgtggccgcaaaaaatatccttcatttttttcttctttctcttctccgccacttcttaaaccctgctcctctgcttcgtggtgccccaaacctcttcttctctttcttttccctctctcaattccaaacagtagatttagacacccttttggaaaaggaagtgttttttgagggaaaggagggaattttttgtgtgggagggtggaacccggaattcggtttggttggggtagagaggatgaagattcagtgtaacgtgtgtgaggctgcggaggccaaggttctttgttgtgctgatgaggctgggctgtgttgggagtgtgatgagaaggttcatgcggctaacaagcttgccagcaagcaccagagagtgcctctttctacctcttcctctcatatgcctaaatgtgacatttgtcaggaagcattaggctattttttctgtctagaagatcgggctctgctctgcagaaaatgtgatgtggcaatacacactgcaaatgcttatgtctctggtcatcagaggtttcttctaactggtgtaagagtaggccttgaagctacagaccctggtgcttcctcaacttctttgaagtcagattctggggagaaagtttcagattcttctgtctctagaaaggtttccacagcgccccagccatcaaattataatgaagtgttacccgctgaagttggaggagttggggagtttccatcagccaaggtgtcctttggtggtggttccacagctggaaatatctcacagtggactattgatgaatttattggcttaaacgagttcagtcagaattatgattacatggaaggatcatcaagggctgacggtggtaagcttggggattctgattctcctgttttaagatctggtgaagaggaaatggaggaggaggatgactatttggaacgcgttccagattcgtcgtggacagtcccgcagatcccttccccacctacttcttctgggttatgctggccaaaagaccatactaatctgtatggttcatctgatagtgttctgtttgttcctgacataagcttctctcacctgcaacaatctcagattagtatttttttgagacgccggaggcagctttaa

SEQ ID NO: 516 - protein, 374 aa, Glycine max
  REGION 1..374: note = GmBBX45 soybean B-box polypeptide
MNLVAAKNILHFFLLSLLRHFLNPAPLLRGAPNLFFSFFSLSQFQTVDLDTLLEKEVFFEGKEGIFCVGGWNPEFGLVGVERMKIQCNVCEAAEAKVLCCADEAGLCWECDEKVHAANKLASKHQRVPLSTSSSHMPKCDICQEALGYFFCLEDRALLCRKCDVAIHTANAYVSGHQRFLLTGVRVGLEATDPGASSTSLKSDSGEKVSDSSVSRKVSTAPQPSNYNEVLPAEVGGVGEFPSAKVSFGGGSTAGNISQWTIDEFIGLNEFSQNYDYMEGSSRADGGKLGDSDSPVLRSGEEEMEEEDDYLERVPDSSWTVPQIPSPPTSSGLCWPKDHTNLYGSSDSVLFVPDISFSHLQQSQISIFLRRRRQL

SEQ ID NO: 517 - DNA, 717 bp, Glycine max
  misc_feature 1..717: note = GmBBX46
  source 1..717: mol_type = unassigned DNA
atgaaaattcagtgcgatgtgtgtgagaaagccccggcaaccgtgatttgctgcgcagatgaggcagctttgtgtgccaaatgtgacgttgaagttcatgctgcaaacaagcttgcaagcaagcaccagaggcttctccttcaatctttatctaacaagcttcccagatgtgacatatgtcaagataagccagctttcatattttgtgttgaggacagagcactcttctgtaaagactgtgatgaacctattcatttagccagtagcctttctgcaaaccaccagcgcttccttgctactggtatccgggtggctttgggttctaattgcaccaaaggcaatgaaaaaggtcacgtggaaccatctaaaccaaaagcacaagaagttcctgcgaaaattccttctcagcaagtgcctagcttcacatcctcttgggcagttgatgacttgttggaattaacagactttgaatcaccagacaaagttcagaagcaatcccttgagtttggagaacttgaatggctagcagatgtaggcctttttggtgaacagtttcctcatgaagctttagcggcggctgaagttcctcagcttccaatgactagcagtgttggctcacacaaagcccccaaatccttgttgtcttacaaaaagcctaggattgaagtcctagatgaagatgatgatgagcacttcaccgtaccagatctcggataa

SEQ ID NO: 518 - protein, 238 aa, Glycine max; identical to SEQ ID NO: 188
  REGION 1..238: note = GmBBX46 soybean B-box polypeptide

SEQ ID NO: 519 - DNA, 717 bp, Glycine max
  misc_feature 1..717: note = GmBBX47
  source 1..717: mol_type = unassigned DNA
atgaagattcagtgtgatgtgtgtgagaaagcccctgcaaccgtgatttgttgcgcagatgaggcagctttgtgtgccaaatgtgacgttgaagttcatgctgcaaacaagcttgcaagcaagcaccagaggcttctccttcaatgtctatcaaacaagcttcccagatgtgacatatgccaagataagccagctttcatattttgtgttgaggacagagcactcttctgtaaagactgtgatgagccaattcatttagccagtagcctttctgcaaaccaccagcgcttcctcgctactggtatcagggtggctttgggttctaattgcaccaaaggccatgaaaaaggtaacatggaaccatctaatccaaatgcacaagaagttcctgtgaaaactccttctcagcaagtgcctagcttcacatcctcttgggcagttgatgacttgttggaattaacagactttgaatcgccagacaaaaagcaatccctggaatttggagaacttgaatggctagcagatgcaggccttttcagtgaacagtttcctcatgaagctttagtcgcagctgaagttcctcagcttccagtgccacacactagcagtgttgcctcacataaagcccccaaatccttggtgtcttacaaaaagcctaggattgaagtcctggatgaagatgatgatgaacactgcactgtcccggatctcggataa

SEQ ID NO: 520 - protein, 92 aa, Glycine max
  REGION 1..92: note = GmBBX47 soybean B-box polypeptide
MKIQCDVCEKAPATVICCADEAALCAKCDVEVHAANKLASKHQRLLLQCLSNKLPRCDICQDKPAFIFCVEDRALFCKDCDEPIHLASSLSA

SEQ ID NO: 521 - DNA, 720 bp, Glycine max
  misc_feature 1..720: note = GmBBX48
  source 1..720: mol_type = unassigned DNA
atgaaaattcagtgtgatgtgtgtgagagagctccggcaacagtgatttgttgtgcggatgaggcagctttgtgtgccaaatgtgacgttgaagttcatgctgcaaacaagcttgcaagcaagcaccaaaggcttcttcttcaatgtctatcaagcaagcttccaagatgtgacatttgccaagacaagccagcattcatattttgtgttgaagacagagcactcttctgtcaggattgtgatgaaccaattcattcagccggaagcctttctgcgaaccaccagcgcttccttgctactggtatccgagtggcatcgagttctaattgcaccaaggacaatgaaaaaagtcacttggagcctcctactaggaatgcacaacaagtttctgcgaaaattcctcctcagcaagtgcctagcttcacttcctcttgggctgttgatgacttattggaactaacagcctttgaatcacctgagaaaaaggaatctctgcagtttggagagctggaatggcttacagatgtgggtatttttggtgaacaatttgctcaggaagccttggctgcagctgaagtacctcagcttccagtaactcataacagtagcagtgttgcttcatacagaacttccaaatcctacatgtcccacaaaaagcctaggattgaagtcctaaatgatgatgatgatgagtattttactgtgcctgatcttggctaa

SEQ ID NO: 522 - protein, 239 aa, Glycine max; identical to SEQ ID NO: 190
  REGION 1..239: note = GmBBX48 soybean B-box polypeptide

SEQ ID NO: 523 - DNA, 723 bp, Glycine max
  misc_feature 1..723: note = GmBBX49
  source 1..723: mol_type = unassigned DNA
atgaaaattcagtgtgacgtgtgtgagagagctccagcaacagtgatttgttgcgcggatgaggctgctttgtgtgccaaatgtgacgttgaagttcatgctgcaaacaagcttgcaagcaagcaccaaagacttcttcttcaatgtctatcaagcaagcttcccacatgtgacatttgccaagacaagccagcattcatattttgtgttgaagacagagcactcttctgtcaggattgtgatgaaccaattcattcagccggaagcctttccgcgaaccaccagcgcttccttgctactggtatccgagtggcctcgagttctaattgcaccaaagacaatgaaaaaagtcactcggagcctcctaataggagtgcacaacaagtttctgcgaaaattcctcctcagcaagtgcctagcttcacatcctcttgggctgttgatgacttattggaactaacaggctttgaatcacctgagaaaaaggaatctctgcagtttggagagctggaatggcttacagatgtgggtattttcggcgaacaatttgctcaggaagccttggctgcagctgaagtacctcagcttcccgaaactcataacagtagcagtgttgcttcatacaaaacctccaaatcctacatgtcccacaaaaagcctaggattgaagtcctaaatgatgatgatgatgatgagtattttactgtgcccgatctcggctaa

SEQ ID NO: 524 - protein, 92 aa, Glycine max
  REGION 1..92: note = GmBBX49 soybean B-box polypeptide
MKIQCDVCERAPATVICCADEAALCAKCDVEVHAANKLASKHQRLLLQCLSSKLPTCDICQDKPAFIFCVEDRALFCQDCDEPIHSAGSLSA

SEQ ID NO: 525 - DNA, 1317 bp, Glycine max
  misc_feature 1..1317: note = GmBBX50
  source 1..1317: mol_type = unassigned DNA
atggagaaggtttgtgaattctgcacagctttgaggccacttgtttactgtaaggctgatgcagcatatctttgtctttcctgtgatgcaaaggtgcatttagcaaatgcagtgtccggccggcacctccggaacctcgtgtgtaactcatgcggataccaccttgcttatgttctgtgtttggaacacaaaatgttgatatgtcgtgactgtgaccaaaagttgcacaacatttctttgccacataagaaacgagctatcagaagtttcatgggttgcccttctgctaaagactttgcagcactctggggggttgaattgaatgagattgaaaacagtgccagtcaggatcagtttgattcagtttcttgtgtttctgcagatttaaatgttgcacaagtttcaggaaagcctggcattcaaactgaggtcccttcaatgctatctggggccaaacttgatgggggaggatcaaccagtcaacaaggtcagatattgtatagtgatcaaaagcgtcaaacgattctgcagcagattattgattttaaatggcttcaacaaaatgaggagattgattattctgcaaagatcaataggctacaagaaaaagattcatctccttcagtatatcagactcttaagaaattggacgacaaattcaatgagcaagcacaaaattcccaagatcttgctactaatgttctggaaaaagactgtcccattatggagctgaatcctgaaactttgccttctacgttttctcaacttgacaatttatattcttcttcaactatcgatcttcccttgcatggagaattgttctggacatgcaaaagtccacttcaaagtaatcagttgtggtcccaaaatattcaagaccttgggatatgcaaagaacttgtttgtcaagatgattttaacatccctgatgttgacttaactttccaaaactatgaggaactatttggaggagatcaagatccaattagaatactgcttgatgatcaagatgtctcctgctcttctttagagaaggataagtcagttgacaagtcagttattgacaatccaagtgcaatggaggaatcttctgcagctgcgtccattactatctcccaatctgatcttgacaacaaggatatgaatcctctcagccagtactgtccaagaagcatggatcctgctcatgcaattcgaccatttgattcaactataccattctctgttctgagattcaatgaggaaagcagttgcacaaaccatgatgacagtgccctttctccctacagtgaagggagggcttattcaggtagttaa

SEQ ID NO: 526 - protein, 172 aa, Glycine max
  REGION 1..172: note = GmBBX50 soybean B-box polypeptide
MEKVCEFCTALRPLVYCKADAAYLCLSCDAKVHLANAVSGRHLRNLVCNSCGYHLAYVLCLEHKMLICRDCDQKLHNISLPHKKRAIRSFMGCPSAKDFAALWGVELNEIENSASQDQFDSVSCVSADLNVAQVSGKPGIQTEVPSMLSGAKLDGGGSTSQQGQILYSDQKR

SEQ ID NO: 527 - DNA, 1317 bp, Glycine max
  misc_feature 1..1317: note = GmBBX51
  source 1..1317: mol_type = unassigned DNA
atggagaaggtttgtgaattctgcacagctttgaggccacttgtttactgtaaggctgatgcagcatatctttgtctatcctgtgattcaaaggtgcatttagcaaatgcagtgtctggtcggcacctccggaactttgtgtgccactcatgcggataccatcttgcttatgttctgtgtttggaacataaaatgttgatctgtcgtgactgtgatcaaaagttgcataacatttctttaccacataagaaacgagctatcagaagtttcatgggctgcccttctgctaaagactttgcagcactctgggggtttgaatcgaatgagattgaaaacagtgccactcaggatcagtttggttcagtttcttgtgtttctgcagatttgaatgttgcacaagtttcaggaaagcctggcattcaaactgggatcccctcaatgccatctggagccaaatttgatgggggaggatcaaccagtcaacaaggtcagatattgtacagtgatcaagagcgacaaactattctgcagcagattattgatttaaaaatgcttcaacaaaatgaggacattgattatcttgcaaagatcaataggctacaagaaaaagatttatctccttcagtatatcacactcttaagaaattggacgacaaattcaatgagcaagcacaaaagtcccaggatcttgctactaatgttctggaaaaagactgtcccactgtggagctgaatcctgaaactttgccttctacgttttctcaatttgacaatttatattcttcttcaattatcgatcttcccttgcatggagagttgttctggacatgccgaagtccacttcaaagtaatcagttgtggtcccaaaatattcaagaccttgggatatgcaaagaacttgtttgtcaagacgattttaacatccctgatgttgacttaactttccaaaactttgaggaactatttggaggagatcaagattcaattagaatactgcttgatgatcaagatgtctcctgctcttctttagagaaggataagtcagttggcaagtcagatattgacaatccaagtgcaatggaggaatcttctgcagctgcgtctattactatctcccaatctgatcttgacaataaggatatgaatcctctcagccagtaccgtccaagaagcatggatcccgcccttgcaattcgaccatttgattcaaccataccattctccgttttgagattcaatgaggaaagcagttgcactagccatgatgacagtgccctttctccctacagtgaaaggaaggcttattcaggtagttaa

SEQ ID NO: 528 - protein, 438 aa, Glycine max
  REGION 1..438: note = GmBBX51 soybean B-box polypeptide
MEKVCEFCTALRPLVYCKADAAYLCLSCDSKVHLANAVSGRHLRNFVCHSCGYHLAYVLCLEHKMLICRDCDQKLHNISLPHKKRAIRSFMGCPSAKDFAALWGFESNEIENSATQDQFGSVSCVSADLNVAQVSGKPGIQTGIPSMPSGAKFDGGGSTSQQGQILYSDQERQTILQQIIDLKMLQQNEDIDYLAKINRLQEKDLSPSVYHTLKKLDDKFNEQAQKSQDLATNVLEKDCPTVELNPETLPSTFSQFDNLYSSSIIDLPLHGELFWTCRSPLQSNQLWSQNIQDLGICKELVCQDDFNIPDVDLTFQNFEELFGGDQDSIRILLDDQDVSCSSLEKDKSVGKSDIDNPSAMEESSAAASITISQSDLDNKDMNPLSQYRPRSMDPALAIRPFDSTIPFSVLRFNEESSCTSHDDSALSPYSERKAYSGS

SEQ ID NO: 529 - DNA, 726 bp, Glycine max
  misc_feature 1..726: note = GmBBX52
  source 1..726: mol_type = unassigned DNA
atgaagggtaagacttgcgagctttgtgatcaacaagcttctctctattgtccctccgattccgcatttctctgctccgactgcgacgccgccgtgcacgccgccaactttctcgtagctcgtcacctccgccgcctcctctgctccaaatgcaaccgtttcgccggatttcacatctcctccggcgctatatcccgccacctctcgtccacctgcagctcttgctccccggagaatccttccgctgactactccgattctctcccttcctcttctacctgcgtctccagttccgagtcttgctccacgaagcagattaaggtggagaagaagaggagttggtcgggttcctccgtgaccgacgacgcatctccggcggcgaagaagcggcagaggagtggaggatcggaggaggtgtttgagaaatggagcagagagatagggttagggttagggttaggggtaaacggaaatcgcgtggcgtcgaacgctctgagtgtgtgcctgggaaagtggaggtggcttccgttcagggtggctgctgcgacgtcgttttggttggggctgagattttgtggggacagagggctggcctcgtgtcagaatctggcgaggttggaggcaatatccggagtgccagttaagctgattctggccgcacatggcgacctggcacgtgtcttcacgcaccgccgcgaattgcaggaaggatggggcgagtcctag

SEQ ID NO: 530 - protein, 241 aa, Glycine max
  REGION 1..241: note = GmBBX52 soybean B-box polypeptide
MKGKTCELCDQQASLYCPSDSAFLCSDCDAAVHAANFLVARHLRRLLCSKCNRFAGFHISSGAISRHLSSTCSSCSPENPSADYSDSLPSSSTCVSSSESCSTKQIKVEKKRSWSGSSVTDDASPAAKKRQRSGGSEEVFEKWSREIGLGLGLGVNGNRVASNALSVCLGKWRWLPFRVAAATSFWLGLRFCGDRGLASCQNLARLEAISGVPVKLILAAHGDLARVFTHRRELQEGWGES

SEQ ID NO: 531 - DNA, 732 bp, Glycine max; identical to SEQ ID NO: 431
  misc_feature 1..732: note = GmBBX53
  source 1..732: mol_type = unassigned DNA

SEQ ID NO: 532 - protein, 243 aa, Glycine max; identical to SEQ ID NO: 432
  REGION 1..243: note = GmBBX53 soybean B-box polypeptide

SEQ ID NO: 533 - DNA, 375 bp, Glycine max
  misc_feature 1..375: note = GmBBX54
  source 1..375: mol_type = unassigned DNA
atgtgcaaaggtgcagaaggagaaaagcaacatggtttctgcagcagctttctacataaagagagtgcaaccagaagtgctatatgttgtgagctatgtgggttacaggcttcattgtattgccaagctgatgatgcctacttgtgtaaaaaatgcgataaacgggttcatgaagctaattttttggcccttaggcacattaggtgctttctgtgcaacacatgtcagaaccttacgcgaagatatctcattggagcatcactagaggtggttcttccggccaatataaactggaccattagaaatctccttaacaacagaggaatctacagaaagtgttcaagaatgcataacagcccttccctctttttataa

SEQ ID NO: 534 - protein, 93 aa, Glycine max
  REGION 1..93: note = GmBBX54 soybean B-box polypeptide
MCKGAEGEKQHGFCSSFLHKESATRSAICCELCGLQASLYCQADDAYLCKKCDKRVHEANFLALRHIRCFLCNTCQNLTRRYLIGASLEVVLP

SEQ ID NO: 535 - DNA, 375 bp, Glycine max
  misc_feature 1..375: note = GmBBX55
  source 1..375: mol_type = unassigned DNA
atgtgcaaaggtgcagaaggagaaaagcaacatggtttctgcagcagctatctacataaagaatgtgcaaccagaagtgctacatgttgtgagctatgtgggttacaggcttcattgtattgtcaagctgatgatgcatacttgtgtagaaaatgtgataaaagggttcatgaagctaattttttggcccttaggcacattaggtgctttctgtgcaacacatgtcaaaaccttacgcgaagatatctcattggagcatcaatagaggtggttcttccagccaacattaactggaccattggaaatctccctagcaacagaggaatccacagaaagtgctcaagaatgcataacaacctttccctcttgttataa

SEQ ID NO: 536 - protein, 92 aa, Glycine max
  REGION 1..92: note = GmBBX55 soybean B-box polypeptide
MCKGAEGEKQHGFCSSYLHKECATRSATCCELCGLQASLYCQADDAYLCRKCDKRVHEANFLALRHIRCFLCNTCQNLTRRYLIGASIEVVL

SEQ ID NO: 537 - DNA, 651 bp, Glycine max
  misc_feature 1..651: note = GmBBX56
  source 1..651: mol_type = unassigned DNA
atggaggaatcatcaacaacatgcgtgttgtgcgagaagagggcaatgatgctctgcgactcggaccaggctaagctatgctgggaatgcgacgagaaagtccacagcgccaatttcttggtcgcgaaacattctagggttcttttatgtcgtttgtgtcactccccgactccgtggaaggcttcggggatgaaactcacgcccactgtgtcgttttgtaaccgctgcgttgcggaacggaacgcgaggtggaaccgattggtgaataatgaaaatgaacatcaacaacagcaacaacaacaacaacagagtgattttgtggtggatgatggtagggaatatggttctgatcatgtttttgatgatgacgatggtgattatagtgatgatagtggtgaagaagaagaagaggacgaggatgatgaagaggagaatgagaatcaagtggttccaatgtcttctggttctgccacgtcaccacctaaggttgcctgtttagcgttgaagcgattgagaaacaactctttttctgtcgattcttcacatgatgagacagcgtgctcttcatctgagatgcttagttctacattgcctagtggtgatgaatcaacctctttgagtagtgtattaaagaggcatcgggttgattag

SEQ ID NO: 538 - protein, 216 aa, Glycine max; identical to SEQ ID NO: 22
  REGION 1..216: note = GmBBX56 soybean B-box polypeptide

SEQ ID NO: 539 - DNA, 465 bp, Glycine max
  misc_feature 1..465: note = GmBBX57
  source 1..465: mol_type = unassigned DNA
atgaggaagtgtgagctctgtaacagtcccgcgaagttgttctgcgaatcagatcaagccagcctctgttggaaatgcgatgctaaggttcacagtgcaaacttcctcgtcaccaaacatcccaggattcttctctgccatgtttgtcaatcactaacagcgtggcacggcacaggacccaagtttgtacccaccatgtcagtttgcaacacttgtgtcaacaacaatagtactgagacctgcagccaacagaatcatgaagacgatgatgatgatggtacgggagaggatcatgcagaaaacgacgatggtggtgtggctgaagatgatgatgatgatgatgatgaagaaaatcaagtggttccatggacatctacaccaccacccccagcttccacttcttcaaatagtgttacaactagttctaccaggttctctgatgttgaagaaggtggctccgattaa

SEQ ID NO: 540 - protein, 88 aa, Glycine max
  REGION 1..88: note = GmBBX57 soybean B-box polypeptide
MRKCELCNSPAKLFCESDQASLCWKCDAKVHSANFLVTKHPRILLCHVCQSLTAWHGTGPKFVPTMSVCNTCVNNNSTETCSQQNHED

SEQ ID NO: 541 - DNA, 459 bp, Glycine max
  misc_feature 1..459: note = GmBBX58
  source 1..459: mol_type = unassigned DNA
atgaggaagtgtgaactctgtaatagtcccgcaaagttgttctgtgaatcagatcaagccagcctctgttgggaatgcgatgctaaggttcacagtgcaaacttcctcgtcaccaaacatcccaggattcttctctgccatgtttgtcaatcactaacggcctggcacggcacgggacccaagtttgtacccaccatgtccgtttgcaacacttgtgtcaacaacaatagtgagacctgcagccaacagaatcatgttgaggaccacgacgacaatcatgatgatggcacgggagaggatcatgtagaaaaccatgatggtggtgtgtctgaagatgatgaagaaaatcaagttgttccatggacatctacaccaccacccccagcttccacttcttcaaatagtgttacaactagttctaccaggttctctaatgttgaagaaggtggctccgattaa

SEQ ID NO: 542 - protein, 152 aa, Glycine max; identical to SEQ ID NO: 38
  REGION 1..152: note = GmBBX58 soybean B-box polypeptide

SEQ ID NO: 543 - DNA, 534 bp, Glycine max
  misc_feature 1..534: note = GmBBX59
  source 1..534: mol_type = unassigned DNA
atgaacaagtgcgaactgtgcaaagttccggcgaggatcttctgcgagtcggaccaggcgagcttatgctgggactgcgacgctaaggttcacagcgccaacttcttggtcgcgagacacgcgcgaaccttactctgccgcacgtgccgctctcccacgccgtggaaggcctccggcgcctcactcgccagcgccgtctccgtctgcgaccgctgcgccgcctccgaagaaccggaagagagtgaagccggcaatgacgacgaaccagacacggaggacgacgacaacgacgacgaggacggcgacaatcaggtcgtgccgtggtcctcctccccgccgccgcctccgccgtcgagctcctccggcagcgaatggtcggagtgcaaaaacgacgacgcttctatttccgcctcggaacaggaaacgacaacatgctcgttgaaacgacagcgcgaggacgatgatgatgcttcttccgatcgtgatcccagggtgttgagaagggttgggcgtctcaacggaggttgcgtgtga

SEQ ID NO: 544 - protein, 177 aa, Glycine max; identical to SEQ ID NO: 24
  REGION 1..177: note = GmBBX59 soybean B-box polypeptide

SEQ ID NO: 545 - DNA, 522 bp, Glycine max
  misc_feature 1..522: note = GmBBX60
  source 1..522: mol_type = unassigned DNA
atgaagaagtgcgaactgtgtaaagttccggcgaggatcttctgcgagtcggaccaggctagcttatgctgggactgcgacgctaaggttcacagcgccaacttcttggtcgcgaggcacgtgcgaaccctactctgccgcacgtgtcgttctcccacgccctggaaggcctccggcgcctcactcgcccacgccgcctccgtctgcgaccgctgcgccgccgcggaagagacggaagagagcgaagccggcaatgacgacgaaccagacaccgaggacgacgacgatgtcgacaaccaggtcgtgccgtggtcctccacgccgccgccgccgccgccgtcgagctcctccggcagcgaatgctcggagtgcaaaaacaacgttgcttctttttccgcctcggaacaggaaacgacaacatcctcgttgaaacgacagcgcgaggacgacgacgatgcttcttccgatcctgatcccagggtgttgagaagggttgggcgtctcaacggaggttgcgtgtga

SEQ ID NO: 546 - protein, 89 aa, Glycine max
  REGION 1..89: note = GmBBX60 soybean B-box polypeptide
MKKCELCKVPARIFCESDQASLCWDCDAKVHSANFLVARHVRTLLCRTCRSPTPWKASGASLAHAASVCDRCAAAEETEESEAGNDDEP

SEQ ID NO: 547 - DNA, 666 bp, Glycine max
  misc_feature 1..666: note = GmBBX61
  source 1..666: mol_type = unassigned DNA
atgaagaactgcgagctatgcaagcttccagctaggaccttctgcgagtcggaccaggctagcttatgctgggactgcgacgccaaggtccacggagcgaacttcctcgtcgcgagacacacgcgcacgctcctctgccacgcgtgccagtcactcacgccgtggaaggcctccggctctgccctcggcaacaccgtctcgctgtgccagagttgcgccggcggaaccaccgaacagggcccagagagccaaggagggaatgacgacgacgacatagacaccgacgatgaggatgactacgatgacgatggtgagagcgaggagaacgaggttgcagccgacgaggaggacggagacaaccaggttgtcccttggtcctccgagccgccgccaccgtccccaagctcttccagcagcgaagagtcgattagccggtgcaacaacgtggacgaggtttcgaccacattgaaacgacgtcgcgagaacgacgattttcagggttcgaattcgaataattggaaatgtgaacggagcgaagtggacagaggatgttggttggctcggttgcggcggagaaccgccgatgatgtggcggttgagcgacggagggctagagcggcgtttccgtacggttgttgtgatgatagagcatctgaagacgtttga

SEQ ID NO: 548 - protein, 221 aa, Glycine max; identical to SEQ ID NO: 32
  REGION 1..221: note = GmBBX61 soybean B-box polypeptide

SEQ ID NO: 549 - DNA, 666 bp, Glycine max
  misc_feature 1..666: note = GmBBX62
  source 1..666: mol_type = unassigned DNA
atgaagaactgcgagttgtgcaagcttccggctcggactttctgcgagtcggaccaggctagcttatgctgggactgcgacgccaaggttcatggagccaacttcctcgtggaaagacacacgcgcacgctcctctgccacgcgtgccagtcgcccacgccgtggaaggcctccggcgccgccctcggaaacaccgtctcgctgtgcgagagatgcgccggcggaaccaccgaacaaggtcaagagagccaaggaggcaatgacgacgacatagacaccgacatagacaccgacgatgaggatgactacgatgagagcgaggacgaggttgccgccgacgaggaggacggagacaaccaggttgtcccttggtcctccgagccgccgccaccagccccgagctcttccagcagcgaagaatcggttagccggtgcaacaacgtggacgaggtttcgaccacattgaaacgccgtcgccaggaggacaatgattttcagggttggaattcgaataattggggatgtgaacggagcgaagtggagagaggaggttggttggttcggttgcggcggagaaccgccgatgatgtggcggttgagcaacggagtgctagagcggcgtctccagacggttgctgtggtgatagagcatctgaagacgtttga

SEQ ID NO: 550 - protein, 88 aa, Glycine max
  REGION 1..88: note = GmBBX62 soybean B-box polypeptide
MKNCELCKLPARTFCESDQASLCWDCDAKVHGANFLVERHTRTLLCHACQSPTPWKASGAALGNTVSLCERCAGGTTEQGQESQGGND

(19) United States
(12) Patent Application Publication
(10) Pub. No.: US 2024/0124885 A1
(43) Pub. Date: Apr. 18, 2024

(54) MANIPULATING PLANT SENSITIVITY TO LIGHT

(71) Applicants: Mendel Biotechnology, Inc., Hayward, CA (US); Monsanto Technology, LLC, St. Louis, MO (US)

(72) Inventors: Rajnish Khanna, Livermore, CA (US); Brent A. Kronmiller, Corvallis, OR (US); Oliver J. Ratcliffe, Hayward, CA (US); T. Lynne Reuber, San Mateo, CA (US); Marie E. Petracek, Glendale, CA (US); Robert J. Meister, St. Peters, MO (US); Sasha Preuss, Webster Groves, MO (US); Amanda McClerren, St. Charles, MO (US); Qungang Qi, Chesterfield, MO (US)

(21) Appl. No.: 18/465,595

(22) Filed: Sep. 12, 2023

Related U.S. Application Data
(63) Continuation of application No. 17/404,842, filed on Aug. 17, 2021, now abandoned.
Application No. 17/404,842 is a continuation of application No. 15/802,397, filed on Nov. 2, 2017, now abandoned.
Application No. 15/802,397 is a division of application No. 14/118,491, filed on Jun. 20, 2014, now abandoned.
(60) Provisional application No. 61/488,592, filed on May 20, 2011.

(51) Int. Cl. C12N 15/82; C07K 14/415

(52) U.S. Cl. CPC C12N 15/8261; C07K 14/415; C12N 15/8223; C12N 15/8225; C12N 15/8229; C12N 15/8241; C12N 15/8243; C12N 15/8245; C12N 15/8246; C12N 15/8271; C12N 15/8273; Y02A 40/146

(57) ABSTRACT

The present disclosure identifies new genes which have the potential to increase broad acre yield in crops. This disclosure is based upon our fundamental knowledge of light signal transduction and our understanding of the roles these genes play in regulating plant growth and development in response to light. Transgenic plants with gain- or loss-of-function of one of these genes, or in combination, are expected to show significant improvements in broad acre yield and stress tolerance.

### JOINT RESEARCH AGREEMENT

The claimed invention, in the field of functional genomics and characterization of plant genes for the improvement of plants, was made by or on behalf of Mendel Biotechnology, Inc. and Monsanto Company as a result of activities undertaken within the scope of a joint research agreement in effect on or before the date the claimed invention was made.

### CROSS REFERENCE TO RELATED APPLICATION

This application is a continuation of U.S. patent application Ser. No. 17/404,842, filed Aug. 17, 2021, which is a continuation of U.S. application Ser. No. 15/802,397, filed Nov. 2, 2017, (abandoned), which application is a divisional of U.S. application Ser. No. 14/118,491, filed Jun. 20, 2014 (abandoned), which application incorporates by reference in its entirety, and is a National Phase of International Patent Application No. PCT/US2012/038719, filed on May 18, 2012 (expired), which claims the benefit of priority to U.S. Provisional Application Ser. No. 61/488,592 (filed on May 20, 2011), each of which is incorporated by reference herein in its entirety.

### RELEVANT FIELD

The present disclosure relates to plant genomics and plant improvement.

### BACKGROUND

A plant's traits, including its biochemical, developmental, or phenotypic characteristics that enhance yield or tolerance to various abiotic stresses, may be controlled through a number of cellular processes. One important way to manipulate that control is through transcriptional regulators-proteins that influence the expression of a particular gene or sets of genes. Transgenic plants that comprise cells having altered levels of at least one selected transcriptional regulator, for example, possess advantageous or desirable traits. Strategies for manipulating traits by altering a plant cell's transcriptional regulator content can therefore result in plants and crops with commercially valuable properties.

### SUMMARY

The present disclosure identifies B-box genes that modify plant sensitivity to light. Manipulating light signaling processes through altering the expression/activities of these genes in plants will lead to enhanced agronomic characteristics, for example, increased crop yield and improved stress tolerance. Altering the expression/activity of the B-box genes of the present disclosure may also provide plants with altered levels of ureides, altered levels of hexose sugars, altered sucrose phosphate synthase (SPS) activity, altered levels of starch, and delayed senescence.

The present disclosure pertains to polynucleotide and polypeptide sequences provided herein and in the Sequence Listing. The present disclosure further pertains to nucleic acid constructs containing and/or expressing or suppressing the polynucleotide sequences provided herein and in the Sequence Listing, individually or in combination, either driven by a ubiquitously expressed, a stress- or a chemical-inducible, a tissue specific, a development specific or a diurnally-regulated promoter.

The present disclosure also pertains to transgenic plants with increased or decreased activities of one or more of the proteins provided herein and in the Sequence Listing, or encoded by the nucleic acid constructs containing and/or expressing or suppressing the polynucleotide sequences provided herein and in the Sequence Listing. The transgenic plants may exhibit changes in light sensitivity, resulting in improved vigor, improved yield and growth, and improved ability to cope with abiotic stresses compared to the wild type or other control plants. This approach can be combined with other growth-promoting factors to expand the benefits.

The present disclosure also provides methods for the selection and production of transgenic plants transformed with the disclosed B-box sequences or closely-related orthologs or paralogs. The selection may be made by identification in a plant (that is, a mature plant, a seed, a plant part, a seedling, plant tissue, plant cell, etc.) of the presence of a transgene or heterologous sequence that was transformed into the transgenic plant, or by an altered trait in the transgenic plant such as improved yield or increased tolerance to an abiotic stress, such as low nitrogen conditions, cold, water deprivation or drought, etc.

The present disclosure is also directed to transgenic seed produced by any of the transgenic plants of the present disclosure, including transgenic plants ectopically expressing or suppressing SEQ ID NO: 2N, where N=1-117, or 218-275, or ectopically expressing a polypeptide that regulates transcription and the polypeptide comprises a conserved domain of any of SEQ ID NO: 235-427, and to methods for selecting and/or making the disclosed transgenic plants and transgenic seeds.

### BRIEF DESCRIPTION OF THE SEQUENCE LISTING AND DRAWINGS

The Sequence Listing provides exemplary polynucleotide and polypeptide sequences. The traits associated with the use of the sequences are included in the Examples.

Incorporation of the Sequence Listing. The file named “MDBT021USD1C2_ST26.xml” contains a computer readable form of the Sequence Listing and was created on Sep. 1, 2023. The file is 900,669 bytes (measured in MS-Windows®), was filed contemporaneously along with this application by electronic submission and is incorporated herein by reference in its entirety.

FIG. 1 shows light-responsiveness of Arabidopsis seedlings transformed with 35S::AtBBX28 (G1481):cMYC (AtBBX28 is provided as SEQ ID NO: 2). Several independently transformed lines were grown in red light and mean hypocotyl lengths of 20-30 seedlings of each line are plotted with the empty-vector control (pMEN 65) and seedlings overexpressing BBX32 (G1988 OE; SEQ ID NO: 430). The dotted line marks the hypocotyl length of the control (pMEN 65; SEQ ID NO: 428).

FIG. 2 shows AtBBX28 (G1481, SEQ ID NO: 2) suppressing transcriptional activity of AtBBX21 (G1482, SEQ ID NO: 56) in protoplast co-transfection assays. Protoplasts were co-transformed with prCHS::GUS reporter and either CAT (control) alone, or HA:AtBBX21 (G1482) with one CAT, HA: BBX32, or HA:AtBBX28 (G1481). Note that GUS is β-glucuronidase, CAT is chloramphenicol acetyltransferase, and HA is hemagglutinin protein-fusion tag (Niman et al., 1983).RGA=relative GUS activity.

In FIGS. 3 and 4, the light-responsiveness of Arabidopsis seedlings of independently transformed lines is plotted as measures of hypocotyl length for the various plant lines. The dotted line marks the hypocotyl length of the control. Vertical axes are measured hypocotyl length (HL; in millimeters). An asterisk above the bars in FIGS. 3 and 4 indicates a p-value <0.05, and a double asterisk a p-value <0.01.

FIG. 3. Arabidopsis plant lines transformed with AtBBX25 (G1894, SEQ ID NO: 154; FIG. 3A), AtBBX24 (G329, SEQ ID NO: 152; FIG. 3B), AtBBX30 (G1478, SEQ ID NO: 40; FIG. 3C) and AtBBX18 (G1881, SEQ ID NO: 202; FIG. 3D) were grown in red light and mean hypocotyl lengths (HL) of 20-30 seedlings of each line are plotted with the control.

FIG. 4. Arabidopsis plant lines transformed with AtBBX7 (G2440, SEQ ID NO: 104; FIG. 4A), AtBBX26 (G1486, SEQ ID NO: 52; FIG. 4B), AtBBX19 (G902, SEQ ID NO: 200; FIG. 4C) and

AtBBX20 (G1888, SEQ ID NO: 54; FIG. 4D) were grown in red light and mean hypocotyl lengths of 20-30 seedlings of each line are plotted with the control.

For FIGS. 5A-5E, FIGS. 6A-6D, FIG. 7, FIGS. 8A-8F, FIGS. 9A-9G and FIGS. 10A-10I, the SEQ ID NOs of the sequences appear in parentheses to the right of the sequence identifiers. In these Figures, an asterisk below any column indicates amino acid identity within the column. A colon indicates that the column is comprised of conservatively-related amino acid residues. A period indicates semi-conservatively-related amino acid residues.

FIGS. 5A-5E present an alignment of AtBBX25 (G1894, AT2G31380.1; SEQ ID NO: 154), AtBBX24 (G329, AT1G06040.1; SEQ ID NO: 152) and closely related sequences.

FIGS. 6A-6D represent an alignment of AtBBX19 (G902, AT4G38960.1; SEQ ID NO: 200), AtBBX18 (G1881, AT2G21320.1; SEQ ID NO: 202) and closely related sequences.

FIG. 7 shows an alignment of AtBBX30 (G1478, AT4G15248.1; SEQ ID NO: 40) and closely related sequences.

FIGS. 8A-8F present an alignment of AtBBX28 (G1481, AT4G27310.1; SEQ ID NO: 2) and AtBBX29 (G900, AT5G54470.1; SEQ ID NO: 4) and closely related sequences.

FIGS. 9A-9G displays an alignment of AtBBX20 (G1888, AT4G39070.1; SEQ ID NO: 54) and AtBBX21 (G1482, AT1G75540.1; SEQ ID NO: 56) and closely related sequences.

FIGS. 10A-10I show an alignment of AtBBX7 (G2440, AT3G07650.1, SEQ ID NO: 104), AtBBX8 (G1479, AT5G48250.1; SEQ ID NO: 106) and closely related sequences.

FIG. 11 shows microarray data from field grown soybean plants. Microarrays performed on tissues sampled throughout the day from the field demonstrated that 219 genes showed 2-fold or greater changes in abundance in transgenic events transformed with At. BBX32 relative to the control and that the majority of these changes occurred around dawn.

FIGS. 12A-12E shows a phylogenetic tree of the Arabidopsis thaliana and Glycine max B-box family, which contains 61 B-box sequences from soybean and 32 from Arabidopsis. Phylogenetic analysis of the B-box sequences shows that the sequences are clustered into structural groups(St) and clades (C).H refers to Arabidopsis homolog, N refers to other names.

FIGS. 13A-13D shows a phylogenetic tree for 61 B-box sequences from soybean. Phylogenetic analysis of the B-box sequences shows that these sequences are clustered into structural groups (St) and clades (C).A Y (yes) under I indicates a putative interactor of AtBBX32 (I (At)) or GmBBX52 (I (Gm)) based on Y2H analyses; T represents tissue of expression where L, 1=leaf; C, c=cotyledon; A, a=all tissues; S, s=seed; F, f=flowers; N, n=nodules; P, p=pod; R, r=root; NR=not represented in TxP, where bold upper-case letters represent high expression; upper-case letters represent moderate expression, and lower-case letters represent average expression. TP refers to time of peak expression. SP refers to whether or not a shift in peak expression was observed in transgenic soybean plants expressing BBX32 (Y for yes and N for no).CD refers to at least 1.5 fold(except where *˜1.2-1.4 fold) change in expression at dawn in transgenic soybean plants expressing BBX32, where I=induced; S=suppressed. The first and second circles represent the first and the second B-box domain respectively; the rectangle represents the CCT domain.

FIG. 14 represents the single B-box Glade in Arabidopsis thaliana and Glycine max. The Arabidopsis thaliana genome contains seven single B-box domain genes while the paleopolyploid Glycine max genome contains thirteen single B-box genes. Phylogenetic analysis indicates that GmBBX52 and GmBBX53 are orthologs of the Arabidopsis thaliana AtBBX32 gene.

FIG. 15 shows AtBBX32 and GmBBX62 interaction in vivo in soybean cotyledon protoplasts. Luminex co-immunoprecipitation (co-IP) was performed using antibody against GFP (A). The reciprocal co-IP was conducted using antibody against Flag tag (B). Components within the co-precipitated protein complexes were probed with biotinylated anti-Flag (A) and anti-GFP (B) antibodies, and subsequently detected with a fluorescently labeled R-phycoerythrin. MFI stands for Median Fluorescence Intensity. ** Mean differs significantly from controls at p<0.05.

FIG. 16 demonstrates that AtBBX32 interacts directly with GmBBX62 via in vitro pull-down assay. Pull-down reactions were carried out using recombinant flag-GmBBX62 and soluble AtBBX32 extracts from wheat germ in vitro translation system. Protein bound to flag-GmBBX62 was washed, separated on SDS-PAGE and immunoblotted with anti-flag antibody (A) and anti-AtBBX32 antibody (B).Lane 1, flag-GmBBX62; la, AtBBX32 wheat germ extract; 2, anti-flag antibody bound beads only; 3, AtBBX32 only, no flag-GmBBX62; 4, flag-GmBBX62+AtBBX32.

FIG. 17 shows AtBBX32 and GmBBX39 interaction in soybean protoplasts. Luminex co-IP assays were performed on extracts from soybean cotyledon protoplasts co-expressing AtBBX32 fused to GFP and GmBBX39 fused to the Myc epitope. A: IP with anti-GFP, detection with anti-Myc. B: IP with anti-Myc, detection with anti-GFP. MFI stands for Median Fluorescence Intensity. * denotes p<0.001.

### DETAILED DESCRIPTION

The present disclosure relates to polynucleotides and polypeptides for modifying phenotypes of plants, particularly those associated with increased abiotic stress tolerance and increased yield with respect to a control plant (for example, a wild-type plant). Throughout this disclosure, various information sources are referred to and/or are specifically incorporated. The information sources include scientific journal articles, patent documents, textbooks, and World Wide Web browser-inactive page addresses. While the reference to these information sources clearly indicates that they can be used by one of skill in the art, each and every one of the information sources cited herein are specifically incorporated in their entirety, whether or not a specific mention of “incorporation by reference” is noted. The contents and teachings of each and every one of the information sources can be relied on, used to make and use the disclosed embodiments.

As used herein and in the appended claims, the singular forms “a”, “an”, and “the” include the plural reference unless the context clearly dictates otherwise. Thus, for example, a reference to “a plant cell” includes a plurality of such plant cells, and a reference to “a stress” is a reference to one or more stresses and equivalents thereof known to those skilled in the art, and so forth.

### Definitions

“Polynucleotide” is a nucleic acid molecule comprising a plurality of polymerized nucleotides, e.g., at least about 15 consecutive polymerized nucleotides. A polynucleotide may be a nucleic acid, oligonucleotide, nucleotide, or any fragment thereof. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like.

The polynucleotide can be single-stranded or double-stranded DNA or RNA. The polynucleotide optionally comprises modified bases or a modified backbone. The polynucleotide can be, e.g., genomic DNA or RNA, a transcript (such as an mRNA), a cDNA, a PCR product, a cloned DNA, a synthetic DNA or RNA, or the like. The polynucleotide can be combined with carbohydrate, lipids, protein, or other materials to perform a particular activity such as transformation or form a useful composition such as a peptide nucleic acid (PNA). The polynucleotide can comprise a sequence in either sense or antisense orientations. “Oligonucleotide” is substantially equivalent to the terms amplimer, primer, oligomer, element, target, and probe and is preferably single-stranded.

A “recombinant polynucleotide” is a polynucleotide that is not in its native state, e.g., the polynucleotide comprises a nucleotide sequence not found in nature, or the polynucleotide is in a context other than that in which it is naturally found, e.g., separated from nucleotide sequences with which it typically is in proximity in nature, or adjacent (or contiguous with) nucleotide sequences with which it typically is not in proximity. For example, the sequence at issue can be cloned into a vector, or otherwise recombined with one or more additional nucleic acid.

An “isolated polynucleotide” is a polynucleotide, whether naturally occurring or recombinant, that is present outside the cell in which it is typically found in nature, whether purified or not. Optionally, an isolated polynucleotide is subject to one or more enrichment or purification procedures, e.g., cell lysis, extraction, centrifugation, precipitation, or the like.

“Gene” or “gene sequence” refers to the partial or complete coding sequence of a gene, its complement, and its 5′ or 3′ untranslated regions. A gene is also a functional unit of inheritance, and in physical terms is a particular segment or sequence of nucleotides along a molecule of DNA (or RNA, in the case of RNA viruses) involved in producing a polypeptide chain. The latter may be subjected to subsequent processing such as chemical modification or folding to obtain a functional protein or polypeptide. A gene may be isolated, partially isolated, or found with an organism's genome. By way of example, a transcriptional regulator gene encodes a transcriptional regulator polypeptide, which may be functional or require processing to function as an initiator of transcription.

Operationally, genes may be defined by the cis-trans test, a genetic test that determines whether two mutations occur in the same gene and that may be used to determine the limits of the genetically active unit (Rieger et al., 1976). A gene generally includes regions preceding (“leaders”; upstream) and following (“trailers”; downstream) the coding region. A gene may also include intervening, non-coding sequences, referred to as “introns”, located between individual coding segments, referred to as “exons”. Most genes have an associated promoter region, a regulatory sequence 5′ of the transcription initiation codon (there are some genes that do not have an identifiable promoter). The function of a gene may also be regulated by enhancers, operators, and other regulatory elements.

A “polypeptide” is an amino acid sequence comprising a plurality of consecutive polymerized amino acid residues e.g., at least about 15 consecutive polymerized amino acid residues. In many instances, a polypeptide comprises a polymerized amino acid residue sequence that is a transcriptional regulator or a domain or portion or fragment thereof. Additionally, the polypeptide may comprise: (i) a localization domain; (ii) an activation domain; (iii) a repression domain; (iv) an oligomerization domain; (v) a protein-protein interaction domain; (vi) a DNA-binding domain; or the like. The polypeptide optionally comprises modified amino acid residues, naturally occurring amino acid residues not encoded by a codon, or non-naturally occurring amino acid residues.

“Protein” refers to an amino acid sequence, oligopeptide, peptide, polypeptide or portions thereof whether naturally occurring or synthetic.

A “recombinant polypeptide” is a polypeptide produced by translation of a recombinant polynucleotide. A “synthetic polypeptide” is a polypeptide created by consecutive polymerization of isolated amino acid residues using methods well known in the art. An “isolated polypeptide,” whether a naturally occurring or a recombinant polypeptide, is more enriched in (or out of) a cell than the polypeptide in its natural state in a wild-type cell, e.g., more than about 5% enriched, more than about 10% enriched, or more than about 20%, or more than about 50%, or more, enriched, i.e., alternatively denoted: 105%, 110%, 120%, 150% or more, enriched relative to wild type standardized at 100%. Such an enrichment is not the result of a natural response of a wild-type plant. Alternatively, or additionally, the isolated polypeptide is separated from other cellular components with which it is typically associated, e.g., by any of the various protein purification methods herein.

“Homology” refers to sequence similarity between a reference sequence and at least a fragment of a newly sequenced clone insert or its encoded amino acid sequence.

“Identity” or “similarity” refers to sequence similarity between two polynucleotide sequences or between two polypeptide sequences, with identity being a more strict comparison. The phrases “percent identity” and “% identity” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value between 0-100%. Identity or similarity can be determined by comparing a position in each sequence that may be aligned for purposes of comparison. When a position in the compared sequence is occupied by the same nucleotide base or amino acid, then the molecules are identical at that position. A degree of similarity or identity between polynucleotide sequences is a function of the number of identical, matching or corresponding nucleotides at positions shared by the polynucleotide sequences. A degree of identity of polypeptide sequences is a function of the number of identical amino acids at corresponding positions shared by the polypeptide sequences. A degree of homology or similarity of polypeptide sequences is a function of the number of amino acids at corresponding positions shared by the polypeptide sequences.

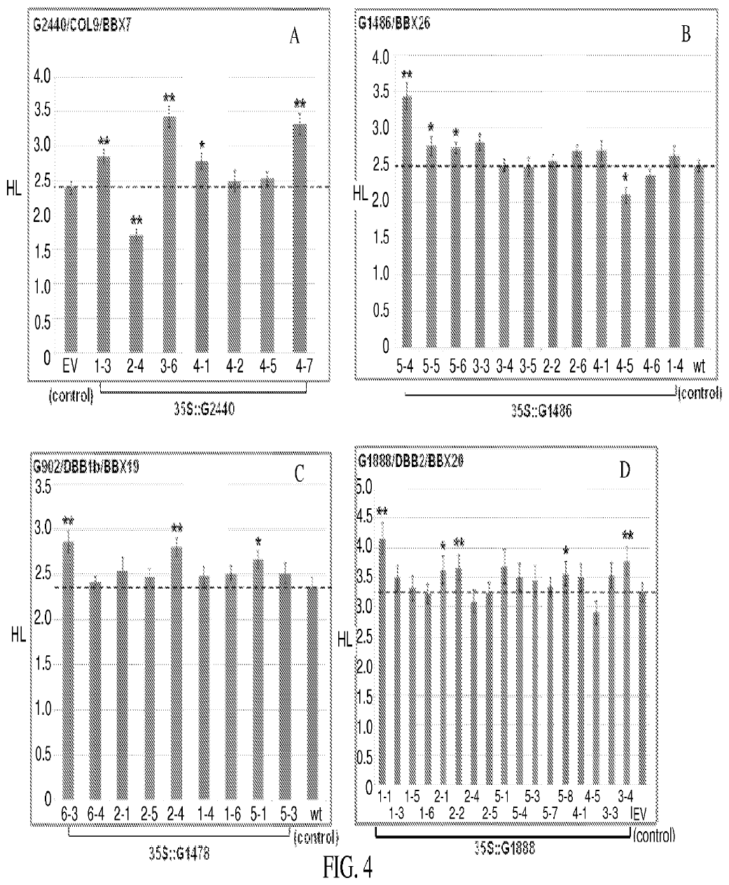

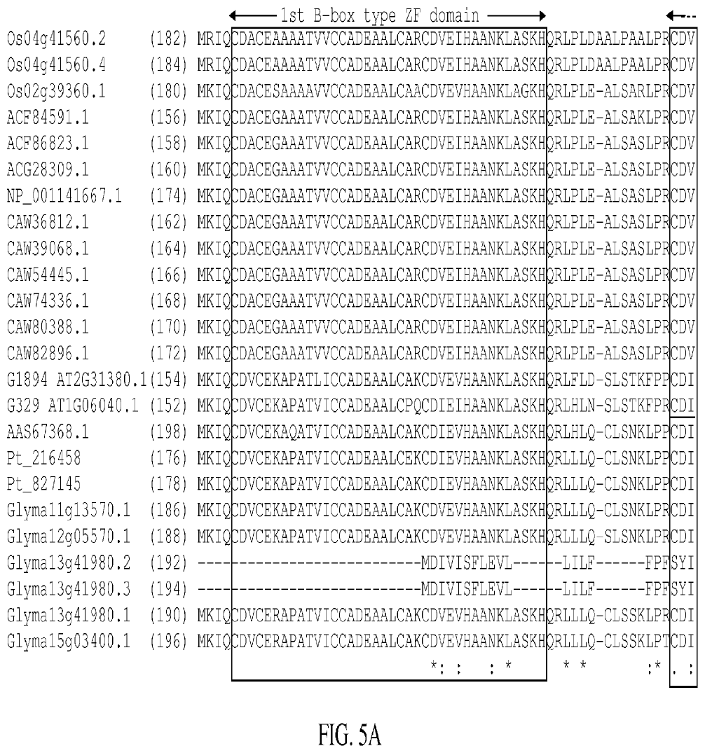

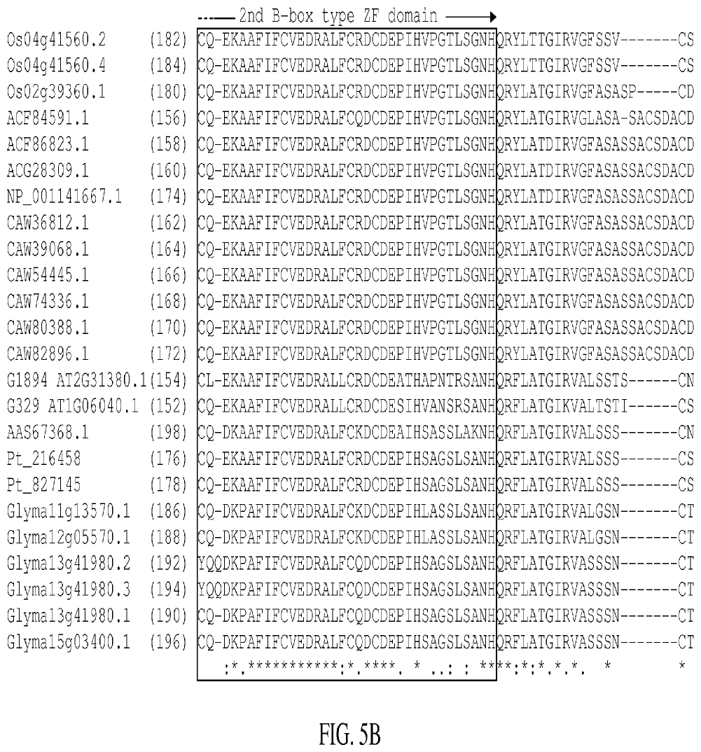

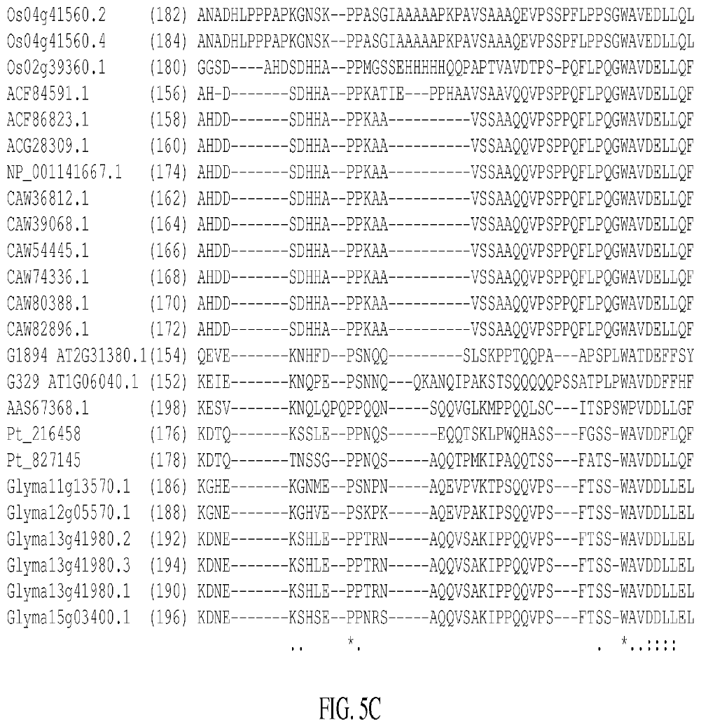

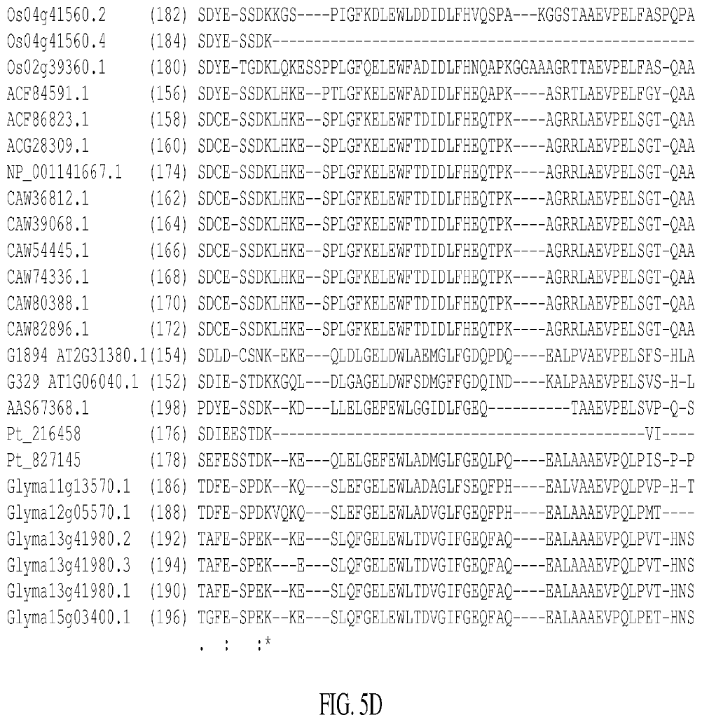

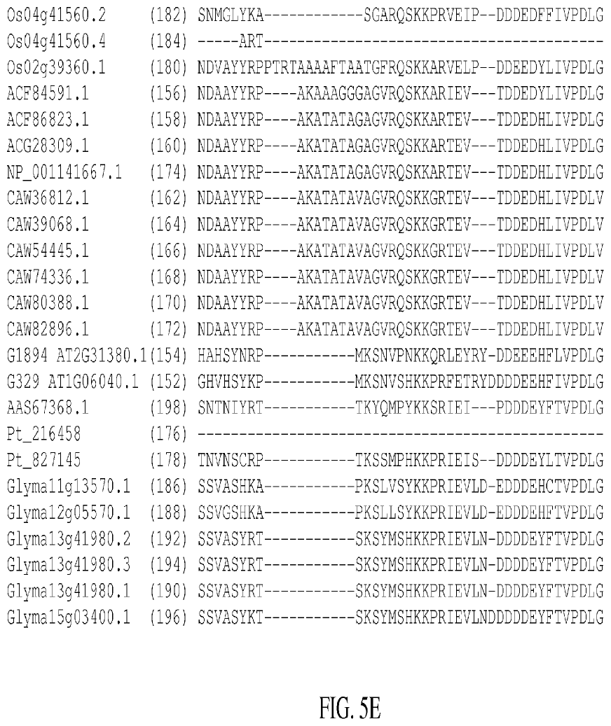

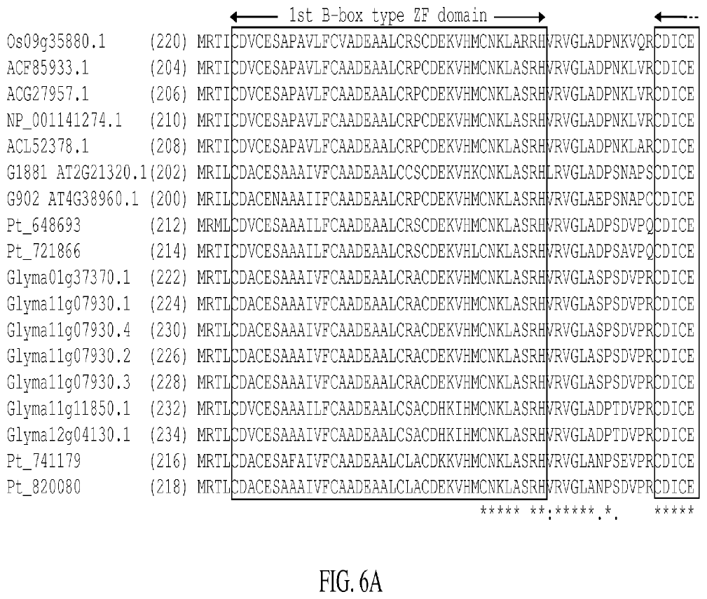

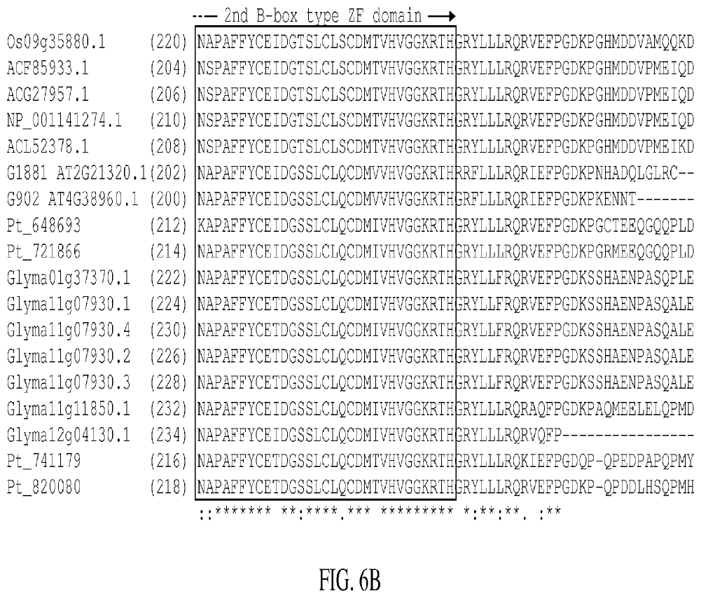

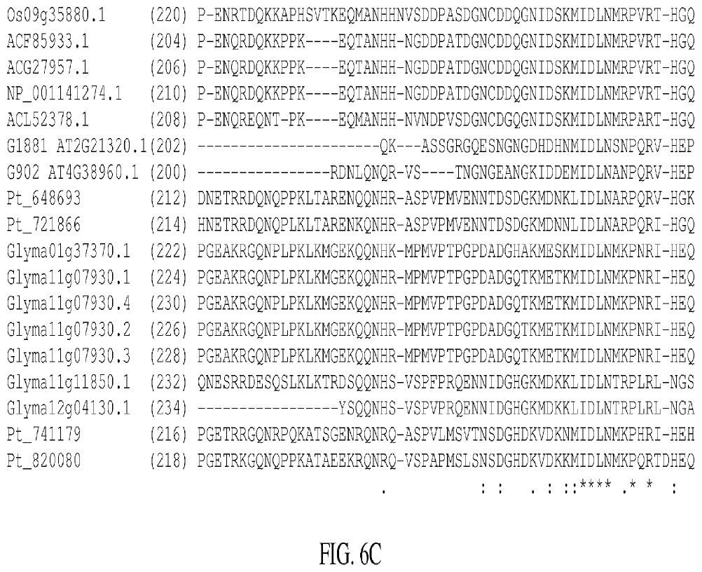

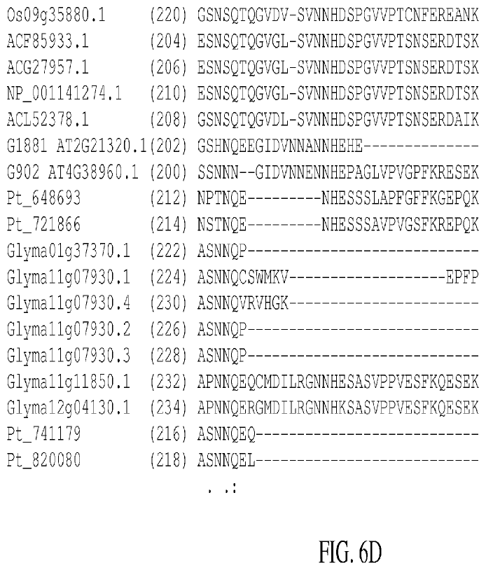

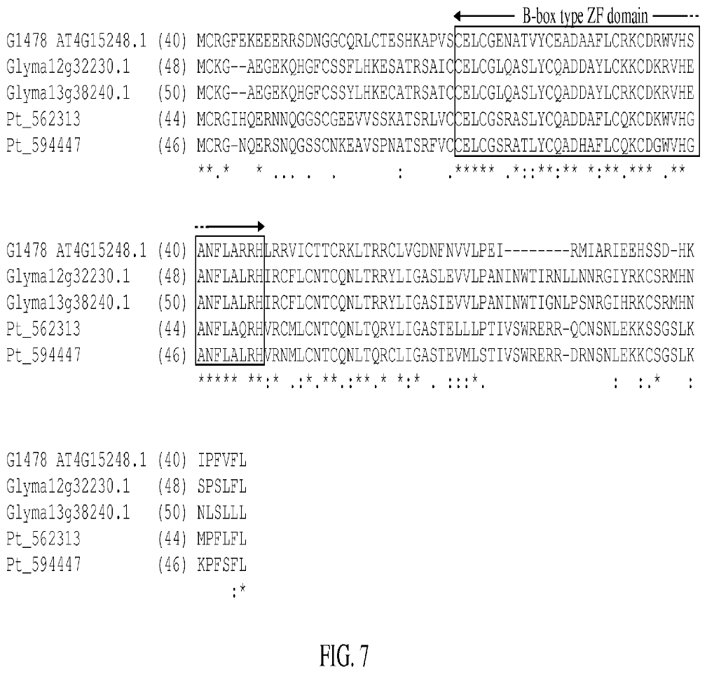

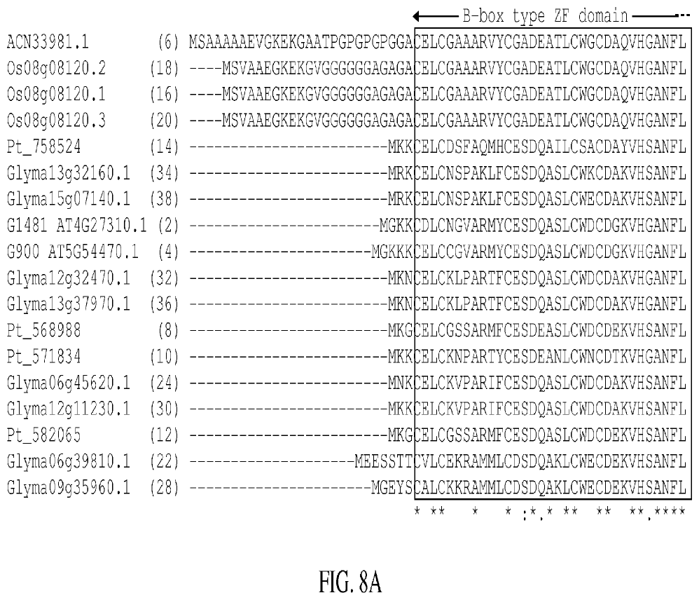

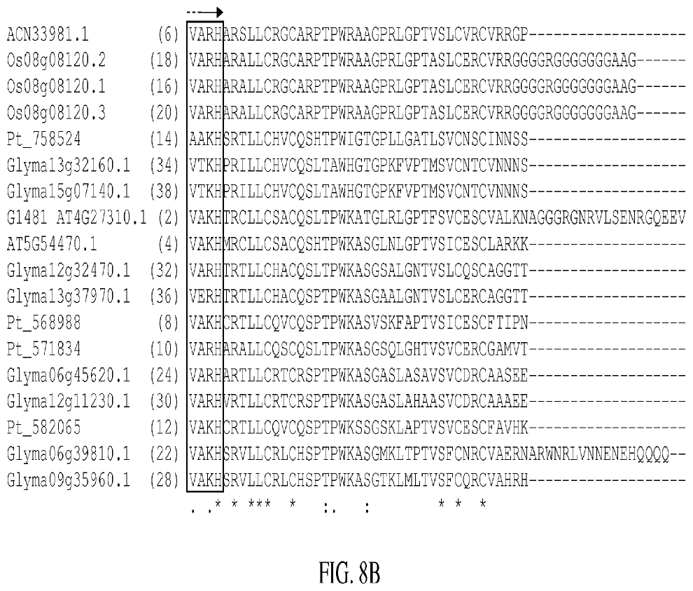

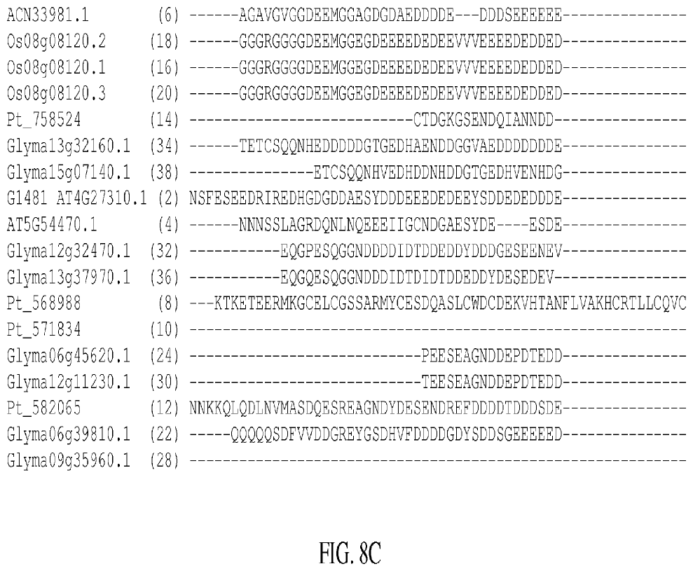

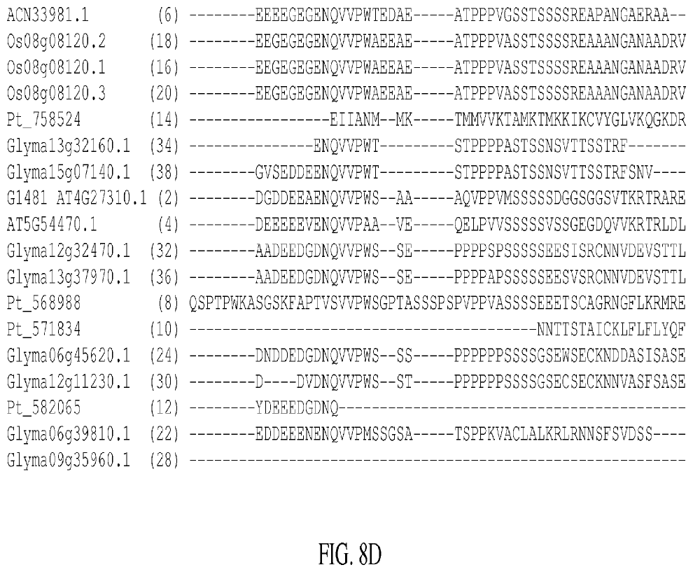

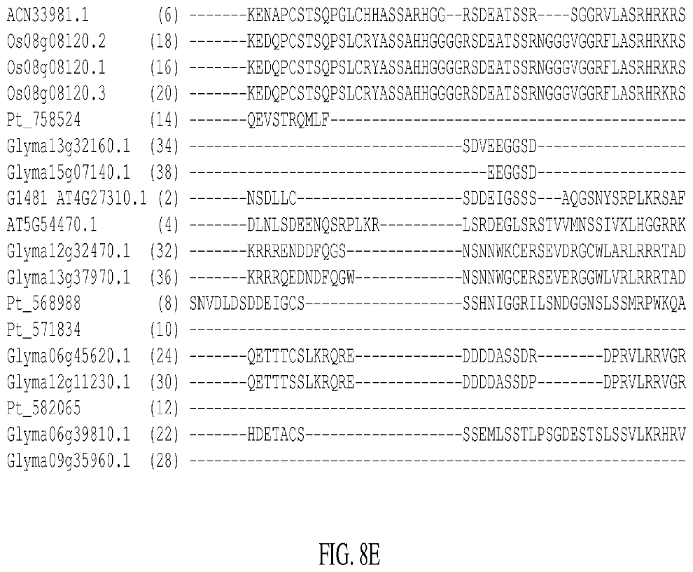

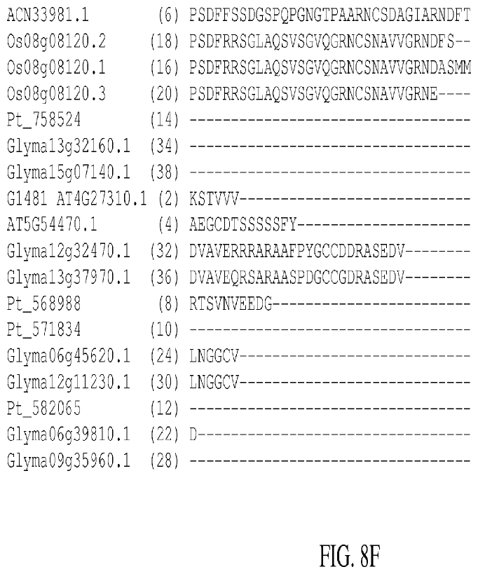

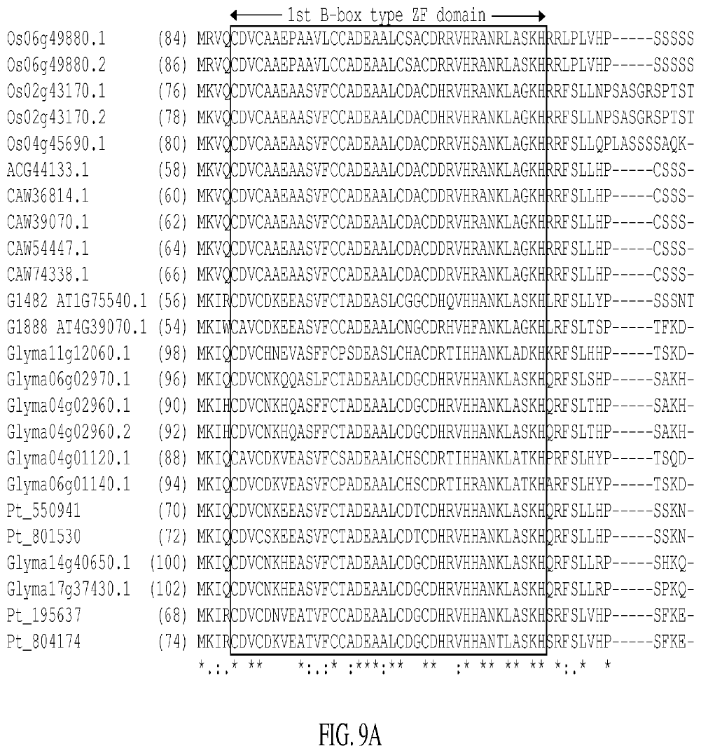

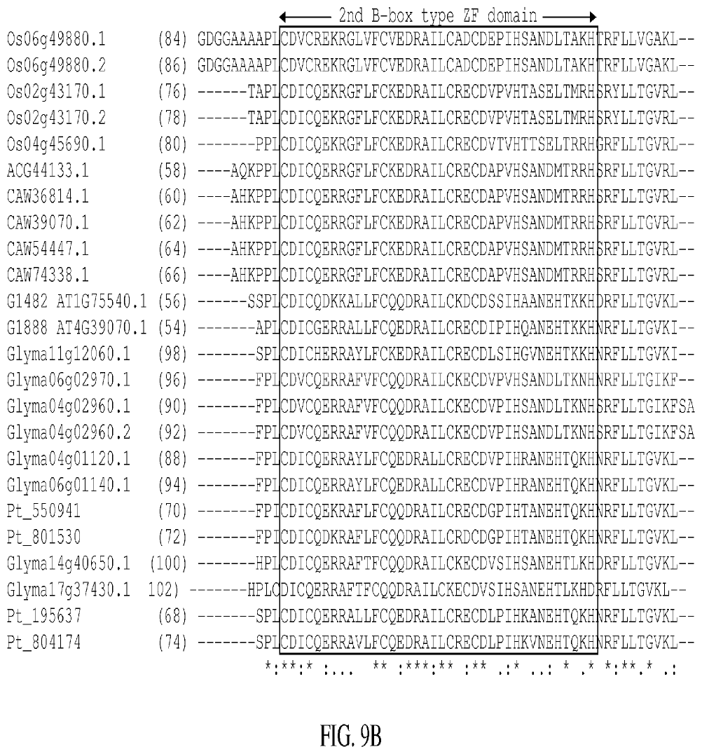

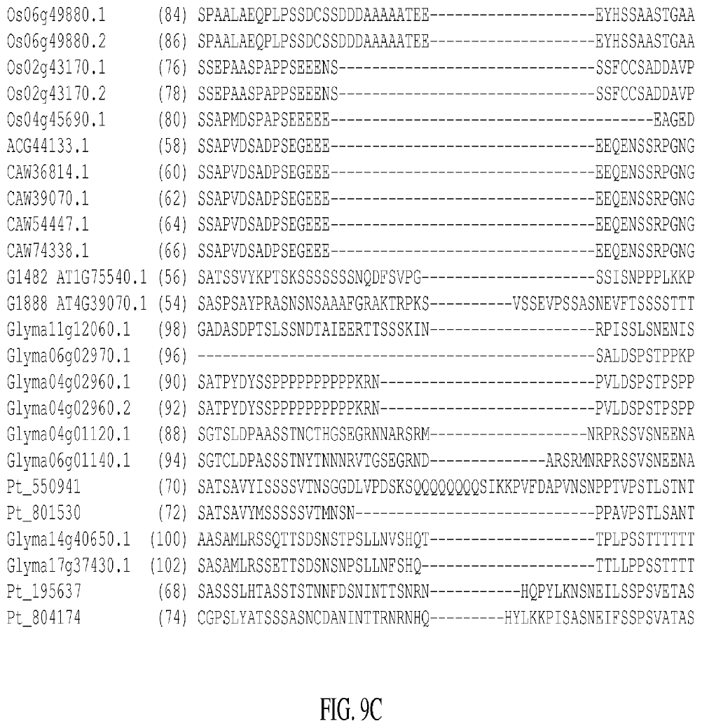

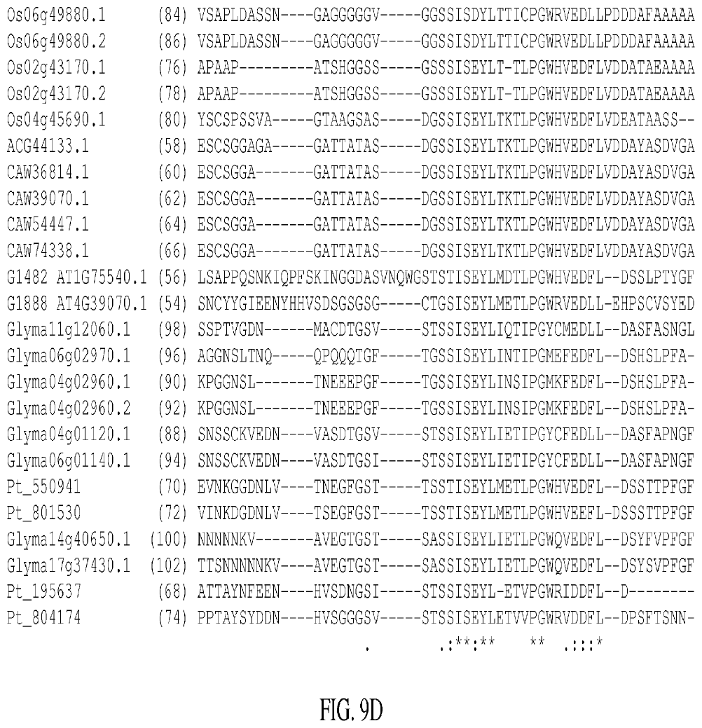

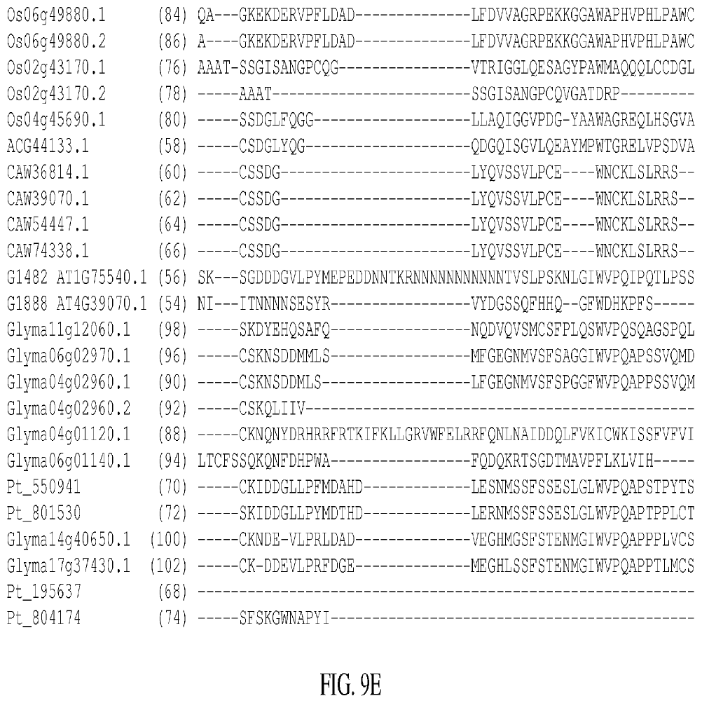

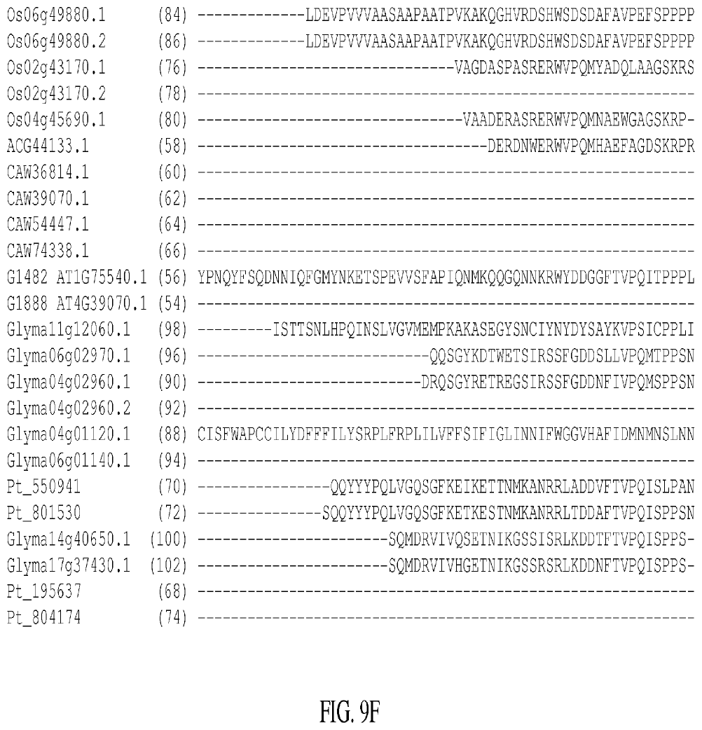

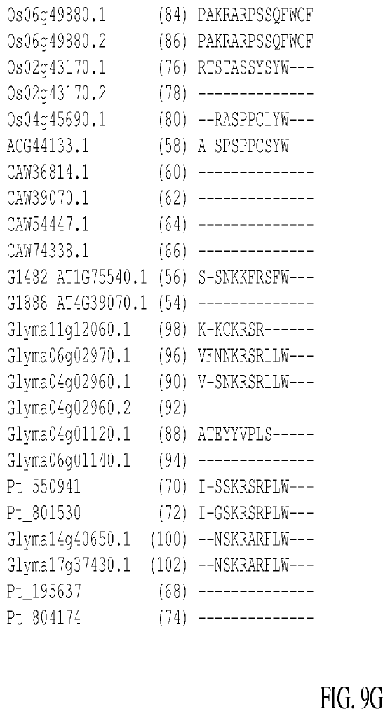

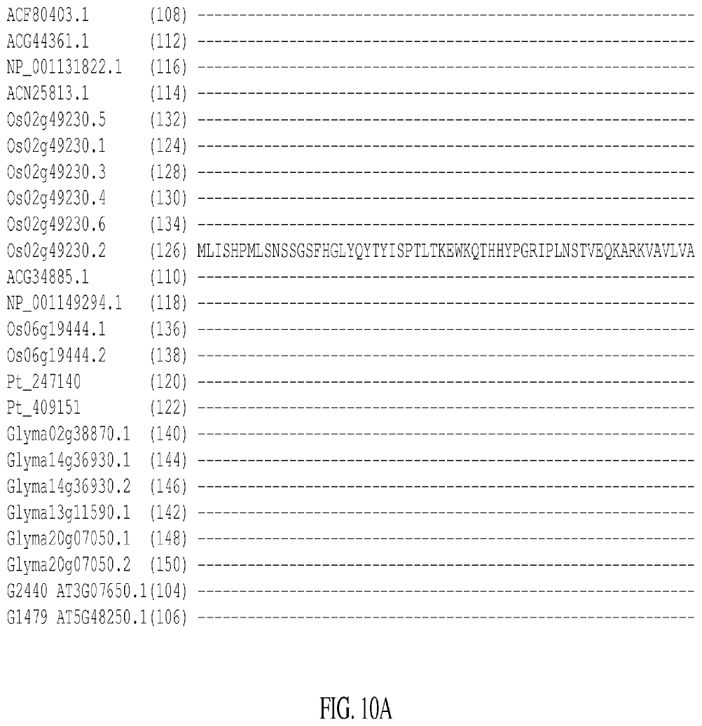

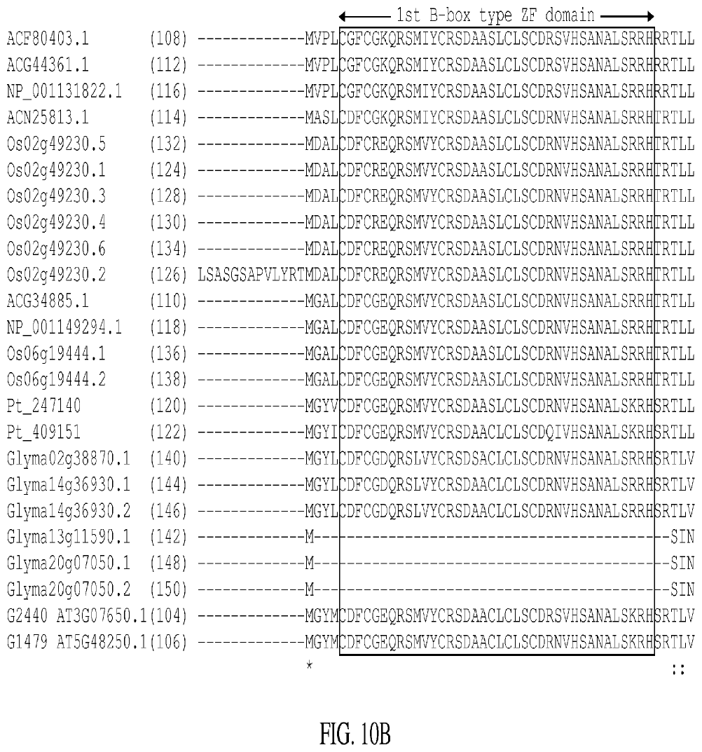

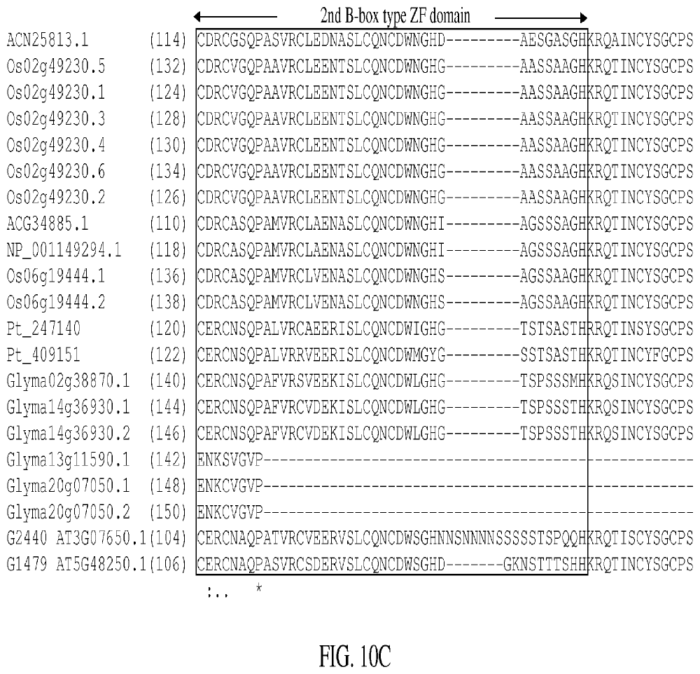

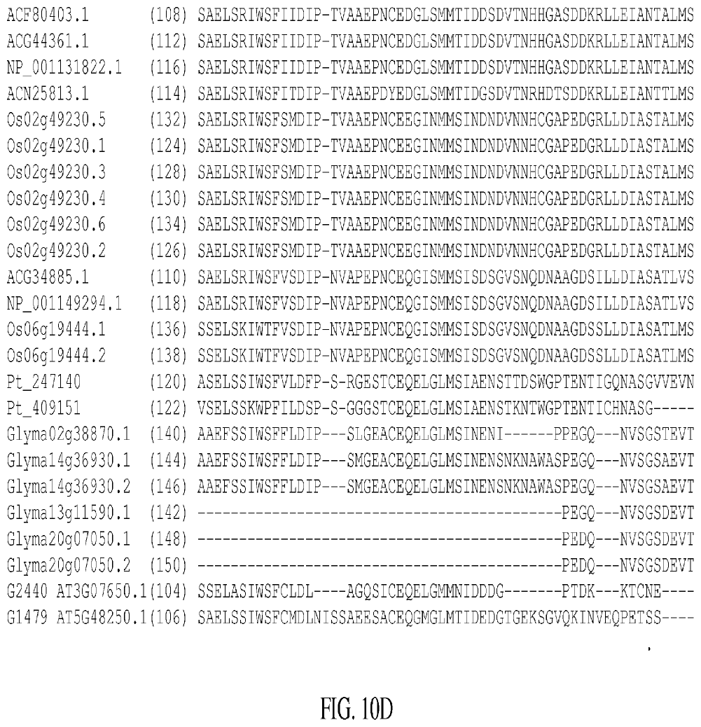

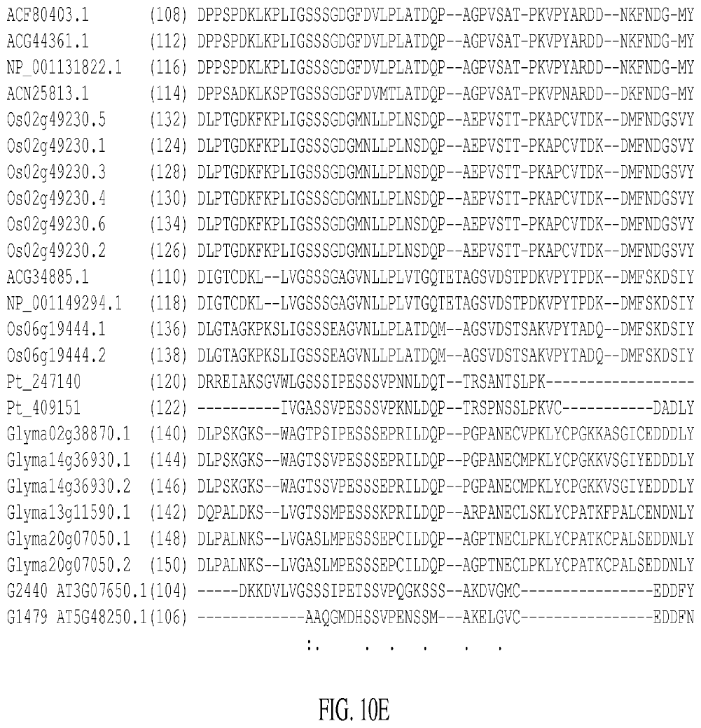

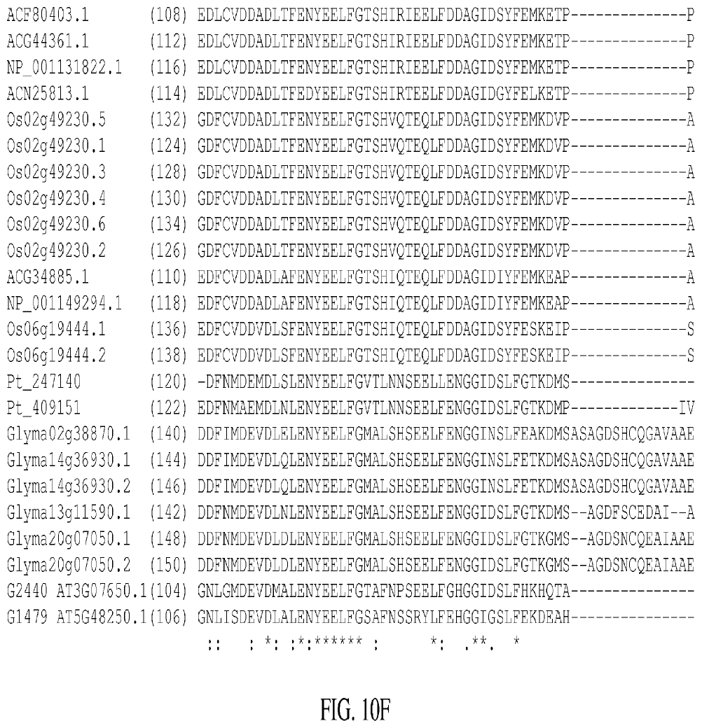

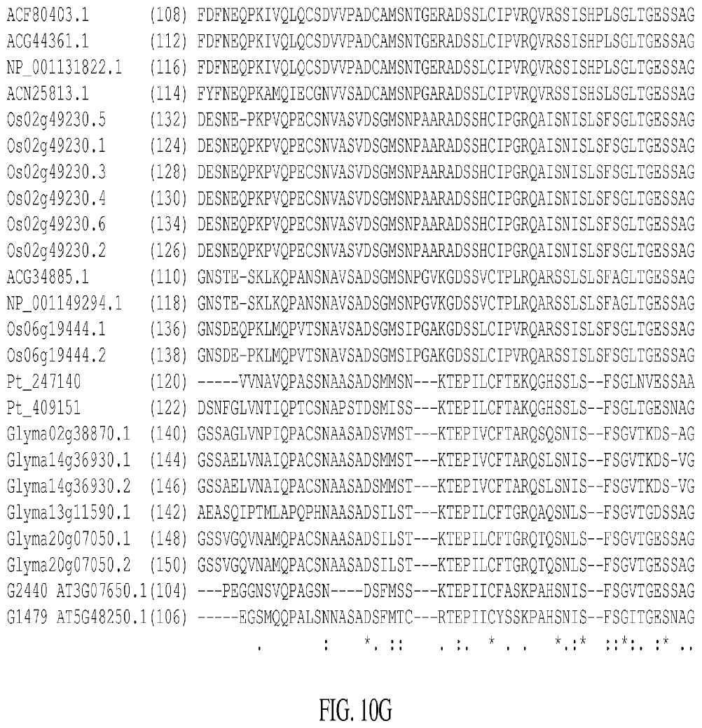

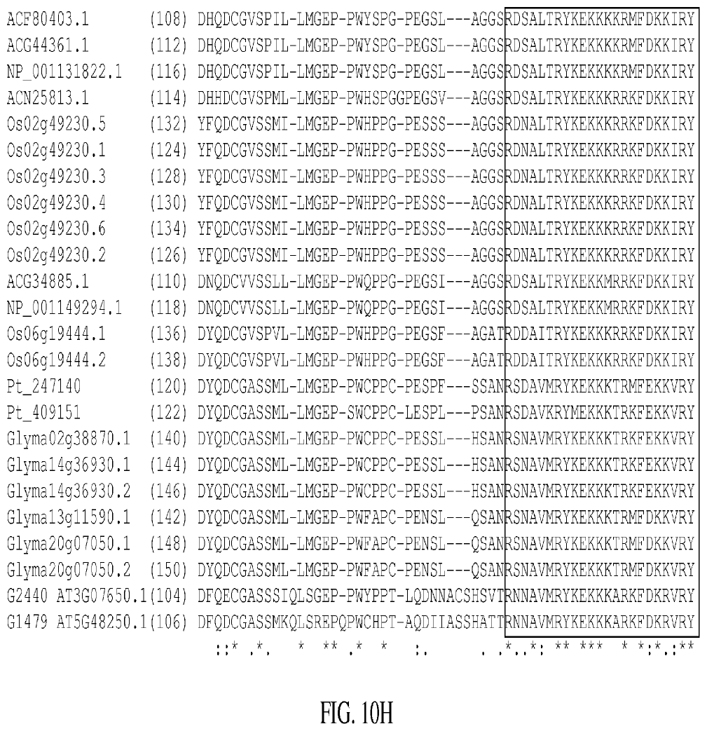

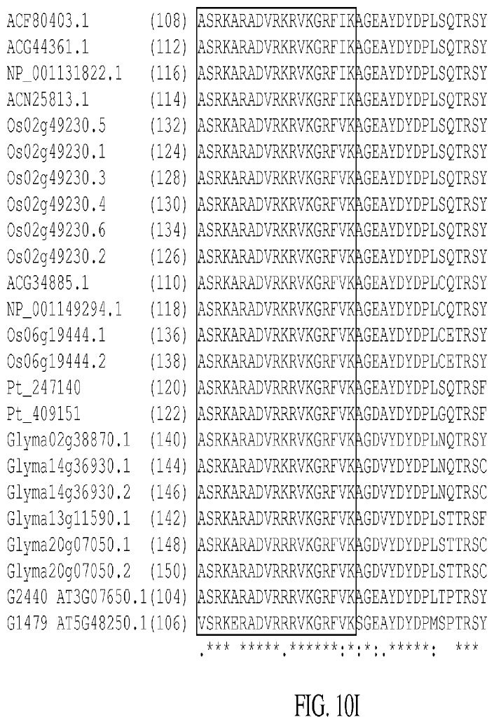

“Alignment” refers to a number of nucleotide bases or amino acid residue sequences aligned by lengthwise comparison so that components in common (i.e., nucleotide bases or amino acid residues at corresponding positions) may be visually and readily identified. The fraction or percentage of components in common is related to the homology or identity between the sequences. Alignments such as those of FIGS. 4A-4F may be used to identify conserved domains and relatedness within these domains. An alignment may suitably be determined by means of computer programs known in the art, such as MACVECTOR software, 1999 (Accelrys, Inc., San Diego, CA).

A “conserved domain” or “conserved region” as used herein refers to a region in heterologous polynucleotide or polypeptide sequences where there is a relatively high degree of sequence identity between the distinct sequences. A “B-box zinc finger” domain, such as is found in a polypeptide member of B-box zinc finger family, is an example of a conserved domain. With respect to polynucleotides encoding presently disclosed polypeptides, a conserved domain is preferably at least nine base pairs (bp) in length. A conserved domain with respect to presently disclosed polypeptides refers to a domain within a polypeptide family that exhibits a higher degree of sequence homology, such as at least about 56%, 57%. 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100% sequence identity to a conserved domain of a disclosed polypeptide (e.g., SEQ ID NOs: 235-427). Sequences that possess or encode for conserved domains that meet these criteria of percentage identity, and that have comparable biological activity to the present polypeptide sequences, thus being members of the BBX32 Glade polypeptides, are encompassed by the present disclosure. A fragment or domain can be referred to as outside a conserved domain, outside a consensus sequence, or outside a consensus DNA-binding site that is known to exist or that exists for a particular polypeptide class, family, or sub-family. In this case, the fragment or domain will not include the exact amino acids of a consensus sequence or consensus DNA-binding site of a transcriptional regulator class, family or sub-family, or the exact amino acids of a particular transcriptional regulator consensus sequence or consensus DNA-binding site. Furthermore, a particular fragment, region, or domain of a polypeptide, or a polynucleotide encoding a polypeptide, can be “outside a conserved domain” if all the amino acids of the fragment, region, or domain fall outside of a defined conserved domain(s) for a polypeptide or protein. Sequences having lesser degrees of identity but comparable biological activity are considered to be equivalents.

As one of ordinary skill in the art recognizes, conserved domains may be identified as regions or domains of identity to a specific consensus sequence (see, for example, Riechmann et al., 2000a, 2000b). Thus, by using alignment methods well known in the art, the conserved domains of the plant polypeptides, for example, for the B-box zinc finger proteins (Putterill et al., 1995), may be determined.

The conserved domains for many of the disclosed polypeptide sequences are listed in Table 1. Also, the polypeptides of Table 1 have conserved domains specifically indicated by amino acid coordinate start and stop sites. A comparison of the regions of these polypeptides allows one of skill in the art (see, for example, Reeves and Nissen, 1990, 1995) to identify domains or conserved domains for any of the polypeptides listed or referred to in this disclosure. “Complementary” refers to the natural hydrogen bonding by base pairing between purines and pyrimidines. For example, the sequence A-C-G-T (5′->3′) forms hydrogen bonds with its complements A-C-G-T (5′->3′) or A-C-G-U (5′->3′). Two single-stranded molecules may be considered partially complementary, if only some of the nucleotides bond, or “completely complementary” if all of the nucleotides bond. The degree of complementarity between nucleic acid strands affects the efficiency and strength of hybridization and amplification reactions. “Fully complementary” refers to the case where bonding occurs between every base pair and its complement in a pair of sequences, and the two sequences have the same number of nucleotides.

The terms “highly stringent” or “highly stringent condition” refer to conditions that permit hybridization of DNA strands whose sequences are highly complementary, wherein these same conditions exclude hybridization of significantly mismatched DNAs. Polynucleotide sequences capable of hybridizing under stringent conditions with the disclosed, orthologous or paralogous polynucleotides may be, for example, variants of the disclosed polynucleotide sequences, including allelic or splice variants, or sequences that encode orthologs or paralogs of presently disclosed polypeptides. Nucleic acid hybridization methods are disclosed in detail by Kashima et al., 1985; Sambrook et al., 1989; and by Haymes et al., 1985; which references are incorporated herein by reference.

In general, stringency is determined by the temperature, ionic strength, and concentration of denaturing agents (e.g., formamide) used in a hybridization and washing procedure (for a more detailed description of establishing and determining stringency, see the section “Identifying Polynucleotides or Nucleic Acids by Hybridization”, below). The degree to which two nucleic acids hybridize under various conditions of stringency is correlated with the extent of their similarity. Thus, similar nucleic acid sequences from a variety of sources, such as within a plant's genome (as in the case of paralogs) or from another plant (as in the case of orthologs) that may perform similar functions can be isolated on the basis of their ability to hybridize with known related polynucleotide sequences. Numerous variations are possible in the conditions and means by which nucleic acid hybridization can be performed to isolate related polynucleotide sequences having similarity to sequences known in the art and are not limited to those explicitly disclosed herein. Such an approach may be used to isolate polynucleotide sequences having various degrees of similarity with disclosed polynucleotide sequences, such as, for example, encoded transcriptional regulators having 56% or greater identity with the conserved domain of disclosed sequences.

The terms “paralog” and “ortholog” are defined below in the section entitled “Orthologs and Paralogs”. In brief, orthologs and paralogs are evolutionarily related genes that have similar sequences and functions. Orthologs are structurally related genes in different species that are derived by a speciation event. Paralogs are structurally related genes within a single species that are derived by a duplication event.

The term “equivalog” describes members of a set of homologous proteins that are conserved with respect to function since their last common ancestor. Related proteins are grouped into equivalog families, and otherwise into protein families with other hierarchically defined homology types. This definition is provided at the Institute for Genomic Research (TIGR) World Wide Web (www) website, “tigr.org” under the heading “Terms associated with TIGRFAMs”.

In general, the term “variant” refers to molecules with some differences, generated synthetically or naturally, in their base or amino acid sequences as compared to a reference (native) polynucleotide or polypeptide, respectively. These differences include substitutions, insertions, deletions or any desired combinations of such changes in a native polynucleotide of amino acid sequence.

With regard to polynucleotide variants, differences between presently disclosed polynucleotides and polynucleotide variants are limited so that the nucleotide sequences of the former and the latter are closely similar overall and, in many regions, identical. Due to the degeneracy of the genetic code, differences between the former and latter nucleotide sequences may be silent (i.e., the amino acids encoded by the polynucleotide are the same, and the variant polynucleotide sequence encodes the same amino acid sequence as the presently disclosed polynucleotide. Variant nucleotide sequences may encode different amino acid sequences, in which case such nucleotide differences will result in amino acid substitutions, additions, deletions, insertions, truncations or fusions with respect to the similar disclosed polynucleotide sequences. These variations may result in polynucleotide variants encoding polypeptides that share at least one functional characteristic. The degeneracy of the genetic code also dictates that many different variant polynucleotides can encode identical and/or substantially similar polypeptides in addition to those sequences illustrated in the Sequence Listing.

Also within the scope of the disclosed compositions and methods is a variant of a nucleic acid listed in the Sequence Listing, that is, one having a sequence that differs from the one of the polynucleotide sequences in the Sequence Listing, or a complementary sequence, that encodes a functionally equivalent polypeptide (i.e., a polypeptide having some degree of equivalent or similar biological activity) but differs in sequence from the sequence in the Sequence Listing, due to degeneracy in the genetic code. Included within this definition are polymorphisms that may or may not be readily detectable using a particular oligonucleotide probe of the polynucleotide encoding polypeptide, and improper or unexpected hybridization to allelic variants, with a locus other than the normal chromosomal locus for the polynucleotide sequence encoding polypeptide.

“Allelic variant” or “polynucleotide allelic variant” refers to any of two or more alternative forms of a gene occupying the same chromosomal locus. Allelic variation arises naturally through mutation, and may result in phenotypic polymorphism within populations. Gene mutations may be “silent” or may encode polypeptides having altered amino acid sequence. “Allelic variant” and “polypeptide allelic variant” may also be used with respect to polypeptides, and in this case the terms refer to a polypeptide encoded by an allelic variant of a gene.

“Splice variant” or “polynucleotide splice variant” as used herein refers to alternative forms of RNA transcribed from a gene. Splice variation naturally occurs as a result of alternative sites being spliced within a single transcribed RNA molecule or between separately transcribed RNA molecules, and may result in several different forms of mRNA transcribed from the same gene. Thus, splice variants may encode polypeptides having different amino acid sequences, which may or may not have similar functions in the organism. “Splice variant” or “polypeptide splice variant” may also refer to a polypeptide encoded by a splice variant of a transcribed mRNA.

As used herein, “polynucleotide variants” may also refer to polynucleotide sequences that encode paralogs and orthologs of the presently disclosed polypeptide sequences. “Polypeptide variants” may refer to polypeptide sequences that are paralogs and orthologs of the presently disclosed polypeptide sequences.

Differences between presently disclosed polypeptides and polypeptide variants are limited so that the sequences of the former and the latter are closely similar overall and, in many regions, identical. Presently disclosed polypeptide sequences and similar polypeptide variants may differ in amino acid sequence by one or more substitutions, additions, deletions, fusions and truncations, which may be present in any combination. These differences may produce silent changes and result in functionally equivalent polypeptides. Thus, it will be readily appreciated by those of skill in the art, that any of a variety of polynucleotide sequences is capable of encoding the disclosed polypeptides and homolog polypeptides. A polypeptide sequence variant may have “conservative” changes, wherein a substituted amino acid has similar structural or chemical properties. Deliberate amino acid substitutions may thus be made on the basis of similarity in polarity, charge, solubility, hydrophobicity, hydrophilicity, and/or the amphipathic nature of the residues, as long as a significant amount of the functional or biological activity of the polypeptide is retained. For example, negatively charged amino acids may include aspartic acid and glutamic acid, positively charged amino acids may include lysine and arginine, and amino acids with uncharged polar head groups having similar hydrophilicity values may include leucine, isoleucine, and valine; glycine and alanine; asparagine and glutamine; serine and threonine; and phenylalanine and tyrosine. More rarely, a variant may have “non-conservative” changes, e.g., replacement of a glycine with a tryptophan. Similar minor variations may also include amino acid deletions or insertions, or both. Related polypeptides may comprise, for example, additions and/or deletions of one or more N-linked or O-linked glycosylation sites, or an addition and/or a deletion of one or more cysteine residues. Guidance in determining which and how many amino acid residues may be substituted, inserted or deleted without abolishing functional or biological activity may be found using computer programs well known in the art, for example, DNASTAR software (see U.S. Pat. No. 5,840,544).

“Fragment”, with respect to a polynucleotide, refers to a clone or any part of a polynucleotide molecule that retains a usable, functional characteristic. Useful fragments include oligonucleotides and polynucleotides that may be used in hybridization or amplification technologies or in the regulation of replication, transcription or translation. A “polynucleotide fragment” refers to any subsequence of a polynucleotide, typically, of at least about 9 consecutive nucleotides, preferably at least about 30 nucleotides, more preferably at least about 50 nucleotides, of any of the sequences provided herein. Exemplary polynucleotide fragments are the first sixty consecutive nucleotides of the polynucleotides listed in the Sequence Listing. Exemplary fragments also include fragments that comprise a region that encodes a conserved domain of a polypeptide. Exemplary fragments also include fragments that comprise a conserved domain of a polypeptide. Exemplary fragments include fragments that comprise a conserved domain of a polypeptide, for example, any of SEQ ID NOs 235-427.

Fragments may also include subsequences of polypeptides and protein molecules, or a subsequence of the polypeptide. Fragments may have uses in that they may have antigenic potential. In some cases, the fragment or domain is a subsequence of the polypeptide which performs at least one biological function of the intact polypeptide in substantially the same manner, or to a similar extent, as does the intact polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, but are preferably at least about 30 amino acid residues in length and more preferably at least about 60 amino acid residues in length.

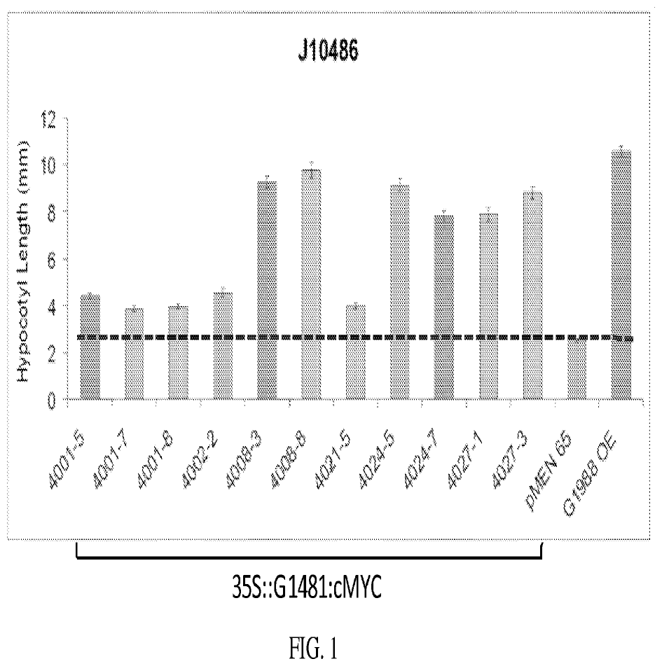

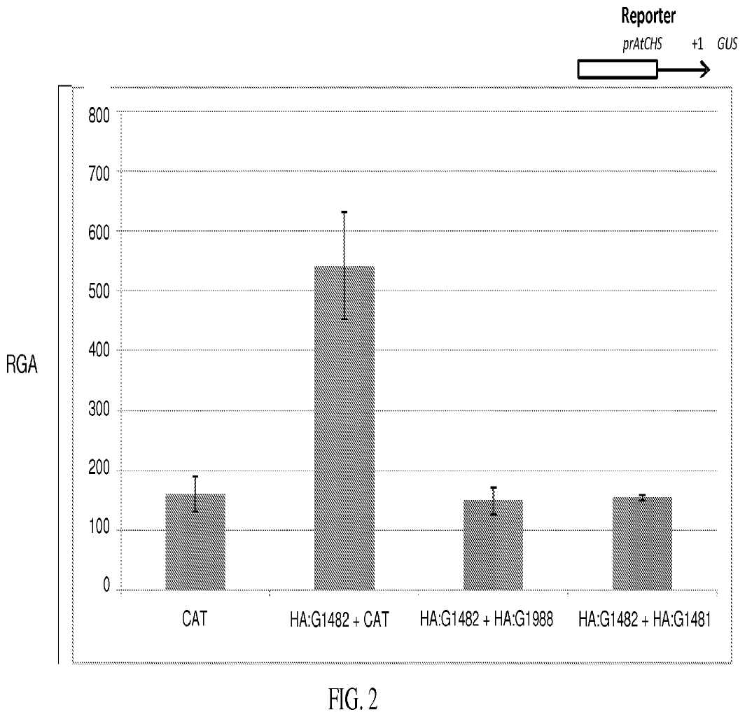

The term “plant” includes whole plants, shoot vegetative organs/structures (for example, leaves, stems and tubers), roots, flowers and floral organs/structures (for example, bracts, sepals, petals, stamens, carpels, anthers and ovules), seed (including embryo, endosperm, and seed coat) and fruit (the mature ovary), plant tissue (for example, vascular tissue, ground tissue, and the like) and cells (for example, guard cells, egg cells, and the like), and progeny of same. The class of plants that can be used in the disclosed methods is generally as broad as the class of higher and lower plants amenable to transformation techniques, including angiosperms (monocotyledonous and dicotyledonous plants), gymnosperms, ferns, horsetails, psilophytes, lycophytes, bryophytes, and multicellular algae (see for example, FIG. 1, adapted from Daly et al., 2001, FIG. 2, adapted from Ku et al., 2000; and see also Tudge, 2000).

A “control plant” as used in the present disclosure refers to a plant cell, seed, plant component, plant tissue, plant organ or whole plant used to compare against transgenic or genetically modified plant for the purpose of identifying an enhanced phenotype in the transgenic or genetically modified plant. A control plant may in some cases be a transgenic plant line that comprises an empty vector or marker gene, but does not contain the recombinant polynucleotide of the present disclosure that is expressed in the transgenic or genetically modified plant being evaluated. A control plant may in other cases be a progeny of a hemizygous transgenic plant line that does not contain the recombinant DNA, known as a negative segregant or negative isoline. In general, a control plant is a plant of the same line or variety as the transgenic or genetically modified plant being tested. A suitable control plant would include a genetically unaltered or non-transgenic plant of the parental line used to generate a transgenic plant herein.

A “transgenic plant” refers to a plant that contains genetic material not found in a wild-type plant of the same species, variety or cultivar. The genetic material may include a transgene, an insertional mutagenesis event (such as by transposon or T-DNA insertional mutagenesis), an activation tagging sequence, a mutated sequence, a homologous recombination event or a sequence modified by chimeraplasty. Typically, the foreign genetic material has been introduced into the plant by human manipulation, but any method can be used as one of skill in the art recognizes.

A transgenic plant may contain an expression vector or cassette. The expression cassette typically comprises a polypeptide-encoding or polypeptide-suppressing sequence operably linked to (i.e., under regulatory control of) appropriate inducible, tissue-enhanced, tissue-specific, diurnally regulated or constitutive regulatory sequences that allow for the controlled expression of the polypeptide. The expression cassette can be introduced into a plant by transformation or by breeding after transformation of a parent plant. A plant refers to a whole plant as well as to a plant part, such as seed, fruit, leaf, or root, plant tissue, plant cells or any other plant material, e.g., a plant explant, as well as to progeny thereof, and to in vitro systems that mimic biochemical or cellular components or processes in a cell.

A constitutive promoter is active under most environmental conditions and in most plant parts.

Tissue-enhanced (also referred to as tissue-preferred), tissue-specific, cell type-specific, and inducible promoters constitute non-constitutive promoters. Promoters under developmental control include promoters that preferentially initiate transcription in certain tissues, such as xylem, leaves, roots, or seeds. Such promoters are examples of tissue-enhanced or tissue-preferred promoters (see U.S. Pat. No. 7,365,186). Tissue-enhanced promoters can be found upstream and operatively linked to DNA sequences normally transcribed in higher levels in certain plant tissues or specifically in certain plant tissues, respectively. “Cell-enhanced”, “tissue-enhanced”, or “tissue-specific” regulation thus refer to the control of gene or protein expression, for example, by a promoter, which drives expression that is not necessarily totally restricted to a single type of cell or tissue, but where expression is elevated in particular cells or tissues to a greater extent than in other cells or tissues within the organism, and in the case of tissue-specific regulation, in a manner that is primarily elevated in a specific tissue. Tissue-enhanced or preferred promoters have been described in, for example, U.S. Pat. No. 7,365,186, or U.S. Pat. No. 7,619,133.

A “condition-enhanced” promoter refers to a promoter that activates a gene in response to a particular environmental stimulus, for example, an abiotic stress, infection caused by a pathogen, light treatment, chemical stimulus, etc., and that drives expression in a unique pattern which may include expression in specific cell and/or tissue types within the organism (as opposed to a constitutive expression pattern in all cell types of an organism at all times).

A “diurnal promoter” is useful for regulating changes in the timing of gene expression to a specific time of day. Usually genes that are diurnally-regulated peak at the same time in the day/night cycle. Genes that are under the regulation of diurnal promoters exhibit altered expression profiles under the control of a circadian oscillator mostly during the switch time of light to dark but also with diurnal oscillations that persist within a period close to 24 hour day/night cycle. Diurnal regulation is subject to environmental inputs such as light and temperature and the coordination by the circadian clock.

“Wild type” or “wild-type”, as used herein, refers to a plant cell, seed, plant component, plant part, plant tissue, plant organ or whole plant that has not been genetically modified or treated in an experimental sense. Wild-type cells, seeds, components, parts, tissues, organs or whole plants may be used as controls to compare levels of expression and the extent and nature of trait modification with cells, tissue or plants of the same species in which a polypeptide's expression is altered, e.g., in that it has been knocked out, overexpressed, or ectopically expressed.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, e.g., by employing Northern analysis, RT-PCR, microarray gene expression assays, or reporter gene expression systems, or by agricultural observations such as hyperosmotic stress tolerance or yield. Any technique can be used to measure the amount of, comparative level of, or difference in any selected chemical compound or macromolecule in the transgenic plants, however.

“Trait modification”, “modified trait” or “altered trait” refers to a detectable difference in a characteristic in a plant ectopically expressing a polynucleotide or polypeptide of the present disclosure relative to a plant not doing so, such as a wild-type plant. In some cases, the trait modification can be evaluated quantitatively. For example, the trait modification can entail at least about a 2% increase or decrease, or an even greater difference, in an observed trait as compared with a control or wild-type plant. It is known that there can be natural variations in the modified trait. Therefore, the trait modification observed entails a change of the normal distribution and magnitude of the trait in the plants as compared to control or wild-type plants.

When two or more plants have “similar morphologies”, “substantially similar morphologies”, “a morphology that is substantially similar”, or are “morphologically similar”, the plants have comparable forms or appearances, including analogous features such as overall dimensions, height, width, mass, root mass, shape, glossiness, leaf color, stem diameter, leaf size, leaf dimension, leaf density, internode distance or length, branching, root branching, number and form of inflorescences, and other macroscopic characteristics, and the individual plants are not readily distinguishable based on morphological characteristics alone.

“Modulates” refers to a change in activity (biological, chemical, or immunological) or lifespan resulting from specific binding between a molecule and either a nucleic acid molecule or a protein.

The term “transcript profile” refers to the expression levels of a set of genes in a cell in a particular state, particularly by comparison with the expression levels of that same set of genes in a cell of the same type in a reference state. For example, the expression level of a set of genes in a cell in which a particular polypeptide is overexpressed or suppressed can be compared to the expression level of the same set of genes in a cell that has normal levels of that polypeptide. The transcript profile can be presented as a list of those genes whose expression level is significantly different between the two treatments, and the difference ratios. Differences and similarities between expression levels may also be evaluated and calculated using statistical and clustering methods.

With regard to gene knockouts as used herein, the term “knockout” or “suppression” refers to a plant, plant cell, or plant tissue having a disruption in at least one gene, where the disruption results in a reduced or abolished expression or activity of the polypeptide encoded by that gene compared to a control plant, cell, or tissue. A knockout or suppression can occur in different ways, including but not limited to: decreasing levels of a protein; suppressing a mutation that has resulted in decreased activity of a protein; suppressing the production of an inhibitory agent; elevating, reducing or eliminating the level of substrate that an enzyme requires for activity; producing a new protein; and activating a normally silent gene, to accumulate a product that does not normally increase under natural conditions. The suppressor can be another mutation on a different gene, a suppressor mutation on the same gene but located some distance from the first mutation, or a suppressor in the cytoplasm that has generated due to a change in non-chromosomal DNA. The knockout can be the result of, for example, genomic disruptions, including transposons, tilling, and homologous recombination, antisense constructs, sense constructs, RNA silencing constructs, or RNA interference. A T-DNA insertion within a gene is an example of a genotypic alteration that may abolish expression of that gene. To knockout or suppress a gene, a recombinant expression cassette can be made. For example, the recombinant cassette can comprise a promoter that is functional in a plant cell and that is operably-linked to a polynucleotide that when expressed in a plant cell is transcribed into a polynucleotide molecule that suppresses the level of an endogenous protein in the plant cell relative to a control. The polynucleotide molecule can be, for example, a dsRNA that is processed into siRNAs that targets a messenger RNA encoding the protein; a miRNA that targets a messenger RNA encoding the protein; a trans-acting small interfering RNA (ta-siRNA) that is processed into siRNAs and that targets a messenger RNA encoding the protein; and a cleavage blocker of a miRNA or a miRNA decoy of a miRNA, which both lead to decreased activity of a miRNA. Examples of such RNAi-mediated gene suppression approaches are disclosed in US Patent Application Publication 2009/61288019, which is incorporated herein by reference.

“Ectopic expression or altered expression” in reference to a polynucleotide indicates that the pattern of expression in, e.g., a transgenic plant or plant tissue, is different from the expression pattern in a wild-type plant or plant tissue or a reference plant or plant tissue of the same species. For example, a polynucleotide or polypeptide is expressed in a cell or tissue type other than a cell or tissue type in which the sequence is expressed in the wild-type plant, or by expression at a time other than at the time the sequence is expressed in the wild-type plant, or by a response to different inducible agents, such as hormones or environmental signals, or at different expression levels (either higher or lower) compared with those found in a wild-type plant. The term also refers to altered expression patterns that are produced by lowering the levels of expression to below the detection level or completely abolishing expression. The resulting expression pattern can be transient or stable, constitutive or inducible. In reference to a polypeptide, the term “ectopic expression or altered expression” further may relate to altered activity levels resulting from the interactions of the polypeptides with exogenous or endogenous modulators or from interactions with factors or as a result of the chemical modification of the polypeptides.

The term “overexpression” as used herein refers to a greater expression level of a gene in a plant, plant cell or plant tissue, compared to expression in a wild-type plant, cell or tissue, at any developmental or temporal stage for the gene. Overexpression can occur when, for example, the genes encoding one or more polypeptides are under the control of a strong promoter (e.g., the cauliflower mosaic virus 35S transcription initiation region). Overexpression may also be under the control of a heterologous promoter, or an inducible or tissue specific promoter. Thus, overexpression may occur throughout a plant, in specific tissues of the plant, or in the presence or absence of particular environmental signals, depending on the promoter used. Overexpression may take place in plant cells normally lacking expression of polypeptides functionally equivalent or identical to the present polypeptides. Overexpression may also occur in plant cells where endogenous expression of the present polypeptides or functionally equivalent molecules normally occurs, but such normal expression is at a lower level. Overexpression thus results in a greater than normal production, or “overproduction” of the polypeptide in the plant, cell or tissue.

The term “transcription regulating region” refers to a DNA regulatory sequence that regulates the expression of one or more genes in a plant with a transcriptional regulator having one or more specific binding domains that binds to a DNA regulatory sequence. Transcriptional regulators possess a conserved domain. The transcriptional regulators also comprise an amino acid subsequence that forms a transcription activation domain that regulates expression of one or more abiotic stress tolerance genes in a plant when the transcriptional regulator binds to the regulating region.

“Increased yield” or “improved yield” or “increased plant yield” refers to increased plant growth, increased crop growth, increased biomass, increased grain yield, and/or increased plant product production, and is dependent to some extent on temperature, plant size, organ size, planting density, light, water and nutrient availability, and how the plant copes with various stresses, such as through temperature acclimation and water or nutrient use efficiency.

“Planting density” refers to the number of plants that can be grown per acre. For crop species, planting or population density varies from crop to crop, from one growing region to another, and from year to year. Using corn as an example, the average prevailing density in 2000 was in the range of 20,000-25,000 plants per acre in Missouri, USA. A desirable higher population density (a measure of yield) would be at least 22,000 plants per acre, and a more desirable higher population density would be at least 28,000 plants per acre, more preferably at least 34,000 plants per acre, and even more preferably at least 40,000 plants per acre. The average prevailing densities per acre of a few other examples of crop plants in the USA in the year 2000 were: wheat 1,000,000-1,500,000; rice 650,000-900,000; soybean 150,000-200,000, canola 260,000-350,000, sunflower 17,000-23,000 and cotton 28,000-55,000 plants per acre (Cheikh et al., 2003, in U.S. Patent Application No. 20030101479). A desirable higher population density for each of these examples, as well as other valuable species of plants, would be at least 10% higher than the average prevailing density or yield.

### Description of the Specific Embodiments

### Transcriptional Regulators Modify Expression of Endogenous Genes

A transcriptional regulator may include, but is not limited to, any polypeptide that can activate or repress transcription of a single gene or a number of genes. As one of ordinary skill in the art recognizes, transcriptional regulators can be identified by the presence of a region or domain of structural similarity or identity to a specific consensus sequence or the presence of a specific consensus DNA-binding motif (see, for example, Riechmann et al., 2000a). The plant transcriptional regulators of the present disclosure belong to the B-box zinc finger family (Putterill et al., 1995) and are putative transcriptional regulators.

Generally, transcriptional regulators are involved in cell differentiation and proliferation and the regulation of growth. Accordingly, one skilled in the art would recognize that by expressing the present sequences in a plant, one may change the expression of autologous genes or induce the expression of introduced genes. By affecting the expression of similar autologous sequences in a plant that have the biological activity of the present sequences, or by introducing the present sequences into a plant, one may alter a plant's phenotype to one with improved traits related to osmotic stresses. The disclosed sequences may also be used to transform a plant and introduce desirable traits not found in the wild-type cultivar or strain. Plants may then be selected for those that produce the most desirable degree of over- or under-expression of target genes of interest and coincident trait improvement.

The sequences of the present disclosure and closely related orthologous or paralogous sequences may be from any species, particularly plant species, in a naturally occurring form or from any source whether natural, synthetic, semi-synthetic or recombinant. The disclosed sequences may also include fragments of the present amino acid sequences. Where “amino acid sequence” is recited to refer to an amino acid sequence of a naturally occurring protein molecule, “amino acid sequence” and like terms are not meant to limit the amino acid sequence to the complete native amino acid sequence associated with the recited protein molecule.

In addition to methods for modifying a plant phenotype by employing one or more of the disclosed polynucleotides and polypeptides, the disclosed polynucleotides and polypeptides have a variety of additional uses. These uses include their use in the recombinant production (i.e., expression) of proteins; as regulators of plant gene expression, as diagnostic probes for the presence of complementary or partially complementary nucleic acids (including for detection of natural coding nucleic acids); as substrates for further reactions, e.g., mutation reactions, PCR reactions, or the like; as substrates for cloning e.g., including digestion or ligation reactions; and for identifying exogenous or endogenous modulators of the transcriptional regulators. The polynucleotide can be, e.g., genomic DNA or RNA, a transcript (such as an mRNA), a cDNA, a PCR product, a cloned DNA, a synthetic DNA or RNA, or the like. The polynucleotide can comprise a sequence in either sense or antisense orientations. Expression of genes that encode polypeptides that modify expression of endogenous genes, polynucleotides, and proteins are well known in the art. In addition, transgenic plants comprising isolated polynucleotides encoding transcriptional regulators may also modify expression of endogenous genes, polynucleotides, and proteins. Examples include Peng et al., 1997, and Peng et al., 1999). In addition, many others have demonstrated that an Arabidopsis transcriptional regulator expressed in an exogenous plant species elicits the same or very similar phenotypic response. See, for example, Fu et al., 2001; Nandi et al., 2000; Coupland, 1995; and Weigel and Nilsson, 1995).

In another example, Mandel et al., 1992b, and Suzuki et al., 2001, teach that a transcriptional regulator expressed in another plant species elicits the same or very similar phenotypic response of the endogenous sequence, as often predicted in earlier studies of Arabidopsis transcriptional regulators in Arabidopsis (see Mandel et al., 1992a; Suzuki et al., 2001). Other examples include Muller et al., 2001; Kim et al., 2001; Kyozuka and Shimamoto, 2002; Boss and Thomas, 2002; He et al., 2000; and Robson et al., 2001).

In yet another example, Gilmour et al., 1998, teach an Arabidopsis AP2 transcriptional regulator, CBF1, which, when overexpressed in transgenic plants, increased plant freezing tolerance. Jaglo et al., 2001) further identified sequences in Brassica napus which encode CBF-like genes and showed that transcripts for these genes accumulated rapidly in response to low temperature. Transcripts encoding CBF-like proteins were also found to accumulate rapidly in response to low temperature in wheat, as well as in tomato. An alignment of the CBF proteins from Arabidopsis, B. napus, wheat, rye, and tomato revealed the presence of conserved consecutive amino acid residues which bracket the AP2/EREBP DNA binding domains of the proteins and distinguish them from other members of the AP2/EREBP protein family (Jaglo et al., 2001).

Transcriptional regulators mediate cellular responses and control traits through altered expression of genes containing cis-acting nucleotide sequences that are targets of the introduced transcriptional regulator. It is well appreciated in the art that the effect of a transcriptional regulator on cellular responses or a cellular trait is determined by the particular genes whose expression is either directly or indirectly (e.g., by a cascade of transcriptional regulator binding events and transcriptional changes) altered by transcriptional regulator binding. In a global analysis of transcription comparing a standard condition with one in which a transcriptional regulator is overexpressed, the resulting transcript profile associated with transcriptional regulator overexpression is related to the trait or cellular process controlled by that transcriptional regulator. For example, the PAP2 gene and other genes in the MYB family have been shown to control anthocyanin biosynthesis through regulation of the expression of genes known to be involved in the anthocyanin biosynthetic pathway (Bruce et al., 2000; and Borevitz et al., 2000). Consequently, it is evident to one skilled in the art that similarity of transcript profile upon overexpression of different transcriptional regulators would indicate similarity of transcriptional regulator function.

### The Disclosed Polypeptides and Polynucleotides

The present disclosure includes putative transcriptional regulators, and isolated or recombinant polynucleotides encoding the polypeptides, or novel sequence variant polypeptides or polynucleotides encoding novel variants of polypeptides derived from the specific sequences provided in the Sequence Listing; the disclosed recombinant polynucleotides may be incorporated in expression vectors for the purpose of producing transformed plants. Also provided are methods for modifying yield from a plant by modifying the mass, size or number of plant organs or seed of a plant by controlling a number of cellular processes, and for increasing a plant's resistance to abiotic stresses. These methods are based on the ability to alter the expression of critical regulatory molecules that may be conserved between diverse plant species. Related conserved regulatory molecules may be originally discovered in a model system such as Arabidopsis and homologous, functional molecules then discovered in other plant species. The latter may then be used to confer increased yield or abiotic stress tolerance in diverse plant species.

Exemplary polynucleotides encoding the polypeptides provided in Table 1 and Table 2 were identified in the Arabidopsis thaliana GenBank database using publicly available sequence analysis programs and parameters, and an in-house proprietary database. Sequences initially identified were then further characterized to identify sequences comprising specified sequence strings corresponding to sequence motifs present in families of known polypeptides. Additional disclosed polynucleotides were identified by screening Arabidopsis thaliana and/or other plant cDNA libraries with probes corresponding to known polypeptides under low stringency hybridization conditions. Additional sequences, including full length coding sequences, were subsequently recovered by the rapid amplification of cDNA ends (RACE) procedure using a commercially available kit according to the manufacturer's instructions. Where necessary, multiple rounds of RACE are performed to isolate 5′ and 3′ ends. The full-length cDNA was then recovered by a routine end-to-end polymerase chain reaction (PCR) using primers specific to the isolated 5′ and 3′ ends. Exemplary sequences are provided in the sequence listing.

Many of the sequences in the Sequence Listing, derived from diverse plant species, have been ectopically expressed in overexpressor plants. The changes in the characteristic(s) or trait(s) of the plants were then observed and found to confer increased yield and/or increased abiotic stress tolerance. Therefore, the polynucleotides and polypeptides can be used to improve desirable characteristics of plants.

The disclosed polynucleotides were also ectopically expressed in overexpressor plant cells and the changes in the expression levels of a number of genes, polynucleotides, and/or proteins of the plant cells observed. Therefore, the polynucleotides and polypeptides can be used to change expression levels of genes, polynucleotides, and/or proteins of plants or plant cells.

### General Considerations Related to Improving Plant Yield

The instant disclosure provides a novel method involving new gene technologies to improve plant yield. It is expected that either individually or in a combination with other genes or promoters, these novel methods will provide significantly higher improvements compared to previous methods.

Plants have evolved to sense the direction, color, intensity and duration of the incumbent light signals. Plants use this information to redirect their growth and developmental processes to optimize light absorption by promoting elongation growth under low light. Several genes have been implicated in playing roles in light signaling mechanisms regulating plant growth and development. Here, light responsiveness of young seedlings overexpressing some of the individual B-box genes to determine their roles in light signaling was examined. There is precedent that suppressing light signaling leads to crop improvement (U.S. Pat. No. 7,692,067).

The instant disclosure identifies several Arabidopsis thaliana B-box genes which can function negatively in light signaling and it is expected that either independently or in combination with other genes (such as with AtBBX32, GmBBX52 or GmBBX53) or specific promoters, these polynucleotides and their encoded polypeptides will provide significantly improved yield benefits and resistance to abiotic stress. It is also expected that these polynucleotides and their encoded polypeptides will provide plants with altered levels of ureides, altered levels of hexose sugars, altered SPS activity, altered levels of starch, and delayed senescence (International Application No. PCT/US2012/029885),It is further expected that the functionally similar orthologs or paralogs of these polynucleotides and their encoded polypeptides, either expressed individually or in combination with other polynucleotides and their encoded polypeptides, will lead to plants with higher yield advantages, improved stress tolerance, altered levels of ureides, altered levels of hexose sugars, altered SPS activity, altered levels of starch, and delayed senescence.

The instant disclosure also identifies several Glycine max B-box genes. Some of the encoded polypeptides of these Glycine max genes may interact with AtBBX32, or may be overexpressed or suppressed, in transgenic soybean plants overexpressing AtBBX32. Some of the encoded polypeptides of these Glycine max genes may interact with GmBBX52 in transgenic soybean plants overexpressing GmBBX52. Since expression of AtBBX32 and its soybean homologs, GmBBX52 and GmBBX53, modulates a variety of traits including yield, it is expected that either independently or in combination with other genes (such as with AtBBX32, GmBBX52 or GmBBX53) or specific promoters, altering the levels of these polynucleotides and their encoded polypeptides will provide plants with significantly improved yield benefits, resistance to abiotic stress, altered levels of ureides, altered levels of hexose sugars, altered SPS activity, altered levels of starch, and delayed senescence. It is also expected that the functionally similar orthologs or paralogs of these polynucleotides and their encoded polypeptides, either expressed individually or in combination with other polynucleotides and their encoded polypeptides, will lead to plants with higher yield advantages, improved stress tolerance, altered levels of ureides, altered levels of hexose sugars, altered SPS activity, altered levels of starch, and delayed senescence.

### Background Information for BBX32, the BBX32 Clade, and Related Sequences

BBX32 is a B-box zinc finger protein with homology to the CONSTANS family of transcriptional regulators. BBX32 is expressed in many tissues and may be diurnally regulated.

As disclosed below in the Examples, constitutive expression of BBX32 in Arabidopsis modulates plant growth processes, including elongation of hypocotyls, extended petioles and upheld leaves, early flowering; enhanced root and/or shoot growth in phosphate-limited media; more secondary roots on control media, enhanced growth and reduced anthocyanin in low nitrogen/high sucrose media supplemented with glutamine, enhanced root growth on salt-containing media, and enhanced root growth on polyethylene glycol-containing media, as compared to control plants. BBX32 overexpression in soybean plants has been shown to result in a statistically significant increase in yield in field trials (see U.S. Pat. No. 7,692,067) as compared to controls that do not contain the BBX32 polynucleotide.

As noted in the Examples presented below, overexpression of BBX32 in Arabidopsis produced several phenotypes consistent with a role of this gene in light regulated development and possibly diurnal or circadian regulated processes. Light exerts a major influence in the initiation and maintenance (re-setting) of the plant circadian processes. Early seedling development is light dependent (de-etiolation) and is marked by inhibition of hypocotyl cell elongation along with cotyledon expansion and greening. Light controls morphological changes throughout the life of the plant, either directly or by regulating circadian gene expression. Some of these changes include petiole development, control of growth rate and flowering. It is likely that the ectopic expression of BBX32 product affects light signaling and/or circadian processes. Based upon the observations described above, it is clear that BBX32 is involved in photomorphogenesis and regulating plant growth and development. Hence, its overexpression may improve plant vigor, thus explaining the yield enhancements seen in 35S::BBX32 soybean plants.

TABLE 1
Conserved domains and potentially valuable morphological traits of B-box sequences

| | | | | | | |
|---|---|---|---|---|---|---|
| | | | | | | Column 7 |
| | | Column 3 | | | | Percent |
| Column 1 | | Percent | | | | identity of |
| Poly- | | identity of | Column 4 | | Column 6 | domain in |
| peptide | | polypeptide | Amino acid | | SEQ ID | column 5 |
| SEQ | Column 2 | to clade | coordinates | Column 5 | NO: of | (no. identical |
| ID NO: | Species/GID No. | identifier | of domain | Domain sequence | domain | residues) |

AtBBX28 (G1481)clade (clade identifier G1481):

| | | | | | | |
|---|---|---|---|---|---|---|
| 2 | At/G1481 | 100.0% to | 5-41 | CDLCNGVARMYCESD | 235 | 100% |
| | | G1481 | | QASLCWDCDGKVHGA | | (37/37) |
| | | | | NFLVAKH | | |
| 4 | At/G900 | 48.4% to | 6-42 | CELCCGVARMYCESD | 236 | 95% |
| | | G1481 | | QASLCWDCDGKVHGA | | (37/37) |
| | | | | NFLVAKH | | |
| 6 | Zm/ACN33981.1 | 35.3% to | 28-64 | CELCGAAARVYCGAD | 237 | 65% |
| | | G1481 | | EATLCWGCDAQVHGA | | (24/37) |
| | | | | NFLVARH | | |
| 8 | Pt/Pt_568988 | 37.1% to | 89-125 | CELCGSSARMYCESD | 238 | 84% |
| | | G1481 | | QASLCWDCDEKVHTA | | (31/37) |
| | | | | NFLVAKH | | |
| 10 | Pt/Pt_571834 | 26.5% to | 4-40 | CELCKNPARTYCESD | 239 | 73% |
| | | G1481 | | EANLCWNCDTKVHGA | | (27/37) |
| | | | | NFLVARH | | |
| 12 | Pt/Pt_582065 | 35.4% to | 4-40 | CELCGSSARMFCESD | 240 | 81% |
| | | G1481 | | QASLCWDCDEKVHSA | | (30/37) |
| | | | | NFLVAKH | | |
| 14 | Pt/Pt_758524 | 28.3% to | 4-40 | CELCDSFAQMHCESD | 241 | 65% |
| | | G1481 | | QAILCSACDAYVHSA | | (24/37) |
| | | | | NFLAAKH | | |
| 16 | Os/Os08g08120.1 | 34.8% to | 24-60 | CELCGAAARVYCGAD | 242 | 65% |
| | | G1481 | | EATLCWGCDAQVHGA | | (24/37) |
| | | | | NFLVARH | | |
| 18 | Os/Os08g08120.2 | 36.0% to | 24-60 | CELCGAAARVYCGAD | 243 | 65% |
| | | G1481 | | EATLCWGCDAQVHGA | | (24/37) |
| | | | | NFLVARH | | |
| 20 | Os/Os08g08120.3 | 36.3% to | 24-60 | CELCGAAARVYCGAD | 244 | 65% |
| | | G1481 | | EATLCWGCDAQVHGA | | (24/37) |
| | | | | VFLVARH | | |
| 22 | Gm/Glyma06g39810.1 | 41.0% to | 8-44 | CVLCEKRAMMLCDSD | 245 | 70% |
| | | G1481 | | QAKLCWECDEKVHSA | | (26/37) |
| | | | | NFLVAKH | | |
| 24 | Gm/Glyma06g45620.1 | 38.9% to | 4-40 | CELCKVPARIFCESD | 246 | 76% |
| | | G1481 | | QASLCWDCDAKVHSA | | (27/37) |
| | | | | NFLVARH | | |
| 26 | Gm/G4015 | 23.7% to | 6-42 | CELCHQLASLYCPSD | 247 | 59% |
| | | G1481 | | SAFLCFHCDAAVHAA | | (22/37) |
| | | | | NFLVARH | | |
| 28 | Gm/Glyma09g35960.1 | 21.0% to | 6-42 | CALCKKRAMMLCDSD | 248 | 70% |
| | | G1481 | | QAKLCWECDEKVHSA | | (26/37) |
| | | | | NFLVAKH | | |
| 30 | Gm/Glyma12g11230.1 | 37.1% to | 4-40 | CELCKVPARIFCESD | 249 | 76% |
| | | G1481 | | QASLCWDCDAKVHSA | | (28/37) |
| | | | | NFLVARH | | |
| 32 | Gm/Glyma12g32470.1 | 41.5% to | 4-40 | CELCKLPARTFCESD | 250 | 78% |
| | | G1481 | | QASLCWDCDAKVHGA | | (29/37) |
| | | | | NFLVARH | | |
| 34 | Gm/G4014 | 38.1% to | 4-40 | CELCNSPAKLFCESD | 251 | 73% |
| | | G1481 | | QASLCWKCDAKVHSA | | (27/37) |
| | | | | NFLVTKH | | |
| 36 | Gm/G4016 | 39.6% to | 4-40 | CELCKLPARTFCESD | 252 | 76% |
| | | G1481 | | QASLCWDCDAKVHGA | | (28/37) |
| | | | | NFLVERH | | |
| 38 | Gm/Glyma15g07140.1 | 37.2% to | 4-40 | CELCNSPAKLFCESD | 253 | 73% |
| | | G1481 | | QASLCWECDAKVHSA | | (27/37) |
| | | | | NFLVTKH | | |

AtBBX25 (G1894) clade (clade identifier G1894):

| | | | | | | |
|---|---|---|---|---|---|---|
| 152 | At/G329 | 69.2% to | 5-42^{a} | CDVCEKAPATVICCA | 254 | 87% |
| | | G1894 | | DEAALCPQCDIEIHA | | (33/38) |
| | | | | ANKLASKH | | |
| 152 | At/G329 | 69.2% to | 57-94^{b} | CDICQEKAAFIFCVE | 255 | 84% |
| | | G1894 | | DRALLCRDCDESIHV | | (32/38) |
| | | | | ANSRSANH | | |
| 154 | At/G1894 | 100.0% to | 5-42^{a} | CDVCEKAPATLICCA | 256 | 100% |
| | | G1894 | | DEAALCAKCDVEVHA | | (38/38) |
| | | | | ANKLASKH | | |
| 154 | At/G1894 | 100.0% to | 57-94^{b} | CDICLEKAAFIFCVE | 257 | 100% |
| | | G1894 | | DRALLCRDCDEATHA | | (38/38) |
| | | | | PNTRSANH | | |
| 156 | Zm/ACF84591.1 | 50.0% to | 5-42^{a} | CDACEGAAATVVCCA | 258 | 82% |
| | | G1894 | | DEAALCARCDVEIHA | | (31/38) |
| | | | | ANKLASKH | | |
| 156 | Zm/ACF84591.1 | 50.0% to | 57-94^{b} | CDVCQEKAAFIFCVE | 259 | 74% |
| | | G1894 | | DRALFCQDCDEPIHV | | (28/38) |
| | | | | PGTLSGNH | | |
| 158 | Zm/ACF86823.1 | 50.4% to | 5-42^{a} | CDACEGAAATVVCCA | 260 | 82% |
| | | G1894 | | DEAALCARCDVEIHA | | (31/38) |
| | | | | ANKLASKH | | |
| 158 | Zm/ACF86823.1 | 50.4% to | 57-94^{b} | CDVCQEKAAFIFCVE | 261 | 76% |
| | | G1894 | | DRALFCRDCDEPIHV | | (29/38) |
| | | | | PGTLSGNH | | |
| 162 | Zm/CAW36812.1 | 50.4% to | 5-42^{a} | CDACEGAAATVVCCA | 262 | 82% |
| | | G1894 | | DEAALCARCDVEIHA | | (31/38) |
| | | | | ANKLASKH | | |
| 162 | Zm/CAW36812.1 | 50.4% to | 57-94^{b} | CDVCQEKAAFIFCVE | 263 | 76% |
| | | G1894 | | DRALFCRDCDEPIHV | | (29/38) |
| | | | | PGTLSGNH | | |
| 176 | Pt/Pt_216458 | 45.4% to | 5-42^{a} | CDVCEKAPATVICCA | 264 | 92% |
| | | G1894 | | DEAALCEKCDIEVII | | (35/38) |
| | | | | AANKLASKH | | |
| 176 | Pt/Pt_216458 | 45.4% to | 57-94^{b} | CDICQEKAAFIFCVE | 265 | 76% |
| | | G1894 | | DRALFCRDCDEPIHS | | (29/38) |
| | | | | AGSLSANH | | |
| 178 | Pt/Pt_827145 | 62.7% to | 5-42^{a} | CDVCEKAPATVICCA | 266 | 95% |
| | | G1894 | | DEAALCAKCDIEVHA | | (36/38) |
| | | | | ANKLASKH | | |
| 178 | Pt/Pt_827145 | 62.7% to | 57-94^{b} | CDICQEKAAFIFCVE | 267 | 76% |
| | | G1894 | | DRALFCRDCDEPIHS | | (29/38) |
| | | | | AGSLSANH | | |
| 180 | Os/Os02g39360.1 | 49.6% to | 5-42^{a} | CDACESAAAAVVCCA | 268 | 79% |
| | | G1894 | | DEAALCAACDVEVHA | | (30/38) |
| | | | | ANKLAGKH | | |
| 180 | Os/Os02g39360.1 | 49.6% to | 57-94^{b} | CDVCQEKAAFIFCVE | 269 | 76% |
| | | G1894 | | DRALFCRDCDEPIHV | | (29/38) |
| | | | | PGTLSGNH | | |
| 182 | Os/Os04g41560.2 | 48.1% to | 5-42^{a} | CDACEAAAATVVCCA | 270 | 82% |
| | | G1894 | | DEAALCARCDVEIHA | | (31/38) |
| | | | | ANKLASKH | | |
| 182 | Os/Os04g41560.2 | 48.1% to | 58-95^{b} | CDVCQEKAAFIFCVE | 271 | 76% |
| | | G1894 | | DRALFCRDCDEPIHV | | (29/38) |
| | | | | PGTLSGNH | | |
| 184 | Os/Os04g41560.4 | 41.8% to | 5-42^{a} | CEACEAAAATVVCCA | 272 | 82% |
| | | G1894 | | DEAALCARCDVEIHA | | (31/38) |
| | | | | ANKLASKH | | |
| 184 | Os/Os04g41560.4 | 41.8% to | 58-95^{b} | CDVCQEKAAFIFCVE | 273 | 76% |
| | | G1894 | | DRALFCRDCDEPIHV | | (29/38) |
| | | | | PGTLSGNH | | |
| 186 | Gm/Glyma11g13570 | 58.9% to | 5-42^{a} | CDVCEKAPATVICCA | 274 | 97% |
| | | G1894 | | DEAALCAKCDVEVHA | | (37/38) |
| | | | | ANKLASKH | | |
| 186 | Gm/Glyma11g13570.1 | 58.9% to | 57-94^{b} | CDICQDKPAFIFCVE | 275 | 68% |
| | | G1894 | | DRALFCKDCDEPIHL | | (26/38) |
| | | | | ASSLSANH | | |
| 188 | Gm/Glyma12g05570.1 | 58.7% to | 5-42^{a} | CDVCEKAPATVICCA | 276 | 97% |
| | | G1894 | | DEAALCAKCDVEVHA | | (37/38) |
| | | | | ANKLASKH | | |
| 188 | Gm/Glyma12g05570.1 | 58.7% to | 57-94^{b} | CDICQDKPAFIFCVE | 277 | 68% |
| | | G1894 | | DRALFCKDCDEPIHL | | (26/38) |
| | | | | ASSLSANH | | |
| 190 | Gm/Glyma13g41980.1 | 58.1% to | 5-42^{a} | CDVCERAPATVICCA | 278 | 95% |
| | | G1894 | | DEAALCAKCDVEVHA | | (36/38) |
| | | | | ANKLASKH | | |
| 190 | Gm/Glyma13g41980.1 | 58.1% to | 57-94^{b} | CDICQDKPAFIFCVE | 279 | 68% |
| | | G1894 | | DRALFCQDCDEPIHS | | (26/38) |
| | | | | AGSLSANH | | |
| 192 | Gm/Glyma13g41908.2 | 39.1% to | 24-57^{a} | QDKPAFIFCVEDRAL | 280 | 35% |
| | | G1894 | | FCQDCDEPIHSAGSL | | (12/34) |
| | | | | SANH | | |
| 192 | Gm/Glyma13g41908.2 | 39.1% to | 25-57^{b} | DKPAFIFCVEDRALF | 281 | 67% |
| | | G1894 | | CQDCDEPIHSAGSLS | | (22/33) |
| | | | | ANH | | |
| 194 | Gm/Glyma13g41980.3 | 39.1% to | 24-57^{a} | QDKPAFIFCVEDRAL | 282 | 35% |
| | | G1894 | | FCQDCDEPIHSAGSL | | (12/34) |
| | | | | SANH | | |
| 194 | Gm/Glyma13g41980.3 | 39.1% to | 25-57^{b} | DKPAFIFCVEDRALF | 283 | 67% |
| | | G1894 | | CQDCDEPIHSAGSLS | | (22/33) |
| | | | | ANH | | |
| 196 | Gm/Glyma15g03400.1 | 57.9% to | 5-42^{a} | CDVCERAPATVICCA | 284 | 95% |
| | | G1894 | | DEAALCAKCDVEVHA | | (36/38) |
| | | | | ANKLASKII | | |
| 196 | Gm/Glyma15g03400.1 | 57.9% to | 57-94^{b} | CDICQDKPAFIFCVE | 285 | 68% |
| | | G1894 | | DRALFCQDCDEPIHS | | (26/38) |
| | | | | AGSLSANH | | |
| 198 | Sl/AAS67368 | 58.2% to | 5-42^{a} | CDVCEKAQATVICCA | 286 | 92% |
| | | G1894 | | DEAALCAKCDIEVHA | | (35/38) |
| | | | | ANKLASKH | | |
| 198 | Sl/AAS67368 | 58.2% to | 57-94^{b} | CDICQDKAAFIFCVE | 287 | 68% |
| | | G1894 | | DRALFCKDCDEAIHS | | (26/38) |
| | | | | ASSLAKNH | | |

AtBBX20 (G1888) clade (clade identifier G1888):

| | | | | | | |
|---|---|---|---|---|---|---|
| 54 | At/G1888 | 100.0% to | 5-42^{a} | CAVCDKEEASVFCCA | 288 | 100% |
| | | G1888 | | DEAALCNGCDRHVHF | | (38/38) |
| | | | | ANKLAGKH | | |
| 54 | At/G1888 | 100.0% to | 58-95^{b} | CDICGERRALLFCQE | 289 | 100% |
| | | G1888 | | DRAILCRECDIPIHQ | | (38/38) |
| | | | | ANEHTKKH | | |
| 56 | At/G1482 | 37.4% to | 5-42^{a} | CDVCDKEEASVFCTA | 290 | 79% |
| | | G1888 | | DEASLCGGCDHQVHH | | (30/38) |
| | | | | ANKLASKH | | |
| 56 | At/G1482 | 37.4% to | 60-97^{b} | CDICQDKKALLFCQQ | 291 | 74% |
| | | G1888 | | DRAILCKDCDSSIHA | | (28/38) |
| | | | | ANEHTKKH | | |
| 58 | Zm/ACG44133.1 | 40.7% to | 5-42^{a} | CDVCAAEAASVFCCA | 292 | 76% |
| | | G1888 | | DEAALCDACDDRVHR | | (29/38) |
| | | | | ANKLAGKH | | |
| 58 | Zm/ACG44133.1 | 40.7% to | 61-98^{b} | CDICQERRGFLFCKE | 293 | 71% |
| | | G1888 | | DRAILCRECDAPVHS | | (27/38) |
| | | | | ANDMTRRH | | |
| 60 | Zm/CAW36814.1 | 44.4% to | 5-42^{a} | CDVCAAEAASVFCCA | 294 | 76% |
| | | G1888 | | DEAALCDACDDRVHR | | (29/38) |
| | | | | ANKLAGKH | | |
| 60 | Zm/CAW36814.1 | 44.4% to | 61-98^{b} | CDICQERRGFLFCKE | 295 | 71% |
| | | G1888 | | DRAILCRECDAPVHS | | (27/38) |
| | | | | ANDMTRRH | | |
| 68 | Pt/Pt_195637 | 51.0% to | 5-42^{a} | CDVCDNVEATVFCCA | 296 | 76% |
| | | G1888 | | DEAALCDGCDHRVHH | | (29/38) |
| | | | | ANKLASKH | | |
| 68 | Pt/Pt_195637 | 51.0% to | 58-95^{b} | CDICQERRALLFCQE | 297 | 89% |
| | | G1888 | | DRAILCRECDLPIHK | | (34/38) |
| | | | | ANEHTQKH | | |
| 70 | Pt/Pt_550941 | 39.5% to | 5-42^{a} | CDVCNKEEASVFCTA | 298 | 76% |
| | | G1888 | | DEAALCDTCDHRVHH | | (29/38) |
| | | | | ANKLASKH | | |
| 70 | Pt/Pt_550941 | 39.5% to | 58-95^{b} | CDICQEKRAFLFCQQ | 299 | 82% |
| | | G1888 | | DRAILCRECDGPIHT | | (31/38) |
| | | | | ANEHTQKH | | |
| 72 | Pt/Pt_801530 | 38.2% to | 5-42^{a} | CDVCSKEEASVFCTA | 300 | 76% |
| | | G1888 | | DEAALCDTCDHRVHH | | (29/38) |
| | | | | ANKLASKH | | |
| 72 | Pt/Pt_801530 | 38.2% to | 58-95^{b} | CDICQDKRAFLFCQQ | 301 | 76% |
| | | G1888 | | DRAILCRDCDGPIHT | | (29/38) |
| | | | | ANEHTQKH | | |
| 74 | Pt/Pt_804174 | 54.5% to | 5-42^{a} | CDVCDKCRATVFCCA | 302 | 76% |
| | | G1888 | | DEAALCDGCDHRVHH | | (29/38) |
| | | | | ANTLASKH | | |
| 74 | Pt/Pt_804174 | 54.5% to | 58-95^{b} | CDICQERRAVLFCQE | 303 | 84% |
| | | G1888 | | DRAILCRECDLPIHK | | (32/38) |
| | | | | VNEHTQKH | | |
| 76 | Os/Os02g43170.1 | 37.5% to | 5-42^{a} | CDVCAAEAASVFCCA | 304 | 76% |
| | | G1888 | | DEAALCDACDIIRVI | | (29/38) |
| | | | | IRANKLAGKH | | |
| 76 | Os/Os02g43170.1 | 37.5% to | 65-102^{b} | CDICQEKRGFLFCKE | 305 | 68% |
| | | G1888 | | DRAILCRECDVPVHT | | (26/38) |
| | | | | ASELTMRH | | |
| 78 | Os/Os02g43170.2 | 43.4% to | 5-42^{a} | CDVCAAEAASVFCCA | 306 | 76% |
| | | G1888 | | DEAALCDACDHRVHR | | (29/38) |
| | | | | ANKLAGKH | | |
| 78 | Os/Os02g43170.2 | 43.4% to | 65-102^{b} | CDICQEKRGFLFCKE | 307 | 68% |
| | | G1888 | | DRAILCRECDVPVHT | | (26/38) |
| | | | | ASELTMRH | | |
| 80 | Os/Os04g45690.1 | 37.7% to | 5-42^{a} | CDVCAAEAASVFCCA | 308 | 79% |
| | | G1888 | | DEAALCDACDRRVHS | | (30/38) |
| | | | | ANKLAGKH | | |
| 80 | Os/Os04g45690.1 | 37.7% to | 63-100^{b} | CDICQEKRGFLFCKE | 309 | 63% |
| | | G1888 | | DRAILCRECDVTVHT | | (24/38) |
| | | | | TSRLTRRH | | |
| 82 | Os/G5159 | 37.7% to | 5-42^{a} | CDVCAAEAASVFCCA | 310 | 79% |
| | | G1888 | | DEAALCDACDRRVHS | | (30/38) |
| | | | | ANKLAGKH | | |
| 82 | Os/G5159 | 37.7% to | 63-100^{b} | CDICQEKRGFLFCKE | 311 | 63% |
| | | G1888 | | DRAILCRECDVTVHT | | (24/38) |
| | | | | TSELTRRH | | |
| 84 | Os/Os06g49880.1 | 31.8% to | 5-42^{a} | CDVCAAEPAAVLCCA | 312 | 68% |
| | | G1888 | | DEAALCSACDRRVHR | | (26/38) |
| | | | | ANRLASKH | | |
| 84 | Os/Os06g49880.1 | 31.8% to | 66-103^{b} | CDVCREKRGLVFCVE | 313 | 66% |
| | | G1888 | | DRAILCADCDEPIHS | | (25/38) |
| | | | | ANDLTAKH | | |
| 86 | Os/Os06g49880.2 | 31.9% to | 5-42^{a} | CDVCAAEPAAVLCCA | 314 | 68% |
| | | G1888 | | DEAALCSACDRRVHR | | (26/38) |
| | | | | ANRLASKH | | |
| 86 | Os/Os06g49880.2 | 31.9% to | 66-103^{b} | CDVCREKRGLVFCVE | 315 | 66% |
| | | G1888 | | DRAILCADCDEPIHS | | (25/38) |
| | | | | ANDLTAKH | | |
| 88 | G/mGlyma04g01120.1 | 40.1% to | 5-42^{a} | CAVCDKVEASVFCSA | 316 | 79% |
| | | G1888 | | DEAALCHSCDRTIHH | | (30/38) |
| | | | | ANKLATKH | | |
| 88 | G/mGlyma04g01120.1 | 40.1% to | 58-95^{b} | CDICQERRAYLFCQE | 317 | 84% |
| | | G1888 | | DRALLCRECDVPIHR | | (32/38) |
| | | | | ANEHTQKH | | |
| 90 | Gm/G5365 | 39.4% to | 5-42^{a} | CDVCNKQASFFCTAD | 318 | 71% |
| | | G1888 | | EAALCDGCDHRVHHA | | (27/38) |
| | | | | NKLASKH | | |
| 90 | Gm/G5365 | | 58-95^{b} | CDVCQERRAFVFCQQ | 319 | |
| | | | | DRAILCKECDVPIHS | | |
| | | | | ANDLTKNH | | |
| 92 | Gm/Glyma04g02960.2 | 41.7% to | 5-42^{a} | CDVCNKHQASFFCTA | 320 | 71% |
| | | G1888 | | DEAALCDGCDHRVHH | | (27/38) |
| | | | | ANKLASKH | | |
| 92 | Gm/Glyma04g02960.2 | 41.7% to | 58-95^{b} | CDVCQERRAFVFCQQ | 321 | 71% |
| | | G1888 | | DRAILCKECDVPIHS | | (27/38) |
| | | | | ANDLTKNH | | |
| 94 | Gm/Glyma06g01140.1 | 49.4% to | 5-42^{a} | CDVCDKVEASVFCPA | 322 | 76% |
| | | G1888 | | DEAALCHSCDRTIHR | | (29/38) |
| | | | | ANKLATKH | | |
| 94 | Gm/Glyma06g01140.1 | 49.4% to | 58-95^{b} | CDICQERRAYLFCQE | 323 | 84% |
| | | G1888 | | DRALLCRECDVPIHR | | (32/38) |
| | | | | ANEHTQKH | | |
| 96 | Gm/G5367 | 39.1% to | 5-42^{a} | CDVCNKQQASLFCTA | 324 | 71% |
| | | G1888 | | DEAALCDGCDHRVHH | | (27/38) |
| | | | | ANKLASKH | | |
| 96 | Gm/G5367 | 39.1% to | 58-95^{b} | CDVCQERRAFVFCQQ | 325 | 68% |
| | | G1888 | | DRAILCKECDVPVHS | | (26/38) |
| | | | | ANDLTKNH | | |
| 98 | Gm/Glyma11g12060.1 | 40.8% to | 5-42^{a} | CDVCHNEVASFFCPS | 326 | 63% |
| | | G1888 | | DEASLCHACDRTIHH | | (24/38) |
| | | | | ANKLADKH | | |
| 98 | Gm/Glyma11g12060.1 | 40.8% to | 58-95^{b} | CDICHERRAYLFCKE | 327 | 82% |
| | | G1888 | | DRAILCRECDLSIHG | | (31/38) |
| | | | | VNEHTKKH | | |
| 100 | Gm/G5396 | 43.0% to | 5-42^{a} | CDVCNKHEASVFCTA | 328 | 76% |
| | | G1888 | | DEAALCDGCDHRVHH | | (29/38) |
| | | | | ANKLASKH | | |
| 100 | Gm/G5396 | 43.0% to | 58-95^{b} | CDICQERRAFTFCQQ | 329 | 76% |
| | | G1888 | | DRAILCKECDVSIHS | | (29/38) |
| | | | | ANEHTLKH | | |
| 102 | Gm/G5400 | 43.0% to | 5-42^{a} | CDVCNKHEASVFCTA | 330 | 76% |
| | | G1888 | | DEAALCDGCDHRVHH | | (29/38) |
| | | | | ANKLASKH | | |
| 102 | Gm/G5400 | 43.0% to | 58-95^{b} | CDICQERRAFTFCQQ | 331 | 76% |
| | | G1888 | | DRAILCKECDVSIHS | | (29/38) |
| | | | | ANEHTLKH | | |

AtBBX26 (G1486) clade (clade identifier G1486):

| | | | | | | |
|---|---|---|---|---|---|---|
| 52 | At/G1486 | 100.0% to | 5-41 | CHTCRHVTAVIHCVT | 332 | 100% |
| | | G1486 | | EALNFCLTCDNLRHH | | (37/37) |
| | | | | NNIHAEH | | |

AtBBX30 (G1478) or AtBBX31 (G1929) clade (clade identifier G1478):

| | | | | | | |
|---|---|---|---|---|---|---|
| 40 | At/G1478 | 100.0% to | 32-68 | CELCGENATVYCEAD | 333 | 100% |
| | | G1478 | | AAFLCRKCDRWVHSA | | (37/37) |
| | | | | NFLARRH | | |
| 42 | At/G1929 | 62.3% to | 31-67 | CELCDGDASVFCEAD | 334 | 78% |
| | | G1478 | | SAFLCRKCDRWVHGA | | (29/37) |
| | | | | NFLAWRII | | |
| 44 | Pt/Pt_562313 | 43.2% to | 32-68 | CELCGSRASLYCQAD | 335 | 73% |
| | | G1478 | | DAFLCQKCDKWVHGA | | (27/37) |
| | | | | NFLAQRH | | |
| 46 | Pt/Pt_594447 | 44.1% to | 31-67 | CELCGSRATLYCQAD | 336 | 76% |
| | | G1478 | | HAFLCQKCDGWVHGA | | (28/37) |
| | | | | NFLALRH | | |
| 48 | Gm/Glyma12g32230.1 | 43.8% to | 30-66 | CELCGLQASLYCQAD | 337 | 68% |
| | | G1478 | | DAYLCKKCDKRVHEA | | (25/37) |
| | | | | NFLALRH | | |
| 50 | Gm/G4019 | 40.6% to | 30-66 | CELCGLQASLYCQAD | 338 | 70% |
| | | G1478 | | DAYLCRKCDKRVHEA | | (26/37) |
| | | | | NFLALRH | | |

AtBBX7 (G2440) or AtBBX8 (G1479) clade (clade identifier G2440):

| | | | | | | |
|---|---|---|---|---|---|---|
| 104 | At/G2440 | 100.0% to | 5-42^{a} | CDFCGEQRSMVYCRS | 339 | 100% |
| | | G2440 | | DAACLCLSCDRSVHS | | (38/38) |
| | | | | ANALSKRH | | |
| 104 | At/G2440 | 100.0% to | 48-94^{b} | CERCNAQPATVRCVE | 340 | 96% |
| | | G2440 | | ERVSLCQNCDWSGHN | | (45/47) |
| | | | | NSNNNNSSSSTSTQQ | | |
| | | | | H | | |
| 104 | At/G2440 | 100.0% to | 315-356^{c} | RNNAVMRYKEKKKAR | 341 | 100% |
| | | G2440 | | KFDKRVRYASRKARA | | (42/42) |
| | | | | DVRRRVKGRFVK | | |
| 106 | At/G1479 | 70.1% to | 5-42^{a} | CDFCGEQRSMVYCRS | 342 | 97% |
| | | G2440 | | DAACLCLSCDRNVHS | | (37/38) |
| | | | | ANALSKRH | | |
| 106 | At/G1479 | 70.1% to | 48-87^{b} | CERCNAQPASVRCSD | 343 | 80% |
| | | G2440 | | ERVSLCQNCDWSGHD | | (32/40) |
| | | | | GKNSTTTSHH | | |
| 106 | At/G1479 | 70.1% to | 316-357^{c} | RNNAVMRYKEKKKAR | 344 | 95% |
| | | G2440 | | KFDKRVRYVSRKERA | | (40/42) |
| | | | | DVRRRVKGRFVK | | |
| 108 | Zm/ACF80403.1 | 49.6% to | 5-42^{a} | CGFCGKQRSMIYCRS | 345 | 87% |
| | | G2440 | | DAASLCLSCDRSVHS | | (33/38) |
| | | | | ANALSRRH | | |
| 108 | Zm/ACF80403.1 | 49.6% to | 48-85^{b} | CDRCGLQPASVRCLE | 346 | 55% |
| | | G2440 | | DNTSLCQNCDWNGHD | | (21/38) |
| | | | | AASGASGH | | |
| 108 | Zm/ACF80403.1 | 49.6% to | 349-390^{c} | RDSALTRYKEKKKKR | 347 | 76% |
| | | G2440 | | MFDKKIRYASRKARA | | (32/42) |
| | | | | DVRKRVKGRFIK | | |
| 110 | Zm/ACG34885.1 | 50.4% to | 5-42^{a} | CDFCGEQRSMVYCRS | 348 | 92% |
| | | G2440 | | DAASLCLSCDRNVHS | | (35/38) |
| | | | | ANALSRRH | | |
| 110 | Zm/ACG34885.1 | 50.4% to | 48-85^{b} | CERCASQPAMVRCLA | 349 | 58% |
| | | G2440 | | ENASLCQNCDWNGHI | | (22/38) |
| | | | | AGSSSAGH | | |
| 110 | Zm/ACG34885.1 | 50.4% to | 350-391^{c} | RDSALTRYKEKKMRR | 350 | 79% |
| | | G2440 | | KFDKKIRYASRKARA | | (33/42) |
| | | | | DVRKRVKGRFVK | | |
| 114 | Zm/ACN25813.1 | 47.1% to | 5-42^{a} | CDFCGKQRSMIYCRS | 351 | 87% |
| | | G2440 | | DAASLCLSCDRNVHS | | (33/38) |
| | | | | ANALSRRH | | |
| 114 | Zm/ACN25813.1 | 47.1% to | 48-85^{b} | CDRCGSQPASVRCLE | 352 | 55% |
| | | G2440 | | DNASLCQNCDWNGHD | | (21/38) |
| | | | | AESGASGH | | |
| 114 | Zm/ACN25813.1 | 47.1% to | 372-391^{c} | YASRKARADVRKRVK | 353 | 90% |
| | | G2440 | | GRFIK | | (18/20) |
| 120 | Pt/Pt_247140 | 59.7% to | 5-42^{a} | CDFCGEQRSMVYCRS | 354 | 95% |
| | | G2440 | | DAASLCLSCDRNVHS | | (36/38) |
| | | | | ANALSKRH | | |
| 120 | Pt/Pt_247140 | 59.7% to | 48-85^{b} | CERCNSQPALVRCAE | 355 | 74% |
| | | G2440 | | ERISLCQNCDWIGHG | | (28/38) |
| | | | | TSTSASTH | | |
| 120 | Pt/Pt_247140 | 59.7% to | 322-363^{c} | RSDAVMRYKEKKKTR | 356 | 86% |
| | | G2440 | | MFEKKVRYASRKARA | | (36/42) |
| | | | | DVRRRVKGRFVK | | |
| 122 | Pt/Pt_409151 | 59.9% to | 5-42^{a} | CDFCGEQRSMVYCRS | 357 | 95% |
| | | G2440 | | DAACLCLSCDQIVHS | | (36/38) |
| | | | | ANALSKRH | | |
| 122 | Pt/Pt_409151 | 59.9% to | 48-85^{b} | CERCNSQPALVRRVE | 358 | 71% |
| | | G2440 | | ERISLCQNCDWMGYG | | (27/38) |
| | | | | SSTSASTH | | |
| 122 | Pt/Pt_409151 | 59.9% to | 372-391^{c} | RSDAVKRYMEKKKTR | 359 | 83% |
| | | G2440 | | KFEKKVRYASRKARA | | (35/42) |
| | | | | DVRRRVKGRFVK | | |
| 124 | Os/Os02g49230.1 | 51.7% to | 5-42^{a} | CDFCREQRSMVYCRS | 360 | 89% |
| | | G2440 | | DAASLCLSCDRNVHS | | (34/38) |
| | | | | ANALSRRH | | |
| 124 | Os/Os02g49230.1 | 51.7% to | 48-85^{b} | CDRCVGQPAAVRCLE | 361 | 61% |
| | | G2440 | | ENTSLCQNCDWNGHG | | (23/38) |
| | | | | AASSAAGH | | |
| 124 | Os/Os02g49230.1 | 51.7% to | 350-391^{c} | RDNALTRYKEKKKRR | 362 | 83% |
| | | G2440 | | KFDKKIRYASRKARA | | (35/42) |
| | | | | DVRKRVKGRFVK | | |
| 126 | Os/Os02g49230.2 | 44.2% to | 78-115^{a} | CDFCREQRSMVYCRS | 363 | 89% |
| | | G2440 | | DAASLCLSCDRNVHS | | (34/38) |
| | | | | ANALSRRH | | |
| 126 | Os/Os02g49230.2 | 44.2% to | 121-158^{b} | CDRCVGQPAAVRCLE | 364 | 61% |
| | | G2440 | | ENTSLCQNCDWNGHG | | (23/38) |
| | | | | AASSAAGH | | |
| 126 | Os/Os02g49230.2 | 44.2% to | 423-464^{c} | RDNALTRYKEKKKRR | 365 | 83% |
| | | G2440 | | KFDKKIRYASRKARA | | (35/42) |
| | | | | DVRKRVKGRFVK | | |
| 128 | Os/Os02g49230.3 | 51.7% to | 5-42^{a} | CDFCREQRSMVYCRS | 366 | 89% |
| | | G2440 | | DAASLCLSCDRNVHS | | (34/38) |
| | | | | ANALSRRH | | |
| 128 | Os/Os02g49230.3 | 51.7% to | 48-85^{b} | CDRCVGQPAAVRCLE | 367 | 61% |
| | | G2440 | | ENTSLCQNCDWNGHG | | (23/38) |
| | | | | AASSAAGH | | |
| 128 | Os/Os02g49230.3 | 51.7% to | 350-391^{c} | RDNALTRYKEKKKRR | 368 | 83% |
| | | G2440 | | KFDKKIRYASRKARA | | (35/42) |
| | | | | DVRKRVKGRFVK | | |
| 130 | Os/Os02g49230.4 | 51.7% to | 5-42^{a} | CDFCREQRSMVYCRS | 369 | 89% |
| | | G2440 | | DAASLCLSCDRNVHS | | (34/38) |
| | | | | ANALSRRH | | |
| 130 | Os/Os02g49230.4 | 51.7% to | 48-85^{b} | CDRCVGQPAAVRCLE | 370 | 61% |
| | | G2440 | | ENTSLCQNCDWNGHG | | (23/38) |
| | | | | AASSAAGH | | |
| 130 | Os/Os02g49230.4 | 51.7% to | 350-391^{c} | RDNALTRYKEKKKRR | 371 | 83% |
| | | G2440 | | KFDKKIRYASRKARA | | (35/42) |
| | | | | DVRKRVKGRFVK | | |
| 132 | Os/Os02g49230.5 | 51.8% to | 5-42^{a} | CDFCREQRSMVYCRS | 372 | 89% |
| | | G2440 | | DAASLCLSCDRNVHS | | (34/38) |
| | | | | ANALSRRH | | |
| 132 | Os/Os02g49230.5 | 51.8% to | 48-85^{b} | CDRCVGQPAAVRCLE | 373 | 61% |
| | | G2440 | | ENTSLCQNCDWNGHG | | (23/38) |
| | | | | AASSAAGH | | |
| 132 | Os/Os02g49230.5 | 51.8% to | 349-390^{c} | RDNALTRYKEKKKRR | 374 | 83% |
| | | G2440 | | KFDKKIRYASRKARA | | (35/42) |
| | | | | DVRKRVKGRFVK | | |
| 134 | Os/Os02g49230.6 | 51.7% to | 5-42^{a} | CDFCREQRSMVYCRS | 375 | 89% |
| | | G2440 | | DAASLCLSCDRNVHS | | (34/38) |
| | | | | ANALSRRH | | |
| 134 | Os/Os02g49230.6 | 51.7% to | 48-85^{b} | CDRCVGQPAAVRCLE | 376 | 61% |
| | | G2440 | | ENTSLCQNCDWNGHG | | (23/38) |
| | | | | AASSAAGH | | |
| 134 | Os/Os02g49230.6 | 51.7% to | 350-391^{c} | RDNALTRYKEKKKRR | 377 | 83% |
| | | G2440 | | KFDKKIRYASRKARA | | (35/42) |
| | | | | DVRKRVKGRFVK | | |
| 136 | Os/Os06g19444.1 | 51.7% to | 5-42^{a} | CDFCGEQRSMVYCRS | 378 | 92% |
| | | G2440 | | DAASLCLSCDRNVHS | | (35/38) |
| | | | | ANALSRRH | | |
| 136 | Os/Os06g19444.1 | 51.7% to | 48-85^{b} | CDRCASQPAMVRCLV | 379 | 58% |
| | | G2440 | | ENASLCQNCDWNGHS | | (22/38) |
| | | | | AGSSAAGH | | |
| 136 | Os/Os06g19444.1 | 51.7% to | 373-392^{c} | YASRKARADVRKRVK | 380 | 95% |
| | | G2440 | | GRFVK | | (19/20) |
| 138 | Os/Os06g19444.2 | 51.8% to | 5-42^{a} | CDFCGEQRSMVYCRS | 381 | 92% |
| | | G2440 | | DAASLCLSCDRNVHS | | (35/38) |
| | | | | ANALSRRH | | |
| 138 | Os/Os06g19444.2 | 51.8% to | 48-85^{b} | CDRCASQPAMVRCLV | 382 | 58% |
| | | G2440 | | ENASLCQNCDWNGHS | | (22/38) |
| | | | | AGSSAAGH | | |
| 138 | Os/Os06g19444.2 | 51.8% to | 372-391^{c} | YASRKARADVRKRVK | 383 | 95% |
| | | G2440 | | GRFVK | | (19/20) |
| 140 | Gm/Glyma02g38870.1 | 56.3% to | 5-42^{a} | CDFCGDQRSLVYCRS | 384 | 87% |
| | | G2440 | | DSACLCLSCDRNVHS | | (33/38) |
| | | | | ANALSRRH | | |
| 140 | Gm/Glyma02g38870.1 | 56.3% to | 48-85^{b} | CERCNSQPAFVRSVE | 385 | 68% |
| | | G2440 | | EKISLCQNCDWLGHG | | (26/38) |
| | | | | TSPSSSMH | | |
| 140 | Gm/Glyma02g38870.1 | 56.3% to | 348-389^{c} | RSNAVMRYKEKKKTR | 386 | 88% |
| | | G2440 | | KFEKKVRYASRKARA | | (37/42) |
| | | | | DVRKRVKGRFVK | | |
| 142 | Gm/Glyma13g1590.1 | 36.6% to | | | | |
| | | G2440 | | | | |
| 142 | Gm/Glyma13g1590.1 | 36.6% to | | | | |
| | | G2440 | | | | |
| 142 | Gm/Glyma13g1590.1 | 36.6% to | 224-365^{c} | RSNAVMRYKEKKKTR | 387 | 90% |
| | | G2440 | | KFDKKVRYASRKARA | | (38/42) |
| | | | | DVRRRVKGRFVK | | |
| 144 | Gm/Glyma14g36930.1 | 55.3% to | 5-42^{a} | CDFCGDQRSLVYCRS | 388 | 89% |
| | | G2440 | | DAACLCLSCDRNVHS | | (34/38) |
| | | | | ANALSRRH | | |
| 144 | Gm/Glyma14g36930.1 | 55.3% to | 48-85^{b} | CERCNSQPAFVRCVD | 389 | 68% |
| | | G2440 | | EKISLCQNCDWLGHG | | (26/38) |
| | | | | TSPSSSTH | | |
| 144 | Gm/Glyma14g36930.1 | 55.3% to | 354-395^{c} | RSNAVMRYKEKKKTR | 390 | 88% |
| | | G2440 | | KFEKKVRYASRKARA | | (37/42) |
| | | | | DVRKRVKGRFVK | | |
| 146 | Gm/Glyma14g36930.2 | 55.3% to | 5-42^{a} | CDFCGDQRSLVYCRS | 391 | 89% |
| | | G2440 | | DAACLCLSCDRNVHS | | (34/38) |
| | | | | ANALSRRH | | |
| 146 | Gm/Glyma14g36930.2 | 55.3% to | 48-85^{b} | CERCNSQPAFVRCVD | 392 | 68% |
| | | G2440 | | EKISLCQNCDWLGHG | | (26/38) |
| | | | | TSPSSSTH | | |
| 146 | Gm/Glyma14g36930.2 | 55.3% to | 354-395^{c} | RSNAVMRYKEKKKTR | 393 | 88% |
| | | G2440 | | KFEKKVRYASRKARA | | (37/42) |
| | | | | KVRKRVKGRFVK | | |
| 148 | Gm/Glyma20g07050.1 | 37.4% to | | | | |
| | | G2440 | | | | |
| 148 | Gm/Glyma20g07050.1 | 37.4% to | | | | |
| | | G2440 | | | | |
| 148 | Gm/Glyma20g07050.1 | 37.4% to | 226-267^{c} | RSNAVMRYKEKKKTR | 394 | 90% |
| | | G2440 | | MFDKKVRYASRKARA | | (38/42) |
| | | | | DVRRRVKGRFVK | | |
| 150 | Gm/Glyma20g07050.2 | 37.4% to | | | | |
| | | G2440 | | | | |
| 150 | Gm/Glyma20g07050.2 | 37.4% to | | | | |
| | | G2440 | | | | |
| 150 | Gm/Glyma20g07050.2 | 37.4% to | 226-267^{c} | RSNAVMRYKEKKKTR | 395 | 90% |
| | | G2440 | | MFDKKVRYASRKARA | | (38/42) |
| | | | | DVRRRVKGRFVK | | |

AtBBX19 (G902) or AtBBX18 (G1881) clade (clade identifier G902):

| | | | | | | |
|---|---|---|---|---|---|---|
| 200 | At/G902 | 100.0% to | 5-42^{a} | CDACENAAAHFCAAD | 396 | 100% |
| | | G902 | | EAALCRPCDEKVHMC | | (38/38) |
| | | | | NKLASRH | | |
| 200 | At/G902 | 100.0% to | 56-91^{b} | CDICENAPAFFYCEI | 397 | 100% |
| | | G902 | | DGSSLCLQCDMVVHV | | (36/36) |
| | | | | GGKRTH | | |
| 202 | At/G1881 | 70.1% to | 5-42^{a} | CDACESAAAIVFCAA | 398 | 87% |
| | | G902 | | DEAALCCSCDEKVHK | | (33/38) |
| | | | | CNKLASRH | | |
| 202 | At/G1881 | 70.1% to | 56-91^{b} | CDICENAPAFFYCEI | 399 | 100% |
| | | G902 | | DGSSLCLQCDMVVHV | | (36/36) |
| | | | | GGKRTH | | |
| 204 | Zm/ACF85933.1 | 57.5% to | 5-42^{a} | CDVCESAPAVLFCAA | 400 | 87% |
| | | G902 | | DEAALCRPCDEKVHM | | (33/38) |
| | | | | CNKLASRH | | |
| 204 | Zm/ACF85933.1 | 57.5% to | 56-91^{b} | CDICENSPAFFYCEI | 401 | 89% |
| | | G902 | | DGTSLCLSCDMTVHV | | (32/36) |
| | | | | GGKRTH | | |
| 208 | Zm/ACL52378.1 | 57.8% to | 5-42^{a} | CDVCESAPAVLFCAA | 402 | 87% |
| | | G902 | | DEAALCRPCDEKVHM | | (33/38) |
| | | | | CNKLASRH | | |
| 208 | Zm/ACL52378.1 | 57.8% to | 56-91^{b} | CDICENSPAFFYCEI | 403 | 89% |
| | | G902 | | DGTSLCLSCDMTVHV | | (32/36) |
| | | | | GGKRTH | | |
| 212 | Pt/Pt_648693 | 61.4% to | 5-42^{a} | CDVCESAAAILFCAA | 404 | 89% |
| | | G902 | | DEAALCRSCDEKVHM | | (34/38) |
| | | | | CNKLASRH | | |
| 212 | Pt/Pt_648693 | 61.4% to | 56-91^{b} | CDICEKAPAFFYCEI | 405 | 94% |
| | | G902 | | DGSSLCLQCDMIVHV | | (34/36) |
| | | | | GGKRTH | | |
| 214 | Pt/Pt_721866 | 61.0% to | 5-42^{a} | CDVCESAAAILFCAA | 406 | 87% |
| | | G902 | | DEAALCRSCDEKVHL | | (33/38) |
| | | | | CNKLASRH | | |
| 214 | Pt/Pt_721866 | 61.0% to | 56-91^{b} | CDICENAPAFFYCEI | 407 | 97% |
| | | G902 | | DGSSLCLQCDMIVHV | | (35/36) |
| | | | | GGKRTH | | |
| 216 | Pt/Pt_741179 | 54.5% to | 5-42^{a} | CDACESAFAIVFCAA | 408 | 84% |
| | | G902 | | DEAALCLACDKKVHM | | (32/38) |
| | | | | CNKLASRH | | |
| 216 | Pt/Pt_741179 | 54.5% to | 56-91^{b} | CDICENAPAFFYCET | 409 | 94% |
| | | G902 | | DGSSLCLQCDMTVHV | | (34/36) |
| | | | | GGKRTH | | |
| 218 | Pt/Pt_820080 | 56.2% to | 5-42^{a} | CDACESAAAIVFCAA | 410 | 89% |
| | | G902 | | DEAALCLACDEKVHM | | (34/38) |
| | | | | CMKLASRH | | |
| 218 | Pt/Pt_820080 | 56.2% to | 56-91^{b} | CDICENAPAFFYCET | 411 | 94% |
| | | G902 | | DGSSLCLQCDMTVHV | | (34/36) |
| | | | | GGKRTH | | |
| 220 | Os/Os09g35880.1 | 57.1% to | 5-42^{a} | CDVCESAPAVLFCVA | 412 | 79% |
| | | G902 | | DEAALCRSCDEKVHM | | (30/38) |
| | | | | CNKLARRH | | |
| 220 | Os/Os09g35880.1 | 57.1% to | 56-91^{b} | CDICENAPAFFYCEI | 413 | 92% |
| | | G902 | | DGTSLCLSCDMTVHV | | (33/36) |
| | | | | GGKRTH | | |
| 222 | Gm/Glyma01g37370.1 | 55.2% to | 5-42^{a} | CDACESAAAIVFCAA | 414 | 92% |
| | | G902 | | DEAALCRACDEKVHM | | (35/38) |
| | | | | CNKLASRH | | |
| 222 | Gm/Glyma01g37370.1 | 55.2% to | 56-91^{b} | CDICENAPAFFYCET | 415 | 94% |
| | | G902 | | DGSSLCLQCDMIVHV | | (34/36) |
| | | | | GGKRTH | | |
| 224 | Gm/Glyma11g07930.1 | 56.7% to | 5-42^{a} | CDACESAAAIVFCAA | 416 | 92% |
| | | G902 | | DEAALCRACDEKVHM | | (35/38) |
| | | | | CNKLASRH | | |
| 224 | Gm/Glyma11g07930.1 | 56.7% to | 56-91^{b} | CDICENAPAFFYCET | 417 | 94% |
| | | G902 | | DGSSLCLQCDMIVHV | | (34/36) |
| | | | | GGKRTH | | |
| 226 | Gm/Glyma11g07930.2 | 55.7% to | 5-42^{a} | CDACESAAAIVFCAA | 418 | 92% |
| | | G902 | | DEAALCRACDEKVHM | | (35/38) |
| | | | | CNKLASRH | | |
| 226 | Gm/Glyma11g07930.2 | 55.7% to | 56-91^{b} | CDICENAPAFFYCET | 419 | 94% |
| | | G902 | | DGSSLCLQCDMIVHV | | (34/36) |
| | | | | GGKRTH | | |
| 228 | Gm/Glyma11g07930.3 | 55.7% to | 5-42^{a} | CDACESAAAIVFCAA | 420 | 92% |
| | | G902 | | DEAALCRACDEKVHM | | (35/38) |
| | | | | CNKLASRH | | |
| 228 | Gm/Glyma11g07930.3 | 55.7% to | 56-91^{b} | CDICENAPAFFYCET | 421 | 94% |
| | | G902 | | DGSSLCLQCDMIVHV | | (34/36) |
| | | | | GGKRTH | | |
| 230 | Gm/Glyma11g07930.4 | 56.2% to | 5-42^{a} | CDACESAAAIVFCAA | 422 | 92% |
| | | G902 | | DEAALCRACDEKVHM | | (35/38) |
| | | | | CMKLASRH | | |
| 230 | Gm/Glyma11g07930.4 | 56.2% to | 56-91^{b} | CDICENAPAFFYCET | 423 | 94% |
| | | G902 | | DGSSLCLQCDMIVHV | | (34/36) |
| | | | | GGKRTH | | |
| 232 | Gm/Glyma11g11850.1 | 58.0% to | 5-42^{a} | CDVCESAAAILFCAA | 424 | 82% |
| | | G902 | | DEAALCSACDHKIHM | | (31/38) |
| | | | | CNKLASRH | | |
| 232 | Gm/Glyma11g11850.1 | 58.0% to | 56-91^{b} | CDICENAPAFFYCEI | 425 | 97% |
| | | G902 | | DGSSLCLQCDMIVHV | | (35/36) |
| | | | | GGKRTH | | |
| 234 | Gm/Glyma12g04130.1 | 62.2% to | 5-42^{a} | CDVCESAAAIVFCAA | 426 | 82% |
| | | G902 | | DEAALCSACDHKIHM | | (31/38) |
| | | | | CNKLASRH | | |
| 234 | Gm/Glyma12g04130.1 | 62.2% to | 56-91^{b} | CDICENAPAFFYCEI | 427 | 97% |
| | | G902 | | DGSSLCLQCDMIVHV | | (35/36) |
| | | | | GGKRTH | | |

Species abbreviations for Table 1: At—Arabidopsis thaliana; Gm—Glycine max; Os—Oryza sativa; Pt—Populus trichocarpa; Sl—Solanum lycopersicum; Zm—Zea mays.
^{a}first B-box type ZF domain
^{b}second B-box type ZF domain
^{c}putative CCT nuclear localization domain

Exemplary polynucleotides encoding the polypeptides of soybean B-box proteins (GmBBX) as represented in Table 2 were identified from yeast two hybrid screens and from phylogenetic analysis.

TABLE 2
Glycine max B-box polynucleotide and polypeptide sequences (GmBBX).

| Column 1 | Column 2 | | |
|---|---|---|---|
| Polynucleotide | Polypeptide | Column 3 | Column 4 |
| SEQ ID NO: | SEQ ID NO: | Gene Name | Domains |
| 435 | 436 | GmBBX1 | zf-B_box[2]::CCT |
| 437 | 438 | GmBBX2 | zf-B_box[2]::CCT |
| 439 | 440 | GmBBX3 | zf-B_box[2]::CCT |
| 441 | 442 | GmBBX4 | zf-B_box[2]::CCT |
| 443 | 444 | GmBBX5 | zf-B_box[2]::CCT |
| 445 | 446 | GmBBX6 | zf-B_box[2]::CCT |
| 447 | 448 | GmBBX7 | zf-B_box[2] |
| 449 | 450 | GmBBX11 | zf-B_box[2]::CCT |
| 451 | 452 | GmBBX12 | zf-B_box[2]::CCT |
| 453 | 454 | GmBBX13 | zf-B_box[1]::CCT |
| 455 | 456 | GmBBX14 | zf-B_box[1]::CCT |
| 457 | 458 | GmBBX15 | zf-B_box[1]::CCT |
| 459 | 460 | GmBBX16 | zf-B_box[1]::CCT |
| 461 | 462 | GmBBX17 | zf-B_box[1]::CCT |
| 463 | 464 | GmBBX18 | zf-B_box[1]::CCT |
| 465 | 466 | GmBBX19 | zf-B_box[2]::CCT |
| 467 | 468 | GmBBX20 | zf-B_box[2]::CCT |
| 469 | 470 | GmBBX21 | zf-B_box[2]::CCT |
| 471 | 472 | GmBBX22 | zf-B_box[2]::CCT |
| 473 | 474 | GmBBX23 | zf-B_box[2] |
| 475 | 476 | GmBBX24 | zf-B_box[1]::CCT |
| 477 | 478 | GmBBX25 | zf-B_box[2]::CCT |
| 479 | 480 | GmBBX26 | zf-B_box[2]::CCT |
| 481 | 482 | GmBBX27 | zf-B_box[2] |
| 483 | 484 | GmBBX28 | zf-B_box[2] |
| 485 | 486 | GmBBX29 | zf-B_box[2]::CCT |
| 487 | 488 | GmBBX30 | zf-B_box[2]::CCT |
| 489 | 490 | GmBBX31 | zf-B_box[2] |
| 491 | 492 | GmBBX33 | zf-B_box[2] |
| 493 | 494 | GmBBX34 | zf-B_box[2] |
| 495 | 496 | GmBBX35 | zf-B_box[2] |
| 497 | 498 | GmBBX36 | zf-B_box[2] |
| 499 | 500 | GmBBX37 | zf-B_box[2] |
| 501 | 502 | GmBBX38 | zf-B_box[2] |
| 503 | 504 | GmBBX39 | zf-B_box[2] |
| 505 | 506 | GmBBX40 | zf-B_box[2] |
| 507 | 508 | GmBBX41 | zf-B_box[2] |
| 509 | 510 | GmBBX42 | zf-B_box[2] |
| 511 | 512 | GmBBX43 | zf-B_box[2] |
| 513 | 514 | GmBBX44 | zf-B_box[2] |
| 515 | 516 | GmBBX45 | zf-B_box[2] |
| 517 | 518 | GmBBX46 | zf-B_box[2] |
| 519 | 520 | GmBBX47 | zf-B_box[2] |
| 521 | 522 | GmBBX48 | zf-B_box[2] |
| 523 | 524 | GmBBX49 | zf-B_box[2] |
| 525 | 526 | GmBBX50 | zf-B_box[1] |
| 527 | 528 | GmBBX51 | zf-B_box[1] |
| 529 | 530 | GmBBX52 | zf-B_box[1] |
| 531 | 532 | GmBBX53 | zf-B_box[1] |
| 533 | 534 | GmBBX54 | zf-B_box[1] |
| 535 | 536 | GmBBX55 | zf-B_box[1] |
| 537 | 538 | GmBBX56 | zf-B_box[1] |
| 539 | 540 | GmBBX57 | zf-B_box[1] |
| 541 | 542 | GmBBX58 | zf-B_box[1] |
| 543 | 544 | GmBBX59 | zf-B_box[1] |
| 545 | 546 | GmBBX60 | zf-B_box[1] |
| 547 | 548 | GmBBX61 | zf-B_box[1] |
| 549 | 550 | GmBBX62 | zf-B_box[1] |

Abbreviations for Table 2
zf-B_box[1]: first B-box type zinc finger domain
zf-B_box[2]: second B-box type zinc finger domain
CCT: nuclear localization domain
Gm: Glycine max

### Orthologs and Paralogs

Homologous sequences as described above can comprise orthologous or paralogous sequences. Several different methods are known by those of skill in the art for identifying and defining these functionally homologous sequences. General methods for identifying orthologs and paralogs, including phylogenetic methods, sequence similarity and hybridization methods, are described herein; an ortholog or paralog, including equivalogs, may be identified by one or more of the methods described below.

As described by Eisen, 1998, evolutionary information may be used to predict gene function. It is common for groups of genes that are homologous in sequence to have diverse, although usually related, functions. However, in many cases, the identification of homologs is not sufficient to make specific predictions because not all homologs have the same function. Thus, an initial analysis of functional relatedness based on sequence similarity alone may not provide one with a means to determine where similarity ends and functional relatedness begins. Fortunately, it is well known in the art that protein function can be classified using phylogenetic analysis of gene trees combined with the corresponding species. Functional predictions can be greatly improved by focusing on how the genes became similar in sequence (i.e., by evolutionary processes) rather than on the sequence similarity itself (Eisen, 1998). In fact, many specific examples exist in which gene function has been shown to correlate well with gene phylogeny (Eisen, 1998). Thus, “[t]he first step in making functional predictions is the generation of a phylogenetic tree representing the evolutionary history of the gene of interest and its homologs. Such trees are distinct from clusters and other means of characterizing sequence similarity because they are inferred by techniques that help convert patterns of similarity into evolutionary relationships . . . After the gene tree is inferred, biologically determined functions of the various homologs are overlaid onto the tree. Finally, the structure of the tree and the relative phylogenetic positions of genes of different functions are used to trace the history of functional changes, which is then used to predict functions of [as yet] uncharacterized genes” (Eisen, supra).

Within a single plant species, gene duplication may cause two copies of a particular gene, giving rise to two or more genes with similar sequence and often similar function known as paralogs. A paralog is therefore a similar gene formed by duplication within the same species. Paralogs typically cluster together or in the same Glade (a group of similar genes) when a gene family phylogeny is analyzed using programs such as CLUSTAL (Thompson et al., 1994; Higgins et al., 1996). Groups of similar genes can also be identified with pair-wise BLAST analysis (Feng and Doolittle, 1987). For example, a Glade of very similar MADS domain transcriptional regulators from Arabidopsis all share a common function in flowering time (Ratcliffe et al., 2001), and a group of very similar AP2 domain transcriptional regulators from Arabidopsis are involved in tolerance of plants to freezing (Gilmour et al., 1998). Analysis of groups of similar genes with similar function that fall within one Glade can yield sub-sequences that are particular to the Glade. These sub-sequences, known as consensus sequences, can not only be used to define the sequences within each Glade, but define the functions of these genes; genes within a Glade may contain paralogous sequences, or orthologous sequences that share the same function (see also, for example, Mount, 2001).

Transcriptional regulator gene sequences are conserved across diverse eukaryotic species lines (Goodrich et al., 1993; Lin et al., 1991; Sadowski et al., 1988). Plants are no exception to this observation; diverse plant species possess transcriptional regulators that have similar sequences and functions. Speciation, the production of new species from a parental species, gives rise to two or more genes with similar sequence and similar function. These genes, termed orthologs, often have an identical function within their host plants and are often interchangeable between species without losing function. Because plants have common ancestors, many genes in any plant species will have a corresponding orthologous gene in another plant species. Once a phylogenic tree for a gene family of one species has been constructed using a program such as CLUSTAL (Thompson et al., 1994; Higgins et al., 1996) potential orthologous sequences can be placed into the phylogenetic tree and their relationship to genes from the species of interest can be determined. Orthologous sequences can also be identified by a reciprocal BLAST strategy. Once an orthologous sequence has been identified, the function of the ortholog can be deduced from the identified function of the reference sequence.

By using a phylogenetic analysis, one skilled in the art would recognize that the ability to predict similar functions conferred by closely-related polypeptides is predictable. This predictability has been confirmed by our own many studies in which we have found that a wide variety of polypeptides have orthologous or closely-related homologous sequences that function as does the first, closely-related reference sequence. For example, distinct transcriptional regulators, including:
(i) AP2 family Arabidopsis G47 (found in U.S. Pat. No. 7,135,616), a phylogenetically-related sequence from soybean, and two phylogenetically-related homologs from rice all can confer greater tolerance to drought, hyperosmotic stress, or delayed flowering as compared to control plants;
(ii) CAAT family Arabidopsis G481 (found in PCT patent publication WO2004076638), and numerous phylogenetically-related sequences from dicots and monocots can confer greater tolerance to drought-related stress as compared to control plants;
(iii) Myb-related Arabidopsis G682 (found in PCT patent publication WO2004076638) and numerous phylogenetically-related sequences from dicots and monocots can confer greater tolerance to heat, drought-related stress, cold, and salt as compared to control plants;
(iv) WRKY family Arabidopsis G1274 (found in U.S. Pat. No. 7,196,245) and numerous closely-related sequences from dicots and monocots have been shown to confer increased water deprivation tolerance, and
(v) AT-hook family soy sequence G3456 (found in US Patent publication US20040128712A1) and numerous phylogenetically-related sequences from dicots and monocots, increased biomass compared to control plants when these sequences are overexpressed in plants.

The polypeptides sequences belong to distinct clades of polypeptides that include members from diverse species. In each case, most or all of the Glade member sequences derived from both dicots and monocots have been shown to confer increased yield or tolerance to one or more abiotic stresses when the sequences were overexpressed. These studies each demonstrate that evolutionarily conserved genes from diverse species are likely to function similarly (i.e., by regulating similar target sequences and controlling the same traits), and that polynucleotides from one species may be transformed into closely-related or distantly-related plant species to confer or improve traits.

As shown in Table 1 and Table 2, polypeptides that are phylogenetically related to disclosed polypeptides, including SEQ ID NOs: 2n, where n=1 to 117 or 218 to 275, may have conserved domains that share at least 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100% amino acid sequence identity to the listed sequences, for example, SEQ ID NOs: 235-427, and have similar functions in that the disclosed and closely related polypeptides may, when overexpressed, confer at least one trait selected from the group consisting of decreased sensitivity to light, increased yield, greater height, greater stem diameter, greater resistance to lodging, increased secondary rooting, greater cold tolerance, greater tolerance to water deprivation, reduced stomatal conductance, altered C/N sensing, increased low nitrogen tolerance, greater late season growth and vigor, greater number of primary nodes, greater late season canopy coverage, increased tolerance to hyperosmotic stress, altered levels of ureides, altered levels of hexose sugars, altered SPS activity, altered levels of starch, and delayed senescence, as compared to a control plant.

At the nucleotide level, sequences that are closely related to the disclosed polynucleotide sequences will typically share at least about 30% or at least 40%, at least 41%, at least 42%, at least 43%, at least 44%, at least 45%, at least 46%, at least 47%, at least 48%, or at least 49% nucleotide sequence identity, preferably at least about 50%, at least 51%, at least 52%, at least 53%, at least 54%, at least 55%, at least 56%, at least 57%, at least 58%, or at least 59% nucleotide sequence identity, or at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or at least 96%, or at least 97%, or at least 98%, or at least 99%, or about 100% sequence identity to one or more of the listed full-length polynucleotide sequences, or to a polynucleotide sequence encoding a conserved domain of a listed polypeptide sequence. The degeneracy of the genetic code enables major variations in the nucleotide sequence of a polynucleotide while maintaining the amino acid sequence of the encoded protein. Polynucleotides that encode polypeptides that are closely-related, that is, orthologous or paralogous, to the disclosed polypeptide sequences will be identifiable by having at least any of the percentage identities provided in this paragraph.

At the polypeptide level, sequences that are closely related to the disclosed polypeptide sequences will typically share at least about 30% or at least 40%, at least 41%, at least 42%, at least 43%, at least 44%, at least 45%, at least 46%, at least 47%, at least 48%, or at least 49% amino acid sequence identity, preferably at least about 50%, at least 51%, at least 52%, at least 53%, at least 54%, at least 55%, at least 56%, at least 57%, at least 58%, or at least 59% nucleotide sequence identity, or at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or at least 96%, or at least 97%, or at least 98%, or at least 99%, or about 100% sequence identity to one or more of the listed full-length polypeptide sequences, or to conserved domain of a listed full length sequence, or to a listed conserved domain, or to a listed polypeptide sequence but excluding or outside of the region(s) encoding a known consensus sequence or consensus DNA-binding site, or outside of the region(s) encoding one or all conserved domains. Polypeptides, or their domains, that are closely-related, that is, orthologous or paralogous, to the disclosed polypeptide sequences, or their domains, will be identifiable by having at least any of the percentage identities provided in this paragraph.

Percent identity can be determined electronically, e.g., by using the MEGALIGN program (DNASTAR, Inc. Madison, Wis.). The MEGALIGN program can create alignments between two or more sequences according to different methods, for example, the clustal method (see, for example, Higgins and Sharp, 1988). The clustal algorithm groups sequences into clusters by examining the distances between all pairs. The clusters are aligned pairwise and then in groups. Other alignment algorithms or programs may be used, including FASTA, BLAST, or ENTREZ, FASTA and BLAST, and which may be used to calculate percent similarity. These are available as a part of the GCG sequence analysis package (University of Wisconsin, Madison, WI), and can be used with or without default settings. ENTREZ is available through the National Center for Biotechnology Information. In one embodiment, the percent identity of two sequences can be determined by the GCG program with a gap weight of 1, e.g., each amino acid gap is weighted as if it were a single amino acid or nucleotide mismatch between the two sequences (see U.S. Pat. No. 6,262,333).

Software for performing BLAST analyses is publicly available, e.g., through the National Center for Biotechnology Information (see internet website at www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul, 1990; Altschul et al., 1993). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, 1989). Unless otherwise indicated for comparisons of predicted polynucleotides, “sequence identity” refers to the % sequence identity generated from a tBlastx using the NCBI version of the algorithm at the default settings using gapped alignments with the filter “off” (see, for example, internet website at www.ncbi.nlm.nih.govf).

Other techniques for alignment are described by Doolittle, 1996. Preferably, an alignment program that permits gaps in the sequence is utilized to align the sequences. The Smith-Waterman is one type of algorithm that permits gaps in sequence alignments (see Shpaer, 1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The percentage similarity between two polypeptide sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no similarity between the two amino acid sequences are not included in determining percentage similarity. Percent identity between polynucleotide sequences can also be counted or calculated by other methods known in the art, e.g., the Jotun Hein method (see, for example, Hein, 1990). Identity between sequences can also be determined by other methods known in the art, e.g., by varying hybridization conditions (see US Patent Application No. 20010010913).

Thus, the present disclosure provides methods for identifying a sequence similar or paralogous or orthologous or homologous to one or more polynucleotides as noted herein, or one or more target polypeptides encoded by the polynucleotides, or otherwise noted herein and may include linking or associating a given plant phenotype or gene function with a sequence. In the methods, a sequence database is provided (locally or across an internet or intranet) and a query is made against the sequence database using the relevant sequences herein and associated plant phenotypes or gene functions.

In addition, one or more polynucleotide sequences or one or more polypeptides encoded by the polynucleotide sequences may be used to search against a BLOCKS (Bairoch et al., 1997), PFAM, and other databases which contain previously identified and annotated motifs, sequences and gene functions. Methods that search for primary sequence patterns with secondary structure gap penalties (Smith et al., 1992) as well as algorithms such as Basic Local Alignment Search Tool (BLAST; Altschul, 1990; Altschul et al., 1993), BLOCKS (Henikoff and Henikoff, 1991), Hidden Markov Models (HMM; Eddy, 1996; Sonnhammer et al., 1997), and the like, can be used to manipulate and analyze polynucleotide and polypeptide sequences encoded by polynucleotides. These databases, algorithms and other methods are well known in the art and are described in Ausubel et al., 1997, and in Meyers, 1995.

A further method for identifying or confirming that specific homologous sequences control the same function is by comparison of the transcript profile(s) obtained upon overexpression or knockout of two or more related polypeptides. Since transcript profiles are diagnostic for specific cellular states, one skilled in the art will appreciate that genes that have a highly similar transcript profile (e.g., with greater than 50% regulated transcripts in common, or with greater than 70% regulated transcripts in common, or with greater than 90% regulated transcripts in common) will have highly similar functions. Fowler and Thomashow, 2002, have shown that three paralogous AP2 family genes (CBF1, CBF2 and CBF3) are induced upon cold treatment, and each of which can condition improved freezing tolerance, and all have highly similar transcript profiles. Once a polypeptide has been shown to provide a specific function, its transcript profile becomes a diagnostic tool to determine whether paralogs or orthologs have the same function.

Furthermore, methods using manual alignment of sequences similar or homologous to one or more polynucleotide sequences or one or more polypeptides encoded by the polynucleotide sequences may be used to identify regions of similarity and B-box zinc finger domains. Such manual methods are well-known of those of skill in the art and can include, for example, comparisons of tertiary structure between a polypeptide sequence encoded by a polynucleotide that comprises a known function and a polypeptide sequence encoded by a polynucleotide sequence that has a function not yet determined. Such examples of tertiary structure may comprise predicted alpha helices, beta-sheets, amphipathic helices, leucine zipper motifs, zinc finger motifs, proline-rich regions, cysteine repeat motifs, and the like.

Orthologs and paralogs of presently disclosed polypeptides may be cloned using compositions provided by the present disclosure according to methods well known in the art. cDNAs can be cloned using mRNA from a plant cell or tissue that expresses one of the present sequences. Appropriate mRNA sources may be identified by interrogating Northern blots with probes designed from the present sequences, after which a library is prepared from the mRNA obtained from a positive cell or tissue. Polypeptide-encoding cDNA is then isolated using, for example, PCR, using primers designed from a presently disclosed gene sequence, or by probing with a partial or complete cDNA or with one or more sets of degenerate probes based on the disclosed sequences. The cDNA library may be used to transform plant cells. Expression of the cDNAs of interest is detected using, for example, microarrays, Northern blots, quantitative PCR, or any other technique for monitoring changes in expression. Genomic clones may be isolated using similar techniques to those.

Examples of orthologs of the Arabidopsis polypeptide sequences and their functionally similar orthologs are listed in Tables 1 and 2 and in the Sequence Listing. In addition to the sequences in Table 1, Table 2, and the Sequence Listing, the present disclosure encompasses isolated nucleotide sequences that are phylogenetically and structurally similar to sequences listed in the Sequence Listing and can function in a plant by increasing yield and/or and abiotic stress tolerance when ectopically expressed in a plant.

Since a significant number of these sequences are phylogenetically and sequentially related to each other and have been shown to increase yield from a plant and/or abiotic stress tolerance, one skilled in the art would predict that other similar, phylogenetically related sequences falling within the present clades of polypeptides would also perform similar functions when ectopically expressed.

### Identifying Polynucleotides or Nucleic Acids by Hybridization

Polynucleotides homologous to the sequences illustrated in the Sequence Listing and tables can be identified, e.g., by hybridization to each other under stringent or under highly stringent conditions.

Polynucleotides that encode polypeptides that are closely-related, that is, orthologous or paralogous, to the disclosed polypeptide sequences may be identifiable by their ability to hybridize to the disclosed polynucleotides under high stringency conditions, including the high stringency conditions provided herein.

Single stranded polynucleotides hybridize when they associate based on a variety of well characterized physical-chemical forces, such as hydrogen bonding, solvent exclusion, base stacking and the like. The stringency of hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations (and number thereof), as described in more detail in the references cited below (e.g., Sambrook et al., 1989; Berger and Kimmel, 1987; and Anderson and Young, 1985).

Encompassed by the present disclosure are polynucleotide sequences that are capable of hybridizing to the claimed polynucleotide sequences, including any of the polynucleotides within the Sequence Listing, and fragments thereof under various conditions of stringency (see, for example, Wahl and Berger, 1987; and Kimmel, 1987). In addition to the nucleotide sequences listed in the Sequence Listing, full length cDNA, orthologs, and paralogs of the present nucleotide sequences may be identified and isolated using well-known methods. The cDNA libraries, orthologs, and paralogs of the present nucleotide sequences may be screened using hybridization methods to determine their utility as hybridization target or amplification probes.

With regard to hybridization, conditions that are highly stringent, and means for achieving them, are well known in the art. See, for example, Sambrook et al., 1989; Berger, 1987, pages 467-469; and Anderson and Young, 1985).

Stability of DNA duplexes is affected by such factors as base composition, length, and degree of base pair mismatch. Hybridization conditions may be adjusted to allow DNAs of different sequence relatedness to hybridize. The melting temperature (T m) is defined as the temperature when 50% of the duplex molecules have dissociated into their constituent single strands. The melting temperature of a perfectly matched duplex, where the hybridization buffer contains formamide as a denaturing agent, may be estimated by the following equations:

Tₘ(° C.)=81.5+16.6(log[Na+])+0.41(% G+C)−0.62(% formamide)−500/L (I) DNA-DNA:

Tₘ(° C.)=79.8+18.5(log[Na+])+0.58(% G+C)+0.12(% G+C)2−0.5(% formamide)−820/L (II) DNA-RNA:

Tₘ(° C.)=79.8+18.5(log[Na+])+0.58(% G+C)+0.12(% G+C)2−0.35(% formamide)−820/L (III) RNA-RNA:

where L is the length of the duplex formed, [Na+] is the molar concentration of the sodium ion in the hybridization or washing solution, and % G+C is the percentage of (guanine+cytosine) bases in the hybrid. For imperfectly matched hybrids, approximately 1° C. is required to reduce the melting temperature for each 1% mismatch.

Hybridization experiments are generally conducted in a buffer of pH between 6.8 to 7.4, although the rate of hybridization is nearly independent of pH at ionic strengths likely to be used in the hybridization buffer (Anderson and Young, 1985). In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments such as genes that duplicate functional enzymes from closely related organisms. The stringency can be adjusted either during the hybridization step or in the post-hybridization washes. Salt concentration, formamide concentration, hybridization temperature and probe lengths are variables that can be used to alter stringency (as described by the formula above). As a general guidelines high stringency is typically performed at Tₘ-5° C. to Tₘ-20° C., moderate stringency at Tₘ-20° C. to Tₘ-35° C. and low stringency at Tₘ-35° C. to Tₘ-50° C. for duplex>150 base pairs. Hybridization may be performed at low to moderate stringency (25-50° C. below T m), followed by post-hybridization washes at increasing stringencies. Maximum rates of hybridization in solution are determined empirically to occur at Tₘ-25° C. for DNA-DNA duplex and Tₘ-15° C. for RNA-DNA duplex. Optionally, the degree of dissociation may be assessed after each wash step to determine the need for subsequent, higher stringency wash steps.

High stringency conditions may be used to select for nucleic acid sequences with high degrees of identity to the disclosed sequences. An example of stringent hybridization conditions obtained in a filter-based method such as a Southern or Northern blot for hybridization of complementary nucleic acids that have more than 100 complementary residues is about 5° C. to 20° C. lower than the thermal melting point (Tₘ) for the specific sequence at a defined ionic strength and pH. Conditions used for hybridization may include about 0.02 M to about 0.15 M sodium chloride, about 0.5% to about 5% casein, about 0.02% SDS or about 0.1% N-laurylsarcosine, about 0.001 M to about 0.03 M sodium citrate, at hybridization temperatures between about 50° C. and about 70° C. More preferably, high stringency conditions are about 0.02 M sodium chloride, about 0.5% casein, about 0.02% SDS, about 0.001 M sodium citrate, at a temperature of about 50° C. Nucleic acid molecules that hybridize under stringent conditions will typically hybridize to a probe based on either the entire DNA molecule or selected portions, e.g., to a unique subsequence, of the DNA.

Stringent salt concentration will ordinarily be less than about 750 mM NaCl and 75 mM trisodium citrate. Increasingly stringent conditions may be obtained with less than about 500 mM NaCl and 50 mM trisodium citrate, to even greater stringency with less than about 250 mM NaCl and 25 mM trisodium citrate. Low stringency hybridization can be obtained in the absence of organic solvent, e.g., formamide, whereas high stringency hybridization may be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. with formamide present. Varying additional parameters, such as hybridization time, the concentration of detergent, e.g., sodium dodecyl sulfate (SDS) and ionic strength, are well known to those skilled in the art. Various levels of stringency are accomplished by combining these various conditions as needed.

The washing steps that follow hybridization may also vary in stringency; the post-hybridization wash steps primarily determine hybridization specificity, with the most critical factors being temperature and the ionic strength of the final wash solution. Wash stringency can be increased by decreasing salt concentration or by increasing temperature. Stringent salt concentration for the wash steps will preferably be less than about 30 mM NaCl and 3 mM trisodium citrate, and most preferably less than about 15 mM NaCl and 1.5 mM trisodium citrate.

Thus, hybridization and wash conditions that may be used to bind and remove polynucleotides with less than the desired homology to the nucleic acid sequences or their complements that encode the present polypeptides include, for example:
6×SSC at 65° C.;
50% formamide, 4×SSC at 42° C.; or
0.5×SSC, 0.1% SDS at 65° C.;
with, for example, two wash steps of 10-30 minutes each. Useful variations on these conditions will be readily apparent to those skilled in the art.

A person of skill in the art would not expect substantial variation among polynucleotide species encompassed within the scope of the present disclosure because the highly stringent conditions set forth in the above formulae yield structurally similar polynucleotides.

If desired, one may employ wash steps of even greater stringency, including about 0.2×SSC, 0.1% SDS at 65° C. and washing twice, each wash step being about 30 minutes, or about 0.1×SSC, 0.1% SDS at 65° C. and washing twice for 30 minutes. The temperature for the wash solutions will ordinarily be at least about 25° C., and for greater stringency at least about 42° C. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C. For identification of less closely related homologs, wash steps may be performed at a lower temperature, e.g., 50° C.

An example of a low stringency wash step employs a solution and conditions of at least 25° C. in 30 mM NaCl, 3 mM trisodium citrate, and 0.1% SDS over 30 minutes. Greater stringency may be obtained at 42° C. in 15 mM NaCl, with 1.5 mM trisodium citrate, and 0.1% SDS over 30 minutes. Even higher stringency wash conditions are obtained at 65° C. −68° C. in a solution of 15 mM NaCl, 1.5 mM trisodium citrate, and 0.1% SDS. Wash procedures will generally employ at least two final wash steps. Additional variations on these conditions will be readily apparent to those skilled in the art (see, for example, US Patent Application No. 20010010913).

Stringency conditions can be selected such that an oligonucleotide that is perfectly complementary to the coding oligonucleotide hybridizes to the coding oligonucleotide with at least about a 5-10× higher signal to noise ratio than the ratio for hybridization of the perfectly complementary oligonucleotide to a nucleic acid encoding a polypeptide known as of the filing date of the application. It may be desirable to select conditions for a particular assay such that a higher signal to noise ratio, that is, about 15× or more, is obtained. Accordingly, a subject nucleic acid will hybridize to a unique coding oligonucleotide with at least a 2× or greater signal to noise ratio as compared to hybridization of the coding oligonucleotide to a nucleic acid encoding known polypeptide. The particular signal will depend on the label used in the relevant assay, e.g., a fluorescent label, a colorimetric label, a radioactive label, or the like. Labeled hybridization or PCR probes for detecting related polynucleotide sequences may be produced by oligolabeling, nick translation, end-labeling, or PCR amplification using a labeled nucleotide.

Encompassed by the present disclosure are polynucleotide sequences that are capable of hybridizing to the claimed polynucleotide sequences, including any of the polynucleotides within the Sequence Listing, and fragments thereof under various conditions of stringency (see, for example, Wahl and Berger, 1987, pages 399-407; and Kimmel, 1987). In addition to the nucleotide sequences in the Sequence Listing, full length cDNA, orthologs, and paralogs of the present nucleotide sequences may be identified and isolated using well-known methods. The cDNA libraries, orthologs, and paralogs of the present nucleotide sequences may be screened using hybridization methods to determine their utility as hybridization target or amplification probes.

### Sequence Variations

It will readily be appreciated by those of skill in the art that the instant disclosure includes any of a variety of polynucleotide sequences provided in the Sequence Listing or capable of encoding polypeptides that function similarly to those provided in the Sequence Listing. Due to the degeneracy of the genetic code, many different polynucleotides can encode identical and/or substantially similar polypeptides in addition to those sequences illustrated in the Sequence Listing. Nucleic acids having a sequence that differs from the sequences shown in the Sequence Listing, or complementary sequences, that encode functionally equivalent peptides (that is, peptides having some degree of equivalent or similar biological activity) but differ in sequence from the sequence shown in the sequence listing due to degeneracy in the genetic code, are also within the scope of the instant disclosure and claims.

Altered polynucleotide sequences encoding polypeptides include those sequences with deletions, insertions, or substitutions of different nucleotides, resulting in a polynucleotide encoding a polypeptide with at least one functional characteristic of the instant polypeptides. Included within this definition are polymorphisms which may or may not be readily detectable using a particular oligonucleotide probe of the polynucleotide encoding the instant polypeptides, and improper or unexpected hybridization to allelic variants, with a locus other than the normal chromosomal locus for the polynucleotide sequence encoding the instant polypeptides.

Sequence alterations that do not change the amino acid sequence encoded by the polynucleotide are termed “silent” variations. With the exception of the codons ATG and TGG, encoding methionine and tryptophan, respectively, any of the possible codons for the same amino acid can be substituted by a variety of techniques, for example, site-directed mutagenesis, available in the art. Accordingly, any and all such variations of a sequence selected from the above tables are a feature of the disclosure.

In addition to silent variations, other conservative variations that alter one, or a few amino acids in the encoded polypeptide, can be made without altering the function of the polypeptide. For example, substitutions, deletions and insertions introduced into the sequences provided in the Sequence Listing are also envisioned. Such sequence modifications can be engineered into a sequence by site-directed mutagenesis (for example, Olson et al., Smith et al., Zhao et al., and other articles in Wu, 1993), or the other methods known in the art or noted herein Amino acid substitutions are typically of single residues; insertions usually will be on the order of about from 1 to 10 amino acid residues; and deletions will range about from 1 to 30 residues. In preferred embodiments, deletions or insertions are made in adjacent pairs, for example, a deletion of two residues or insertion of two residues. Substitutions, deletions, insertions or any combination thereof can be combined to arrive at a sequence. The mutations that are made in the polynucleotide encoding the transcriptional regulator should not place the sequence out of reading frame and should not create complementary regions that could produce secondary mRNA structure. Preferably, the polypeptide encoded by the DNA performs the desired function.

Conservative substitutions are those in which at least one residue in the amino acid sequence has been removed and a different residue inserted in its place. Such substitutions generally are made in accordance with the Table 3 when it is desired to maintain the activity of the protein. Table 3 shows amino acids which can be substituted for an amino acid in a protein and which are typically regarded as conservative substitutions.

TABLE 3
Possible conservative amino acid substitutions

| Amino Acid | |
|---|---|
| Residue | Conservative substitutions |
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

The polypeptides provided in the Sequence Listing have a novel activity, such as, for example, regulatory activity. Although all conservative amino acid substitutions (for example, one basic amino acid substituted for another basic amino acid) in a polypeptide will not necessarily result in the polypeptide retaining its activity, it is expected that many of these conservative mutations would result in the polypeptide retaining its activity. Most mutations, conservative or non-conservative, made to a protein but outside of a conserved domain required for function and protein activity will not affect the activity of the protein to any great extent.

### EXAMPLES

It is to be understood that the present disclosure is not limited to the particular devices, machines, materials and methods described. Although particular embodiments are described, equivalent embodiments may be used to produce the instant compositions and practice the instant methods.

The disclosure, now being generally described, will be more readily understood by reference to the following examples, which are included merely for purposes of illustration of certain aspects and embodiments of the present disclosure and are not intended to limit claims to the instant compositions or methods. It will be recognized by one of skill in the art that a polypeptide that is associated with a particular first trait may also be associated with at least one other, unrelated and inherent second trait which was not predicted by the first trait.

### Example I. Vector and Cloning Information for Arabidopsis

A number of constructs were used to modulate the activity of the disclosed sequences. An individual project was defined as the analysis of lines for a particular construct (for example, this might include any of the disclosed lines that constitutively overexpress a disclosed B-box polypeptide sequence). In the present study, a full-length wild-type version of a gene was directly fused to a promoter that drove its expression in transgenic plants. Such a promoter could be the native promoter of that gene, or the CaMV 35S promoter. Alternatively, a promoter that drives tissue specific or conditional expression could be used in similar studies.

In the present study, expression of a given polynucleotide from a particular promoter was achieved by either a direct-promoter fusion construct in which that sequence was cloned directly behind the promoter of interest, or by a two-component expression system. A direct fusion approach has the advantage of allowing for simple genetic analysis if a given promoter-polynucleotide line is to be crossed into different genetic backgrounds at a later date. The two-component method, on the other hand, potentially allows for greater versatility in the creation of various promoter-gene combinations, and has the potential for generating stronger expression to be obtained via an amplification of transcription.

A list of constructs (PIDs) used to transform plants, indicating the promoter fragment that was used to drive the transgene and the cloning vector backbone, is provided in Table 4. Compilations of the sequences of promoter fragments and the expressed transgene sequences within the PIDs are provided in the Sequence Listing. The list of constructs presented in Table 4 is not intended to limit the instant description; other constructs that may be used to transform plants, and which may comprise useful promoter-gene combinations that are active in various tissues or in response to various or particular environmental conditions may also be envisioned.

TABLE 4
Sequences of promoter fragments and the expressed transgene sequences

| | SEQ ID | Plant from | | | |
|---|---|---|---|---|---|
| | NO: of | which | | | |
| Gene | Gene | sequence | Construct | Promoter:: | Expression |
| Identifier | Identifier | derived | (PID) | Leader | construct(s) |
| AT4G27310; | 1 | Arabidopsis | pMON125158 | 35S::Hsp70L | Direct promoter-fusion |
| or AtBBX28 | | | | | |
| (G1481) | | | | | |
| AT2G31380; | 153 | Arabidopsis | pMON125664 | 35S::Hsp70L | Direct promoter-fusion |
| or AtBBX25 | | | | | |
| (G1894) | | | | | |
| AT1G06040 | 151 | Arabidopsis | pMON125195 | 35S::Hsp70L | Direct promoter-fusion |
| or AtBBX24 | | | | | |
| (G329) | | | | | |
| AT4G15248; | 39 | Arabidopsis | pMON125199 | 35S::Hsp70L | Direct promoter-fusion |
| or AtBBX30 | | | | | |
| (G1478) | | | | | |
| At2G21320; | 201 | Arabidopsis | pMON125196 | 35S::Hsp70L | Direct promoter-fusion |
| or AtBBX18 | | | | | |
| (G1881) | | | | | |
| AT3G07650 | 103 | Arabidopsis | pMON125230 | 35S::Hsp70L | Direct promoter-fusion |
| or AtBBX7 | | | | | |
| (G2440) | | | | | |
| AT1G60250 | 51 | Arabidopsis | pMON125732 | 35S::Hsp70L | Direct promoter-fusion |
| or AtBBX26 | | | | | |
| (G1486) | | | | | |
| At4G38960 | 199 | Arabidopsis | pMON125730 | 35S::Hsp70L | Direct promoter-fusion |
| or AtBBX19 | | | | | |
| (G9902) | | | | | |
| AT4G39070 | 53 | Arabidopsis | pMON125197 | 35S::Hsp70L | Direct promoter-fusion |
| or AtBBX20 | | | | | |
| (G1888) | | | | | |

### Example II. Transformation

Gruber et al., 1993, described several expression vectors and culture methods that may be used for cell or tissue transformation and subsequent regeneration.

Dicot and monocot expression vectors Transformation of plants and plant parts by Agrobacterium-mediated transformation can be practiced using a recombinant DNA construct, vector or cassette with the genetic elements as shown in Table 5 for dicots and Table 6 for monocots. The disclosed recombinant polynucleotides in the Sequence Listing “MBI_0120 PCT_ST25” may be incorporated in such recombinant DNA constructs. The elements in a recombinant DNA construct provided in Tables 5 and 6 are only examples of different elements that can be used for the purpose of producing transgenic plants that have incorporated the polynucleotides. Other elements can be used as well in combination with the polynucleotides of the present disclosure to achieve trait modification.

A base recombinant DNA construct specifically useful for inserting a recombinant expression cassette into a chromosome in a nucleus in a dicot plant by Agrobacterium-mediated transformation is provided in Table 5. In Table 5, column 1 describes the function of the segment of the vector, column 2 provides a short name of a discrete genetic element and column 3 provides a more detailed description of the element.

TABLE 5
Example of an expression vector useful for transformation of dicot plants and plant parts.

| Function | Name | Element Description |
|---|---|---|
| Agrobacterium T-DNA | B-AGRtu.left border | Agro left border sequence, essential for transfer of T- |
| transfer | | DNA. |
| Plant selectable marker | P-At.Act7 | Promoter from the Arabidopsis actin 7 gene |
| expression cassette | L-At.Act7 | 5′UTR of Arabidopsis Act7 gene |
| | I-At.Act7 | Intron from the Arabidopsis actin7 gene |
| | TS-At.ShkG-CTP2 | Transit peptide region of Arabidopsis EPSPS |
| | CR-AGRtu.aroA- | Codon modified CP4 coding region |
| | CP4.nno_At | |
| | T-AGRtu.nos | A 3′ non-translated region of the nopaline synthase |
| | | gene of Agrobacterium tumefaciens Ti plasmid which |
| | | functions to direct polyadenylation of the mRNA. |
| Gene of interest | P-CaMV.35S-cnh | Promoter for 35S RNA from CaMV containg a |
| expression cassette | | duplication of the −90 to −350 region. |
| | GOI | A polynucleodtide or polypeptide-coding sequence in |
| | | Tables 1 or 2 |
| | T-Gb.E6-3b | 3′ untranslated region from the fiber protein E6 gene of |
| | | sea-island cotton. |
| Agrobacterium T-DNA | B-AGRtu.right border | Agro right border sequence, essential for transfer of T- |
| transfer | | DNA. |
| Maintenance in E. coli | OR-Ec.oriV-RK2 | The vegetative origin of replication from plasmid RK2. |
| | CR-Ec.rop | Coding region for repressor of primer from the ColE1 |
| | | plasmid. Expression of this gene product interferes with |
| | | primer binding at the origin of replication, keeping |
| | | plasmid copy number low. |
| | OR-Ec.ori-ColE1 | The minimal origin of replication from the E. coli |
| | | plasmid ColE1. |
| | P-Ec.aadA-SPC/STR | Promoter for Tn7 adenylytransferase (AAD (3″)) |
| | CR-Ec.aadA-SPC/STR | Coding region for Tn7 adenylyltransferase (AAD (3″)) |
| | | conferring spectinomycin and streptomycin resistance. |
| | T-Ec.aadA-SPC/STR | 3′ UTR from the Tn7 adenylyltransferase (AAD (3″)) |
| | | gene of E. coli. |

An example of an expression vector that may be used to transform a monocot plant by Agrobacterium-mediated transformation is provided in Table 6. In Table 6, column 1 describes the function of the segment of the vector, column 2 provides a short name of the discrete genetic elements and column 3 provides a more detailed description of the elements.

TABLE 6
Example of an expression vector useful for transformation of monocot plants and plant parts.

| Function | Name | Element Description |
|---|---|---|
| Agrobacterium T- | B-AGRtu.right border | Agrobacterium right border sequence essential for |
| DNA trasfer | | transfer of T-DNA. |
| Gene of interest | E-Os.Act1 | Upstream promoter region of the rice actin 1 gene |
| expression cassette | E-CaMV.35S.2xA1-B3 | Duplicated35S A1-B3 domain without TATA box |
| | P-Os.Act1 | Promoter region of the rice actin 1 gene |
| | L-Ta.Lhcb1 | 5′ untranslated leader of wheat major chlorophyll a/b |
| | | binding protein |
| | I-Os.Act1 | First intron and flanking UTR exon sequences from the |
| | | rice actin 1 gene |
| | GOI | A polynucleotide or polypeptide-coding sequence in |
| | | Table 1 and Table 2 |
| | T-St.Pis4 | 3′ non-translated region of the potato proteinase |
| | | inhibitor II gene which functions to direct |
| | | polyadenylation of the mRNA |
| Plant selectable | P-Os.Act1 | Promoter from the rice actin 1 gene |
| marker expression | L-Os.Act1 | First exon of the rice actin 1 gene |
| cassette | I-Os.Act1 | First intron and flanking UTR exon sequences from the |
| | | rice actin 1 gene |
| | TS-At.ShkG-CTP2 | Transit peptide region of Arabidopsis EPSPS |
| | CR-AGRtu.aroA-CP4.nat | Coding region for bacterial strain CP4 native aroA |
| | | gene. |
| | T-AGRtu.nos | A 3′ non-trranslated region of the nopaline synthase |
| | | gene of Argrobacterium tumefaciens Ti plasmid which |
| | | functions to direct polyadenylation of the mRNA. |
| Agrobacterium T- | B-AGRtu.left border | Agro left border sequence, essential for transger of T- |
| DNA transfer | | DNA. |
| Maintenance in E. | OR-Ec.oriV-RK2 | The vegetative origin of replication from plasmid RK2. |
| coli | CR-Ec.rop | Coding region for repressor of primer from the ColE1 |
| | | plasmid. Expression of this gene product interferes |
| | | with primer binding at the origin of replication, |
| | | keeping plasmid copy number low. |
| | OR-Ec.ori-ColE1 | The minimal origin of replication from the E. coli |
| | | plasmid ColE1. |
| | P.Ec.aadA-SPC/STR | Promoter for Tn7 adenylyltransferase (AAD (3″)) |
| | CR-Ec.aadA-SPC/STR | Coding region for Tn7 adenylyltransferase (AAD (3″)) |
| | | conferring spectinomycin and streptomycin resistance. |
| | T-Ec.aadA-SPC/STR | 3′ UTR from the Tn7 adenylyltransferase (AAD (3″)) |
| | | gene of E. coli. |

To construct expression vectors for expressing a protein identified in Table 1 and Table 2, primers for PCR amplification of the protein coding nucleotides are designed at or near the start and stop codons of the coding sequence, in order to eliminate most of the 5′ and 3′ untranslated regions. The protein coding nucleotides are inserted into the base vector in the gene of interest expression cassette at an insertion site, i.e. between the intron element and the polyadenylation element (Table 5 or Table 6, depending on whether the plant to be transformed is a dicot or monocot, respectively).

To construct expression vectors for suppressing a protein identified in Tables 1 and 2, the amplified protein coding nucleotides may be assembled in a sense and antisense arrangement and inserted into the base expression vector at the insertion site in the gene of interest expression cassette (Table 5 or Table 6, depending on whether the plant to be transformed is a dicot or monocot, respectively) to provide transcribed RNA that will form a double-stranded RNA for RNA interference suppression of the protein. Expression vectors for suppressing a protein identified in Tables 1 and 2 may also be designed to insert an expression cassette that expresses (a) a miRNA that targets the gene for suppression, (b) a messenger RNA that is translated to the target protein and has a synthetic miRNA recognition site that would result in down-modulation of the target protein, (c) an RNA that forms a dsRNA and that is processed into siRNAs that effect down regulation of the target protein, (d) a ssRNA that forms a ta-siRNA which results in the production of siRNAs that effect down-modulation of the target protein.

Transformation of Dicots or Eudicots to Produce Increased Yield and/or Abiotic Stress Tolerance

Crop species that overexpress or suppress the disclosed and closely related polypeptides may produce plants with increased water deprivation or drought tolerance, cold and/or nutrient tolerance and/or yield in both stressed and non-stressed conditions. Thus, polynucleotide sequences listed in the Sequence Listing recombined into, for example, one of the disclosed expression vector, or another suitable expression vector such as one comprising a sequence that is closely related to the instantly disclosed sequences, may be transformed into a plant for the purpose of modifying plant traits such as improved yield and/or quality. The expression vector may contain a constitutive promoter, an inducible promoter, a diurnally-regulated promoter, a tissue-enhanced promoter, a tissue-preferred promoter, or a tissue-specific promoter operably linked to the polynucleotide.

The expression vector may be introduced into a variety of plants by means well known in the art such as, for example, direct DNA transfer or Agrobacterium tumefaciens-mediated transformation. It is now a routine to produce transgenic plants from most dicot plants (see Weissbach and Weissbach, 1989; Gelvin et al., 1990; Herrera-Estrella et al., 1983; Bevan, 1984; and Klee, 1985). Methods for analysis of traits are routine in the art and examples are disclosed above. There are a substantial number of alternatives to Agrobacterium-mediated transformation protocols. One such method is microprojectile-mediated transformation, in which DNA on the surface of microprojectile particles is driven into plant tissues with a biolistic device (see, for example, Sanford et al., 1987; Christou et al., 1992; Sanford, 1993; Klein et al., 1987; U.S. Pat. No. 5,015,580 (Christou et al, issued May 14, 1991); and U.S. Pat. No. 5,322,783 (Tomes et al., issued Jun. 21, 1994)). Alternatively, sonication methods (see, for example, Zhang et al., 1991); direct uptake of DNA into protoplasts using CaCl₂) precipitation, polyvinyl alcohol or poly-L-ornithine (see, for example, Hain et al., 1985; Draper et al., 1982); liposome or spheroplast fusion (see, for example, Deshayes et al., 1985; Christou et al., 1987); and electroporation of protoplasts and whole cells and tissues (see, for example, Donn et al., 1990; D'Halluin et al., 1992; and Spencer et al., 1994) have been used to introduce foreign DNA and expression vectors into plants.

### Transformation of Arabidopsis

Transformation of Arabidopsis was performed by an Agrobacterium-mediated protocol based on the method of Bechtold and Pelletier, 1998. Unless otherwise specified, all experimental work was done using the Columbia ecotype.

Plant preparation. Arabidopsis seeds were sown on mesh covered pots. The seedlings were thinned so that 6-10 evenly spaced plants remained on each pot 10 days after planting. The primary bolts were cut off a week before transformation to break apical dominance and encourage auxiliary shoots to form. Transformation was typically performed at 4-5 weeks after sowing.

Bacterial culture preparation. Agrobacterium stocks were inoculated from single colony plates or from glycerol stocks and grown with the appropriate antibiotics until saturation. On the morning of transformation, the saturated cultures were centrifuged and bacterial pellets were re-suspended in Infiltration Media (0.5 X MS, 1X B5 Vitamins, 5% sucrose, 1 mg/ml benzylaminopurine riboside, 200 μl/L Silwet L77) until an A600 reading of 0.8 was reached.

Transformation and seed harvest. The Agrobacterium solution was poured into dipping containers. All flower buds and rosette leaves of the plants were immersed in this solution for 30 seconds. The plants were laid on their side and wrapped to keep the humidity high. The plants were kept this way overnight at 4° C. and then the pots were turned upright, unwrapped, and moved to the growth racks.

The plants were maintained on the growth rack under 24-hour light until seeds were ready to be harvested. Seeds were harvested when 80% of the siliques of the transformed plants were ripe (approximately 5 weeks after the initial transformation). This seed was deemed TO seed, since it was obtained from the TO generation, and was later plated on selection plates (with either kanamycin or sulfonamide, see Example VI). Resistant plants that were identified on such selection plates comprised the T1 generation.

Several independently transformed lines with each gene were grown on MS-agar plates without sucrose. After stratification, a 3h white light treatment was used to synchronize germination, followed by 21 h of dark treatment, before transferring the plates to continuous red light (30-40 μmoles/m 2/s). Hypocotyl lengths were measured from digital images of 20-30 seedlings per line and compared to appropriate controls. Mean values of hypocotyl length were plotted with standard errors to determine whether increased expression of each of the B-box genes resulted in elongated hypocotyls, suggesting a negative role in light signaling.

### Transformation of Tomato Plants

Transformation of tomato plants may be conducted using the protocols of Koornneef et al, 1986, and in U.S. Pat. No. 6,613,962. The latter method is described briefly here. Eight day old cotyledon explants are precultured for 24 hours in Petri dishes containing a feeder layer of Petunia hybrida suspension cells plated on MS medium with 2% (w/v) sucrose and 0.8% agar supplemented with 10 nM α-naphthalene acetic acid and 4.4 nM 6-benzylaminopurine. The explants are then infected with a diluted overnight culture of Agrobacterium tumefaciens containing an expression vector comprising a disclosed or orthologous or paralogous polynucleotide for 5-10 minutes, blotted dry on sterile filter paper and cocultured for 48 hours on the original feeder layer plates. Overnight cultures of Agrobacterium tumefaciens are diluted in liquid MS medium with 2% (w/v) sucrose, pH 5.7) to an OD600 of 0.8.

Following cocultivation, the cotyledon explants are transferred to Petri dishes with selective medium comprising MS medium with 4.56 nM zeatin, 67.3 nM vancomycin, 418.9 nM cefotaxime and 171.6 nM kanamycin sulfate, and cultured under the culture conditions described above. The explants are subcultured every three weeks onto fresh medium. Emerging shoots are dissected from the underlying callus and transferred to glass jars with selective medium without zeatin to form roots. The formation of roots in a kanamycin sulfate-containing medium is a positive indication of a successful transformation.

### Transformation of Soybean Plants

Transformation of soybean plants may be conducted using the methods found in, for example, Mild et al., 1993, and U.S. Pat. Nos. 5,914,451, 5,824,877 and 6,384,301.

For Agrobacterium mediated transformation, soybean seeds are imbibed overnight and the meristem explants excised. Soybean explants are mixed with induced Agrobacterium cells from a strain containing plasmid DNA with the gene of interest cassette and a plant selectable marker cassette no later than 14 hours from the time of initiation of seed imbibition, and wounded using sonication. Following wounding, explants are placed in co-culture for 2-5 days at which point they are transferred to selection media for 6-8 weeks to allow selection and growth of transgenic shoots. Resistant shoots are harvested approximately 6-8 weeks and placed into selective rooting media for 2-3 weeks. Shoots producing roots are transferred to the greenhouse and potted in soil. Shoots that remain healthy on selection, but do not produce roots are transferred to non-selective rooting media for an additional two weeks. Roots from any shoots that produce roots off selection are tested for expression of the plant selectable marker before they are transferred to the greenhouse and potted in soil.

### Transformation of Cotton Plants

Transformation of cotton plants may be performed as generally described in WO0036911, U.S. Pat. Nos. 5,846,797, 7,790,460 and 7,947,869.

### Transformation of Canola Plants

Transformation of rapeseed/canola plants may be performed as described in U.S. Patent Publication No. 2010/0218271. Alternatively, transformation of cotyledonary petioles and hypocotyls of 5-6 day old young canola seedlings are used as explants for tissue culture and transformed according to Babic et al., 1998.

Tissues from in vitro grown canola seedlings are prepared and inoculated with overnight-grown Agrobacterium cells containing plasmid DNA with the gene of interest cassette and a plant selectable marker cassette. Following co-cultivation with Agrobacterium, the infected tissues are allowed to grow on selection to promote growth of transgenic shoots, followed by growth of roots from the transgenic shoots. The selected plantlets are then transferred to the greenhouse and potted in soil. Molecular characterizations are performed to confirm the presence of the gene of interest, and its expression in transgenic plants and progenies. Progeny transgenic plants are selected from a population of transgenic canola events under specified growing conditions and are compared with control canola plants. Control canola plants are substantially the same canola genotype but without the recombinant DNA, for example, either a parental canola plant of the same genotype that is not transformed with the identical recombinant DNA or a negative isoline of the transformed plant.

### Obtaining and Screening Events

The above process is repeated to produce multiple events of transgenic soybean, cotton and canola plant cells that are transformed with recombinant DNA of the present disclosure. Progeny transgenic plants and seed of the transformed plant cells are screened for decreased sensitivity to light, increased yield, greater height, greater stem diameter, greater resistance to lodging, increased secondary rooting, greater cold tolerance, greater tolerance to water deprivation or drought, reduced stomatal conductance, altered C/N sensing, increased tolerance to nitrogen limited conditions, greater late season growth and vigor, greater number of primary nodes, greater late season canopy coverage, increased tolerance to hyperosmotic stress, altered levels of ureides, altered levels of hexose sugars, altered SPS activity, altered levels starch, and delayed senescence.

After a plant or plant cell is transformed (and the latter regenerated into a plant), the transformed plant may be crossed with itself or a plant from the same line, a non-transformed or wild-type plant, or another transformed plant from a different transgenic line of plants. Crossing provides the advantages of producing new and often stable transgenic varieties. Genes and their conferred traits may be moved into distinct lines of plants using traditional backcrossing techniques well known in the art.

### Transformation of Monocots to Produce Increased Yield or Abiotic Stress Tolerance

Cereal plants such as, but not limited to, corn, wheat, rice, sorghum, sugarcane, or barley, may be transformed with the present polynucleotide sequences, including monocot or dicot-derived sequences such as those presented in the present Tables, cloned into an expression vector such as the one described in Table 6 or pGA643 containing a kanamycin-resistance marker or a herbicide-tolerance marker such as a glyphosate-tolerance marker, and expressed constitutively under, for example, the CaMV 35S or COR15 promoters, or with tissue-specific, tissue-enhanced, tissue preferred, diurnally-regulated or inducible promoters. The expression vectors may be one found in the Sequence Listing (for example, pMEN65, SEQ ID NO: 428), or any other suitable expression vector may be similarly used.

The expression vector may be introduced into a variety of cereal plants by means well known in the art including direct DNA transfer or Agrobacterium tumefaciens-mediated transformation. The latter approach may be accomplished by a variety of means, including, for example, that of U.S. Pat. No. 5,591,616, in which monocotyledon callus is transformed by contacting the dedifferentiating tissue with the Agrobacterium containing the expression vector.

The sample tissues are immersed in a suspension of 3×10 ⁻⁹ cells of Agrobacterium containing the expression vector for 3-10 minutes. The callus material is cultured on solid medium at 25° C. in the dark for several days. The calli grown on this medium are transferred to fresh Regeneration medium at 2-3 week intervals(2 or 3 times) until shoots develop. Shoots are then transferred to Shoot-Elongation medium every 2-3 weeks. Healthy-looking shoots are transferred to rooting medium and after roots have developed, the plants are placed into moist potting soil.

For Agrobacterium-mediated transformation of corn embryo cells, corn plants of a readily transformable line are grown in the greenhouse and ears are harvested when the embryos are 1.5 to 2.0 mm in length. Ears are surface sterilized by spraying or soaking the ears in 80% ethanol, followed by air drying. Immature embryos are isolated from individual kernels on surface sterilized ears. Prior to inoculation of maize cells, Agrobacterium cells are grown overnight at room temperature. Immature maize embryo cells are inoculated with Agrobacterium shortly after excision, and incubated at room temperature with Agrobacterium for 5-20 minutes. Immature embryo plant cells are then co-cultured with Agrobacterium for 1 to 3 days at 23° C. in the dark. Co-cultured embryos are transferred to selection media and cultured for approximately two weeks to allow embryogenic callus to develop. Embryogenic callus is transferred to culture medium containing a selective agent and subcultured at about two week intervals. Transformed plant cells are recovered 6 to 8 weeks after initiation of selection. To regenerate transgenic corn plants a callus of transgenic plant cells resulting from transformation and selection is placed on media to initiate shoot development into plantlets which are transferred to potting soil for initial growth in a growth chamber at 26° C. followed by a mist bench before transplanting to 5 inch pots where plants are grown to maturity. The regenerated plants are self-fertilized and seed is harvested for use in one or more methods to select seeds, seedlings or progeny second generation transgenic plants (R2 plants) or hybrids, e.g. by selecting transgenic plants exhibiting an enhanced trait as compared to a control plant.

The transformed plants are analyzed for the presence of the NPTII or other marker protein by ELISA, using the ELISA NPTII kit from 5Prime-3Prime Inc. (Boulder, CO), or for the presence of the transgene by PCR.

Examples of wheat transformation are illustrated in U.S. Pat. Nos. 6,153,812 (microprojectile bombardment method) and 7,026,528 (Agrobacterium-mediated method).

It is also routine to use other methods to produce transgenic plants of most cereal crops (Vasil, 1994) such as corn, wheat, rice, sorghum (Cassas et al., 1993), and barley (Wan and Lemeaux, 1994). DNA transfer methods such as the microprojectile method can be used for corn (Fromm et al., 1990; Gordon-Kamm et al., 1990; Ishida, 1990), wheat (Vasil et al., 1992; Vasil et al., 1993; Weeks et al., 1993), and rice (Christou, 1991; Hiei et al., 1994; Aldemita and Hodges, 1996; and Hiei et al., 1997). For most cereal plants, embryogenic cells derived from immature scutellum tissues are the preferred cellular targets for transformation (Hiei et al., 1997; Vasil, 1994). After microprojectile bombardment the tissues are selected on selection medium to identify the transgenic embryogenic cells (Gordon-Kamm et al., 1990). Transgenic plants are regenerated by standard corn regeneration techniques (Fromm et al., 1990; Gordon-Kamm et al., 1990).

### Example III. Stacking of the Disclosed Polynucleotides with Other Sequences of Interest Including Other Disclosed Polynucleotides

It is envisioned that one or more of the polynucleotides in the instant sequence listing can be introduced into a plant in combination with other disclosed polynucleotide sequences, or with other polynucleotide sequences of interest (that is, the polynucleotides may be “stacked” within the plant) for the purpose of producing plants with enhanced or multiple useful traits. A single plant may thus comprise and ectopically express, overexpress, or suppress one or more of the disclosed polynucleotide sequences and/or their encoded polypeptides. For example, since it was shown that AtBBX32 was physically associated with GmBBX62 in vivo (Example V), introduction and co-expression of both AtBBX32 and GmBBX62 in a transgenic plant may provide the transgenic plant with enhanced traits. Therefore, stacking of the disclosed polynucleotide sequences may be used to create one or more transgenic plants with at least one useful trait, said traits including, for example (but not limited to), decreased sensitivity to light, increased yield, greater height, greater stem diameter, greater resistance to lodging, increased secondary rooting, greater cold tolerance, greater tolerance to water deprivation or drought, reduced stomatal conductance, altered C/N sensing, increased tolerance to nitrogen limiting conditions, improved late season growth and vigor, greater number of nodes, greater late season canopy coverage, increased tolerance to hyperosmotic stress, altered levels of ureides, altered levels of hexose sugars, altered SPS activity, altered levels of starch, and delayed senescence, as compared to a control plant that does not ectopically express or overexpress the disclosed polynucleotide sequences.

The combinations of polynucleotides introduced into target plants may also include multiple copies of any one of the disclosed polynucleotides. The disclosed polynucleotides may also be stacked with any other polynucleotide or combination of polynucleotide to produce transgenic plants with one or more useful trait combinations, including, for example (but not limited to), one or more of the traits listed in the previous paragraph, and possibly herbicide resistance (e.g., glyphosate resistance), disease resistance or tolerance, increased tolerance to heat, increased tolerance to oxidative stress, increased oil content, male sterility, lodging resistance, early flowering and/or development, delayed flowering and/or development, extended flowering and/or development time, traits desirable for chemical processing, and/or improved digestibility.

The combinations of polynucleotides within an individual plant can be introduced through a variety of means. This may include, for example, transforming a target plant with two or more nucleic acid sequences of interest at the same time or at different times, or introducing the two or more nucleic acid sequences of interest by different means such as a combination of transformation and breeding, where the nucleic acid sequences may be integrated into one or more loci. The two sequence cassettes can be contained in separate expression vectors (trans), where one of the expression vectors comprises a trans-acting element. A trans-acting element is a DNA sequence that controls transcriptional activity of a target gene through a diffusible gene product such as a protein, microRNA, or other diffusible repressor or activator. The regulated target gene may include a polynucleotide of the instant Sequence Listing. Alternatively, the two sequence cassettes can be contained in the same expression vector (cis). Introducing the sequences at the same time into a plant may be referred to as co-transformation, simultaneous transformation, or parallel transformation. Combinations of recombinant polynucleotides can also be introduced into a plant through subsequent, serial or super-transformation or re-transformation, that is, by providing a transgenic plant previously transformed with one polynucleotide of interest, and transforming the same plant with one or more distinct recombinant polynucleotides. In this method, a transgenic plant exhibiting one or more desired traits can be used as a target plant to introduce additional traits by super-transformation.

Thus, if the polynucleotide sequences (including one or more of the disclosed polynucleotide sequences) are introduced into plants by genetic transformation, the polynucleotide sequences can be transformed into the plant at any time and in any order.

Another method of stacking genes in a plant combines transformation with breeding methods. A plant that has been transformed with at least one polynucleotide sequence of interest (including one or more of the disclosed polynucleotide sequences) may be cross-bred with a transgenic or non-transformed plant in order to stack more than one gene in the plant.

Expression of the polynucleotide sequences can be regulated by the same promoter or by different promoters.

It may also be desirable to introduce an expression vector or cassette that will suppress the expression of a polynucleotide of interest. This may be combined with any combination of other suppression vectors or cassettes or overexpression vectors or cassettes to generate a desirable combination of traits in the plant, including traits disclosed in this Example.

### Example IV: Expression and Analysis of Increased Yield and/or Abiotic Stress Tolerance in non-Arabidopsis Species or Crop Plants

It has been shown that overexpression of a B-box polypeptide, BBX32 (At.BBX32; SEQ ID NO: 430), decreases light sensitivity of plants when the polypeptide is overexpressed in the plants, including in plants other than Arabidopsis (e.g., crop plants). BBX32 has been shown to improve crop performance and acts to repress the transcriptional activity of another B-box protein, AtBBX21 (G1482, SEQ ID NO: 56) on its native target promoter (prCHS) sequences (unpublished data). The instantly disclosed results provide evidence that HA:AtBBX28 (G1481) inhibited the transcriptional activity of HA:AtBBX21 to levels similar to the repression by HA:BBX32 (FIG. 2), suggesting that AtBBX28 is able to act as a repressor like BBX32. AtBBX28 may have common targets with BBX32 (FIG. 2) or it may act on specific targets independently of BBX32.

In fact, a number of the disclosed polypeptide sequences, including those found in the Sequence Listing, including SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234,436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548 or 550 or orthologous sequences (as defined or claimed herein) that function in the same manner, may alter light sensitivity of plants when the sequences are overexpressed in the plants. It is expected that structurally similar orthologs and paralogs of the disclosed polypeptide sequences, which may be derived from diverse plant species, will also alter light sensitivity of plants when these orthologous and paralogous sequences are overexpressed. Furthermore, it is expected that the disclosed B-box polypeptide sequences, and structurally similar orthologs and paralogs of the disclosed B-box polypeptide sequences, can, at least in part and as a result of their altering light sensitivity, confer increased yield or increased tolerance to a number of abiotic stresses, including water deprivation or drought, cold, and nitrogen-limiting conditions, altered levels of ureides, altered levels of hexose sugars, altered SPS activity, altered levels of starch, and delayed senescence, relative to control plants, and thus may increase yield of crop or other commercially important plant species.

Relationships between the disclosed sequences in the present disclosure may be recognized by comparing structural similarity, for example, determined by a disclosed percentage identity to full length protein or their conserved domains, or by hybridization to a disclosed polynucleotide sequence.

Northern blot analysis, RT-PCR or microarray analysis of the regenerated, transformed plants may be used to show expression of a polypeptide of the present disclosure, or related orthologous or paralogous sequences that are capable of inducing increased yield or increased tolerance to abiotic stresses, including water deprivation, cold, and low nitrogen conditions.

After a eudicot plant, dicot plant, monocot plant or plant cell has been transformed (and the latter regenerated into a plant) and shown to have increased tolerance to abiotic stresses, including water deprivation or drought, cold, and nitrogen-limiting conditions, that is, able to tolerate greater planting density with a coincident increase in yield, or tolerance to abiotic stress, or produce greater yield relative to a control plant in the absence of stress conditions or under stress conditions, the transformed plant may be crossed with itself or a plant from the same line, a non-transformed or wild-type plant, or another transformed plant from a different transgenic line of plants.

The functions of specific polypeptides, including closely-related orthologs, may be analyzed and may be further characterized and incorporated into crop plants. The ectopic overexpression of these sequences may be regulated using constitutive, inducible, or tissue specific regulatory elements. Genes that have been examined and have been shown to modify plant traits (including altered light sensitivity with predicted increased yield and/or abiotic stress tolerance) encode polypeptides found in the Sequence Listing. In addition to these sequences, it is expected that newly discovered polynucleotide and polypeptide sequences closely related to polynucleotide and polypeptide sequences found in the Sequence Listing can also confer alteration of traits in a similar manner to the sequences found in the Sequence Listing, when transformed into any of a considerable variety of plants of different species, and including dicots and monocots. The polynucleotide and polypeptide sequences derived from monocots (e.g., the rice sequences) may be used to transform both monocot and dicot plants, and those derived from dicots (e.g., the Arabidopsis and soy genes) may be used to transform either group, although it is expected that some of these sequences will function best if the gene is transformed into a plant from the same group from which the sequence is derived.

As an example of a first step to determine water deprivation-related tolerance, seeds of these transgenic plants may be subjected to germination assays to measure sucrose sensing, severe desiccation or drought. Methods that may be used to measure relative sucrose sensing, severe desiccation tolerance or drought tolerance in genetically altered (for example, over-expressing) and control plants are as follows. For sucrose sensing, germination assays may be conducted in growth media containing 9.4% sucrose with Arabidopsis overexpressors of BBX32 and closely-related sequences. Growing the plants under controlled temperature and humidity on sterile medium produces uniform plant material that has not been exposed to additional stresses (such as water stress) which could cause variability in the results obtained. Where possible, assay conditions are originally tested in a blind experiment with controls that have phenotypes related to the condition tested.

Prior to plating, seed for all experiments are surface sterilized in the following manner: (1) 5 minute incubation with mixing in 70% ethanol, (2) 20 minute incubation with mixing in 30% bleach, 0.01% triton-X 100, (3) 5×rinses with sterile water, (4) Seeds are re-suspended in 0.1% sterile agarose and stratified at 4° C. for 3-4 days. Germination assays follow modifications of the same basic protocol. Sterile seeds are sown on the conditional media that has a basal composition of 80% MS+Vitamins Plates are incubated at 22° C. under 24-hour light (120-130 μE m⁻²S⁻¹) in a growth chamber. Evaluation of germination and seedling vigor is performed five days after planting.

For severe desiccation (plate-based water deprivation) assays, seedlings are grown for 14 days on MS+Vitamins+1% Sucrose at 22° C. Plates are opened in a sterile hood for 3 hr for hardening and then seedlings are removed from the media and dried for two hours in the sterile hood. After this time the plants are transferred back to plates and incubated at 22° C. for recovery. The plants are then evaluated after five days.

The soil drought assay (performed in clay pots) is based on that described by Haake et al., 2002.

Previously, we have performed clay-pot assays on segregating T2 populations, sown directly to soil. However, in the current procedure, seedlings are first germinated on selection plates containing either kanamycin or sulfonamide.

Seeds are sterilized by a 2 minute ethanol treatment followed by 20 minutes in 30% bleach/0.01% Tween and five washes in distilled water. Seeds were sown to MS agar in 0.1% agarose and stratified for three days at 4° C., before transfer to growth cabinets with a temperature of 22° C. After seven days of growth on selection plates, seedlings are transplanted to 3.5 inch diameter clay pots containing 80 g of a 50:50 mix of vermiculite:perlite topped with 80 g of ProMix. Typically, each pot contains 14 seedlings, and plants of the transgenic line being tested are in separate pots to the wild-type controls. Pots containing the transgenic line versus control pots are interspersed in the growth room, maintained under 24-hour light conditions (18-23° C., and 90-100 μE m⁻²S⁻¹) and watered for a period of 14 days. Water is then withheld and pots are placed on absorbent paper for a period of 8-10 days in to apply a drought treatment. After this period, a visual qualitative “drought score” from 0-6 is assigned to record the extent of visible drought stress symptoms. A score of “6” corresponds to no visible symptoms whereas a score of “0” corresponds to extreme wilting and the leaves having a “crispy” texture. At the end of the drought period, pots are re-watered and scored after 5-6 days; the number of surviving plants in each pot is counted, and the proportion of the total plants in the pot that survived is calculated.

In a given experiment, six or more pots of a transgenic line are compared with six or more pots of the appropriate control. The mean drought score and mean proportion of plants surviving (survival rate) are calculated for both the transgenic line and the wild-type pots. In each case a p-value* is calculated, which indicates the significance of the difference between the two mean values. The results for each transgenic line across each planting for a particular project are then presented in a results table.

Calculation of p-values. For the assays where control and experimental plants were in separate pots, survival is analyzed with a logistic regression to account for the fact that the random variable is a proportion between 0 and 1. The reported p-value is the significance of the experimental proportion contrasted to the control, based upon regressing the logit-transformed data.

Drought score, being an ordered factor with no real numeric meaning, is analyzed with a non-parametric test between the experimental and control groups. The p-value is calculated with a Mann-Whitney rank-sum test.

Plants overexpressing the disclosed, orthologous or paralogous sequences may be found to be more tolerant to high sucrose by having better germination, longer radicles, and more cotyledon expansion.

Disclosed or orthologous or paralogous sequences, or other sequences closely related to the disclosed B-box polynucleotides and B-box polypeptides, may also be used to generate transgenic plants that are more tolerant to nitrogen-limiting conditions or cold than control plants.

All of these abiotic stress tolerances conferred by disclosed B-box polynucleotides and B-box polypeptides may contribute to increased yield of commercially available plants. BBX32 overexpressors have been shown to increase yield of plants in the apparent absence of significantly obvious abiotic stress, as evidenced by including increased height, increased early season vigor and estimated stand count, and increased late season canopy coverage observed in soy plants overexpressing BBX32. Thus, it is expected that disclosed B-box polynucleotides and B-box polypeptides, and closely related orthologs and paralogs, will also improve yield in plants relative to control plants, including in leguminous species, even in the absence of overt abiotic stresses.

It is expected that the disclosed methods may be applied to identify other useful and valuable sequences of the present polypeptide clades, and the sequences may be derived from a diverse range of plant species. These sequences and/or the sequences provided in the Sequence Listing may be introduced into plants other than Arabidopsis (e.g., crop plants), and by expressing said sequences may thus be made more tolerant than controls to water deprivation assays, nitrogen-limiting conditions or cold, and/or produce greater yield. It is expected that said plants under these conditions are greener, more vigorous, will have better survival rates than controls, or will recover better from these treatments than control plants.

### Example V. Results

The data presented herein present the results obtained in experiments with polynucleotides and polypeptides that may be expressed in plants in order to obtain advantageous traits in said plants including improved yield under non-stressed or low stress conditions, or for reduced yield losses under biotic and abiotic stress conditions.

We have previously demonstrated that under a variety of conditions, including unstressed, nutrient stress, or osmotic stress conditions, Arabidopsis seedlings ectopically expressing AtBBX32 showed increased hypocotyl growth rates and altered transcription of genes associated with light regulated signal transduction and photosynthesis. Soybean plants ectopically expressing AtBBX32 were taller, tended to have more nodes, pods, and flowers, greater late season canopy coverage, improved late season vigor, in some cases had thicker stems, and ultimately had significantly higher broad acre yield than wild-type soybean plants did (e.g., see Creelman et al., U.S. Pat. No. 7,692,067). Based on a phylogenetic analysis of the soybean B-box family, G4004 (also referred to as Glycine max BBX53 or GmBBX53, and listed as SEQ ID NO: 432 or 532), GmBBX52 variant (SEQ ID NO: 434) and GmBBX52 (SEQ ID NO: 530) were identified as the soybean orthologs of the Arabidopsis AtBBX32 gene. Like AtBBX32, both soybean genes contain a single N-terminus B-box domain. Both soybean genes had diurnal oscillations in transcript abundance. Arabidopsis plants overexpressing either GmBBX52 or GmBBX53 displayed increased hypocotyl growth rates and altered transcription of genes associated with light regulated signal transduction and photosynthesis, similar to plant lines overexpressing AtBBX32. In order to better understand the biological role of GmBBX52 and GmBBX53 genes on soybean yield, we generated constructs that would constitutively express either GmBBX52 or GmBBX53 or knockdown both GmBBX52 and GmBBX53 transcript levels. Eight independently generated plants from both the GmBBX52 constitutive over-expression construct and the GmBBX52/GmBBX53-miRNA constructs and four independent events from the GmBBX53 over-expression construct were tested in broad acre yield trials. Lines ectopically over-expressing GmBBX52 yielded, on average, 6.1% more bushels per acre than did wild-type control plants, while the top performing line yielded 9.0% more bushels per acre. Lines ectopically over-expressing GmBBX53 yielded 4.1% higher than the wild-type control, while the top event improved yield by 6.7% over the wild-type control. In contrast, miRNA mediated suppression of GmBBX52 and GmBBX53 transcript levels led to a significant decrease in GmBBX52 RNA levels and decreased yield.

GmBBX52/GmBBX53-miRNA lines yielded, on average, 5.5% fewer bushels per acre than control lines while the lowest yielding line across the eight produced 11.8% fewer bushels per acre relative to controls. These data demonstrate that the ectopic expression of the soybean homologs of AtBBX32, GmBBX52 and GmBBX53, led to yield improvements similar to that of soybean plants expressing the Arabidopsis gene.

We then examined several independently transformed Arabidopsis lines overexpressing other disclosed B-box polynucleotides. The level of gene expression is expected to vary in each line, which is likely to result in a range of hypocotyl lengths. Lines with hypocotyl lengths similar to the controls are likely to lack any significant expression from the transgene. On the other hand, an increase in hypocotyl length may be attributed to the transgene, either through an indirect effect or directly through overexpression of the specific B-box transgene. Increased hypocotyl lengths of multiple independent transformants of the same gene can be used to correlate the reduced light responsiveness to increased gene expression. Using this method, a number of independent Arabidopsis transgenic lines carrying 35S::AtBBX28:cMYC were found to have longer hypocotyls than control lines transformed with an empty vector control (pMEN 65), and some of the 35S::AtBBX28:cMYC lines had hypocotyl lengths similar to BBX32-over-expressing seedlings (FIG. 1). These data suggest that seedlings overexpressing AtBBX28 are hyposensitive to red light implying that AtBBX28 functions negatively in light signal transduction.

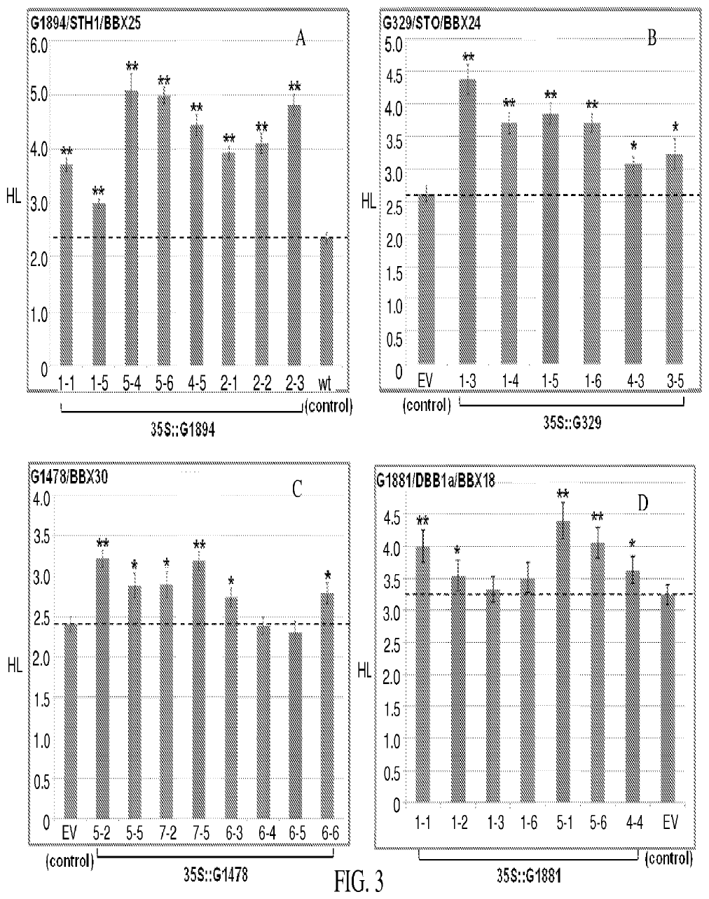

BBX32 improves crop performance and is thought to repress the transcriptional activity of another B-box protein, AtBBX21 (SEQ ID NO: 56) on its native target promoter (prCHS) sequences (unpublished data). To test whether AtBBX28 (SEQ ID NO: 2) can also repress gene expression, we co-transformed prCHS::GUS reporter constructs with HA:AtBBX21 or in combination with HA:AtBBX28, HA: BBX32 or CAT (used as control) in Arabidopsis. Our results show that HA:AtBBX28 inhibited the transcriptional activity of HA:AtBBX21 to levels similar to the repression by HA: BBX32 (FIG. 2). These data suggest that AtBBX28 is able to act as a repressor like BBX32. AtBBX28 may have common targets with BBX32 (FIG. 2) or it may act on specific targets independently of BBX32. Other B-box proteins were analyzed for their potential roles in light signaling. Seedlings of several independently transformed Arabidopsis lines transformed with B-box genes were grown under red light to examine the effects on hypocotyl length. As seen in FIGS. 3 and 4, we identified 8 additional B-box genes which, when constitutively expressed, produced longer hypocotyls in light. The levels of expression of these genes in the transgenic lines are not yet known, but as expected the magnitude of the hypocotyl length phenotype varied greatly between independent transformants of the same gene. These results indicate that AtBBX25 (G1894, SEQ ID NO: 154), AtBBX26 (G1486, SEQ ID NO: 52), AtBBX30 (G1478, SEQ ID NO: 40), AtBBX7 (G2440, SEQ ID NO: 104), AtBBX24 (G329, SEQ ID NO: 152), AtBBX20 (G1888, SEQ ID NO: 54), AtBBX19 (G902, SEQ ID NO: 200), and AtBBX18 (G1881, SEQ ID NO: 202) function to suppress light signaling. One or more of these genes can be used to alter light sensitivity leading to increased crop yield and stress tolerance.

AtBBX32 modulates expression of soybean BBX proteins

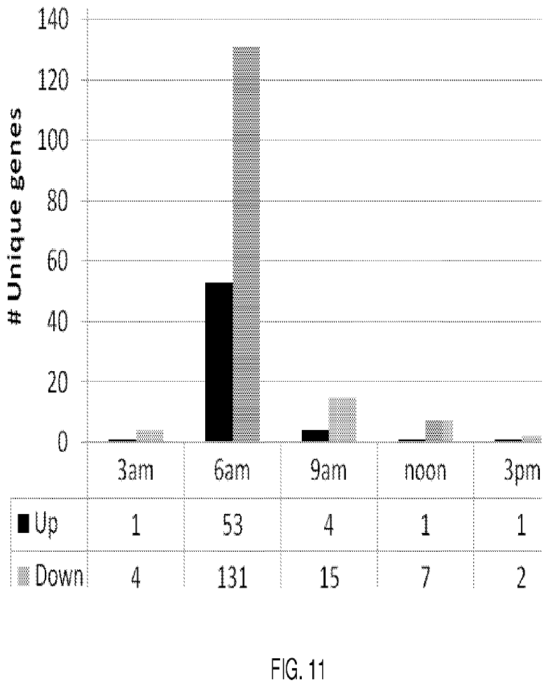

A microarray analysis was performed to examine the expression of AtBBX32 on the modulation of gene expression in field grown soybean sampled at five time points: 3:00 am, 6:00 am (dawn), 9:00 am, noon, and 3:00 pm. The expression of AtBBX32 in soybean affected the abundance of specific gene transcripts with the majority of these changes in gene expression occurring at dawn as shown in FIG. 11.

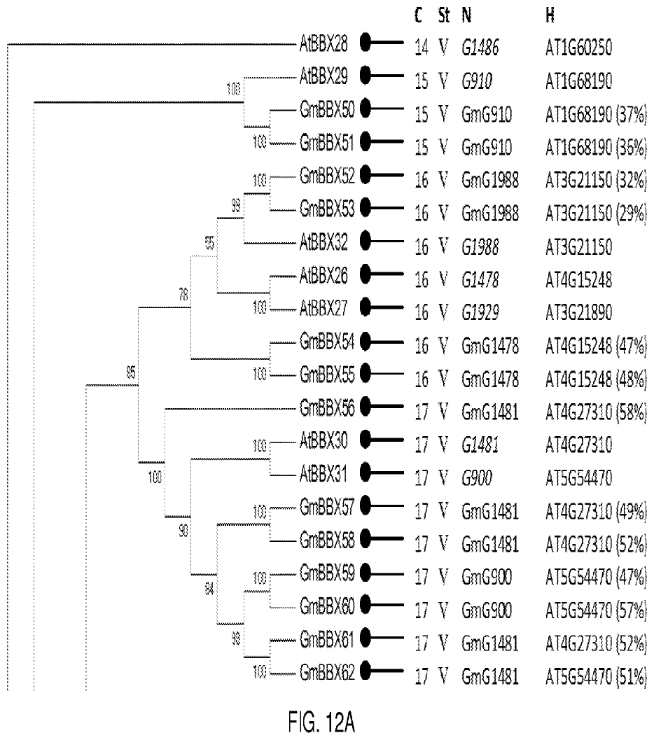

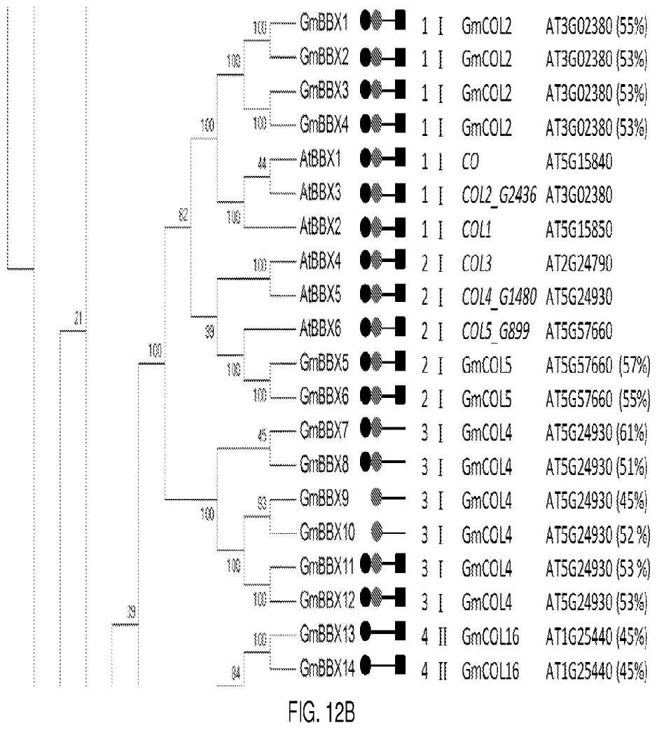

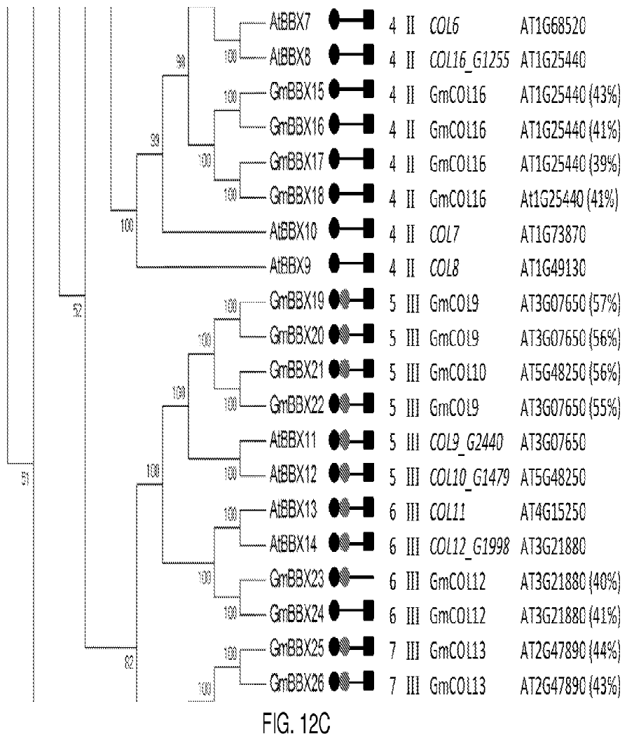

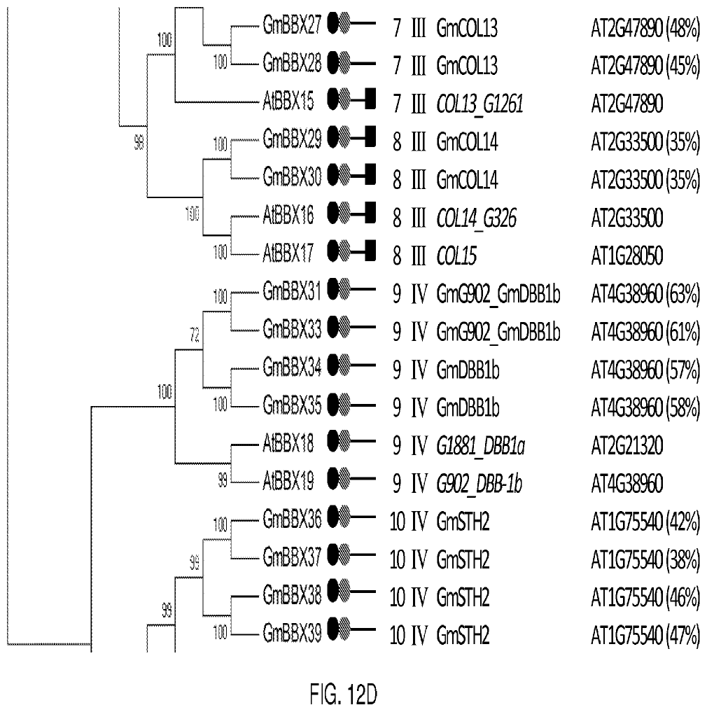

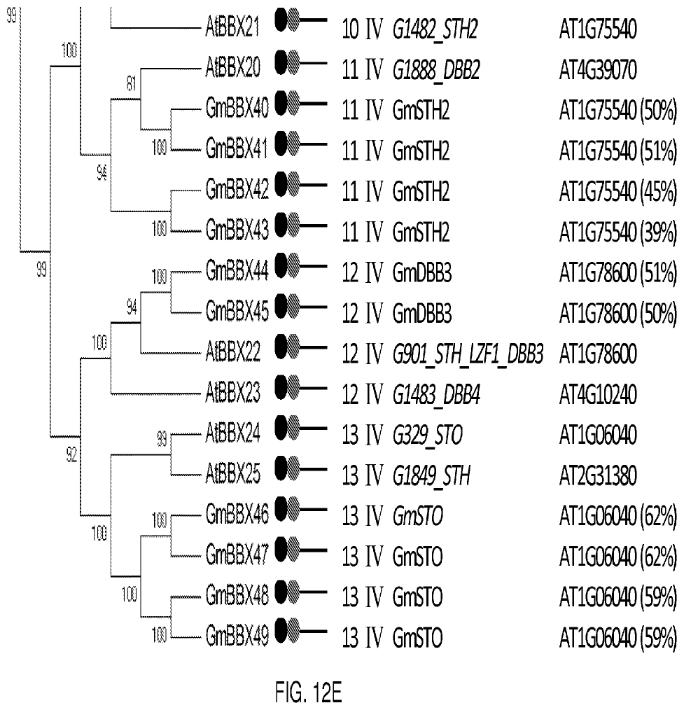

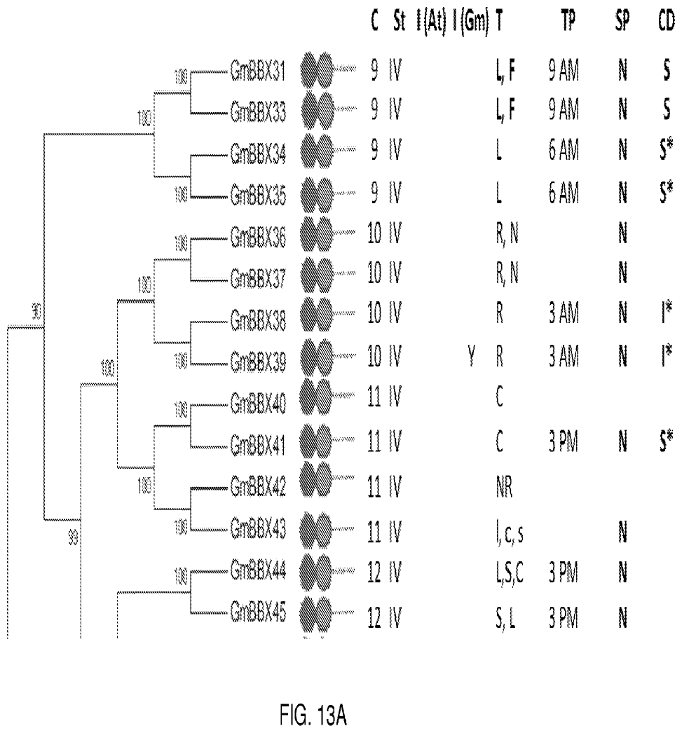

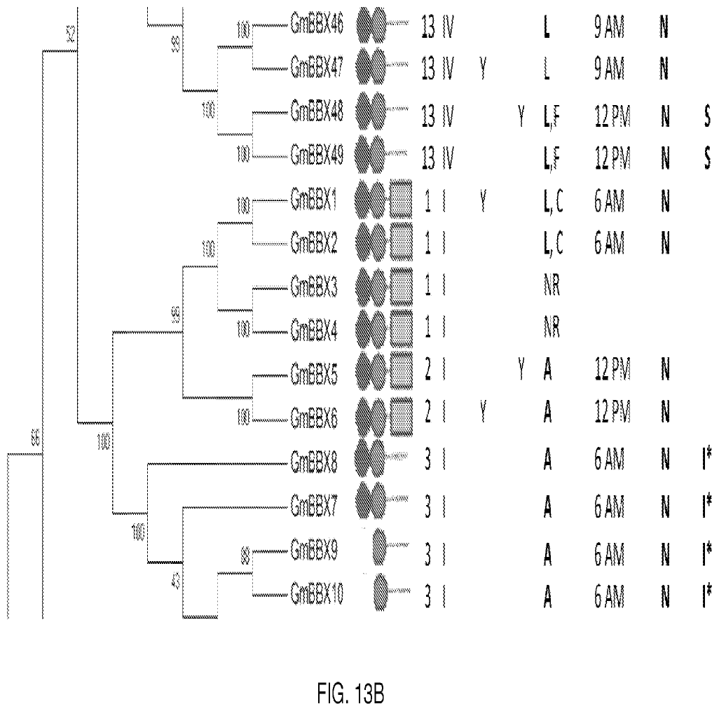

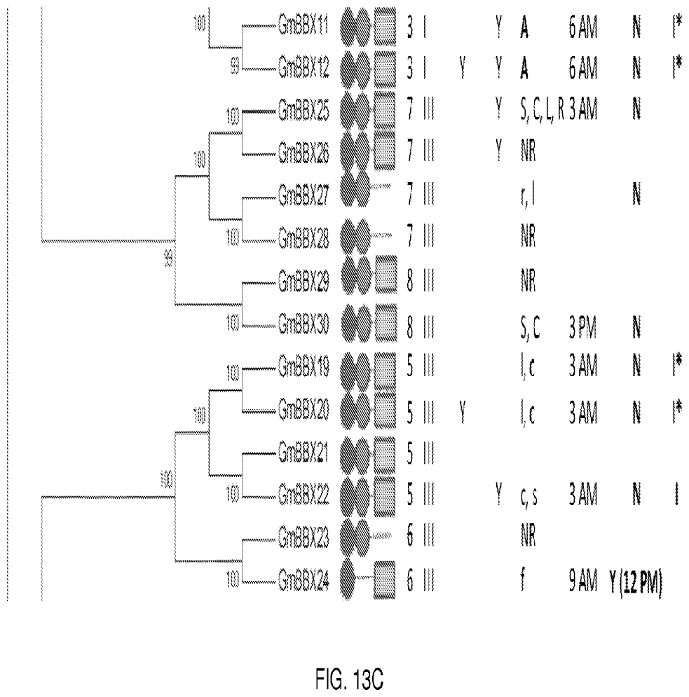

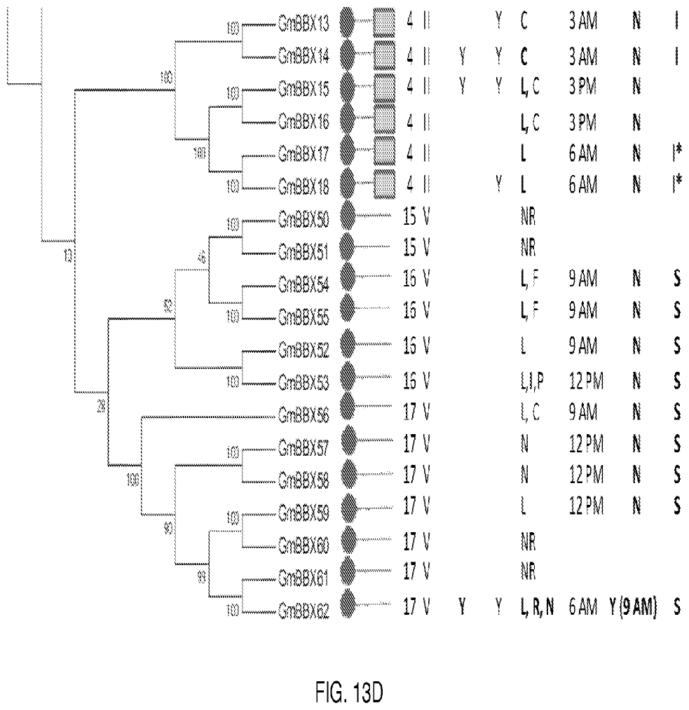

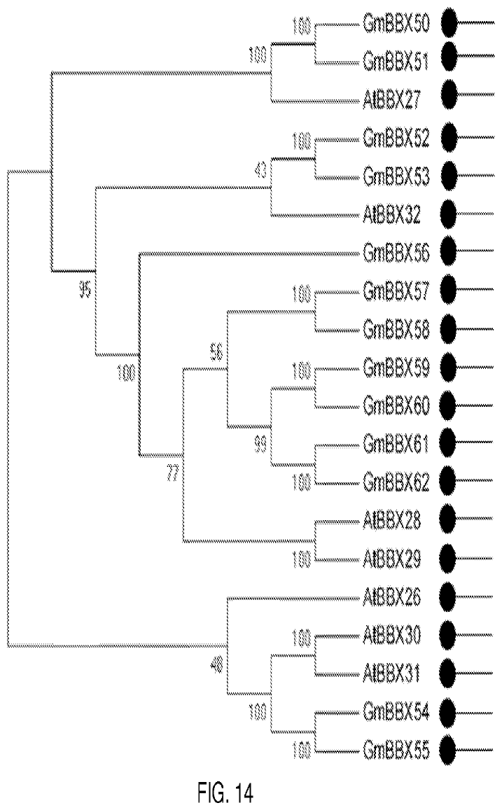

Phylogenetic analyses of the Arabidopsis thaliana and Glycine max B-box gene families were conducted for the purpose of identifying functional similarities of proteins in the B-box family (FIGS. 12, 13 and 14). The phyogenetic trees were generated using an in-house proprietary database and clustalW analysis clusters to group sequences based on the domain structure of the proteins. The genomes of both species contain large families of B-box domain genes; the Arabidopsis genome contains 32 predicted B-box domain proteins, while the paleopolyploid soybean genome contains 61 B-box containing genes. The phylogenetic analysis conducted using 32 B-box sequences from Arabidopsis thaliana and 61 B-box sequences from Glycine max grouped the Arabidopsis and soybean sequences into five structural groups and seventeen clades (FIG. 12). A phylogenetic tree specific for the 61 B-box sequences from soybean is shown in FIG. 13. Two of the soybean B-box sequences showed a shift in the time of peak expression. Three sequences were induced significantly at dawn and thirteen were down-regulated in the At.BBX32 transgenic soybean plants (FIG. 13). A single B-box Glade is further represented in FIG. 14 for Arabidopsis thaliana and Glycine max sequences. Within the phylogenetic grouping of AtBBX32-like single B-box domain containing genes, thirteen B-box genes from Glycine max grouped with seven Arabidopsis single B-box genes. Phylogenetic analysis indicates that GmBBX52 and GmBBX53 are orthologs of the Arabidopsis thaliana AtBBX32 gene.

### Utilities of B-Box Polynucleotides, Polypeptides and Phylogenetically-Related Sequences

Based on the data obtained in the instantly disclosed Examples, a total of nine Arabidopsis B-box genes and several GmBBX genes that function negatively in light signaling have been identified, and it is expected that either independently or in combination with other genes or specific promoters (for example, heterologous promoters, or constitutive, inducible, diurnally-regulated, tissue-enhanced, tissue-preferred, or tissue-specific promoters), these genes will provide significantly improved yield benefits and resistance to abiotic stress.

Sequences that are orthologous, i.e., full length sequences or modified sequences that share significant sequence identity or similarity, to those provided in the Sequence Listing, may be derived from any of a wide variety of plants, including but not limited to, the plant families Myrtaceae, Pinaceae, Salicaceae, Leguminosae, Umbelliferae, Cruciferae, Curcurbitaceae, Solanaceae, Brassicaceae, or from the plants Arabidopsis thaliana, soybean, potato, cotton, rape, oilseed rape (including canola), sunflower, alfalfa, clover; or fruits and vegetables, such as banana, blackberry, blueberry, strawberry, and raspberry, cantaloupe, carrot, cauliflower, coffee, cucumber, eggplant, mint, grapes, honeydew, lettuce, mango, melon, onion, papaya, peas, beans, peppers, pineapple, pumpkin, spinach, squash, tobacco, tomato, tomatillo, watermelon, rosaceous fruits (such as apple, peach, pear, cherry and plum), vegetable brassicas (such as broccoli, cabbage, cauliflower, Brussels sprouts, and kohlrabi), barley; rye; millet; sorghum; currant; avocado; citrus fruits such as oranges, lemons, grapefruit and tangerines, artichoke, cherries; nuts such as the walnut and peanut; endive; leek; roots such as arrowroot, beet, cassava, turnip, radish, yam, sweet potato, wheat, corn or sweet corn (maize), rice, wild rice, sugarcane, bamboo, oats, turfgrass, brome-grass, Miscanthus, pampas grass, or switchgrass (Panicum). In addition, homologous sequences may be derived from plants that are evolutionarily-related to crop plants, but which may not have yet been used as crop plants. Examples include deadly nightshade (Atropa belladona), related to tomato; jimson weed (Datura strommium), related to peyote; and teosinte (Zea species), related to corn (maize).

Transgenic plants (or plant cells, or plant explants, or plant tissues) incorporating the disclosed polynucleotides and expressing the disclosed polypeptides or orthologs of the disclosed polypeptides can be produced by a variety of well-established techniques as described in the Examples. After construction of a nucleic acid vector, including an expression vector or cassette, including one or more polynucleotides encoding one or more B-box transcriptional regulators or B-box transcriptional regulator orthologs, standard techniques can be used to introduce the vector into a plant, a plant cell, a plant explant or a plant tissue of interest. Optionally, the plant cell, explant or tissue can be regenerated to produce a transgenic plant.

The plant can be any higher plant, including gymnosperms, monocotyledonous and dicotyledonous plants. Suitable protocols are available for Leguminosae (alfalfa, soybean, clover, etc.), Umbelliferae (carrot, celery, parsnip), Cruciferae (cabbage, radish, rapeseed, broccoli, etc.), Curcurbitaceae (melons and cucumber), Poaceae (formerly Gramineae, including wheat, corn, rice, barley, millet, etc.), Solanaceae (potato, tomato, tobacco, peppers, etc.), and various other crops. See protocols described in Ammirato et al., 1984; Shimamoto et al. 1989; Fromm et al., 1990; and Vasil et al., 1990).

Identification of interacting partners with AtBBX32

Genetic and biochemical approaches were employed to identify proteins that may interact with AtBBX32 in transgenic soybean plants. Engineering of crop plants with polynucleotides that encode or suppress such proteins may improve yield and other agronomic traits.

### Yeast 2-Hybrid Screen

In order to identify BBX32 binding partners that may be involved in the regulation of AtBBX32 in overexpressing transgenic soybean, a yeast 2-hybrid (Y2H) screen was carried out using full length and truncatedAtBBX32 and the soy orthologGmBBX52 as baits in a total of 6 screens against a soybean cDNA library (Table 7). The six BBX32 baits from Table 7 were subcloned into pUC vectors and then recloned as LexA fusion constructs for use in Y2H screens. Most bait constructs were prepared in the pB27 background, which placed the LexA gene at the N terminus of the bait sequence. Other vector backbones include pB29 for C terminal LexA fusions and pB6 for N terminal Gal4 fusions. Two control screens were also performed using GmCOP1-like protein and GmPABP-like (Poly A binding protein) protein as baits (data not shown).

A random primed library was made from total RNA isolated from wild-type non-transgenic V5 soy leaves harvested at 4 hr intervals during a 14 hr diurnal cycle as well as from dark germinated soy seedlings. The two uppermost leaf tri-foliates, shoot, and meristem tissues were harvested from V5 stage soy plants at 3 am, 7 am, 11 am, 3 pm, 7 pm, and 11 pm. Whole seedlings were collected at a single time point. All tissues were harvested from plants grown in the growth chamber where lights were turned on at 5 am and off at 7 pm. RNA was isolated from all tissues for preparation of a cDNA library in the appropriate prey vector. The two-hybrid screens were performed by Hybrigenics Services.

Interacting clones were isolated and sequenced. Combining all six screens, approximately 471 interacting contigs were identified. Contigs are defined as an interacting fragment of a particular sequence. Separate contigs may represent overlapping portions of the same mRNA. Furthermore, some contigs identified in separate screens may be identical, thus reducing the total number of contigs identified. Translated BLAST searches against the public database revealed approximately 45 groups of contigs and 57 single contigs (including 333 different contigs) with similarity to Arabidopsis genes. An additional three contig groups did not show significant homology to publicly available gene sequences. Six contig groups did not show significant homology to genes within the Arabidopsis genome nor to the map to known soy genes. Of the remaining single contigs that map to soy gene sequences, approximately 107 showed no matches to known plant genes.

TABLE 7
Bait insert sequences tested against the soy DNA library
described above. Italicized sequence in N and C terminal baits
contained overlap regions pesent in both bait constructs.

| Bait | Sequence | Nucleotides |
|---|---|---|
| N-terminal | MVSFCELCGAEADLHCAADSAFLCRSCDAKFHASNFLFARHFRRVICPNC | 1-222 |
| At.BBX32 | KSLTQNFVSGPLLPWPPRTTCCSE | |
| C-terminal | TQNFVSGPLLPWPPRTTCCSESSSSCCSSLDCVSSSELSSTTRDVNRARG | 160-678 |
| At.BBX32 | RENRVNAKAVAVTVADGIFVNWCGKLGLNRDLTNAVVSYASLALAVETRP | |
| | RATKRVFLAAAFWFGVKNTTTWQNLKKVEDVTGVSAGMIRAVESKLARAM | |
| | TQQLRRWRVDSEEGWAENDNV | |
| Full length | MVSFCELCGAEADLHCAASSAFLCRSCDAKFHASNFLFARHFRRVICPNC | 1-678 |
| At.BBX32 | KSLTQNFVSGPLLPWPPRTTCCSESSSSSCCSSLDCVSSSELSSTTRDVN | |
| | RARGRENRVNAKAVAVTVADGIFVNWCGKLGLNRDLTNAVVSYASLALAV | |
| | ETRPRATKRVFLAAAFWFGVKNTTTWQNLKKVEDVTGVSAGMIRAVESKL | |
| | ARAMTQQLRRWRVDSEEGWAENDNV | |
| N-terminal | MKPKTCELCHQLASLYCPSDSAFLCFHCDAAVHAANFLVARHLRRLLCSK | 1-228 |
| Gm.BBX52 | CNRFAAIHISGAISRHLSSTCTSCSL | |
| C-terminal | AAIHISGAISRHLSSTCTSCSLEIPSADSDSLPSSSTCVSSSESCSTNQI | 163-732 |
| Gm.BBX52 | KAEKKRRRRRRSFSSSSVTDDASPAAKKRRRNGGSVAEVFEKWSREIGLG | |
| | LGVNGNRVASNALSVCLGKWRSLPFRVAAATSFWLGLRFCGDRGLATCQN | |
| | LARLEAISGVPAKLILGAHANLARVFTHRRELQEGWGES | |
| Full length | MKPKTCELCHQLASLYCPSDSAFLCFHCDAAVHAANFLVARHLRRLLCSK | 1-732 |
| Gm.BBX52 | CNRFAAIHISGAISRHLSSTCTSCSLEIPSADSDSLPSSSTCVSSSESCS | |
| | TNQIKAEKKRRRRRRSFSSSSVTDDASPAAKKRRRNGGSVAEVFEKWSRE | |
| | IGLGLGVNGNRVASNALSVCLGKWRSLPFRVAAATSFWLGLRFCGDRGLA | |
| | TCQNLARLEAISGVPAKLILGAHANLARVFTHRRELQEGWGES | |

Analysis of the Y2H prey identified in each screen revealed several consistent patterns of potential interaction. In general, the N-terminal, B-Box containing region of both the AtBBX32 and Gm BBX32 proteins showed potential interaction with other B-Box proteins. Many of the potentially interacting proteins are expressed during a timeframe in which AtBBX32 overexpression has been shown to influence expression of the most mRNAs in growth chamber experiments (2 hr predawn and dawn). At a molecular level, AtBBX32 overexpression resulted in marked changes in mRNA expression for a number of nuclear genes, suggesting that AtBBX32 plays a role in transcriptional regulation (FIG. 11). As a putative component of the transcriptional network, it is likely that BBX32 functions through interactions with other proteins. Taken together, these data indicate that, like BBX32, these genes may modulate a variety of traits, including yield, when overexpressed or suppressed in plants.

AtBBX32 putative interacting proteins from the yeast 2 hybrid screens were identified and are further described in the phylogenetic tree in FIGS. 12 and 13. Several possible interacting proteins of GmBBX52 were also identified from yeast 2 hybrid screens. For example, GmG1481(GmBBX62) was identified and is proposed to function in protein-protein stabilization and could facilitate light signaling in leaves, roots and nodules. The interaction of BBX32 with B-box proteins may also help in stabilizing the nuclear complex. Together such a complex could lead to altered gene expression of key regulatory and metabolic genes resulting in enhanced vigor, growth and yield.

### AtBBX32 and GmBBX62 are Physically Associated In Vivo

To confirm the interaction between AtBBX32 and GmBBX62 identified through Y2H screening: the ability of AtBBX32 to associate with GmBBX62 in vivo was tested. AtBBX32::GFP and Flag::GmBBX62 fusion proteins were transiently co-expressed in soybean protoplasts. Soybean protoplasts were isolated from 4-6 mm cotyledons and transformed as described by Abel and Theologis, 1994. Approximately 1×10⁶ protoplasts were transformed in 15 mL Falcon conical tubes with 90 ng of Qiagen prepped plasmid DNA. Protoplasts were then incubated at 22° C. for 18 to 24 hours, and harvested at 150×g for 3 minutes and lysed with lysis buffer (50 mM Tris pH 7.8, 150 mM NaCl, 1% Triton-X 100 and Complete Protease Inhibitor (Roche) for 1 hr on ice with vortex mixing every 15 min. The lysate was centrifuged for 5 mM at 3000×g and soluble fractions were retained for use in Luminex-based co-immunoprecipitation assays.

Luminex co-immunoprecipitation (co-IP) assay was carried out using the miniaturized sandwich immunoassay and co-IP method with modifications, Poetz et al., 2009. This combination of co-IP and sandwich immunoassay allows the relative quantification of components of the complex. Briefly, antibodies for GFP (MBL International, D153-3) and FLAG (Bethyl Laboratories, A190-101A) were covalently coupled to carboxylated fluorescent microspheres (Luminex) according to the manufacturer's protocol. Fifty n1 of protoplast cell lysate (above) was aliquoted to three different wells in a 96 well clear flat-bottom plate (Bio-Rad) for Luminex assays. To each sample, 50 n1 of conjugated beads was added to each appropriate well and incubated for 30 minutes with shaking. Forward and reverse co-IPs were performed with anti-GFP conjugated beads and anti-FLAG conjugated beads. Beads were captured for 2 minutes and washed three times with PBST (137 mM NaCl, 8.1 mM Na2HPO4, 2.68 mM KCl, 1.47 mM KH₂PO₄, and 0.05% Tween-20). Biotinylated GFP (ab6658, 1:1000; Abcam) and FLAG (A190-101B, 1:1000; Bethyl) antibodies were added to appropriate wells. The plate was incubated on a shaker for 30 minutes. After washing, 100 n1 of reporter NeutrAvidin R-phycoerythrin at a 1:1000 dilution was added to each well and incubated for 30 minutes with subsequent washing. Each well was resuspended in 100 n1 of PBST buffer before plates were analyzed using FLEX MAP 3D system (Luminex Corp, Austin, TX). Approximately 100 beads were measured per sample to determine the median fluorescence intensity (MFI).

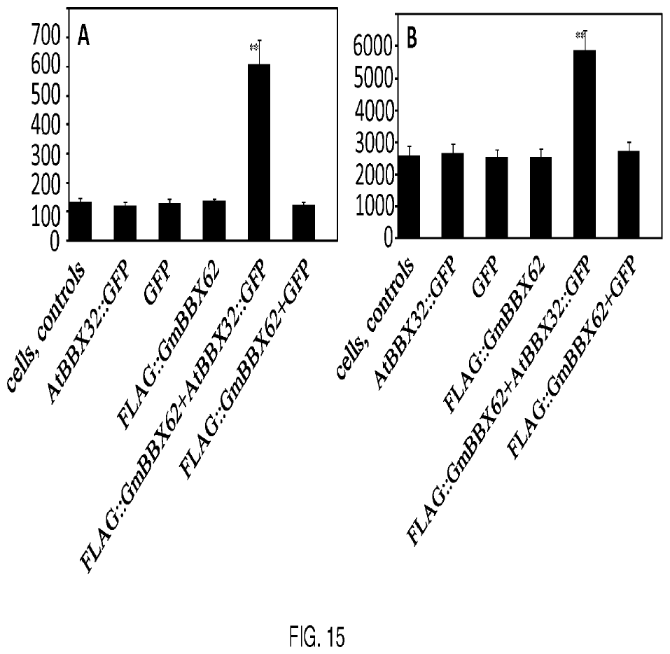

A co-immunoprecipitation (co-IP) experiment was performed with the co-expressed soybean protoplast extracts using an antibody against GFP. The protein complex was captured via the immobilized GFP antibody and the associated components within the complex were detected via anti-FLAG antibody and visualized using phycoerythrin-conjugated reporter molecules. As shown in FIG. 15A, the median fluorescence intensity (MFI) from cell extracts co-expressing AtBBX32::GFP and Flag::GmBBX62 is significantly higher (6-fold) than the control samples, including non-transfected cells and cell extracts lacking either AtBBX32::GFP or FLAG::GmBBX62. As expected, low fluorescence signals for the single gene construct samples did not show a significant difference from non-transfected cell extract, suggesting that these signals represent non-specific binding. A reciprocal co-IP experiment with anti-FLAG antibody-immobilized beads was also performed. The presence of AtBBX32::GFP within the complex was detected by anti-GFP antibody. Similarly, the fluorescence intensity for the co-expressed cell extract exhibited a 5-fold increase compared to the controls (FIG. 15B). The results indicate that AtBBX32 and GmBBX62 physically interact in soybean cells, further demonstrating the biological relevance of GmBBX62 as a partner of AtBBX32.

### AtBBX32 Binds GmBBX62 In Vitro

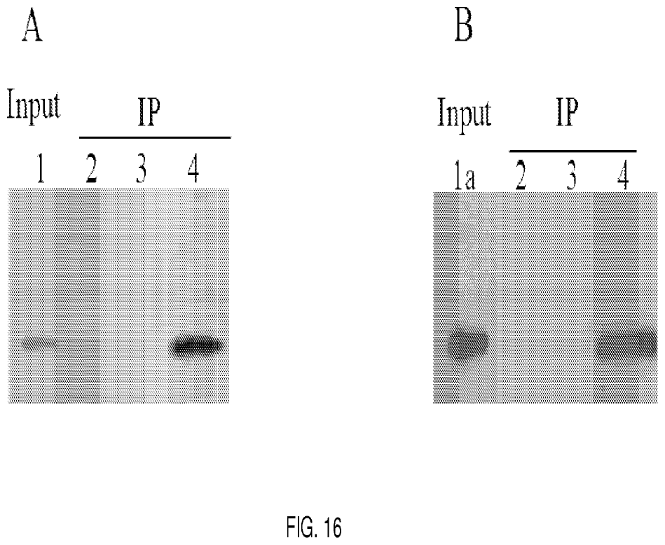

In vitro pull-down and ELISA-based protein-protein interaction assays were also conducted to confirm the binding of AtBBX32 with GmBBX62. Purified FLAG::GmBBX62 was bound to anti-FLAG M2 agarose resin and incubated with soluble protein extracts from wheat germ lysate expressing AtBBX32. The beads were pelleted and washed, and the bound protein complexes were eluted using SDS-PAGE loading buffer. The eluted protein complexes were identified by western blotting analysis. As expected, FLAG::GmBBX62 was detected with anti-Flag antibody from FLAG-GmBBX62-bound beads (FIG. 16A), confirming the presence of the GmBBX62 protein in reactions. AtBBX32 within the protein complex was detected with anti-AtBBX32 antibody from FLAG::GmBBX62 beads but not from the negative controls (FIG. 16B), demonstrating that FLAG::GmBBX62 can bind to AtBBX32 in vitro.

Co-Immunoprecipitation of AtBBX32 with GmBBX39 and Bead-Based Fluorescence Detection

Bead-based co-IP assays with Luminex on-bead detection method was used to confirm the interaction between the AtBBX32 and GmBBX39 proteins. Pairs of potential interacting proteins were co-expressed in soybean cotyledon protoplasts as described above.

Capture antibodies for GFP (MBL International, D153-3),Myc (Bethyl Laboratories cat #A190-104A)and FLAG (Bethyl Laboratories, A190-101A) were covalently coupled to carboxylated fluorescent microspheres (Luminex) according to the manufacturer's protocol Luminex two-step Carbodimide coupling protocol was modified as follows: 3.5×10⁶ beads were conjugated to 1514 of antibody and resuspended in coupling buffer containing 100 mM MES pH 6. Biotinylated antibodies for GFP (Abcam, ab6658), Myc (Bethyl Laboratories cat #A190-104B)and FLAG (Bethyl laboratories, A190-101B) were used for detection of interacting proteins in the miniaturized sandwich immunoassay. NeutrAvidin R-phycoerythrin was used as the reporter assay reagent (Invitrogen, A2660).

Protein input for BBX32::GFP and interactions were detected using the miniaturized sandwich immunoassay and co-IP method (Poetz, et al., 2009).Protein complexes were captured from extracts on antibody-conjugated beads, and the bound partner proteins were detected with biotinylated antibodies. NeutrAvidin R-phycoerythrin provided a fluorescence read-out for the quantity of bound detection antibody. Interacting proteins were defined as pairs of potential interacting proteins that yielded a greater median fluorescence intensity (MFI) than any of the controls (single protein expressed or untransformed cells) with a p-value <0.001 and a change in MFI that was 4-fold or greater when compared to each of the controls.

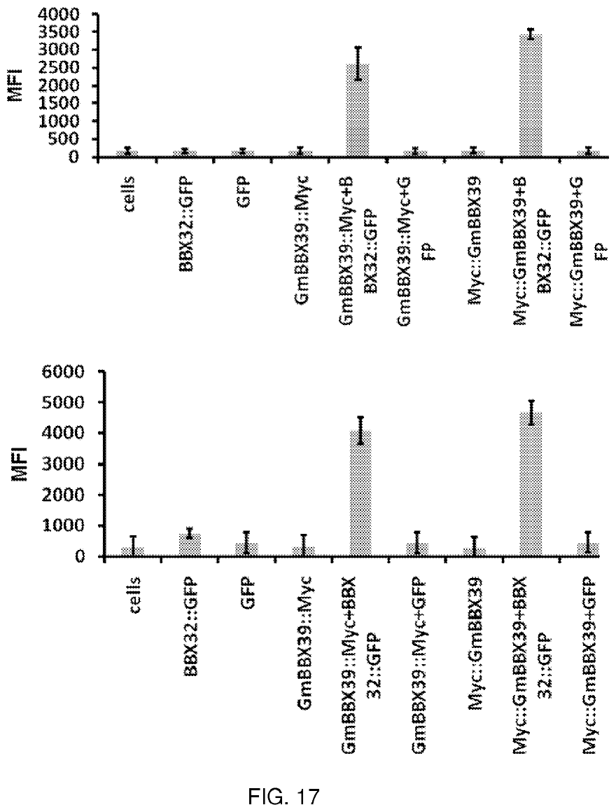

To assess the potential interaction between AtBBX32 and GmBBX39, two experiments were conducted consisting of a total of three repetitions (exp. 1, n=1; exp. 2, n=2). BBX32::GFP was co-expressed with FLAG::GmBBX39 in soybean cotyledon protoplasts. Expression analyses on one replicate of each treatment using either Luminex (BBX32::GFP) or Western blot (FLAG::BBX39) detection confirmed that each protein was expressed at detectable levels (data not shown). When co-expressed in protoplasts, BBX32::GFP and FLAG::BBX39 co-precipitated with either anti-GFP or anti-FLAG beads (FIG. 17). BBX32::GFP did not co-IP with anti-FLAG beads in the absence of FLAG::BBX39 and FLAG::BBX39 did not co-IP with anti-GFP beads in the absence of BBX32::GFP. These results demonstrate that AtBBX32 and GmBBX39 physically interact in soybean protoplasts. Similar experiments also demonstrated physical interactions between GmBBX39 and GmBBX62, between GmBBX52 and GmBBX39, and between GmBBX52 and GmBBX62 (data not shown).

### REFERENCES CITED

Abel and Theologis (1994) Plant J. 5: 421-427
Aldemita and Hodges (1996) Planta 199: 612-617
Altschul (1990) J. Mol. Biol. 215: 403-410
Altschul (1993) J. Mol. Evol. 36: 290-300
Ammirato et al., eds. (1984), Handbook of Plant Cell Culture —Crop Species, Macmillan Publ. Co., New York, NY
Anderson and Young (1985) “Quantitative Filter Hybridisation”, In: Hames and Higgins, ed., Nucleic
Acid Hybridisation, A Practical Approach. Oxford, IRL Press, 73-111
Ausubel et al. (1997) Short Protocols in Molecular Biology, John Wiley & Sons, New York, NY, unit 7.7
Babic et al. (1998) Plant Cell Reports 17: 183-188
Bairoch et al. (1997) Nucleic Acids Res. 25: 217-221
Bechtold and Pelletier (1998) Methods Mol. Biol. 82: 259-266
Berger and Kimmel (1987), “Guide to Molecular Cloning Techniques”, in Methods in Enzymology, vol. 152, Academic Press, Inc., San Diego, CA
Bevan (1984) Nucleic Acids Res. 12: 8711-8721
Borevitz et al. (2000) Plant Cell 12: 2383-2393
Boss and Thomas (2002) Nature, 416: 847-850
Bruce et al. (2000) Plant Cell 12: 65-79
Cassas et al. (1993) Proc. Natl. Acad. Sci. USA 90: 11212-11216
Cheikh et al. (2003) U.S. Patent Application No. US20030101479
Christou et al. (1987) Proc. Natl. Acad. Sci. USA 84: 3962-3966
Christou (1991) Bio/Technol. 9:957-962
Christou et al. (1992) Plant. J. 2: 275-281
D'Halluin et al. (1992) Plant Cell 4: 1495-1505
Daly et al. (2001) Plant Physiol. 127: 1328-1333
Deshayes et al. (1985) EMBO J., 4: 2731-2737
Donn et al.(1990) in Abstracts of VIIth International Congress on Plant Cell and Tissue Culture IAPTC, A2-38: 53
Doolittle, ed. (1996) Methods in Enzymology, vol. 266: “Computer Methods for Macromolecular Sequence Analysis” Academic Press, Inc., San Diego, Calif., USA
Draper et al. (1982) Plant Cell Physiol. 23: 451-458
Eddy (1996) Curr. Opin. Str. Biol. 6: 361-365
Eisen (1998) Genome Res. 8: 163-167
Feng and Doolittle (1987) J. Mol. Evol. 25: 351-360
Fowler and Thomashow (2002) Plant Cell 14: 1675-1690
Fromm et al. (1990) Bio/Technol. 8: 833-839
Fu et al. (2001) Plant Cell 13: 1791-1802
Gelvin et al. (1990) Plant Molecular Biology Manual, Kluwer Academic Publishers
Gilmour et al. (1998) Plant J. 16: 433-442
Goodrich et al. (1993) Cell 75: 519-530
Gordon-Kamm et al. (1990) Plant Cell 2: 603-618
Gruber et al., in Glick and Thompson (1993) Methods in Plant Molecular Biology and Biotechnology. eds., CRC Press, Inc., Boca Raton
Haake et al. (2002) Plant Physiol. 130: 639-648
Hain et al. (1985) Mol. Gen. Genet. 199: 161-168
Haymes et al. (1985) Nucleic Acid Hybridization: A Practical Approach, IRL Press, Washington, D.C.
He et al. (2000) Transgenic Res. 9: 223-227
Hein (1990) Methods Enzymol. 183: 626-645
Henikoff and Henikoff (1989) Proc. Natl. Acad. Sci. USA 89:10915
Henikoff and Henikoff (1991) Nucleic Acids Res. 19: 6565-6572
Herrera-Estrella et al. (1983) Nature 303: 209
Hiei et al. (1994) Plant J. 6:271-282
Hiei et al. (1997) Plant Mol. Biol. 35:205-218
Higgins and Sharp (1988) Gene 73: 237-244
Higgins et al. (1996) Methods Enzymol. 266: 383-402
Ishida (1990) Nature Biotechnol. 14:745-750
Jaglo et al. (2001) Plant Physiol. 127: 910-917
Kashima et al. (1985) Nature 313: 402-404
Khanna et al. (2006) Plant Cell 18, 2157-2171
Kim et al. (2001) Plant J. 25: 247-259
Kimmel (1987) Methods Enzymol. 152: 507-511
Klee (1985) Bio/Technology 3: 637-642
Klein et al. (1987) Nature 327: 70-73
Koornneef et al (1986) In Tomato Biotechnology: Alan R. Liss, Inc., 169-178
Ku et al. (2000) Proc. Natl. Acad. Sci. USA 97: 9121-9126
Kyozuka and Shimamoto (2002) Plant Cell Physiol. 43: 130-135
Lin et al. (1991) Nature 353: 569-571
Mandel (1992a) Nature 360: 273-277
Mandel et al. (1992b) Cell 71-133-143
Meyers (1995) Molecular Biology and Biotechnology, Wiley VCH, New York, NY, p 856-853
Mild et al. (1993) in Methods in Plant Molecular Biology and Biotechnology, p. 67-88, Glick and Thompson, eds., CRC Press, Inc., Boca Raton
Mount (2001), in Bioinformatics: Sequence and Genome Analysis, Cold Spring Harbor Laboratory Press, Cold Spring Harbor, New York, p. 543
Miller et al. (2001) Plant J. 28: 169-179
Nandi et al. (2000) Curr. Biol. 10: 215-218
Niman et al. (1983). Proc. Natl. Acad. Sci. U.S.A. 80: 4949-4953
Peng et al. (1997) Genes Development 11: 3194-3205
Peng et al. (1999) Nature 400: 256-261
Poetz, O., K. Luckert, et al. (2009) Anal. Biochem. 395:244-248
Putterill et al. (1995) Cell 80: 847-857
Ratcliffe et al. (2001) Plant Physiol. 126: 122-132
Reeves and Nissen (1990) J. Biol. Chem. 265, 8573-8582
Reeves and Nissen (1995). Prog. Cell Cycle Res. 1: 339-349
Riechmann et al. (2000a) Science 290, 2105-2110
Riechmann and Ratcliffe (2000b) Curr. Opin. Plant Biol. 3, 423-434
Rieger et al. (1976) Glossary of Genetics and Cytogenetics: Classical and Molecular, 4th ed., Springer Verlag, Berlin
Robson et al. (2001) Plant J. 28: 619-631
Sadowski et al. (1988) Nature 335: 563-564
Sambrook et al. (1989) Molecular Cloning: A Laboratory Manual, 2nd Ed., Cold Spring Harbor Laboratory, Cold Spring Harbor, N.Y.
Sanford et al. (1987) Part. Sci. Technol. 5:27-37
Sanford (1993) Methods Enzymol. 217: 483-509
Shimamoto et al. 1989; Nature 338: 274-276
Shpaer(1997) Methods Mol. Biol. 70: 173-187
Smith et al. (1992) Protein Engineering 5: 35-51
Sonnhammer et al. (1997) Proteins 28: 405-420
Spencer et al. (1994) Plant Mol. Biol. 24: 51-61
Suzuki et al. (2001) Plant J. 28: 409-418
Thompson et al. (1994) Nucleic Acids Res. 22: 4673-4680
Tudge (2000) in The Variety of Life, Oxford University Press, New York, NY pp. 547-606
Vasil et al. (1990) Bio/Technol. 8: 429-434
Vasil et al. (1992) Bio/Technol. 10:667-674
Vasil et al. (1993) Bio/Technol. 11:1553-1558
Vasil (1994) Plant Mol. Biol. 25: 925-937
Wahl and Berger (1987) Methods Enzymol. 152: 399-407
Wan and Lemeaux (1994) Plant Physiol. 104: 37-48
Weeks et al. (1993) Plant Physiol. 102:1077-1084
Weigel and Nilsson (1995) Nature 377: 482-500
Weissbach and Weissbach (1989) Methods for Plant Molecular Biology, Academic Press
Wu (ed.) Meth. Enzymol. (1993) vol. 217, Academic Press
Zhang et al. (1991) Bio/Technology 9: 996-997

All publications and patent applications mentioned in this specification are herein incorporated by reference to the same extent as if each individual publication or patent application was specifically and individually indicated to be incorporated by reference.

The present disclosure is not limited by the specific embodiments described herein. The disclosure now being fully described, it will be apparent to one of ordinary skill in the art that many changes and modifications can be made thereto without departing from the spirit or scope of the appended claims. Modifications that become apparent from the foregoing description and accompanying figures fall within the scope of the claims.

1. A method for modifying a trait in a plant, the method comprising the steps of:
introducing into a target plant at least one recombinant polynucleotide comprising at least one nucleotide sequence that encodes a polypeptide, wherein the nucleotide sequence is selected from the group consisting of:
(a) a nucleotide sequence set forth as SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233; 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, or 549;
(b) a nucleotide sequence encoding SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, or any of SEQ ID NO: 235 to 427; and
(c) a nucleotide sequence encoding a polypeptide wherein the polypeptide has a percentage identity to at least one of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 436, 438, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, or any of SEQ ID NO: 235 to 427;
wherein the nucleotide sequence is operably linked to a promoter that is expressed in a plant cell;
wherein the target plant overexpresses the polypeptide and said overexpression results in the target plant expressing the modified trait;
wherein the percentage identity is selected from the group consisting of at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% and 100%;
wherein the modified trait is selected from the group consisting of decreased sensitivity to light, increased yield, greater height, greater stem diameter, greater resistance to lodging, increased branching, increased secondary rooting, greater cold tolerance, greater tolerance to water deprivation, reduced stomatal conductance, altered C/N sensing, increased tolerance to nitrogen limiting conditions, improved late season growth and vigor, greater number of primary nodes, greater late season canopy coverage, increased tolerance to abiotic stress. increased tolerance to hyperosmotic stress, altered levels of ureides, altered levels of hexose sugars, altered sucrose phosphate synthase (SPS) activity, altered levels of starch, and delayed senescence, as compared to a control plant that does not comprise the recombinant polynucleotide; and,
optionally, selecting a transgenic plant having at least one of the modified traits as compared to the control plant.

2. The method of claim 1,
wherein the target plant comprises a second recombinant polynucleotide comprising a trans-acting element that controls expression of the polypeptide;
wherein the target plant expresses a protein encoded by a nucleic acid selected from the group consisting of: SEQ ID NO: 429, SEQ ID NO: 431, SEQ ID NO: 433, and SEQ ID NO: 529;
or wherein the target plant is stably transformed with the at least one recombinant polynucleotide.

3-4. (canceled)

5. A method for modifying a trait in a plant, the method comprising the steps of:
introducing into a target plant at least one recombinant polynucleotide that suppresses the expression of at least a polypeptide, wherein the polypeptide is encoded by a polynucleotide comprising a nucleotide sequence selected from the group consisting of:
(a) a nucleotide sequence set forth as SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233; 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, or 549;
(b) a nucleotide sequence encoding SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, or any of SEQ ID NO: 235 to 427;
(c) a nucleotide sequence encoding a polypeptide wherein the polypeptide has a percentage identity to at least one of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 436, 438, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, or any of SEQ ID NO: 235 to 427; and
wherein the percentage identity is selected from the group consisting of at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% and 100%;
wherein the nucleotide sequence is operably linked to a promoter that is expressed in a plant cell;
wherein the target plant overexpresses the polypeptide and said overexpression results in the target plant expressing the modified trait wherein the modified trait is selected from the group consisting of decreased sensitivity to light, increased yield, greater height, greater stem diameter, greater resistance to lodging, increased branching, increased secondary rooting, greater cold tolerance, greater tolerance to water deprivation, reduced stomatal conductance, altered C/N sensing, increased tolerance to nitrogen limiting conditions, improved late season growth and vigor, greater number of primary nodes, greater late season canopy coverage, increased tolerance to abiotic stress, increased tolerance to hyperosmotic stress, altered levels of ureides, altered levels of hexose sugars, altered SPS activity, altered levels of starch, and delayed senescence, as compared to a control plant that does not comprise the recombinant polynucleotide; and
optionally, selecting a transgenic plant having at least one of the modified traits as compared to the control plant.

6. The method of claim 5,
wherein the target plant comprises a second recombinant polynucleotide comprising a trans-acting element that controls expression of the polypeptide,
wherein the target plant expresses a protein encoded by a nucleic acid selected from the group consisting of: SEQ ID NO: 429, SEQ ID NO: 431, SEQ ID NO: 433, and SEQ ID NO: 529; or
wherein the target plant is stably transformed with the at least one recombinant polynucleotide.

7-8. (canceled)

9. The method of claim 1, wherein the promoter is a constitutive, inducible, tissue-specific, tissue-enhanced, light-regulated, stress-inducible, diurnally-regulated, chemically-inducible, or cell-specific promoter.

10. (canceled)

11. The method of claim 1, the method steps further comprising:
crossing the target plant with itself, a second plant from the same line as the target plant, a non-transgenic plant, a wild-type plant, or a transgenic plant from a different line of plants, to produce a transgenic seed.

12. (canceled)

13. A transgenic plant produced by the method of claim 1, wherein the transgenic plant comprises the at least one recombinant polynucleotide.

14. The transgenic plant of claim 13, wherein the transgenic plant is a monocot, dicot or a conifer.

15. The transgenic plant of claim 14, wherein the dicot or conifer is a: member of the family Pinaceae, cedar plant, fir plant, hemlock plant, larch plant, pine plant, spruce plant, member of the family Amaranthaceae, spinach plant, member of the family Anacardiaceae, mango plant, member of the family Asteraceae, sunflower plant, endive plant, lettuce plant, artichoke plant, member of the family Brassicaceae, Arabidopsis thaliana plant, rape plant, oilseed rape plant, broccoli plant, Brussels sprouts plant, cabbage plant, canola plant, cauliflower plant, kohlrabi plant, turnip plant, radish plant, member of the family Bromeliaceae, pineapple plant, member of the family Caricaceae, papaya plant, member of the family Chenopodiaceae, beet plant, member of the family Curcurbitaceae, melon plant, cantaloupe plant, squash plant, watermelon plant, honeydew plant, cucumber plant, pumpkin plant, member of the family Dioscoreaceae, yam plant, member of the family Ericaceae, blueberry plant, member of the family Euphorbiaceae, cassava plant, member of the family Fabaceae, alfalfa plant, clover plant, peanut plant, member of the family Grossulariaceae, currant plant, member of the family Juglandaceae, walnut plant, member of the family Lamiaceae, mint plant, member of the family Lauraceae, avocado plant, member of the family Leguminosae, soybean plant, bean plant, pea plant, member of the family Malvaceae, cotton plant, member of the family Marantaceae, arrowroot plant, member of the family Myrtaceae, guava plant, eucalyptus plant, member of the family Rosaceae, peach plant, apple plant, cherry plant, plum plant, pear plant, prune plant, blackberry plant, raspberry plant, strawberry plant, member of the family Rubiaceae, coffee plant, member of the family Rutaceae, citrus plant, orange plant, lemon plant, grapefruit plant, tangerine plant, member of the family Salicaceae, poplar plant, willow plant, member of the family Solanaceae, potato plant, sweet potato plant, tomato plant, Capsicum plant, tobacco plant, tomatillo plant, eggplant plant, Atropa belladona plant, Datura stramonium plant, member of the family Vitaceae, grape plant, member of the family Umbelliferae, carrot plant and/or member of the family Musaceae, banana plant, or wherein the monocot is a: member of the family Poaceae; maize plant; rice plant; wild rice plant, wheat plant; barley plant; sorghum plant; rye plant; oat plant; millet plant; turfgrass plant; sugarcane plant, bamboo plant, brome-grass plant, pampas grass plant, Miscanthus plant; switchgrass plant, teosinte plant; and/or member of the family Alliaceae, onion, leak and garlic.

16-17. (canceled)

18. A tissue culture produced from protoplasts or cells from the transgenic plant of claim 13, wherein said cells or protoplasts are produced from a plant part selected from the group consisting of leaf, pollen, ovule, embryo, cotyledon, hypocotyl, meristematic cell, root, root tip, pistil, anther, flower, seed, shoot, stem, pod and petiole.

19. A regenerated plant regenerated from the tissue culture of claim 18.

20. A seed from the transgenic plant of claim 13.

21. A method for producing a transgenic seed, the method comprising crossing two plants and harvesting resultant seed, wherein at least one plant is the transgenic plant of claim 13.

22. A transgenic seed produced by the method of claim 1.

23. A transgenic progeny plant, or a part thereof, produced by growing the seed of claim 22.

24. The transgenic progeny plant of claim 23, wherein the transgenic progeny plant has a modified trait relative to a control plant,
wherein the modified trait is selected from the group consisting of decreased sensitivity to light, increased yield, greater height, greater stem diameter, greater resistance to lodging, increased branching, increased secondary rooting, greater cold tolerance, greater tolerance to water deprivation, reduced stomatal conductance, altered C/N sensing, increased tolerance to nitrogen limiting conditions, improved late season growth and vigor, greater number of primary nodes, greater late season canopy coverage, increased tolerance to abiotic stress, increased tolerance to hyperosmotic stress, altered levels of ureides, altered levels of hexose sugars, altered SPS activity, altered levels of starch, and delayed senescence, as compared to a control plant that does not comprise the recombinant polynucleotide.

25. A recombinant polynucleotide comprising a nucleotide sequence that encodes a polypeptide, wherein the nucleotide sequence is selected from the group consisting of:
(a) a nucleotide sequence set forth as SEQ ID NO: 447, 461, 473, 481, 483, 485, 509;
(b) a nucleotide sequence encoding SEQ ID NO: 448, 462, 474, 482, 484, 486, 510;
(c) a nucleotide sequence wherein the nucleotide sequence has a percentage identity to at least one of SEQ ID NO: 447, 461, 473, 481, 483, 485, 509; and
(d) a nucleotide sequence encoding a polypeptide wherein the polypeptide has a percentage identity to at least one of SEQ ID NO: 448, 462, 474, 482, 484, 486, 510;
wherein the percentage identity is selected from the group consisting of at least 85%, at least 86%, at least 87%, at least 88%, at least 89%, at least 90%, at least 91%, at least 92%, at least 93%, at least 94%, at least 95%, at least 96%, at least 97%, at least 98%, at least 99% and 100%.

26. A recombinant DNA construct comprising a promoter that is functional in a plant cell and that is operably linked to the recombinant polynucleotide of claim 25.

27. A plant cell comprising at least one recombinant polynucleotide of claim 25 or comprising at least one recombinant polynucleotide that suppresses expression of a polypeptide that is encoded by the recombinant polynucleotide.

28. (canceled)

29. A plant or seed comprising the plant cell of claim 27.

30-31. (canceled)
